(19) United States
(12) Patent Application Publication
(10) Pub. No.: US 2026/0098262 A9
(43) Pub. Date: Apr. 9, 2026

(54) COMPOUNDS AND METHODS FOR SKIPPING EXON 44 IN DUCHENNE MUSCULAR DYSTROPHY

(71) Applicant: Entrada Therapeutics, Inc., Boston, MA (US)

(72) Inventors: Xiang Li, Belmont, MA (US); Ziqing Qian, Wellesley, MA (US); Mahboubeh Kheirabadi, Natick, MA (US); Mark Wysk, Leominster, MA (US); Natarajan Sethuraman, Hanover, NH (US); Wenlong Lian, Wayland, MA (US); Mahsweta Girgenrath, Medway, MA (US); Nelsa Estrella, North Andover, MA (US)

(21) Appl. No.: 18/688,083

(22) PCT Filed: Aug. 30, 2022
(86) PCT No.: PCT/US2022/075691

§ 371 Date: Feb. 29, 2024

Related U.S. Application Data
(60) Provisional application No. 63/239,645, filed on Sep. 1, 2021.
(60) Provisional application No. 63/239,671, filed on Sep. 1, 2021.
(60) Provisional application No. 63/290,960, filed on Dec. 17, 2021.
(60) Provisional application No. 63/292,685, filed on Dec. 22, 2021.
(60) Provisional application No. 63/298,565, filed on Jan. 11, 2022.
(60) Provisional application No. 63/268,577, filed on Feb. 25, 2022.
(60) Provisional application No. 63/268,580, filed on Feb. 25, 2022.
(60) Provisional application No. 63/362,294, filed on Mar. 31, 2022.
(60) Provisional application No. 63/362,423, filed on Apr. 4, 2022.
(60) Provisional application No. 63/337,560, filed on May 2, 2022.
(60) Provisional application No. 63/354,456, filed on Jun. 22, 2022.

(51) Int. Cl. C12N 15/113; A61K 47/64

(52) U.S. Cl. CPC C12N 15/113; A61K 47/64; C12N 2310/3233; C12N 2310/3513

(57) ABSTRACT

Described herein in various embodiments are compositions comprising (a) a cyclic peptide; and (b) an antisense compound, wherein the antisense compound targets exon 44 of the DMD gene in a pre-mRNA sequence.

This application claims benefit of priority to the filing dates of U.S. Provisional Application Ser. No. 63/239,645 filed Sep. 1, 2021, U.S. Provisional Application Ser. No. 63/292,685 filed Dec. 22, 2022, U.S. Provisional Application Ser. No. 63/268,580 filed Feb. 25, 2022, U.S. Provisional Application Ser. No. 63/362,294 filed Mar. 31, 2022, U.S. Provisional Application Ser. No. 63/362,423 filed Apr. 4, 2022, U.S. Provisional Application Ser. No. 63/337,560 filed May 2, 2022, U.S. Provisional Application Ser. No. 63/354,456 filed Jun. 22, 2022, U.S. Provisional Application Ser. No. 63/239,671 filed Sep. 1, 2021, U.S. Provisional Application Ser. No. 63/290,960 filed Dec. 17, 2021, U.S. Provisional Application Ser. No. 63/298,565 filed Jan. 11, 2022, and U.S. Provisional Application Ser. No. 63/268,577 filed Feb. 25, 2022, the contents of which are specifically incorporated herein by reference in their entireties.

### BACKGROUND

Duchenne Muscular Dystrophy (DMD) is a genetic disorder characterized by progressive muscle degeneration and weakness due to alterations of the protein dystrophin. Genetic modifications in DMD, the gene that encodes dystrophin, cause DMD. These genetic modifications shift the reading frame of DMD leading to a nonfunctional truncated DMD protein. One method for treating DMD patients entails delivering to a patient a compound which restores the reading frame of DMD. Antisense compounds can restore the reading frame of DMD by skipping an internal exon associated with the shift in the reading frame of DMD that leads to the nonfunctional truncated DMD protein. Exon skipping produces dystrophin proteins which retain functionality that is lost in the disease state.

A significant problem with the use of antisense oligonucleotide therapeutics is their limited ability to gain access to the intracellular compartment when administered systemically. Intracellular delivery of antisense compounds can be facilitated by using of carrier systems such as polymers, cationic liposomes or by chemical modification of the construct, for example by the covalent attachment of cholesterol molecules. However, intracellular delivery efficiency remains low and there remains a need for improved delivery systems to increase the potency of these antisense compounds.

There is an unmet need for effective compositions to deliver antisense compounds to intracellular compartments to treat diseases caused by, e.g., aberrant gene transcription, splicing and/or translation.

### SUMMARY

Compounds for delivering nucleic acids are described herein. In embodiments, the nucleic acids are antisense compounds (AC). In embodiments, the antisense compounds target exon 44 in a subject with Duchenne muscular dystrophy (DMD).

The disclosure relates to compounds comprising:
(a) a cell penetrating peptide (CPP) sequence (e.g., cyclic peptide); and
(b) an antisense compound (AC) that is complementary to a target sequence comprising at least a portion of exon 44 of DMD gene in a pre-mRNA sequence. In embodiments, the AC is complementary to a target sequence comprising at least a portion of exon 44 of DMD gene in a pre-mRNA sequence, at least a portion of an intronic sequence flanking exon 44 of DMD gene in a pre-mRNA sequence, or both. In embodiments, hybridization of the AC with the target sequence alters the splicing pattern of the DMD pre-mRNA to restore the reading frame and enable production of a functional dystrophin protein.

In embodiments, the AC comprises at least one modified nucleotide or nucleic acid selected from a phosphorothioate (PS) nucleotide, a phosphorodiamidate morpholino (PMO) nucleotide, a locked nucleic acid (LNA), a peptide nucleic acid (PNA), a nucleotide comprising a 2′-O-methyl (2′-OMe) modified backbone, a 2′O-methoxy-ethyl (2′-MOE) nucleotide, a 2′,4′ constrained ethyl (cEt) nucleotide, and a 2′-deoxy-2′-fluoro-beta-D-arabinonucleic acid (2′F-ANA). In embodiments, the AC comprises at least one PMO (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 PMO, inclusive of all ranges therein). In embodiments, each nucleotide in the AC is a PMO.

In embodiments, the AC comprises the sequence:

5′-AAA CGC CGC CAT TTC TCA ACA GAT C-3′.

In embodiments, the cyclic peptide is FGFGRGRQ. In embodiments, the cyclic peptide is GfFGrGrQ. In embodiments, the cyclic peptide is FfΦGRGRQ.

In embodiments, the EEV is: Ac-PKKKRKV-AEEA-Lys-(cyclo[FGFGRGRQ])-PEG12-OH.

The disclosure relates to a pharmaceutical composition comprising a compound described herein.

The disclosure relates to a cell comprising a compound described herein.

The disclosure relates to a method of treating DMD comprising administering a compound described herein to a patient.

### BRIEF DESCRIPTION OF THE DRAWINGS

FIGS. 1A and 1B shows the conjugation chemistry for connecting a therapeutic moiety, e.g., an antisense compound (AC), to a cell penetrating peptide (CPP). The CPP can be conjugated to the 5′ end, the 3′ end or the backbone of the AC.

FIGS. 2A and 2B show conjugation chemistries for connecting a cell penetrating peptide (CPP), shown as Peptide, and an antisense compound (AC), wherein the CPP includes a PEG4 linker and the AC is shown without (FIG. 2A) and with (FIG. 2B) a linker containing a polyethylene glycol (PEG2 or miniPeG) moiety. “R” in the figure represents a palmitoyl group.

FIG. 3 shows examples of endosomal escape vehicle (EEV) design using a representative CPP. It is understood that the CPP can include any of the CPP disclosed herein.

FIG. 4A shows a schematic of preparation of EEV-PMO-MDX-23-1. FIG. 4B is a RT-PCR analysis that shows that, in comparison to mice treated with PMO-MDX-23-1, mice treated with EEV-PMO-MDX-23-1 produced dystrophin lacking the internal exon, exon 23. FIG. 4C shows dystrophin exon skipping products in various treated muscle groups after administration of PMO-MDX-23-1 and EEV-PMO-MDX-23-1.

FIGS. 5A-5D show the percentage of exon skipping in MDX mice in the quadriceps (FIG. 5A), tibialis anterior (TA) (FIG. 5B), diaphragm (FIG. 5C), and heart (FIG. 5D) after delivery of PMO-MDX-23-1 or EEV-PMO-MDX-23-1.

FIGS. 6A-6D show the percentage of exon 23 splicing in MDX mice in the tibialis anterior (TA) (FIG. 6A), quadriceps (FIG. 6B), diaphragm (FIG. 6C), and heart (FIG. 6D) after delivery of EEV-PMO-MDX-23-1.

FIGS. 7A-7D show the amount of exon 23 corrected dystrophin detected by Western Blot in the quadriceps (FIG. 7A), tibialis anterior (TA) (FIG. 7B), diaphragm (FIG. 7C), and heart (FIG. 7D) after delivery of PMO-MDX-23-1 or EEV-PMO-MDX-23-1.

FIGS. 8A-8D show Western Blots of exon 23 corrected dystrophin and α-actinin in the diaphragm (FIG. 8A), heart (FIG. 8B), quadriceps (FIG. 8C), and tibialis anterior (FIG. 8D) after intravenous delivery of 10 mpk or 30 mpk EEV-PMO-MDX-23-1.

FIGS. 9A-9B show the dystrophin levels in MDX mice two weeks (FIG. 9A) and four weeks (FIG. 9B) after treatment with 30 mpk EEV-PMO-MDX-23-1 or 30 mpk PMO-MDX-23-1.

FIGS. 10A-10D show the percentage of exon 23 correction in tibialis anterior (FIG. 10A), quadriceps (FIG. 10B), diaphragm (FIG. 10C), and heart (FIG. 10D) in MDX mice that were administered either 30 mpk of PMO-MDX-23-1 or 30 mpk of EEV-PMO-MDX-23-1. Mice administered EEV-PMO-MDX-23-1 exhibited enhanced splicing correction, compared to mice administered PMO-MDX-23-1 alone.

FIGS. 11A-11C shows exon 23 skipping and dystrophin correction in heart (FIG. 11A), tibialis anterior (FIG. 11B), and diaphragm (FIG. 11C) observed up to 8 weeks after a single IV dose (40 mg/kg) of EEV-PMO-MDX-23-1 in mdx mice.

FIGS. 12A-12D show exon 23 skipping after repeat doses (20 mg/kg) of EEV-PMO-MDX-23-2 in the D2-mdx model. FIG. 12A (heart); FIG. 12B (diaphragm); FIG. 12C (tibialis anterior); FIG. 12D (triceps)

FIGS. 13A-13C D2-mdx mice showed normalized serum creatine kinase (CK) levels (FIG. 12A) and significant improvement in muscle function (FIGS. 12B-12C) when treated monthly with 20 mg/kg EEV-PMO-MDX-23-2 as compared to PMO-MDX-23 alone. (ns not significant, *p<0.05, **p<0.01, ***p<0.001, ****p<0.0001).

FIGS. 14A-14D shows dose dependent exon skipping 1 week post injection with EEV-PMO-MDX-23-2 as assessed by 2-Step RT-PCR. FIG. 14A (triceps); FIG. 14B (tibialis anterior); FIG. 14C (diaphragam); FIG. 15D (heart).

FIG. 15A-15D shows the duration of effect after 80 mpk EEV-PMO-MDX-23-2 administration. FIG. 15A (triceps); FIG. 15B (tibialis anterior); FIG. 15C (diaphragm); FIG. 15D (heart).

FIG. 16 shows a cumulative exon skipping in all 4 tissues (triceps, tibialis anterior, diaphragm and heart).

FIG. 17 shows D2.mdx wire hang data. After 12 weeks of treatment, animals treated with EEV-PMO-MDX23-1 80 mpk Q2W had a wire hang time that was statistically indistinguishable from the WT animals. DBA WT Vehicle (saline); D2.mdx Vehicle (saline); D2.mdx EEV-PMO-MDX23-1; D2.mdx EEV-PMO-MDX23-2; D2.mdx PMO-MDX23 (5′-GGCCAAACCTCGGCTTACCTGAAAT-3′)

FIGS. 18A-18D show creatine kinase activity in D2 MDX mice pre-dosing (FIG. 18A), and at 4 weeks (FIG. 18B), 8 weeks (FIG. 18C) and 12 weeks (FIG. 18D) post-dosing.

FIGS. 19A-19B show grip strength of D2MDX mice pre-dosing (FIG. 19A) and 12 weeks post-dosing (FIG. 19B).

FIGS. 20A-20D show the synthetic schemes for EEV-PMO-DMD44-1 (FIG. 5A), EEV-PMO-DMD44-2 (FIG. 5B), EEV-PMO-DMD44-3 (FIG. 5C) and EEV-PMO-DMD44-4 (FIG. 5D).

FIG. 21 shows the dystrophin protein restoration in DMDA45 muscle cells after treatment with 1, 3 or 10 μM EEV-PMO-DMD44-1; EEV-PMO-DMD44-2, or EEV-PMO-DMD-3.

FIGS. 22A and 22B show exon skipping and drug concentration in tissues of hDMD mice treated with EEV-PMO-DMD44-1 (FIG. 7A) and EEV-PMO-DMD44-2 (FIG. 7B) via IV injection.

FIGS. 23A-23B depict exon skipping (FIG. 23A) and drug exposure (FIG. 23B) for EEV-PMO-DMD44-1 in a NHP model.

FIGS. 24A-24B depict exon skipping (FIG. 24A) and drug exposure (FIG. 24B) for EEV-PMO-DMD44-2 in a NHP model.

FIGS. 25A-25B show exon skipping (FIG. 25A) and restoration of dystrophin (FIG. 25B) in DMD patient-derived muscle cells treated with EEV-PMO-DMD44-1.

FIG. 26A-26C dose-dependent tissue exposure and exon skipping was observed in cardiac (FIG. 26A) and skeletal muscle (FIGS. 26B and 26C) of hDMD transgenic mice after intravenous (IV) administration of EEV-PMO-DMD44-1 at 10, 20, 40 and 80 mg/kg.

FIG. 27. Shows that EEV-PMO-DMD44-1 has an extended circulating half-life when administered to non-human primates (NHP).

FIG. 28 shows that a single dose of EEV-PMO-DMD44-1 resulted in meaningful levels of exon skipping in both skeletal muscles and the heart of NIP 7 days post 1 hour IV infusion at 30 mg/kg.

FIGS. 29A-29C depict exon skipping in the heart (FIG. 29A), diaphragm (FIG. 29B) and triceps (FIG. 29C) of hDystrophin mice after a single IV dose (15 mg/kg) of EEV-PMO-DMD44-1 or R6 (polyarginine) conjugated exon 44 skipping PMO.

FIG. 30A-30E depict exon skipping in hDMD mice for up to 12 weeks as detected by 1-STEP RT-PCR in heart (FIG. 30A), diaphragm (FIG. 30B), tibialis anterior (FIG. 30C), gastrocnemius (FIG. 30D) and triceps (FIG. 30E).

FIG. 31 shows exon skipping in NHP for up to 12 weeks after a singe IV dose.

FIGS. 32A-32C show the localization of PMO vs EEV-PMO vs EEV-NLS-PMO in THP cells as determined by LC-MS/MS: whole cell uptake (FIG. 32A); subcellular localization (FIG. 32B); and nuclear uptake (FIG. 32C).

### DETAILED DESCRIPTION

### Compounds

Disclosed herein, are compounds for treating Duchenne Muscular Dystrophy (DMD). In embodiments, DMD is caused by a mutation in exon 44. In embodiments, the compounds are designed to deliver an antisense compound (AC) that is complementary to a target sequence of a DMD gene in a pre-mRNA sequence, wherein the target sequence comprises at least a portion of the 5′ flanking intron of exon 44, at least a portion of exon 44, at least a portion of the 3′ flanking intron of exon 44, or a combination thereof. In embodiments, the compounds are designed to deliver an antisense compound (AC) intracellularly to subjects in need thereof.

In embodiments, the compounds alter the splicing pattern of the target pre-mRNA to which the AC binds, resulting in the formation of re-spliced target protein. In one embodiment, re-spliced target protein is more functional than the target protein produced by the splicing of the target pre-mRNA in the absence of the AC. In embodiments, the re-spliced target protein increases target protein function by at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, at least about 100%, at least about 150%, at least about 200%, at least about 250%, at least about 300%, at least about 350%, at least about 400%, at least about 450%, at least about 500%, or more, compared to the function of the target protein produced by splicing. In embodiments, the re-spliced target protein increases target protein function by about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, about 100%, about 150%, about 200%, about 250%, about 300%, about 350%, about 400%, about 450%, about 500%, or more, compared to the function of the target protein produced by splicing, inclusive of all values and ranges therebetween. In embodiments, the re-spliced target protein restores function to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99%, and up to about 100% of the function of a wild type target protein. In embodiments, the re-spliced target protein restores function to about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or about 100% of the function of a wild type target protein, inclusive of all values and ranges therebetween.

In various embodiments, the compounds disclosed herein have an AC moiety and cell penetrating peptide (CPP) moiety. In embodiments, the CPP moeity is cyclic (referred to herein as a cyclic peptide). In embodiments, the compounds are able to traverse the cell membrane and bind to target pre-mRNA in vivo. In embodiments, the compounds comprise: a) at least one CPP moiety; and b) at least one AC, wherein the CPP is coupled directly, or indirectly (e.g., via a linker), to the AC. In embodiments, the compounds comprise 1, 2, 3, 4, 5, 6, 7, 8, 9, 10, or more AC moieties. In embodiments, the compounds comprise 1, 2, 3, 4, 5, 6, 7, 8, 9, 10, or more CPP moieties. In embodiments, the compounds comprise one AC moiety. In embodiments, the compounds comprise two AC moieties. As used herein, “coupled” can refer to a covalent or non-covalent association between the CPP to the AC, including fusion of the CPP to the AC and chemical conjugation of the CPP to the AC. A non-limiting example of a means to non-covalently attach the CPP to the AC is through the streptavidin/biotin interaction, e.g., by conjugating biotin to CPP and fusing AC to streptavidin. In the resulting compound, the CPP is coupled to the AC via non-covalent association between biotin and streptavidin.

In embodiments, the CPP is conjugated directly, or indirectly via a linker, to the AC to thereby form a CPP-AC conjugate. Conjugation of the AC to the CPP may occur at any appropriate site on these moieties. In embodiments, the 5′ or the 3′ end of the AC may be conjugated to the C-terminus, the N-terminus, or a side chain of an amino acid in the CPP. In embodiments, the CPP is a cyclic peptide.

In embodiments, the AC may be chemically conjugated to the CPP through a moiety on the 5′ or 3′ end of the AC. In embodiments, the AC may be conjugated to the CPP through a side chain of an amino acid on the CPP. Any amino acid side chain on the CPP which is capable of forming a covalent bond, or which may be so modified, can be used to link AC to the CPP. The amino acid on the CPP can be a natural or non-natural amino acid. In embodiments, the amino acid on the CPP used to conjugate the AC is aspartic acid, glutamic acid, glutamine, asparagine, lysine, ornithine, 2,3-diaminopropionic acid, or analogs thereof. In embodiments, the side chain is substituted with a bond to the AC or linker. In embodiments, the amino acid is lysine, or an analog thereof. In embodiments, the amino acid is glutamic acid, or an analog thereof. In embodiments, the amino acid is aspartic acid, or an analog thereof. In embodiments, the CPP is a cyclic peptide.

### Endosomal Escape Vehicles (EEVs)

An endosomal escape vehicle (EEV) is provided herein that can be used to transport an AC across a cellular membrane, for example, to deliver the AC to the cytosol or nucleus of a cell. The EEV can comprise a cell penetrating peptide (CPP), for example, a cyclic cell penetrating peptide (cCPP), which is conjugated to an exocyclic peptide (EP). The EP can be referred to interchangeably as a modulatory peptide (MP). The EP can comprise a sequence of a nuclear localization signal (NLS). The EP can be coupled to the AC. The EP can be coupled to the cCPP. The EP can be coupled to the AC and the cCPP. Coupling between the EP, AC, cCPP, or combinations thereof, may be non-covalent or covalent. The EP can be attached through a peptide bond to the N-terminus of the cCPP. The EP can be attached through a peptide bond to the C-terminus of the cCPP. The EP can be attached to the cCPP through a side chain of an amino acid in the cCPP. The EP can be attached to the cCPP through a side chain of a lysine which can be conjugated to the side chain of a glutamine in the cCPP. The EP can be conjugated to the 5′ or 3′ end of an AC. The EP can be coupled to a linker. The exocyclic peptide can be conjugated to an amino group of the linker. The EP can be coupled to a linker via the C-terminus of an EP and a cCPP through a side chain on the cCPP and/or EP. For example, an EP may comprise a terminal lysine which can then be coupled to a cCPP containing a glutamine through an amide bond. When the EP contains a terminal lysine, and the side chain of the lysine can be used to attach the cCPP, the C- or N-terminus may be attached to a linker on the AC.

### Exocyclic Peptides

The exocyclic peptide (EP) can comprise from 2 to 10 amino acid residues e.g., 2, 3, 4, 5, 6, 7, 8, 9, or 10 amino acid residues, inclusive of all ranges and values therebetween. The EP can comprise 6 to 9 amino acid residues. The EP can comprise from 4 to 8 amino acid residues.

Each amino acid in the exocyclic peptide may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be the D-isomer of the natural amino acids. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein. For example, the amino acids can be A, G, P, K, R, V, F, H, Nal, or citrulline.

The EP can comprise at least one positively charged amino acid residue, e.g., at least one lysine residue and/or at least one amine acid residue comprising a side chain comprising a guanidine group, or a protonated form thereof. The EP can comprise 1 or 2 amino acid residues comprising a side chain comprising a guanidine group, or a protonated form thereof. The amino acid residue comprising a side chain comprising a guanidine group can be an arginine residue. Protonated forms can mean salt thereof throughout the disclosure.

The EP can comprise at least two, at least three or at least four or more lysine residues. The EP can comprise 2, 3, or 4 lysine residues. The amino group on the side chain of each lysine residue can be substituted with a protecting group, including, for example, trifluoroacetyl (—COCF₃), allyloxycarbonyl (Alloc), 1-(4,4-dimethyl-2,6-dioxocyclohexylidene)ethyl (Dde), or (4,4-dimethyl-2,6-dioxocyclohex-1-ylidene-3)-methylbutyl (ivDde) group. The amino group on the side chain of each lysine residue can be substituted with a trifluoroacetyl (—COCF₃) group. The protecting group can be included to enable amide conjugation. The protecting group can be removed after the EP is conjugated to a cCPP.

The EP can comprise at least 2 amino acid residues with a hydrophobic side chain. The amino acid residue with a hydrophobic side chain can be selected from valine, proline, alanine, leucine, isoleucine, and methionine. The amino acid residue with a hydrophobic side chain can be valine or proline.

The EP can comprise at least one positively charged amino acid residue, e.g., at least one lysine residue and/or at least one arginine residue. The EP can comprise at least two, at least three or at least four or more lysine residues and/or arginine residues.

The EP can comprise KK, KR, RR, HH, HK, HR, RH, KKK, KGK, KBK, KBR, KRK, KRR, RKK, RRR, KKH, KHK, HKK, HRR, HRH, HHR, HBH, HHH, HHHH, KHKK, KKHK, KKKH, KHKH, HKHK, KKKK, KKRK, KRKK, KRRK, RKKR, RRRR, KGKK, KKGK, HBHBH, HBKBH, RRRRR, KKKKK, KKKRK, RKKKK, KRKKK, KKRKK, KKKKR, KBKBK, RKKKKG, KRKKKG, KKRKKG, KKKKRG, RKKKKB, KRKKKB, KKRKKB, KKKKRB, KKKRKV, RRRRRR, HHHHHH, RHRHRH, HRHRHR, KRKRKR, RKRKRK, RBRBRB, KBKBKB, PKKKRKV, PGKKRKV, PKGKRKV, PKKGRKV, PKKKGKV, PKKKRGV or PKKKRKG, wherein B is beta-alanine. The amino acids in the EP can have D or L stereochemistry.

The EP can comprise KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can comprise PKKKRKV, RR, RRR, RHR, RBR, RBRBR, RBHBR, or HBRBH, wherein B is beta-alanine. The amino acids in the EP can have D or L stereochemistry.

The EP can consist of KK, KR, RR, KKK, KGK, KBK, KBR, KRK, KRR, RKK, RRR, KKKK, KKRK, KRKK, KRRK, RKKR, RRRR, KGKK, KKGK, KKKKK, KKKRK, KBKBK, KKKRKV, PKKKRKV, PGKKRKV, PKGKRKV, PKKGRKV, PKKKGKV, PKKKRGV or PKKKRKG. The EP can consist of PKKKRKV, RR, RRR, RHR, RBR, RBRBR, RBHBR, or HBRBH, wherein B is beta-alanine. The amino acids in the EP can have D or L stereochemistry.

The EP can comprise an amino acid sequence identified in the art as a nuclear localization sequence (NLS). The EP can consist of an amino acid sequence identified in the art as a nuclear localization sequence (NLS). The EP can comprise an NLS comprising the amino acid sequence PKKKRKV. The EP can consist of an NLS comprising the amino acid sequence PKKKRKV. The EP can comprise an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK. The EP can consist of an NLS comprising an amino acid sequence selected from NLSKRPAAIKKAGQAKKKK, PAAKRVKLD, RQRRNELKRSF, RMRKFKNKGKDTAELRRRRVEVSVELR, KAKKDEQILKRRNV, VSRKRPRP, PPKKARED, PQPKKKPL, SALIKKKKKMAP, DRLRR, PKQKKRK, RKLKKKIKKL, REKKKFLKRR, KRKGDEVDGVDEVAKKKSKK and RKCLQAGMNLEARKTKK

All exocyclic sequences can also contain an N-terminal acetyl group. Hence, for example, the EP can have the structure: Ac-PKKKRKV.

### Cell Penetrating Peptides (CPP)

The cell penetrating peptide (CPP) can comprise 6 to 20 amino acid residues. The cell penetrating peptide can be a cyclic cell penetrating peptide (cCPP). The cCPP is capable of penetrating a cell membrane. An exocyclic peptide (EP) can be conjugated to the cCPP, and the resulting construct can be referred to as an endosomal escape vehicle (EEV). The cCPP can direct an AC to penetrate the membrane of a cell. The cCPP can deliver the AC to the cytosol of the cell. The cCPP can deliver the AC to a cellular location where a target (e.g., pre-mRNA) is located. To conjugate the cCPP to an AC at least one bond or lone pair of electrons on the cCPP can be replaced.

The total number of amino acid residues in the cCPP is in the range of from 6 to 20 amino acid residues, e.g., 6, 7, 8, 9, 10, 11, 12, 13, 14, 15, 16, 17, 18, 19, or 20 amino acid residues, inclusive of all ranges and subranges therebetween. The cCPP can comprise 6 to 13 amino acid residues. The cCPP disclosed herein can comprise 6 to 10 amino acids. By way of example, cCPP comprising 6-10 amino acid residues can have a structure according to any of Formula I-A to I-E:

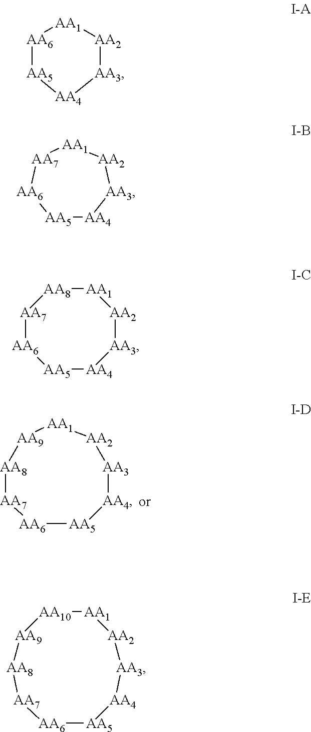

wherein AA₁, AA₂, AA₃, AA₄, AA₅, AA₆, AA₇, AA₈, AA₉, and AA₁₀ are amino acid residues.

The cCPP can comprise 6 to 8 amino acids. The cCPP can comprise 8 amino acids.

Each amino acid in the cCPP may be a natural or non-natural amino acid. The term “non-natural amino acid” refers to an organic compound that is a congener of a natural amino acid in that it has a structure similar to a natural amino acid so that it mimics the structure and reactivity of a natural amino acid. The non-natural amino acid can be a modified amino acid, and/or amino acid analog, that is not one of the 20 common naturally occurring amino acids or the rare natural amino acids selenocysteine or pyrrolysine. Non-natural amino acids can also be a D-isomer of a natural amino acid. Examples of suitable amino acids include, but are not limited to, alanine, allosoleucine, arginine, citrulline, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative thereof, or combinations thereof. These, and others amino acids, are listed in the Table 1 along with their abbreviations used herein.

TABLE 1
Amino Acid Abbreviations

| | Abbreviations* | Abbreviations* |
|---|---|---|
| Amino Acid | L-amino acid | D-amino acid |
| Alanine | Ala (A) | ala (a) |
| Allo-isoleucine | Aile | Aile |
| Arginine | Arg (R) | arg (r) |
| Asparagine | Asn (N) | asn (n) |
| Aspartic acid | Asp (D) | asp (d) |
| Cysteine | Cys (C) | cys (c) |
| Citrulline | Cit | Cit |
| Cyclohexylalanine | Cha | cha |
| 2,3-diaminopropionic acid | Dap | dap |
| 4-fluorophenylalanine | Fpa (Σ) | pfa |
| Glutamic acid | Glu (E) | glu (e) |
| Glutamine | Gln (Q) | gln (q) |
| Glycine | Gly (G) | gly (g) |
| Histidine | His (H) | his (h) |
| Homoproline (aka pipecolic acid) | Pip (Θ) | pip (θ) |
| Isoleucine | Ile (I) | ile (i) |
| Leucine | Leu (L) | leu (l) |
| Lysine | Lys (K) | lys (k) |
| Methionine | Met (M) | met (m) |
| 3-(2-naphthyl)-alanine | Nal (Φ) | nal (φ) |
| 3-(1-naphthyl)-alanine | 1-Nal | 1-nal |
| Norleucine | Nle (Ω) | nle |
| Phenylalanine | Phe (F) | phe (f) |
| Phenylglycine | Phg (Ψ) | phg |
| 4-(phosphonodifluoro- | F₂Pmp (Λ) | f₂pmp |
| methyl)phenylalanine | | |
| Proline | Pro (P) | pro (p) |
| Sarcosine | Sar (Ξ) | sar |
| Selenocysteine | Sec (U) | sec (u) |
| Serine | Ser (S) | ser (s) |
| Threonine | Thr (T) | thr (y) |
| Tyrosine | Tyr (Y) | tyr (y) |
| Tryptophan | Trp (W) | trp (w) |
| Valine | Val (V) | val (v) |
| Tert-butyl-alanine | Tle | tle |
| Penicillamine | Pen | Pen |
| Homoarginine | HomoArg | homoarg |
| Nicotinyl-lysine | Lys(NIC) | lys(NIC) |
| Triflouroacetyl-lysine | Lys(TFA) | lys(TFA) |
| Methyl-leucine | MeLeu | meLeu |
| 3-(3-benzothienyl)-alanine | Bta | bta |

*single letter abbreviations: when shown in capital letters herein it indicates the L-amino acid form, when shown in lower case herein it indicates the D-amino acid form.

The cCPP can comprise 4 to 20 amino acids, wherein: (i) at least one amino acid has a side chain comprising a guanidine group, or a protonated form thereof, (ii) at least one amino acid has no side chain or a side chain comprising

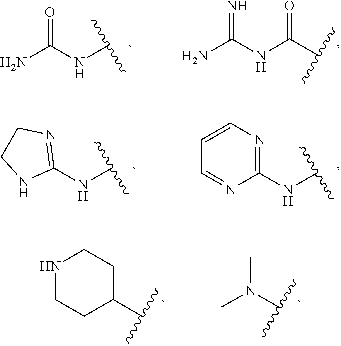

or a protonated form thereof; and (iii) at least two amino acids independently have a side chain comprising an aromatic or heteroaromatic group.

At least two amino acids can have no side chain or a side chain comprising

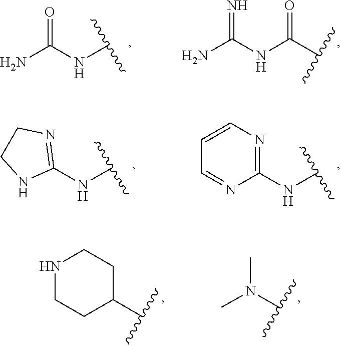

or a protonated form thereof. As used herein, when no side chain is present, the amino acid has two hydrogen atoms on the carbon atom(s) (e.g., —CH₂—) linking the amine and carboxylic acid.

The amino acid having no side chain can be glycine or β-alanine.

The cCPP can comprise from 6 to 20 amino acid residues which form the cCPP, wherein: (i) at least one amino acid can be glycine, β-alanine, or 4-aminobutyric acid residues; (ii) at least one amino acid can have a side chain comprising an aryl or heteroaryl group; and (iii) at least one amino acid has a side chain comprising a guanidine group,

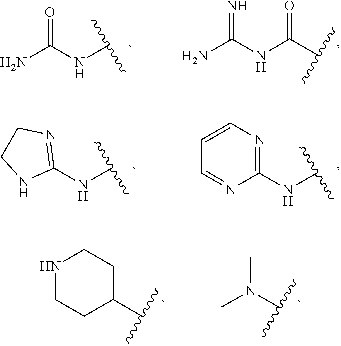

or a protonated form thereof.

The cCPP can comprise from 6 to 20 amino acid residues which form the cCPP, wherein: (i) at least two amino acids can independently beglycine, β-alanine, or 4-aminobutyric acid residues; (ii) at least one amino acid can have a side chain comprising an aryl or heteroaryl group; and (iii) at least one amino acid has a side chain comprising a guanidine group,

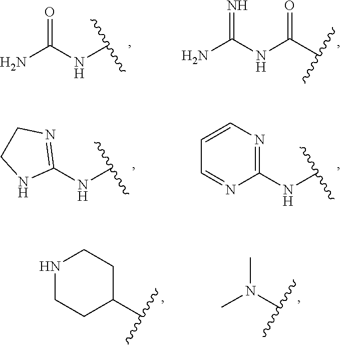

or a protonated form thereof.

The cCPP can comprise from 6 to 20 amino acid residues which form the cCPP, wherein: (i) at least three amino acids can independently be glycine, β-alanine, or 4-aminobutyric acid residues; (ii) at least one amino acid can have a side chain comprising an aromatic or heteroaromatic group; and (iii) at least one amino acid can have a side chain comprising a guanidine group,

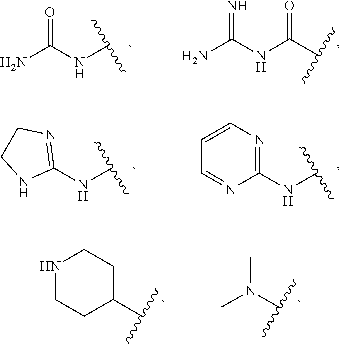

or a protonated form thereof

### Glycine and Related Amino Acid Residues

The cCPP can comprise (i) 1, 2, 3, 4, 5, or 6 glycine, β-alanine, 4-aminobutyric acid residues, or combinations thereof. The cCPP can comprise (i) 2 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, 3-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, β-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise (i) 1, 2, 3, 4, 5, or 6 glycine residues. The cCPP can comprise (i) 2 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues. The cCPP can comprise (i) 2 or 3 glycine residues. The cCPP can comprise (i) 1 or 2 glycine residues.

The cCPP can comprise (i) 3, 4, 5, or 6 glycine, β-alanine, 4-aminobutyric acid residues, or combinations thereof. The cCPP can comprise (i) 3 glycine, β-alanine, 4-aminobutyric acid residues, or combinations thereof. The cCPP can comprise (i) 4 glycine, β-alanine, 4-aminobutyric acid residues, or combinations thereof. The cCPP can comprise (i) 5 glycine, β-alanine, 4-aminobutyric acid residues, or combinations thereof. The cCPP can comprise (i) 6 glycine, (3-alanine, 4-aminobutyric acid residues, or combinations thereof. The cCPP can comprise (i) 3, 4, or 5 glycine, β-alanine, 4-aminobutyric acid residues, or combinations thereof. The cCPP can comprise (i) 3 or 4 glycine, β-alanine, 4-aminobutyric acid residues, or combinations thereof.

The cCPP can comprise at least three glycine residues. The cCPP can comprise (i) 3, 4, 5, or 6 glycine residues. The cCPP can comprise (i) 3 glycine residues. The cCPP can comprise (i) 4 glycine residues. The cCPP can comprise (i) 5 glycine residues. The cCPP can comprise (i) 6 glycine residues. The cCPP can comprise (i) 3, 4, or 5 glycine residues. The cCPP can comprise (i) 3 or 4 glycine residues

In embodiments, none of the glycine, β-alanine, or 4-aminobutyric acid residues in the cCPP are contiguous. Two or three glycine, β-alanine, 4- or aminobutyric acid residues can be contiguous. Two glycine, β-alanine, or 4-aminobutyric acid residues can be contiguous.

In embodiments, none of the glycine residues in the cCPP are contiguous. Each glycine residues in the cCPP can be separated by an amino acid residue that cannot be glycine. Two or three glycine residues can be contiguous. Two glycine residues can be contiguous

Amino Acid Side Chains with an Aromatic or Heteroaromatic Group

The cCPP can comprise (ii) 2, 3, 4, 5 or 6 amino acid residues independently having a side chain comprising an aromatic or heteroaromatic group. The cCPP can comprise (ii) 2 amino acid residues independently having a side chain comprising an aromatic or heteroaromatic group. The cCPP can comprise (ii) 3 amino acid residues independently having a side chain comprising an aromatic or heteroaromatic group. The cCPP can comprise (ii) 4 amino acid residues independently having a side chain comprising an aromatic or heteroaromatic group. The cCPP can comprise (ii) 5 amino acid residues independently having a side chain comprising an aromatic or heteroaromatic group. The cCPP can comprise (ii) 6 amino acid residues independently having a side chain comprising an aromatic or heteroaromatic group. The cCPP can comprise (ii) 2, 3, or 4 amino acid residues independently having a side chain comprising an aromatic or heteroaromatic group. The cCPP can comprise (ii) 2 or 3 amino acid residues independently having a side chain comprising an aromatic or heteroaromatic group.

The cCPP can comprise (ii) 2, 3, 4, 5 or 6 amino acid residues independently having a side chain comprising an aromatic group. The cCPP can comprise (ii) 2 amino acid residues independently having a side chain comprising an aromatic group. The cCPP can comprise (ii) 3 amino acid residues independently having a side chain comprising an aromatic group. The cCPP can comprise (ii) 4 amino acid residues independently having a side chain comprising an aromatic group. The cCPP can comprise (ii) 5 amino acid residues independently having a side chain comprising an aromatic group. The cCPP can comprise (ii) 6 amino acid residues independently having a side chain comprising an aromatic group. The cCPP can comprise (ii) 2, 3, or 4 amino acid residues independently having a side chain comprising an aromatic group. The cCPP can comprise (ii) 2 or 3 amino acid residues independently having a side chain comprising an aromatic group.

The aromatic group can be a 6- to 14-membered aryl. Aryl can be phenyl, naphthyl or anthracenyl, each of which is optionally substituted. Aryl can be phenyl or naphthyl, each of which is optionally substituted. The heteroaromatic group can be a 6- to 14-membered heteroaryl having 1, 2, or 3 heteroatoms selected from N, O, and S. Heteroaryl can be pyridyl, quinolyl, or isoquinolyl.

The amino acid residue having a side chain comprising an aromatic or heteroaromatic group can each independently be bis(homonaphthylalanine), homonaphthylalanine, naphthylalanine, phenylglycine, bis(homophenylalanine), homophenylalanine, phenylalanine, tryptophan, 3-(3-benzothienyl)-alanine, 3-(2-quinolyl)-alanine, O-benzylserine, 3-(4-(benzyloxy)phenyl)-alanine, S-(4-methylbenzyl)cysteine, N-(naphthalen-2-yl)glutamine, 3-(1,1′-biphenyl-4-yl)-alanine, 3-(3-benzothienyl)-alanine or tyrosine, each of which is optionally substituted with one or more substituents. The amino acid having a side chain comprising an aromatic or heteroaromatic group can each independently be selected from:

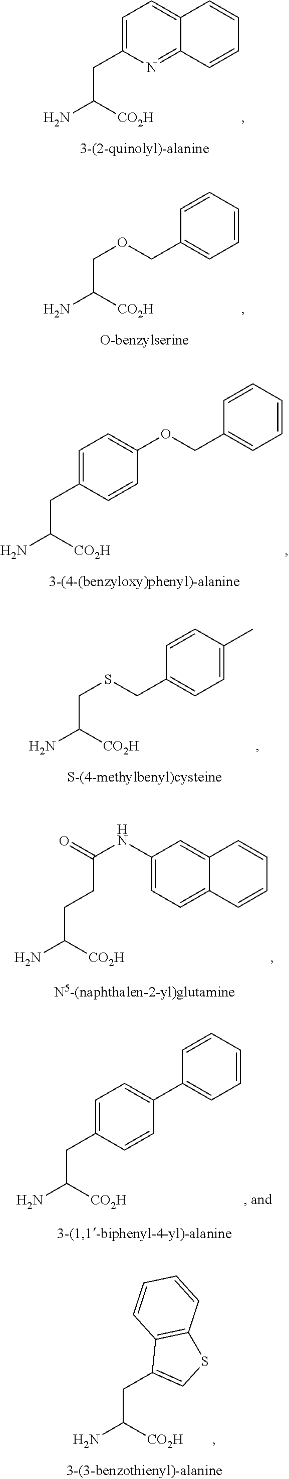

wherein the H on the N-terminus and/or the H on the C-terminus are replaced by a peptide bond.

The amino acid residue having a side chain comprising an aromatic or heteroaromatic group can each be independently a residue of phenylalanine, naphthylalanine, phenylglycine, homophenylalanine, homonaphthylalanine, bis(homophenylalanine), bis-(homonaphthylalanine), tryptophan, or tyrosine, each of which is optionally substituted with one or more substituents. The amino acid residue having a side chain comprising an aromatic group can each independently be a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine. The amino acid residue having a side chain comprising an aromatic group can each independently be a residue of phenylalanine, naphthylalanine, phenylglycine, homophenylalanine, or homonaphthylalanine, each of which is optionally substituted with one or more substituents. The amino acid residue having a side chain comprising an aromatic group can each be independently a residue of phenylalanine, naphthylalanine, homophenylalanine, homonaphthylalanine, bis(homonaphthylalanine), or bis(homonaphthylalanine), each of which is optionally substituted with one or more substituents. The amino acid residue having a side chain comprising an aromatic group can each be independently a residue of phenylalanine or naphthylalanine, each of which is optionally substituted with one or more substituents. At least one amino acid residue having a side chain comprising an aromatic group can be a residue of phenylalanine. At least two amino acid residues having a side chain comprising an aromatic group can be residues of phenylalanine. Each amino acid residue having a side chain comprising an aromatic group can be a residue of phenylalanine.

In embodiments, none of the amino acids having the side chain comprising the aromatic or heteroaromatic group are contiguous. Two amino acids having the side chain comprising the aromatic or heteroaromatic group can be contiguous. Two contiguous amino acids can have opposite stereochemistry. The two contiguous amino acids can have the same stereochemistry. Three amino acids having the side chain comprising the aromatic or heteroaromatic group can be contiguous. Three contiguous amino acids can have the same stereochemistry. Three contiguous amino acids can have alternating stereochemistry.

The amino acid residues comprising aromatic or heteroaromatic groups can be L-amino acids. The amino acid residues comprising aromatic or heteroaromatic groups can be D-amino acids. The amino acid residues comprising aromatic or heteroaromatic groups can be a mixture of D- and L-amino acids.

The optional substituent can be any atom or group which does not significantly reduce (e.g., by more than 50%) the cytosolic delivery efficiency of the cCPP, e.g., compared to an otherwise identical sequence which does not have the substituent. The optional substituent can be a hydrophobic substituent or a hydrophilic substituent. The optional substituent can be a hydrophobic substituent. The substituent can increase the solvent-accessible surface area (as defined herein) of the hydrophobic amino acid. The substituent can be halogen, alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio. The substituent can be halogen.

While not wishing to be bound by theory, it is believed that amino acids having an aromatic or heteroaromatic group having higher hydrophobicity values (i.e., amino acids having side chains comprising aromatic or heteroaromatic groups) can improve cytosolic delivery efficiency of a cCPP relative to amino acids having a lower hydrophobicity value. Each hydrophobic amino acid can independently have a hydrophobicity value greater than that of glycine. Each hydrophobic amino acid can independently be a hydrophobic amino acid having a hydrophobicity value greater than that of alanine. Each hydrophobic amino acid can independently have a hydrophobicity value greater or equal to phenylalanine. Hydrophobicity may be measured using hydrophobicity scales known in the art. Table 2 lists hydrophobicity values for various amino acids as reported by Eisenberg and Weiss (Proc. Natl. Acad. Sci. U.S.A 1984; 81(1):140-144), Engleman, et al. (Ann. Rev, of Biophys. Biophys. Chem. 1986; 1986(15):321-53), Kyte and Doolittle. Mol. Biol. 1982; 157(1):105-132), Hoop and Woods (Proc. Natl. Acad. Sci. U.S.A 1981; 78(6):3824-3828), and Janin (Nature. 1979; 277(5696):491-492), the entirety of each of which is herein incorporated by reference. Hydrophobicity can be measured using the hydrophobicity scale reported in Engleman, et al.

TABLE 2
Amino Acid Hydrophobicity

| Ami- | | Eisenberg | | Kyrie | Hoop | |
|---|---|---|---|---|---|---|
| no | | and | Engleman | and | and | |
| Acid | Group | Weiss | et al. | Doolittle | Woods | Janin |
| Ile | Nonpolar | 0.73 | 3.1 | 4.5 | −1.8 | 0.7 |
| Phe | Nonpolar | 0.61 | 3.7 | 2.8 | −2.5 | 0.5 |
| Val | Nonpolar | 0.54 | 2.6 | 4.2 | −1.5 | 0.6 |
| Leu | Nonpolar | 0.53 | 2.8 | 3.8 | −1.8 | 0.5 |
| Trp | Nonpolar | 0.37 | 1.9 | −0.9 | −3.4 | 0.3 |
| Met | Nonpolar | 0.26 | 3.4 | 1.9 | −1.3 | 0.4 |
| Ala | Nonpolar | 0.25 | 1.6 | 1.8 | −0.5 | 0.3 |
| Gly | Nonpolar | 0.16 | 1.0 | −0.4 | 0.0 | 0.3 |
| Cys | Unch/Polar | 0.04 | 2.0 | 2.5 | −1.0 | 0.9 |
| Tyr | Unch/Polar | 0.02 | −0.7 | −1.3 | −2.3 | −0.4 |
| Pro | Nonpolar | −0.07 | −0.2 | −1.6 | 0.0 | −0.3 |
| Thr | Unch/Polar | −0.18 | 1.2 | −0.7 | −0.4 | −0.2 |
| Ser | Unch/Polar | −0.26 | 0.6 | −0.8 | 0.3 | −0.1 |
| His | Charged | −0.40 | −3.0 | −3.2 | −0.5 | −0.1 |
| Glu | Charged | −0.62 | −8.2 | −3.5 | 3.0 | −0.7 |
| Asn | Unch/Polar | −0.64 | −4.8 | −3.5 | 0.2 | −0.5 |
| Gln | Unch/Polar | −0.69 | −4.1 | −3.5 | 0.2 | −0.7 |
| Asp | Charged | −0.72 | −9.2 | −3.5 | 3.0 | −0.6 |
| Lys | Charged | −1.10 | −8.8 | −3.9 | 3.0 | −1.8 |
| Arg | Charged | −1.80 | −12.3 | −4.5 | 3.0 | −1.4 |

The size of the aromatic or heteroaromatic groups may be selected to improve cytosolic delivery efficiency of the cCPP. While not wishing to be bound by theory, it is believed that a larger aromatic or heteroaromatic group on the side chain of amino acid may improve cytosolic delivery efficiency compared to an otherwise identical sequence having a smaller hydrophobic amino acid. The size of the hydrophobic amino acid can be measured in terms of molecular weight of the hydrophobic amino acid, the steric effects of the hydrophobic amino acid, the solvent-accessible surface area (SASA) of the side chain, or combinations thereof. The size of the hydrophobic amino acid can be measured in terms of the molecular weight of the hydrophobic amino acid, and the larger hydrophobic amino acid has a side chain with a molecular weight of at least about 90 g/mol, or at least about 130 g/mol, or at least about 141 g/mol. The size of the amino acid can be measured in terms of the SASA of the hydrophobic side chain. The hydrophobic amino acid can have a side chain with a SASA of greater than or equal to alanine, or greater than or equal to glycine. Larger hydrophobic amino acids can have a side chain with a SASA greater than alanine, or greater than glycine. The hydrophobic amino acid can have an aromatic or heteroaromatic group with a SASA greater than or equal to about piperidine-2-carboxylic acid, greater than or equal to about tryptophan, greater than or equal to about phenylalanine, or greater than or equal to about naphthylalanine. A first hydrophobic amino acid (AA_{H1}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². A second hydrophobic amino acid (AA_{H2}) can have a side chain with a SASA of at least about 200 Å², at least about 210 Å², at least about 220 Å², at least about 240 Å², at least about 250 Å², at least about 260 Å², at least about 270 Å², at least about 280 Å², at least about 290 Å², at least about 300 Å², at least about 310 Å², at least about 320 Å², or at least about 330 Å². The side chains of AA_{H1} and AA_{H2} can have a combined SASA of at least about 350 Å², at least about 360 Å², at least about 370 Å², at least about 380 Å2, at least about 390 Å2, at least about 400 Å², at least about 410 Å2, at least about 420 Å², at least about 430 Å2, at least about 440 Å², at least about 450 Å², at least about 460 Å², at least about 470 Å², at least about 480 Å², at least about 490 Å² greater than about 500 Å², at least about 510 Å², at least about 520 Å², at least about 530 Å², at least about 540 Å², at least about 550 Å², at least about 560 Å², at least about 570 Å², at least about 580 Å², at least about 590 Å², at least about 600 Å², at least about 610 Å², at least about 620 Å², at least about 630 Å², at least about 640 Å², greater than about 650 Å², at least about 660 Å², at least about 670 Å², at least about 680 Å², at least about 690 Å², or at least about 700 Å². AA_{H2} can be a hydrophobic amino acid residue with a side chain having a SASA that is less than or equal to the SASA of the hydrophobic side chain of AA_{H1}. By way of example, and not by limitation, a cCPP having a Nal-Arg motif may exhibit improved cytosolic delivery efficiency compared to an otherwise identical cCPP having a Phe-Arg motif, a cCPP having a Phe-Nal-Arg motif may exhibit improved cytosolic delivery efficiency compared to an otherwise identical cCPP having a Nal-Phe-Arg motif, and a phe-Nal-Arg motif may exhibit improved cytosolic delivery efficiency compared to an otherwise identical cCPP having a nal-Phe-Arg motif.

As used herein, “hydrophobic surface area” ˜ or “SASA” refers to the surface area (reported as square Ångstroms; Å²) of an amino acid side chain that is accessible to a solvent, SASA can be calculated using the ‘rolling ball’ algorithm developed by Shrake & Rupley (J Mol Biol. 79 (2): 351-71), which is herein incorporated by reference in its entirety for all purposes. This algorithm uses a “sphere” of solvent of a particular radius to probe the surface of the molecule. A typical value of the sphere is 1.4 Å, which approximates to the radius of a water molecule.

SASA values for certain side chains are shown below in Table 3. The SASA values described herein are based on the theoretical values listed in Table 3 below, as reported by Tien, et al. (PLOS ONE 8(11): e80635. https://doi.org/10.1371/joumnal.pone.0080635), which is herein incorporated by reference in its entirety for all purposes.

TABLE 3
Amino Acid SASA Values

| | | | Miller et al. | Rose et al. |
|---|---|---|---|---|
| Residue | Theoretical | Empirical | (1987) | (1985) |
| Alanine | 129.0 | 121.0 | 113.0 | 118.1 |
| Arginine | 274.0 | 265.0 | 241.0 | 256.0 |
| Asparagine | 195.0 | 187.0 | 158.0 | 165.5 |
| Aspartate | 193.0 | 187.0 | 151.0 | 158.7 |
| Cysteine | 167.0 | 148.0 | 140.0 | 146.1 |
| Glutamate | 223.0 | 214.0 | 183.0 | 186.2 |
| Glutamine | 225.0 | 214.0 | 189.0 | 193.2 |
| Glycine | 104.0 | 97.0 | 85.0 | 88.1 |
| Histidine | 224.0 | 216.0 | 194.0 | 202.5 |
| Isoleucine | 197.0 | 195.0 | 182.0 | 181.0 |
| Leucine | 201.0 | 191.0 | 180.0 | 193.1 |
| Lysine | 236.0 | 230.0 | 211.0 | 225.8 |
| Methionine | 224.0 | 203.0 | 204.0 | 203.4 |
| Phenylalanine | 240.0 | 228.0 | 218.0 | 222.8 |
| Proline | 159.0 | 154.0 | 143.0 | 146.8 |
| Serine | 155.0 | 143.0 | 122.0 | 129.8 |
| Threonine | 172.0 | 163.0 | 146.0 | 152.5 |
| Tryptophan | 285.0 | 264.0 | 259.0 | 266.3 |
| Tyrosine | 263.0 | 255.0 | 229.0 | 236.8 |
| Valine | 174.0 | 165.0 | 160.0 | 164.5 |

### Amino Acid Residues Having a Side Chain Comprising a Guanidine Group, Guanidine Replacement Group, or Protonated Form Thereof

As used herein, guanidine refers to the structure:

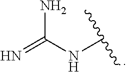 [SMILES: *NC(=N)N]

As used herein, a protonated form of guanidine refers to the structure:

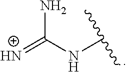 [SMILES: *NC(N)=[NH2+]]

Guanidine replacement groups refer to functional groups on the side chain of amino acids that will be positively charged at or above physiological pH or those that can recapitulate the hydrogen bond donating and accepting activity of guanidinium groups.

The guanidine replacement groups facilitate cell penetration and delivery of therapeutic agents while reducing toxicity associated with guanidine groups or protonated forms thereof. The cCPP can comprise at least one amino acid having a side chain comprising a guanidine or guanidinium replacement group. The cCPP can comprise at least two amino acids having a side chain comprising a guanidine or guanidinium replacement group. The cCPP can comprise at least three amino acids having a side chain comprising a guanidine or guanidinium replacement group

The guanidine or guanidinium group can be an isostere of guanidine or guanidinium. The guanidine or guanidinium replacement group can be less basic than guanidine.

As used herein, a guanidine replacement group refers to

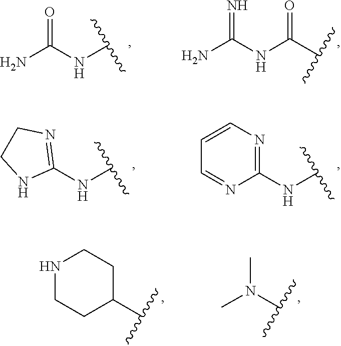

or a protonated form thereof.

The disclosure relates to a cCPP comprising from 4 to 20 amino acids residues, wherein: (i) at least one amino acid has a side chain comprising a guanidine group, or a protonated form thereof; (ii) at least one amino acid residue has no side chain or a side chain comprising

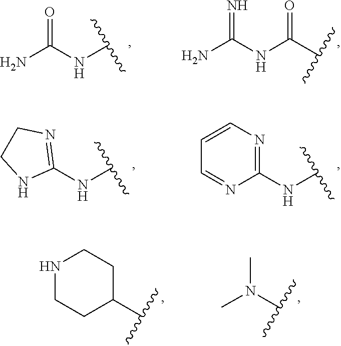

or a protonated form thereof; and (iii) at least two amino acids residues independently have a side chain comprising an aromatic or heteroaromatic group.

At least two amino acids residues can have no side chain or a side chain comprising

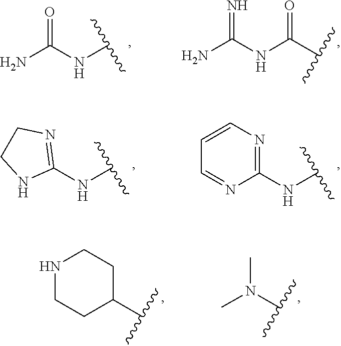

or a protonated form thereof. As used herein, when no side chain is present, the amino acid residue have two hydrogen atoms on the carbon atom(s) (e.g., —CH₂—) linking the amine and carboxylic acid.

The cCPP can comprise at least one amino acid having a side chain comprising one of the following moieties:

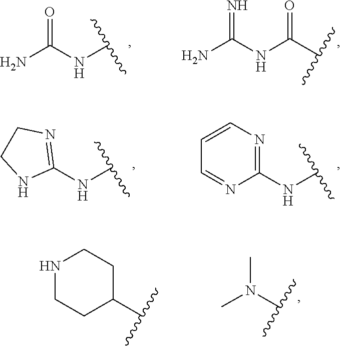

or a protonated form thereof.

The cCPP can comprise at least two amino acids each independently having one of the following moieties

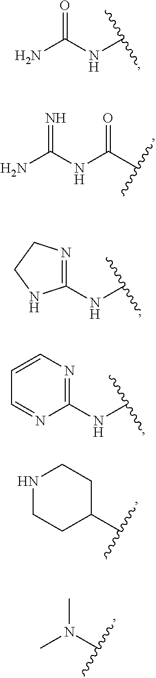

or a protonated form thereof. At least two amino acids can have a side chain comprising the same moiety selected from:

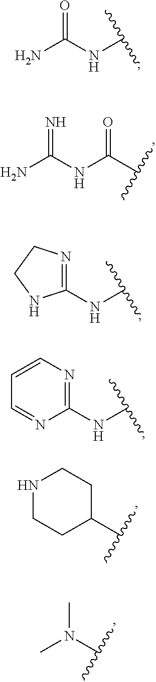

or a protonated form thereof. At least one amino acid can have a side chain comprising

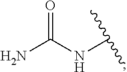

or a protonated form thereof. At least two amino acids can have a side chain comprising

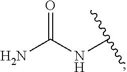

or a protonated form thereof. One, two, three, or four amino acids can have a side chain comprising

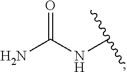

or a protonated form thereof. One amino acid can have a side chain comprising

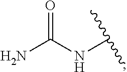

or a protonated form thereof. Two amino acids can have a side chain comprising

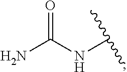

or a protonated form thereof.

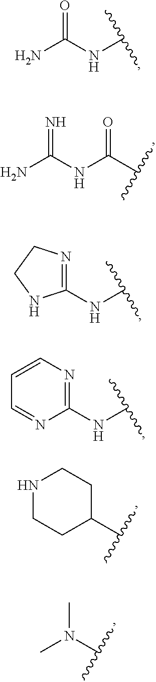

or a protonated form thereof, can be attached to the terminus of the amino acid side chain.

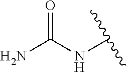 [SMILES: *NC(N)=O]

can be attached to the terminus of the amino acid side chain.

The cCPP can comprise (iii) 2, 3, 4, 5 or 6 amino acid residues independently having a side chain comprising a guanidine group, guanidine replacement group, or a protonated form thereof. The cCPP can comprise (iii) 2 amino acid residues independently having a side chain comprising a guanidine group, guanidine replacement group, or a protonated form thereof. The cCPP can comprise (iii) 3 amino acid residues independently having a side chain comprising a guanidine group, guanidine replacement group, or a protonated form thereof. The cCPP can comprise (iii) 4 amino acid residues independently having a side chain comprising a guanidine group, guanidine replacement group, or a protonated form thereof. The cCPP can comprise (iii) 5 amino acid residues independently having a side chain comprising a guanidine group, guanidine replacement group, or a protonated form thereof. The cCPP can comprise (iii) 6 amino acid residues independently having a side chain comprising a guanidine group, guanidine replacement group, or a protonated form thereof. The cCPP can comprise (iii) 2, 3, 4, or 5 amino acid residues independently having a side chain comprising a guanidine group, guanidine replacement group, or a protonated form thereof. The cCPP can comprise (iii) 2, 3, or 4 amino acid residues independently having a side chain comprising a guanidine group, guanidine replacement group, or a protonated form thereof. The cCPP can comprise (iii) 2 or 3 amino acid residues independently having a side chain comprising a guanidine group, guanidine replacement group, or a protonated form thereof. The cCPP can comprise (iii) at least one amino acid residue having a side chain comprising a guanidine group or protonated form thereof. The cCPP can comprise (iii) two amino acid residues having a side chain comprising a guanidine group or protonated form thereof. The cCPP can comprise (iii) three amino acid residues having a side chain comprising a guanidine group or protonated form thereof.

The amino acid residues can independently have the side chain comprising the guanidine group, guanidine replacement group, or the protonated form thereof that are not contiguous. Two amino acid residues can independently have the side chain comprising the guanidine group, guanidine replacement group, or the protonated form thereof can be contiguous. Three amino acid residues can independently have the side chain comprising the guanidine group, guanidine replacement group, or the protonated form thereof can be contiguous. Four amino acid residues can independently have the side chain comprising the guanidine group, guanidine replacement group, or the protonated form thereof can be contiguous. The contiguous amino acid residues can have the same stereochemistry. The contiguous amino acids can have alternating stereochemistry.

The amino acid residues independently having the side chain comprising the guanidine group, guanidine replacement group, or the protonated form thereof, can be L-amino acids. The amino acid residues independently having the side chain comprising the guanidine group, guanidine replacement group, or the protonated form thereof, can be D-amino acids. The amino acid residues independently having the side chain comprising the guanidine group, guanidine replacement group, or the protonated form thereof, can be a mixture of L- or D-amino acids.

Each amino acid residue having the side chain comprising the guanidine group, or the protonated form thereof, can independently be a residue of arginine, homoarginine, 2-amino-3-propionic acid, 2-amino-4-guanidinobutyric acid or a protonated form thereof. Each amino acid residue having the side chain comprising the guanidine group, or the protonated form thereof, can independently be a residue of arginine or a protonated form thereof.

Each amino acid having the side chain comprising a guanidine replacement group, or protonated form thereof, can independently be

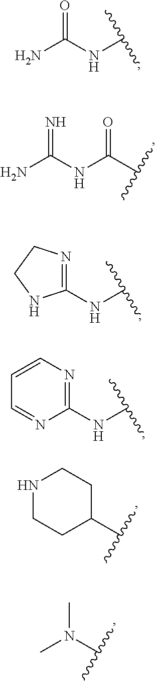

or a protonated form thereof.

Without being bound by theory, it is hypothesized that guanidine replacement groups have reduced basicity, relative to arginine and in some cases are uncharged at physiological pH (e.g., a —N(H)C(O)), and are capable of maintaining the bidentate hydrogen bonding interactions with phospholipids on the plasma membrane that is believed to facilitate effective membrane association and subsequent internalization. The removal of positive charge is also believed to reduce toxicity of the cCPP.

Those skilled in the art will appreciate that the N- and/or C-termini of the above non-natural aromatic hydrophobic amino acids, upon incorporation into the peptides disclosed herein, form amide bonds.

The cCPP can comprise a first amino acid having a side chain comprising an aromatic or heteroaromatic group and a second amino acid having a side chain comprising an aromatic or heteroaromatic group, wherein an N-terminus of a first glycine forms a peptide bond with the first amino acid having the side chain comprising the aromatic or heteroaromatic group, and a C-terminus of the first glycine forms a peptide bond with the second amino acid having the side chain comprising the aromatic or heteroaromatic group. Although by convention, the term “first amino acid” often refers to the N-terminal amino acid of a peptide sequence, as used herein “first amino acid” is used to distinguish the referent amino acid from another amino acid (e.g., a “second amino acid”) in the cCPP such that the term “first amino acid” may or may refer to an amino acid located at the N-terminus of the peptide sequence.

The cCPP can comprise an N-terminus of a second glycine forms a peptide bond with an amino acid having a side chain comprising an aromatic or heteroaromatic group, and a C-terminus of the second glycine forms a peptide bond with an amino acid having a side chain comprising a guanidine group, or a protonated form thereof.

The cCPP can comprise a first amino acid having a side chain comprising a guanidine group, or a protonated form thereof, and a second amino acid having a side chain comprising a guanidine group, or a protonated form thereof, wherein an N-terminus of a third glycine forms a peptide bond with a first amino acid having a side chain comprising a guanidine group, or a protonated form thereof, and a C-terminus of the third glycine forms a peptide bond with a second amino acid having a side chain comprising a guanidine group, or a protonated form thereof.

The cCPP can comprise a residue of asparagine, aspartic acid, glutamine, glutaminc acid, or homoglutamine. The cCPP can comprise a residue of asparagine. The cCPP can comprise a residue of glutamine.

The cCPP can comprise a residue of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

While not wishing to be bound by theory, it is believed that the chirality of the amino acids in the cCPPs may impact cytosolic uptake efficiency. The cCPP can comprise at least one D amino acid. The cCPP can comprise one to fifteen D amino acids. The cCPP can comprise one to ten D amino acids. The cCPP can comprise 1, 2, 3, or 4 D amino acids. The cCPP can comprise 2, 3, 4, 5, 6, 7, or 8 contiguous amino acids having alternating D and L chirality. The cCPP can comprise three contiguous amino acids having the same chirality. The cCPP can comprise two contiguous amino acids having the same chirality. At least two of the amino acids can have the opposite chirality. The at least two amino acids having the opposite chirality can be adjacent to each other. At least three amino acids can have alternating stereochemistry relative to each other. The at least three amino acids having the alternating chirality relative to each other can be adjacent to each other. At least four amino acids have alternating stereochemistry relative to each other. The at least four amino acids having the alternating chirality relative to each other can be adjacent to each other. At least two of the amino acids can have the same chirality. At least two amino acids having the same chirality can be adjacent to each other. At least two amino acids have the same chirality and at least two amino acids have the opposite chirality. The at least two amino acids having the opposite chirality can be adjacent to the at least two amino acids having the same chirality. Accordingly, adjacent amino acids in the cCPP can have any of the following sequences: D-L; L-D; D-L-L-D; L-D-D-L; L-D-L-L-D; D-L-D-D-L; D-L-L-D-L; or L-D-D-L-D. The amino acid residues that form the cCPP can all be L-amino acids. The amino acid residues that form the cCPP can all be D-amino acids.

At least two of the amino acids can have a different chirality. At least two amino acids having a different chirality can be adjacent to each other. At least three amino acids can have different chirality relative to an adjacent amino acid. At least four amino acids can have different chirality relative to an adjacent amino acid. At least two amino acids have the same chirality and at least two amino acids have a different chirality. One or more amino acid residues that form the cCPP can be achiral. The cCPP can comprise a motif of 3, 4, or 5 amino acids, wherein two amino acids having the same chirality can be separated by an achiral amino acid. The cCPPs can comprise the following sequences: D-X-D; D-X-D-X; D-X-D-X-D; L-X-L; L-X-L-X; or L-X-L-X-L, wherein X is an achiral amino acid. The achiral amino acid can be glycine.

An amino acid having a side chain comprising:

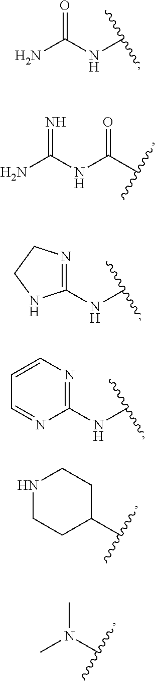

protonated form thereof, can be adjacent to an amino acid having a side chain comprising an aromatic or heteroaromatic group. An amino acid having a side chain comprising:

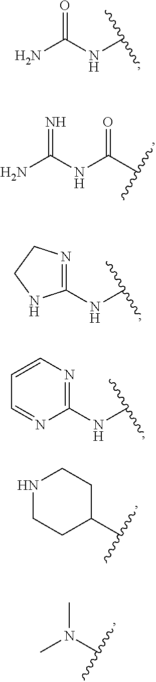

or a protonated form thereof, can be adjacent to at least one amino acid having a side chain comprising a guanidine or protonated form thereof. An amino acid having a side chain comprising a guanidine or protonated form thereof can be adjacent to an amino acid having a side chain comprising an aromatic or heteroaromatic group. Two amino acids having a side chain comprising:

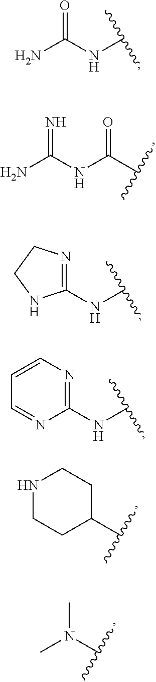

or protonated forms there, can be adjacent to each other. Two amino acids having a side chain comprising a guanidine or protonated form thereof are adjacent to each other. The cCPPs can comprise at least two contiguous amino acids having a side chain can comprise an aromatic or heteroaromatic group and at least two non-adjacent amino acids having a side chain comprising:

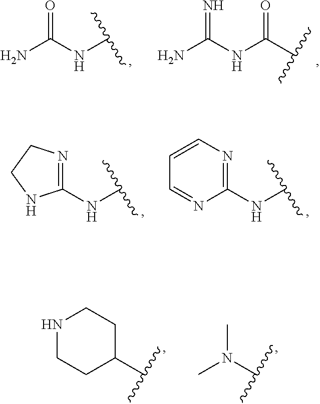

or a protonated form thereof. The cCPPs can comprise at least two contiguous amino acids having a side chain comprising an aromatic or heteroaromatic group and at least two non-adjacent amino acids having a side chain comprising

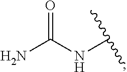

or a protonated form thereof. The adjacent amino acids can have the same chirality. The adjacent amino acids can have the opposite chirality. Other combinations of amino acids can have any arrangement of D and L amino acids, e.g., any of the sequences described in the preceding paragraph.

At least two amino acids having a side chain comprising:

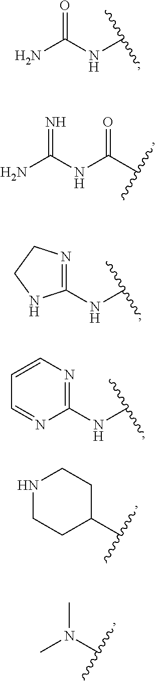

or a protonated form thereof, are alternating with at least two amino acids having a side chain comprising a guanidine group or protonated form thereof.

The cCPP can comprise the structure of Formula (A):

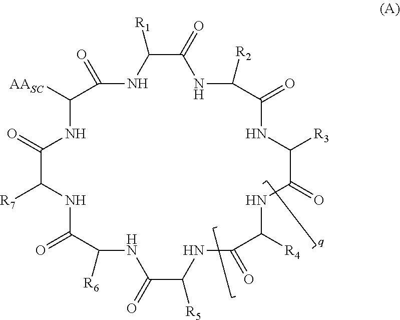

or a protonated form thereof,
wherein:
   R₁, R₂, and R₃ are each independently H or an aromatic or heteroaromatic side chain of an amino acid;
at least one of R₁, R₂, and R₃ is an aromatic or heteroaromatic side chain of an amino acid;
R₄, R₅, R₆, R₇ are independently H or an amino acid side chain;
at least one of R₄, R₅, R₆, R₇ is the side chain of 3-guanidino-2-aminopropionic acid, 4-guanidino-2-aminobutanoic acid, arginine, homoarginine, N-methylarginine, N,N-dimethylarginine, 2,3-diaminopropionic acid, 2,4-diaminobutanoic acid, lysine, N-methyllysine, N,N-dimethyllysine, N-ethyllysine, N,N,N-trimethyllysine, 4-guanidinophenylalanine, citrulline, N,N-dimethyllysine, 0-homoarginine, 3-(1-piperidinyl)alanine;
AA_{SC} is an amino acid side chain; and
q is 1, 2, 3 or 4.

In embodiments, at least one of R₄, R₅, R₆, R₇ are independently a uncharged, non-aromatic side chain of an amino acid. In embodiments, at least one of R₄, R₅, R₆, R₇ are independently H or a side chain of citrulline.

In embodiments, compounds are provided that include a cyclic peptide having 6 to 12 amino acids, wherein at least two amino acids of the cyclic peptide are charged amino acids, at least two amino acids of the cyclic peptide are aromatic hydrophobic amino acids and at least two amino acids of the cyclic peptide are uncharged, non-aromatic amino acids. In embodiments, at least two charged amino acids of the cyclic peptide are arginine. In embodiments, at least two aromatic, hydrophobic amino acids of the cyclic peptide are phenylalanine, naphtha alanine (3-Naphth-2-yl-alanine) or a combination thereof. In embodiments, at least two uncharged, non-aromatic amino acids of the cyclic peptide are citrulline, glycine or a combination thereof. In embodiments, the compound is a cyclic peptide having 6 to 12 amino acids wherein two amino acids of the cyclic peptide are arginine, at least two amino acids are aromatic, hydrophobic amino acids selected from phenylalanine, naphtha alanine and combinations thereof, and at least two amino acids are uncharged, non-aromatic amino acids selected from citrulline, glycine and combinations thereof.

In embodiments, the cyclic peptide of Formula (A) is not a cyclic peptide having a sequence of:

CPP sequences

| | | |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | fΦRrRrRQ |
| RRRΦFQ | RRΦFRRQ | FΦRRRRRQ |
| RRRRΦF | CRRRRFWQ | RRRRΦFDΩC |
| FΦRRRR | FfΦRrRrQ | FΦRRR |
| FφrRrRq | FFΦRRRRQ | FWRRR |
| FφrRrRQ | RFRFRΦRQ | RRRΦF |
| FΦRRRRQ | URRRRFWQ | RRRWF |
| fΦRrRrQ | CRRRRFWQ | |

where F is L-phenylalanine, f is D-phenylalanine, Φ is L-3-(2-naphthyl)-alanine, Φ is D-3-(2-naphthyl)-alanine, R is L-arginine, r is D-arginine, Q is L-glutamine, q is D-glutamine, C is L-cysteine, U is L-selenocysteine, W is L-tryptophan, K is L-lysine, D is L-aspartic acid, and Q is L-norleucine.

The cCPP can comprise the structure of Formula (I):

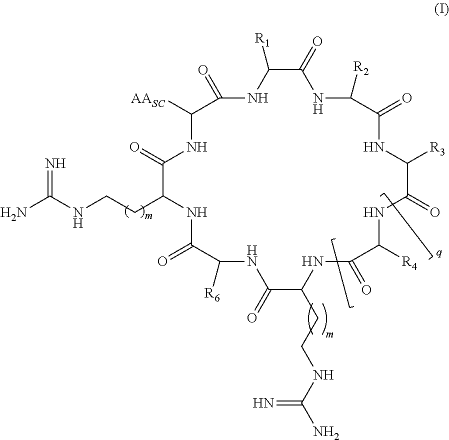

or a protonated form thereof,
wherein:
   R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4; and
each m is independently an integer 0, 1, 2, or 3.

R₁, R₂, and R₃ can each independently be H, -alkylene-aryl, or -alkylene-heteroaryl. R₁, R₂, and R₃ can each independently be H, —C₁₋₃alkylene-aryl, or —C₁₋₃alkylene-heteroaryl. R₁, R₂, and R₃ can each independently be H or -alkylene-aryl. R₁, R₂, and R₃ can each independently be H or —C₁₋₃alkylene-aryl. C₁₋₃alkylene can be methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can be phenyl. Heteroaryl can be pyridyl, quinolyl, and isoquinolyl. R₁, R₂, and R₃ can each independently be H, —C₁₋₃alkylene-Ph or —C₁₋₃alkylene-Naphthyl. R₁, R₂, and R₃ can each independently be H, —CH₂Ph, or —CH₂Naphthyl. R₁, R₂, and R₃ can each independently be H or —CH₂Ph.

R₁, R₂, and R₃ can each independently be the side chain of tyrosine, phenylalanine, 1-naphthylalanine, 2-naphthylalanine, tryptophan, 3-benzothienylalanine, 4-phenylphenylalanine, 3,4-difluorophenylalanine, 4-trifluoromethylphenylalanine, 2,3,4,5,6-pentafluorophenylalanine, homophenylalanine, 0-homophenylalanine, 4-tert-butyl-phenylalanine, 4-pyridinylalanine, 3-pyridinylalanine, 4-methylphenylalanine, 4-fluorophenylalanine, 4-chlorophenylalanine, 3-(9-anthryl)-alanine.

R₁ can be the side chain of tyrosine. R₁ can be the side chain of phenylalanine. R₁ can be the side chain of 1-naphthylalanine. R₁ can be the side chain of 2-naphthylalanine. R₁ can be the side chain of tryptophan. R₁ can be the side chain of 3-benzothienylalanine. R₁ can be the side chain of 4-phenylphenylalanine. R₁ can be the side chain of 3,4-difluorophenylalanine. R₁ can be the side chain of 4-trifluoromethylphenylalanine. R₁ can be the side chain of 2,3,4,5,6-pentafluorophenylalanine. R₁ can be the side chain of homophenylalanine. R₁ can be the side chain of β-homophenylalanine. R₁ can be the side chain of 4-tert-butyl-phenylalanine. R₁ can be the side chain of 4-pyridinylalanine. R₁ can be the side chain of 3-pyridinylalanine. R₁ can be the side chain of 4-methylphenylalanine. R₁ can be the side chain of 4-fluorophenylalanine. R₁ can be the side chain of 4-chlorophenylalanine. R₁ can be the side chain of 3-(9-anthryl)-alanine.

R₂ can be the side chain of tyrosine. R₂ can be the side chain of phenylalanine. R₂ can be the side chain of 1-naphthylalanine. R₁ can be the side chain of 2-naphthylalanine. R₂ can be the side chain of tryptophan. R₂ can be the side chain of 3-benzothienylalanine. R₂ can be the side chain of 4-phenylphenylalanine. R₂ can be the side chain of 3,4-difluorophenylalanine. R₂ can be the side chain of 4-trifluoromethylphenylalanine. R₂ can be the side chain of 2,3,4,5,6-pentafluorophenylalanine. R₂ can be the side chain of homophenylalanine. R₂ can be the side chain of β-homophenylalanine. R₂ can be the side chain of 4-tert-butyl-phenylalanine. R₂ can be the side chain of 4-pyridinylalanine. R₂ can be the side chain of 3-pyridinylalanine. R₂ can be the side chain of 4-methylphenylalanine. R₂ can be the side chain of 4-fluorophenylalanine. R₂ can be the side chain of 4-chlorophenylalanine. R₂ can be the side chain of 3-(9-anthryl)-alanine.

R₃ can be the side chain of tyrosine. R₃ can be the side chain of phenylalanine. R₃ can be the side chain of 1-naphthylalanine. R₃ can be the side chain of 2-naphthylalanine. R₃ can be the side chain of tryptophan. R₃ can be the side chain of 3-benzothienylalanine. R₃ can be the side chain of 4-phenylphenylalanine. R₃ can be the side chain of 3,4-difluorophenylalanine. R₃ can be the side chain of 4-trifluoromethylphenylalanine. R₃ can be the side chain of 2,3,4,5,6-pentafluorophenylalanine. R₃ can be the side chain of homophenylalanine. R₃ can be the side chain of β-homophenylalanine. R₃ can be the side chain of 4-tert-butyl-phenylalanine. R₃ can be the side chain of 4-pyridinylalanine. R₃ can be the side chain of 3-pyridinylalanine. R₃ can be the side chain of 4-methylphenylalanine. R₃ can be the side chain of 4-fluorophenylalanine. R₃ can be the side chain of 4-chlorophenylalanine. R₃ can be the side chain of 3-(9-anthryl)-alanine.

R₄ can be H, -alkylene-aryl, -alkylene-heteroaryl. R₄ can be H, —C₁₋₃alkylene-aryl, or —C₁₋₃alkylene-heteroaryl. R₄ can be H or -alkylene-aryl. R₄ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₄ can be H, —C₁₋₃alkylene-Ph or —C₁₋₃alkylene-Naphthyl. R₄ can be H or the side chain of an amino acid in Table 1 or Table 3. R₄ can be H or an amino acid residue having a side chain comprising an aromatic group. R₄ can be H, —CH₂Ph, or —CH₂Naphthyl. R₄ can be H or —CH₂Ph.

R₅ can be H, -alkylene-aryl, -alkylene-heteroaryl. R₅ can be H, —C₁₋₃alkylene-aryl, or —C₁₋₃alkylene-heteroaryl. R₅ can be H or -alkylene-aryl. R₅ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₅ can be H, —C₁₋₃alkylene-Ph or —C₁₋₃alkylene-Naphthyl. R₅ can be H or the side chain of an amino acid in Table 1 or Table 3. R₄ can be H or an amino acid residue having a side chain comprising an aromatic group. R₅ can be H, —CH₂Ph, or —CH₂Naphthyl. R₄ can be H or —CH₂Ph.

R₆ can be H, -alkylene-aryl, -alkylene-heteroaryl. R₆ can be H, —C₁₋₃alkylene-aryl, or —C₁₋₃alkylene-heteroaryl. R₆ can be H or -alkylene-aryl. R₆ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₆ can be H, —C₁₋₃alkylene-Ph or —C₁₋₃alkylene-Naphthyl. R₆ can be H or the side chain of an amino acid in Table 1 or Table 3. R₆ can be H or an amino acid residue having a side chain comprising an aromatic group. R₆ can be H, —CH₂Ph, or —CH₂Naphthyl. R₆ can be H or —CH₂Ph.

R₇ can be H, -alkylene-aryl, -alkylene-heteroaryl. R₇ can be H, —C₁₋₃alkylene-aryl, or —C₁₋₃alkylene-heteroaryl. R₇ can be H or -alkylene-aryl. R₇ can be H or —C₁₋₃alkylene-aryl. C₁₋₃alkylene can be a methylene. Aryl can be a 6- to 14-membered aryl. Heteroaryl can be a 6- to 14-membered heteroaryl having one or more heteroatoms selected from N, O, and S. Aryl can be selected from phenyl, naphthyl, or anthracenyl. Aryl can be phenyl or naphthyl. Aryl can phenyl. Heteroaryl can be pyridyl, quinolyl, and isoquinolyl. R₇ can be H, —C₁₋₃alkylene-Ph or —C₁₋₃alkylene-Naphthyl. R₇ can be H or the side chain of an amino acid in Table 1 or Table 3. R₇ can be H or an amino acid residue having a side chain comprising an aromatic group. R₇ can be H, —CH₂Ph, or —CH₂Naphthyl. R₇ can be H or —CH₂Ph.

One, two or three of R₁, R₂, R₃, R₄, R₅, R₆, and R₇ can be —CH₂Ph. One of R₁, R₂, R₃, R₄, R₅, R₆, and R₇ can be —CH₂Ph. Two of R₁, R₂, R₃, R₄, R₅, R₆, and R₇ can be —CH₂Ph. Three of R₁, R₂, R₃, R₄, R₅, R₆, and R₇ can be —CH₂Ph. At least one of R₁, R₂, R₃, R₄, R₅, R₆, and R₇ can be —CH₂Ph. No more than four of R₁, R₂, R₃, R₄, R₅, R₆, and R₇ can be —CH₂Ph.

One, two or three of R₁, R₂, R₃, and R₄ are —CH₂Ph. One of R₁, R₂, R₃, and R₄ is —CH₂Ph. Two of R₁, R₂, R₃, and R₄ are —CH₂Ph. Three of R₁, R₂, R₃, and R₄ are —CH₂Ph. At least one of R₁, R₂, R₃, and R₄ is —CH₂Ph.

One, two or three of R₁, R₂, R₃, R₄, R₅, R₆, and R₇ can be H. One of R₁, R₂, R₃, R₄, R₅, R₆, and R₇ can be H. Two of R₁, R₂, R₃, R₄, R₅, R₆, and R₇ are H. Three of R₁, R₂, R₃, R₅, R₆, and R₇ can be H. At least one of R₁, R₂, R₃, R₄, R₅, R₆, and R₇ can be H. No more than three of R₁, R₂, R₃, R₄, R₅, R₆, and R₇ can be —CH₂Ph.

One, two or three of R₁, R₂, R₃, and R₄ are H. One of R₁, R₂, R₃, and R₄ is H. Two of R₁, R₂, R₃, and R₄ are H. Three of R₁, R₂, R₃, and R₄ are H. At least one of R₁, R₂, R₃, and R₄ is H.

At least one of R₄, R₅, R₆, and R₇ can be side chain of 3-guanidino-2-aminopropionic acid. At least one of R₄, R₅, R₆, and R₇ can be side chain of 4-guanidino-2-aminobutanoic acid. At least one of R₄, R₅, R₆, and R₇ can be side chain of arginine. At least one of R₄, R₅, R₆, and R₇ can be side chain of homoarginine. At least one of R₄, R₅, R₆, and R₇ can be side chain of N-methylarginine. At least one of R₄, R₅, R₆, and R₇ can be side chain of N,N-dimethylarginine. At least one of R₄, R₅, R₆, and R₇ can be side chain of 2,3-diaminopropionic acid. At least one of R₄, R₅, R₆, and R₇ can be side chain of 2,4-diaminobutanoic acid, lysine. At least one of R₄, R₅, R₆, and R₇ can be side chain of N-methyllysine. At least one of R₄, R₅, R₆, and R₇ can be side chain of N,N-dimethyllysine. At least one of R₄, R₅, R₆, and R₇ can be side chain of N-ethyllysine. At least one of R₄, R₅, R₆, and R₇ can be side chain of N,N,N-trimethyllysine, 4-guanidinophenylalanine. At least one of R₄, R₅, R₆, and R₇ can be side chain of citrulline. At least one of R₄, R₅, R₆, and R₇ can be side chain of N,N-dimethyllysine, 0-homoarginine. At least one of R₄, R₅, R₆, and R₇ can be side chain of 3-(1-piperidinyl)alanine.

At least two of R₄, R₅, R₆, and R₇ can be side chain of 3-guanidino-2-aminopropionic acid. At least two of R₄, R₅, R₆, and R₇ can be side chain of 4-guanidino-2-aminobutanoic acid. At least two of R₄, R₅, R₆, and R₇ can be side chain of arginine. At least two of R₄, R₅, R₆, and R₇ can be side chain of homoarginine. At least two of R₄, R₅, R₆, and R₇ can be side chain of N-methylarginine. At least two of R₄, R₅, R₆, and R₇ can be side chain of N,N-dimethylarginine. At least two of R₄, R₅, R₆, and R₇ can be side chain of 2,3-diaminopropionic acid. At least two of R₄, R₅, R₆, and R₇ can be side chain of 2,4-diaminobutanoic acid, lysine. At least two of R₄, R₅, R₆, and R₇ can be side chain of N-methyllysine. At least two of R₄, R₅, R₆, and R₇ can be side chain of N,N-dimethyllysine. At least two of R₄, R₅, R₆, and R₇ can be side chain of N-ethyllysine. At least two of R₄, R₅, R₆, and R₇ can be side chain of N,N,N-trimethyllysine, 4-guanidinophenylalanine. At least two of R₄, R₅, R₆, and R₇ can be side chain of citrulline. At least two of R₄, R₅, R₆, and R₇ can be side chain of N,N-dimethyllysine, 0-homoarginine. At least two of R₄, R₅, R₆, and R₇ can be side chain of 3-(1-piperidinyl)alanine.

At least three of R₄, R₅, R₆, and R₇ can be side chain of 3-guanidino-2-aminopropionic acid. At least three of R₄, R₅, R₆, and R₇ can be side chain of 4-guanidino-2-aminobutanoic acid. At least three of R₄, R₅, R₆, and R₇ can be side chain of arginine. At least three of R₄, R₅, R₆, and R₇ can be side chain of homoarginine. At least three of R₄, R₅, R₆, and R₇ can be side chain of N-methylarginine. At least three of R₄, R₅, R₆, and R₇ can be side chain of N,N-dimethylarginine. At least three of R₄, R₅, R₆, and R₇ can be side chain of 2,3-diaminopropionic acid. At least three of R₄, R₅, R₆, and R₇ can be side chain of 2,4-diaminobutanoic acid, lysine. At least three of R₄, R₅, R₆, and R₇ can be side chain of N-methyllysine. At least three of R₄, R₅, R₆, and R₇ can be side chain of N,N-dimethyllysine. At least three of R₄, R₅, R₆, and R₇ can be side chain of N-ethyllysine. At least three of R₄, R₅, R₆, and R₇ can be side chain of N,N,N-trimethyllysine, 4-guanidinophenylalanine. At least three of R₄, R₅, R₆, and R₇ can be side chain of citrulline. At least three of R₄, R₅, R₆, and R₇ can be side chain of N,N-dimethyllysine, 0-homoarginine. At least three of R₄, R₅, R₆, and R₇ can be side chain of 3-(1-piperidinyl)alanine.

AA_{SC} can be a side chain of a residue of asparagine, glutamine, or homoglutamine. AA_{SC} can be a side chain of a residue of glutamine. The cCPP can further comprise a linker conjugated the AA_{SC}, e.g., the residue of asparagine, glutamine, or homoglutamine. Hence, the cCPP can further comprise a linker conjugated to the asparagine, glutamine, or homoglutamine residue. The cCPP can further comprise a linker conjugated to the glutamine residue.

q can be 1, 2, or 3. q can 1 or 2. q can be 1. q can be 2. q can be 3. q can be 4.

m can be 1-3. m can be 1 or 2. m can be 0. m can be 1. m can be 2. m can be 3.

The cCPP of Formula (A) can comprise the structure of Formula (I)

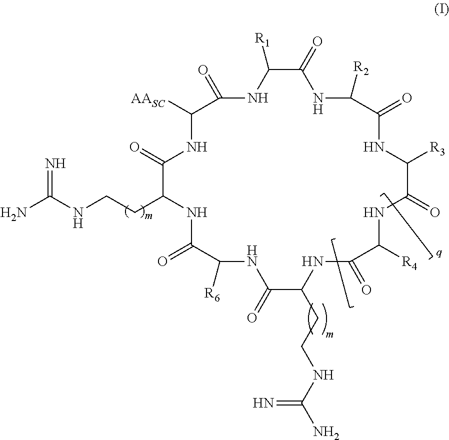

or protonated form thereof, wherein AA_{SC}, R₁, R₂, R₃, R₄, R₇, m and q are as defined herein

The cCPP of Formula (A) can comprise the structure of Formula (I-a) or Formula (I-b):

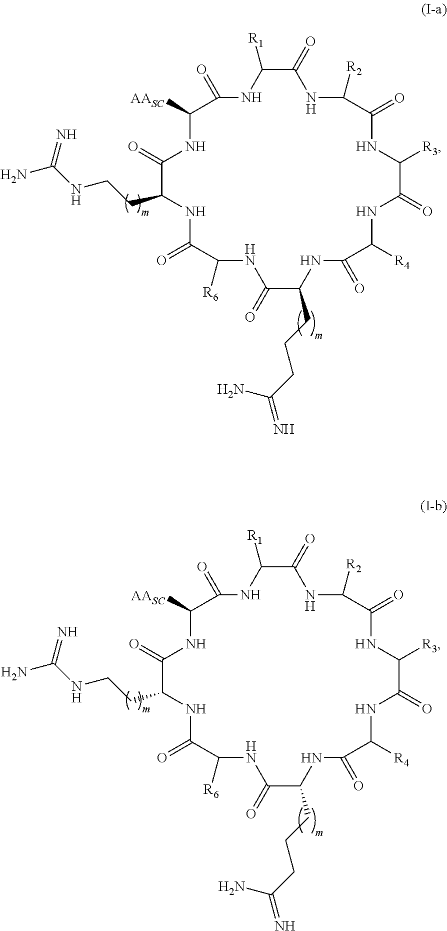

or protonated form thereof, wherein AA_{SC}, R₁, R₂, R₃, R₄, and m are as defined herein.

The cCPP of Formula (A) can comprise the structure of Formula (I-1), (I-2), (I-3) or (I-4):

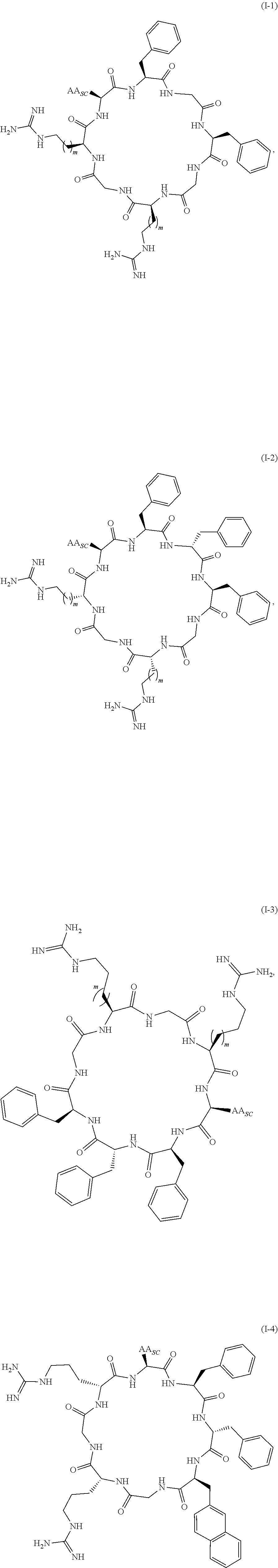

or N protonated from thereof, wherein AA_{SC} and m are as defined herein.

The cCPP of Formula (A) can comprise the structure of Formula (I-5) or (I-6):

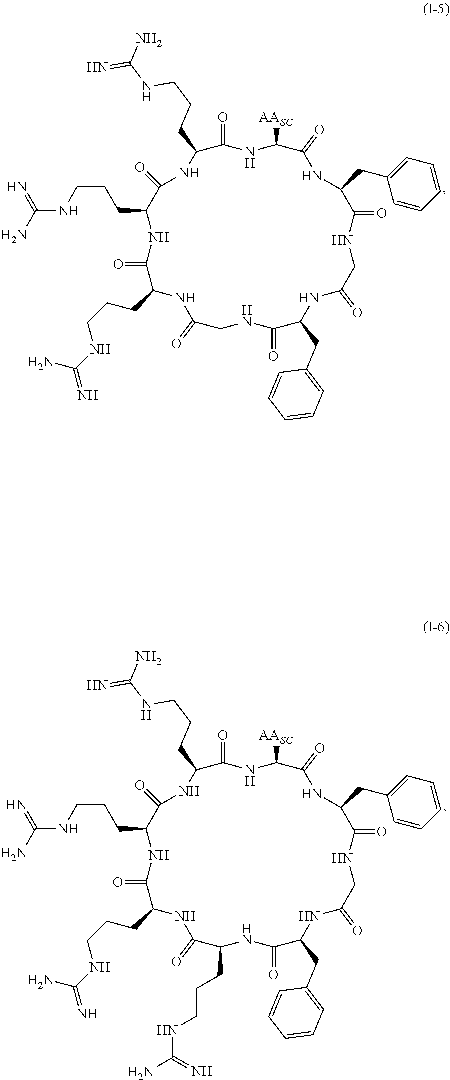

or protonated form thereof, wherein AA_{SC} is as defined herein.

The cCPP of Formula (A) can comprise the structure of Formula (I-1):

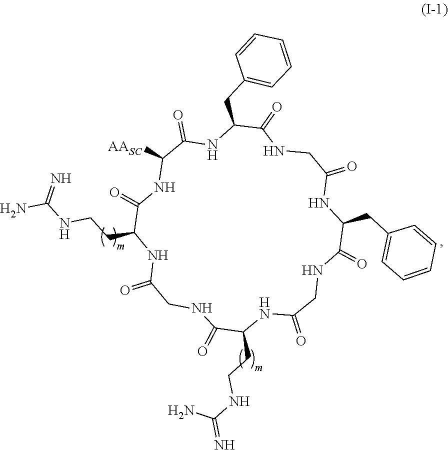

or a protonated form thereof,
wherein AA_{SC} and m are as defined herein.

The cCPP of Formula (A) can comprise the structure of Formula (I-2):

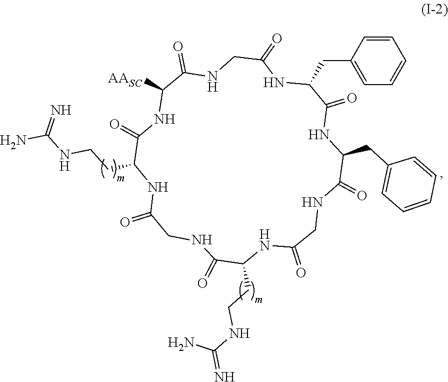

or a protonated form thereof,
wherein AA_{SC} and m are as defined herein.

The cCPP of Formula (A) can comprise the structure of Formula (I-3):

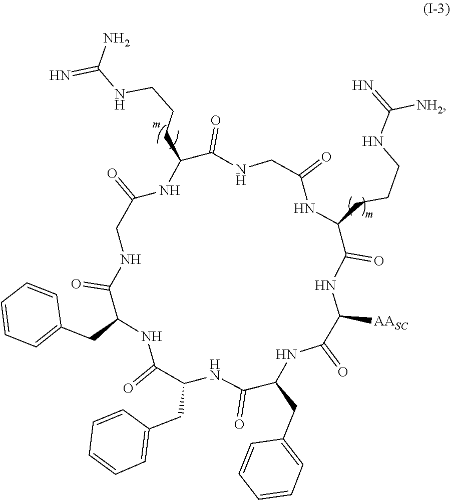

or a protonated form thereof,
wherein AA_{SC} and m are as defined herein.

The cCPP of Formula (A) can comprise the structure of Formula (I-4):

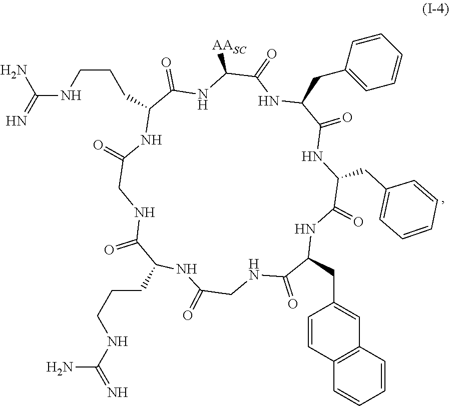

or a protonated form thereof,
wherein AA_{SC} and m are as defined herein.

The cCPP of Formula (A) can comprise the structure of Formula (I-5):

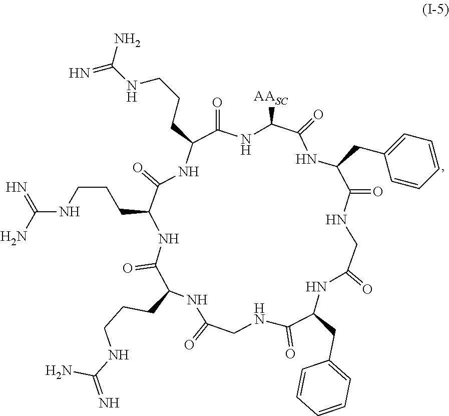

or a protonated form thereof,
wherein AA_{SC} and m are as defined herein.

The cCPP of Formula (A) can comprise the structure of Formula (I-6):

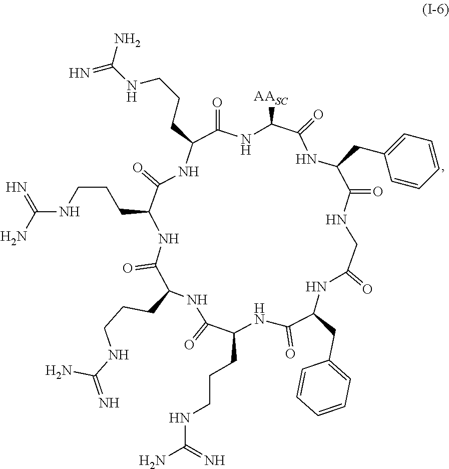

or a protonated form thereof, wherein AA_{SC} and m are as defined herein.

The cCPP can comprise one of the following sequences: FGFGRGR; GfFGrGr, FfΦGRGR; FfFGRGR; or FfΦGrGr. The cCPP can have one of the following sequences: FGFGRGRQ; GfFGrGrQ, FfDGRGRQ; FfFGRGRQ; or FfΦGrGrQ.

The disclosure also relates to a cCPP having the structure of Formula (II):

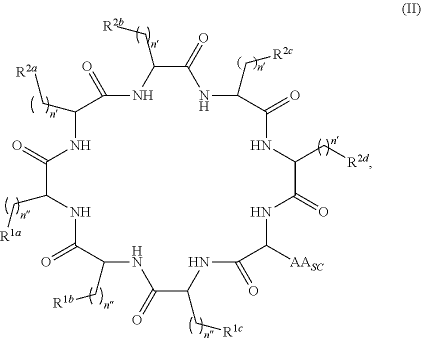

wherein:
AA_{SC} is an amino acid side chain;
R^{1a}, R^{1b}, and R^{1c} are each independently a 6- to 14-membered aryl or a 6- to 14-membered heteroaryl;
R^{2a}, R^{2b}, R^{2c} and R^{2d} are independently an amino acid side chain;
at least one of R^{2a}, R^{2b}, R^{2c} and R^{2d} is

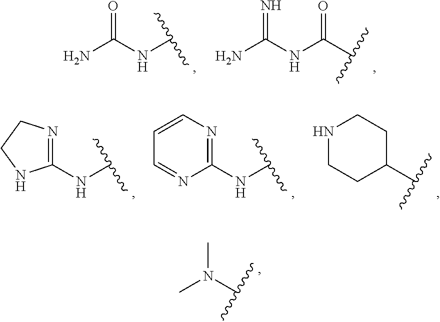

or a protonated form thereof,
at least one of R^{2a}, R^{2b}, R^{2c} and R^{2d} is guanidine or a protonated form thereof,
each n″ is independently an integer 0, 1, 2, 3, 4, or 5;
each n′ is independently an integer from 0, 1, 2, or 3; and
if n′ is 0 then R^{2a}, R^{2b}, R^{2b} or R^{2d} is absent.

At least two of R^{2a}, R^{2b}, R^{2c} and R^{2d} can be

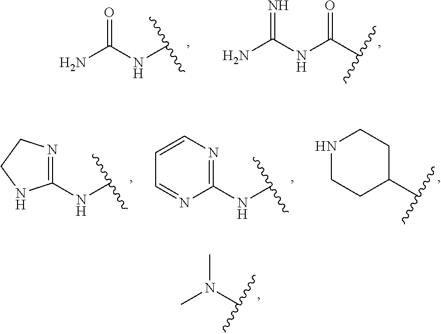

or a protonated form thereof. Two or three of R^{2a}, R^{2b}, R^{2c} and R^{2d} can be

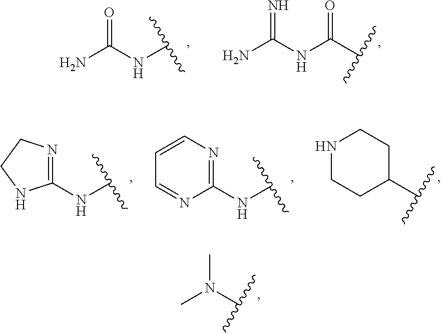

or a protonated form thereof one of R^{2a}, R^{2b}, R^{2c} and R^{2d} can be

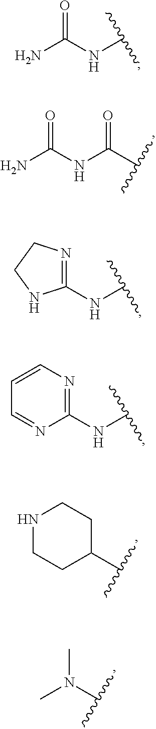

or a protonated form thereof. At least one of R^{2a}, R^{2b}, R^{2c} and R^{2d} can be

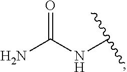

or a protonated form thereof, and the remaining of R^{2a}, R^{2b}R^{2c} and R^{2d} can be guanidine or a protonated form thereof. At least two of R^{2a}, R^{2b}, R^{2c} and R^{2d} can be

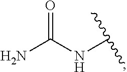

or a protonated form thereof, and the remaining of R^{2a}, R^{2b}, R^{2c} and R^{2d} can be guanidine, or a protonated form thereof.

All of R^{2a}, R^{2b}, R^{2c} and R^{2d} can be

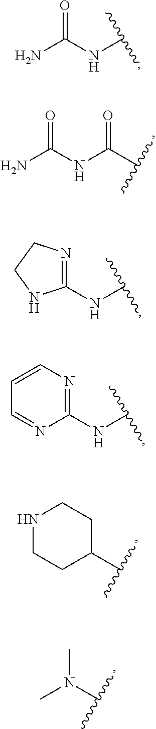

or a protonated form thereof. At least of R^{2a}, R^{2b}, R^{2c} and R^{2d} can be

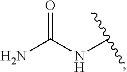

or a protonated form thereof, and the remaining of R^{2a}, R^{2b}, R^{2c} and R^{2d} can be guaninide or a protonated form thereof. At least two R^{2a}, R^{2b}, R^{2c} and R^{2d} groups can be

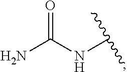

or a protonated form thereof, and the remaining of R^{2a}, R^{2b}, R^{2c} and R^{2d} are guanidine, or a protonated form thereof.

Each of R^{2a}, R^{2b}, R^{2c} and R^{2d} can independently be 2,3-diaminopropionic acid, 2,4-diaminobutyric acid, the side chains of ornithine, lysine, methyllysine, dimethyllysine, trimethyllysine, homo-lysine, serine, homo-serine, threonine, allo-threonine, histidine, 1-methylhistidine, 2-aminobutanedioic acid, aspartic acid, glutamic acid, or homo-glutamic acid.

AA_{SC} can be

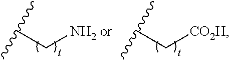

wherein t can be an integer from 0 to 5. AA_{SC} can be

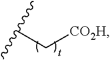

wherein t can be an integer from 0 to 5. t can be 1 to 5. t is 2 or 3. t can be 2. t can be 3.

The AC described herein can be coupled to AA_{SC}. In embodiments, a linker (L) couples the AC to AA_{SC}. In embodiments, a linker (L) is covalently bound to the backbone of the AC.
The AA_{SC} can be a side chain of a residue of asparagine, glutamine, or homoglutamine. The AA_{SC} can be a side chain of a residue of glutamine. The cyclic peptide can comprise a linker conjugated to the AA_{SC}, e.g., the residue of asparagine, glutamine, or homoglutamine.

R^{1a}, R^{1b}, and R^{1c} can each independently be 6- to 14-membered aryl. R^{1a}, R^{1b}, and R^{1c} can be each independently a 6- to 14-membered heteroaryl having one or more heteroatoms selected from N, O, or S. R^{1a}, R^{1b}, and R^{1c} can each be independently selected from phenyl, naphthyl, anthracenyl, pyridyl, quinolyl, or isoquinolyl. R^{1a}, R^{1b}, and R^{1c} can each be independently selected from phenyl, naphthyl, or anthracenyl. R^{1a}, R^{1b}, and R^{1c} can each be independently phenyl or naphthyl. R^{1a}, R^{1b}, and R^{1c} can each be independently selected pyridyl, quinolyl, or isoquinolyl.

Each n′ can independently be 1 or 2. Each n′ can be 1. Each n′ can be 2. At least one n′ can be 0. At least one n′ can be 1. At least one n′ can be 2. At least one n′ can be 3. At least one n′ can be 4. At least one n′ can be 5.

Each n″ can independently be an integer from 1 to 3. Each n″ can independently be 2 or 3. Each n″ can be 2. Each n″ can be 3. At least one n″ can be 0. At least one n″ can be 1. At least one n″ can be 2. At least one n″ can be 3.

Each n″ can independently be 1 or 2 and each n′ can independently be 2 or 3. Each n″ can be 1 and each n′ can independently be 2 or 3. Each n″ can be 1 and each n′ can be 2. Each n″ is 1 and each n′ is 3.

The cCPP of Formula (II) can have the structure of Formula (II-1):

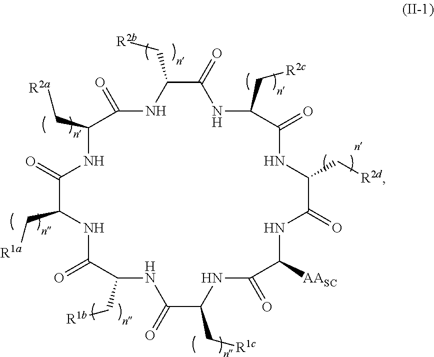

wherein R^{1a}, R^{1b}, R^{1c}, R^{2a}, R^{2b}, R^{2c}, R^{2d}, AA_{SC}, n′ and n″ are as defined herein.

The cCPP of Formula (II) can have the structure of Formula (IIa):

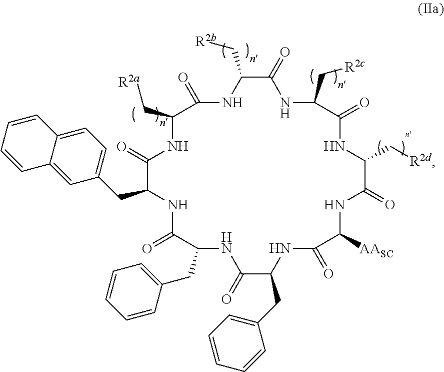

wherein R^{1a}, R^{1b}, R^{1c}, R^{2a}, R^{2b}, R^{2c}, R^{2d}, AA_{SC} and n′ are as defined herein.

The cCPP of formula (II) can have the structure of Formula (IIb):

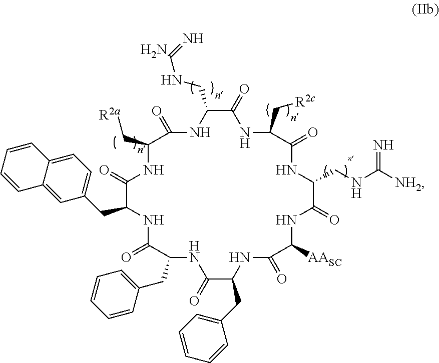

wherein R^{2a}, R^{2b}, AA_{SC}, and n′ are as defined herein.

The cCPP can have the structure of Formula (IIb):

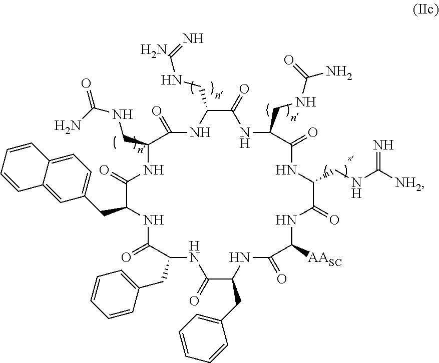

or a protonated form thereof,
wherein:
AA_{SC} and n′ are as defined herein.

The cCPP of Formula (IIa) has one of the following structures:

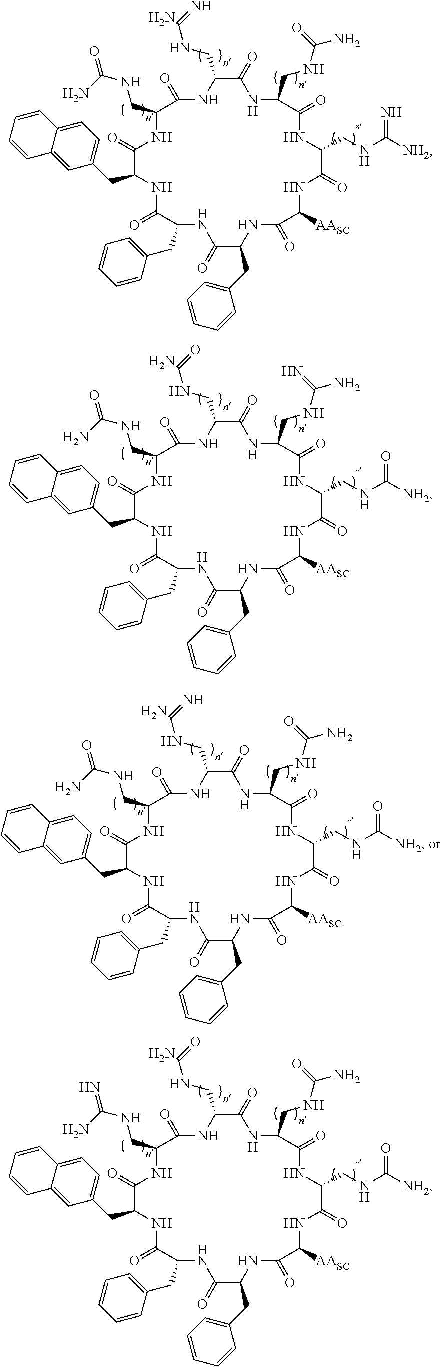

wherein AA_{SC} and n are as defined herein.

The cCPP of Formula (IIa) has one of the following structures:

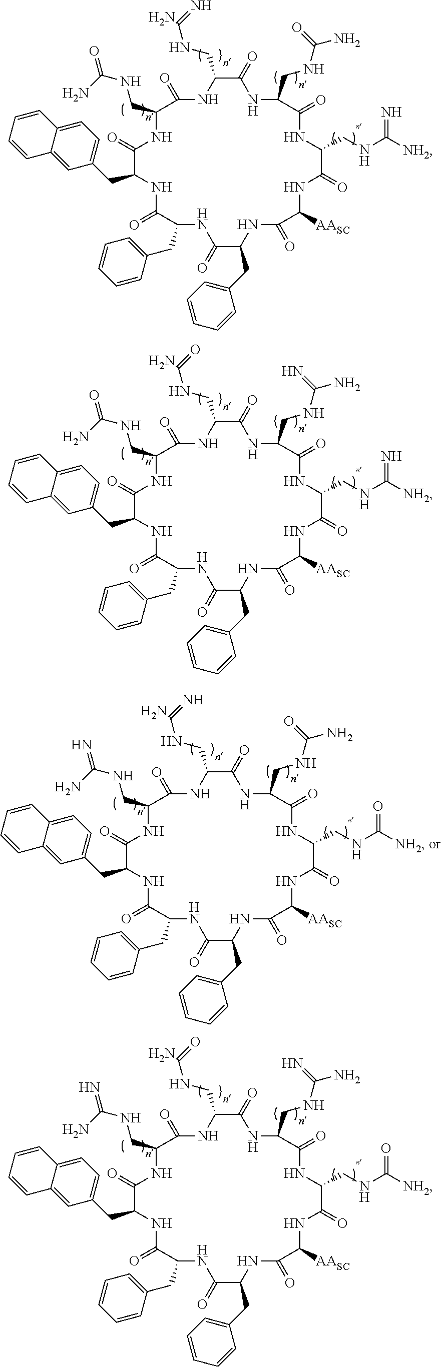

wherein AA_{SC} and n are as defined herein

The cCPP of Formula (IIa) has one of the following structures:

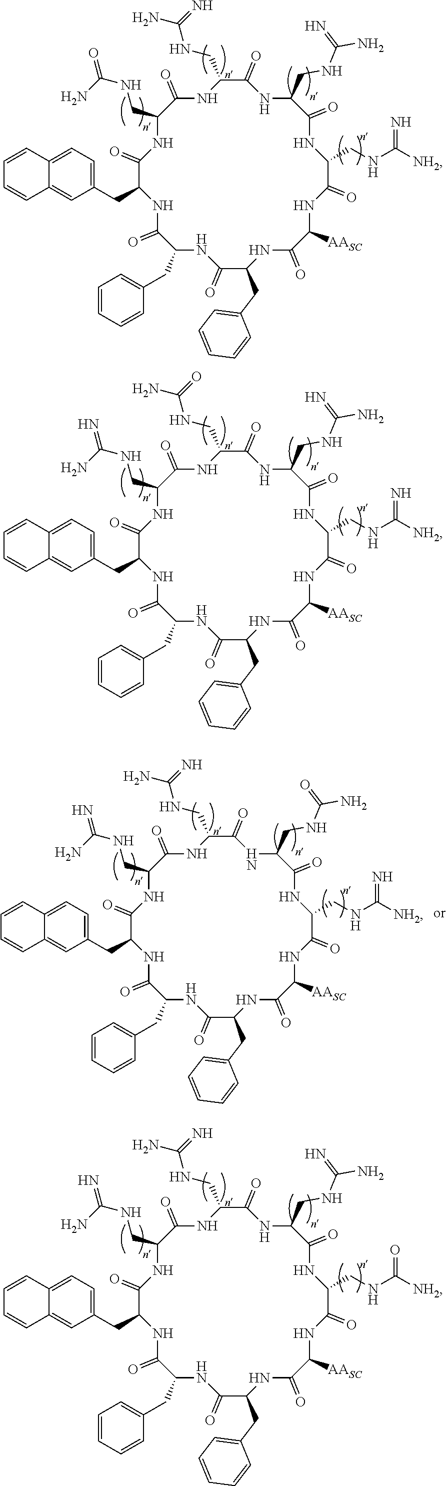

wherein AA_{SC} and n are as defined herein.

The cCPP of Formula (II) can have the structure:

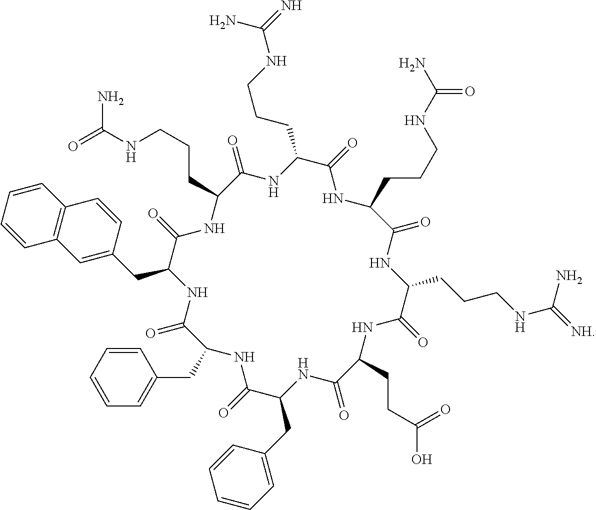 [SMILES: N=C(N)NCCC[C@H]1NC(=O)[C@H](CCCNC(N)=O)NC(=O)[C@@H](CCCNC(=N)N)NC(=O)[C@H](CCCNC(N)=O)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O]

The cCPP of Formula (II) can have the structure:

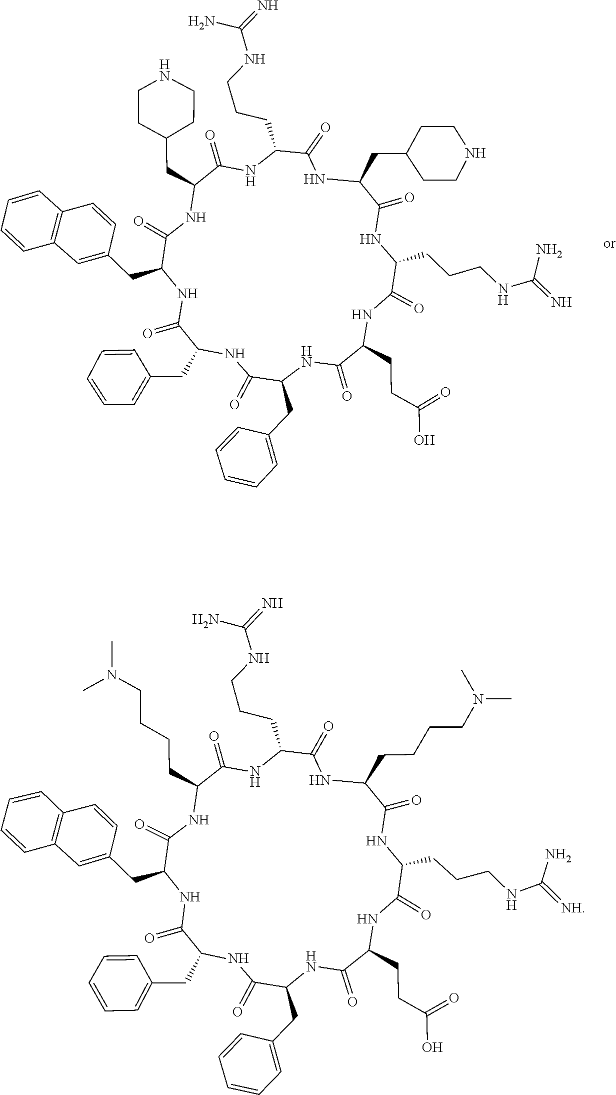 [SMILES: N=C(N)NCCC[C@H]1NC(=O)[C@H](CC2CCNCC2)NC(=O)[C@@H](CCCNC(=N)N)NC(=O)[C@H](CC2CCNCC2)NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC1=O ; CN(C)CCCC[C@@H]1NC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)[C@H](CCCCN(C)C)NC(=O)[C@@H](CCCNC(=N)N)NC1=O]

The cCPP can have the structure of Formula (III):

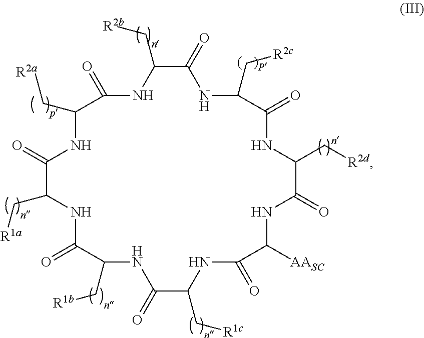

wherein:
AA_{SC} is an amino acid side chain;
R^{1a}, R^{1b}, and R^{1c} are each independently a 6- to 14-membered aryl or a 6- to 14-membered heteroaryl;
R^{2a} and R^{2c} are each independently H,

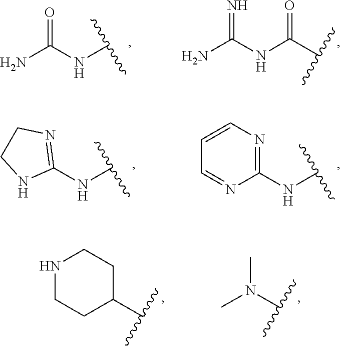

or a protonated form thereof;
R^{2b} and R^{2d} are each independently guanidine or a protonated form thereof,
each n″ is independently an integer from 1 to 3;
each n′ is independently an integer from 1 to 5; and
each p′ is independently an integer from 0 to 5.

The AC described herein can be coupled to an AA_{SC}. A linker can couple the AC to AA_{SC}. The linker can be covalently bound to the backbone of the AC, the 5′ end of the AC, or the 3′ end of the AC.

The cCPP of Formula (III) can have the structure of Formula (III-1):

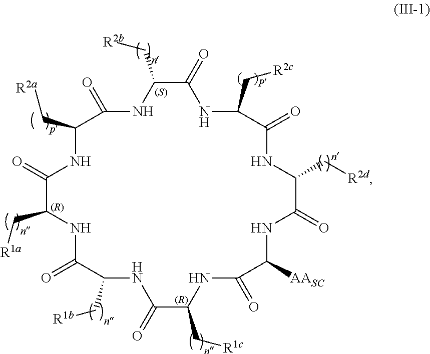

wherein:
AA_{SC}, R^{1a}, R^{1b}, R^{1c}, R^{2a}, R^{2c}, R^{2b}, R^{2d} n′, n″, and p′ are as defined herein.

The cCPP of Formula (III) can have the structure of Formula (IIIa):

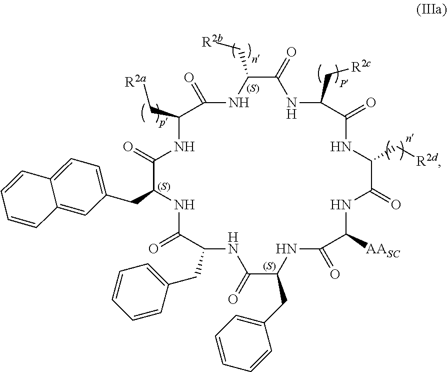

wherein:
AA_{SC}, R^{2a}, R^{2c}, R^{2c}, R^{2d} n′, n″, and p′ are as defined herein.

In Formulas (III), (III-1), and (IIIa), R^{a} and R^{c} can be H. R^{a} and R^{c} can be H and R^{b} and R^{d} can each independently be guanidine or protonated form thereof. R^{a} can be H. R^{b} can be H. p′ can be 0. R^{a} and R^{c} can be H and each p′ can be 0.

In Formulas (III), (III-1), and (IIIa), R^{a} and R^{c} can be H, R^{b} and R^{d} can each independently be guanidine or protonated form thereof, n″ can be 2 or 3, and each p′ can be 0.
p′ can 0. p′ can 1. p′ can 2. p′ can 3. p′ can 4. p′ can be 5.

The cCPP can have the structure:

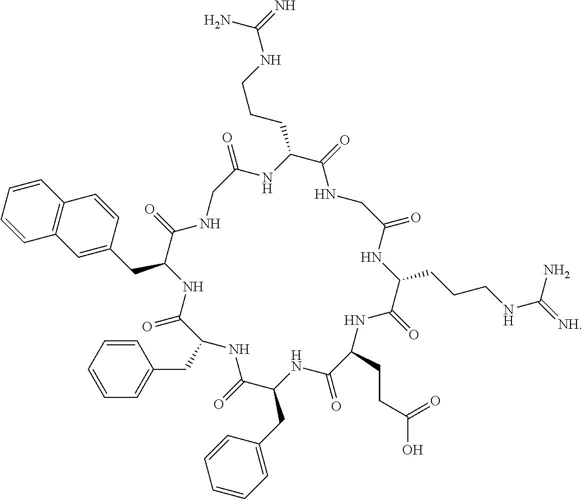 [SMILES: N=C(N)NCCC[C@H]1NC(=O)CNC(=O)[C@H](Cc2ccc3ccccc3c2)NC(=O)[C@@H](Cc2ccccc2)NC(=O)[C@H](Cc2ccccc2)NC(=O)[C@H](CCC(=O)O)NC(=O)[C@@H](CCCNC(=N)N)NC(=O)CNC1=O]

The cCPP of Formula (A) can be selected from:

CPP Sequence

(FfΦRrRrQ)
(FfΦCit-r-Cit-rQ)
(FfΦGrGrQ)
(FfFGRGRQ)
(FGFGRGRQ)
(GfFGrGrQ)
(FGFGRRRQ)
(FGFRRRRQ)

The cCPP of Formula (A) can be selected from:

CPP Sequence

FΦRRRRQ
fΦRrRrQ
FfΦRrRrQ
FfΦCit-r-Cit-rQ
FfΦGrGrQ
FfΦRGRGQ
FfFGRGRQ
FGFGRGRQ
GfFGrGrQ
FGFGRRRQ
FGFRRRRQ

In embodiments, the cCPP is selected from:

| CPP sequences | CPP sequences | CPP sequences |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | fΦRrRrRQ |
| RRRΦFQ | RRΦFRRQ | FΦRRRRRQ |
| RRRRΦF | CRRRRFWQ | RRRRΦFDΩC |
| FΦRRRR | FfΦRrRrQ | FΦRRR |
| FφrRrRq | FFΦRRRRQ | FWRRR |
| FφrRrRQ | RFRFRΦRQ | RRRΦF |
| FΦRRRRQ | URRRRFWQ | RRRWF |
| fΦRrRrQ | CRRRRFWQ | |

ΦL-naphthylalanine; φ=D-naphthylalanine; Ω=L-norleucine

In embodiments, the cCPP is not selected from:

| CPP sequences | CPP sequences | CPP sequences |
|---|---|---|
| FΦRRRQ | RRFRΦRQ | FΦRRRRQK |
| FΦRRRC | FRRRRΦQ | FΦRRRRQC |
| FΦRRRU | rRFRΦRQ | fΦRrRrRQ |
| RRRΦFQ | RRΦFRRQ | FΦRRRRRQ |
| RRRRΦF | CRRRRFWQ | RRRRΦFDΩC |
| FΦRRRR | FfΦRrRrQ | FΦRRR |
| FφrRrRq | FFΦRRRRQ | FWRRR |
| FφrRrRQ | RFRFRΦRQ | RRRΦF |
| FΦRRRRQ | URRRRFWQ | RRRWF |
| fΦRrRrQ | CRRRRFWQ | |

Φ = L-naphthylalanine; φ = D-naphthylalanine; Ω = L-norleucine

The cCPP can comprise the structure of Formula (D):

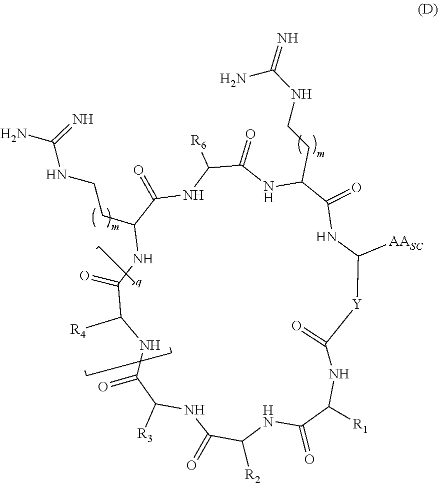

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;

### Y is

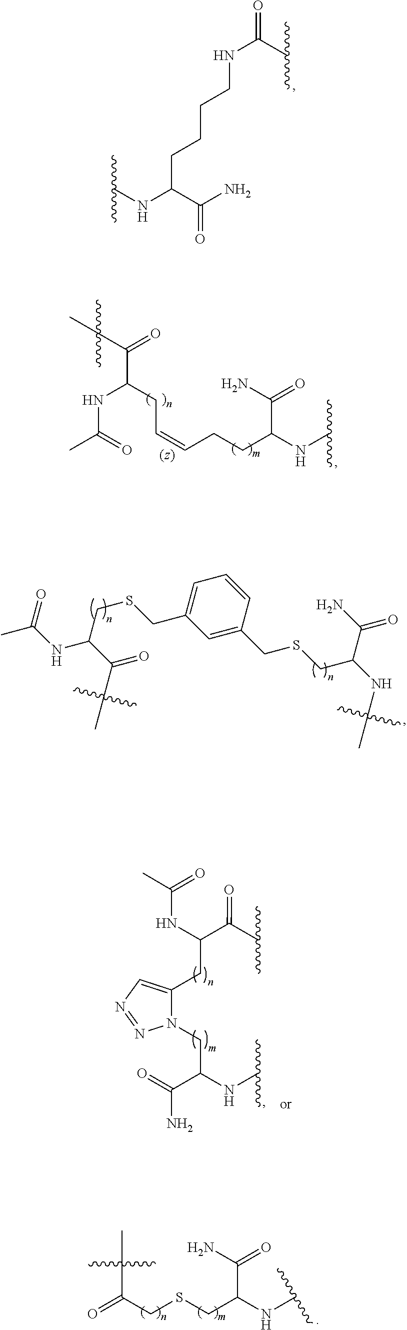

q is 1, 2, 3 or 4;
each m is independently an integer 0, 1, 2, or 3, and
each n is independently an integer 0, 1, 2, or 3.

The cCPP of Formula (D) can have the structure of Formula (D-I):

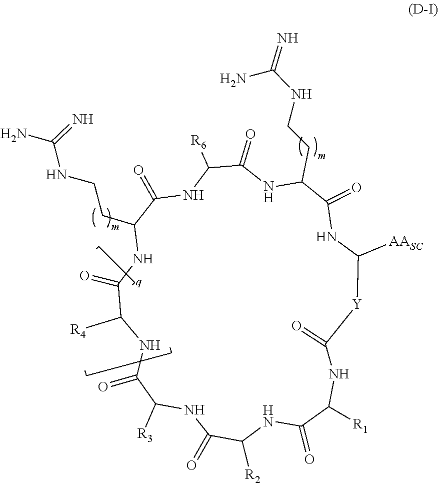

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and
Y is

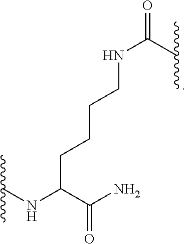 [SMILES: *NC(CCCCNC(*)=O)C(N)=O]

The cCPP of Formula (D) can have the structure of Formula (D-II):

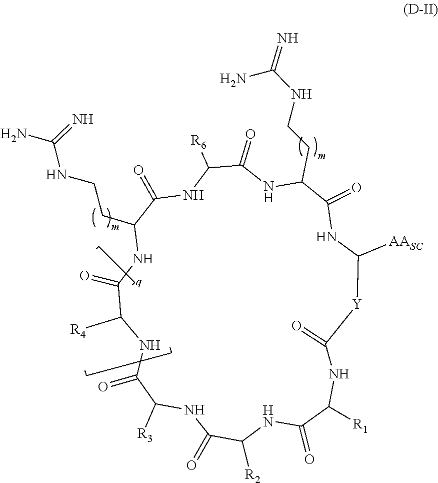

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, each n is independently an integer 0, 1, 2, or 3, and

### Y is

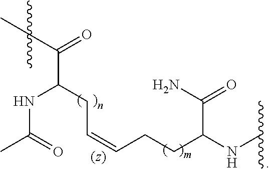

The cCPP of Formula (D) can have the structure of Formula (D-III):

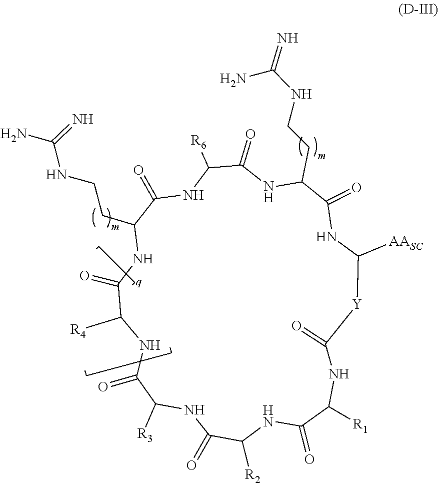

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3,
each n is independently an integer 0, 1, 2, or 3, and

### Y is

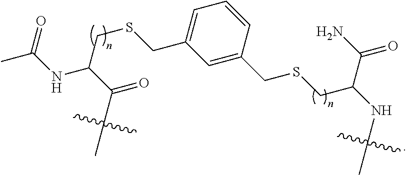

The cCPP of Formula (D) can have the structure of Formula (D-IV):

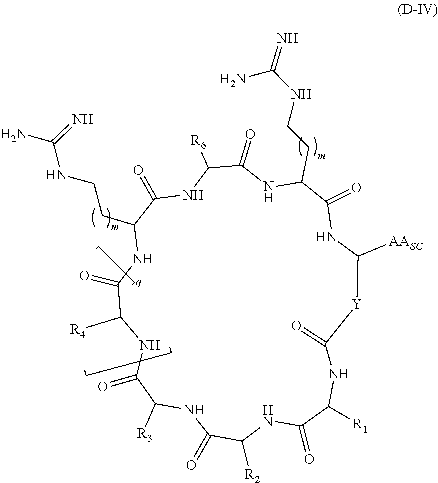

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
each m is independently an integer 0, 1, 2, or 3, and

### Y is

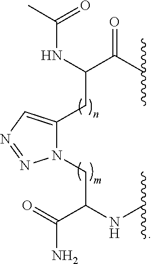

The cCPP of Formula (D) can have the structure of Formula (D-V):

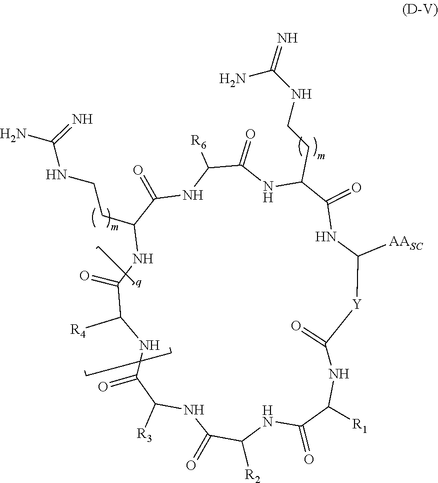

or a protonated form thereof,
wherein:
R₁, R₂, and R₃ can each independently be H or an amino acid residue having a side chain comprising an aromatic group;
at least one of R₁, R₂, and R₃ is an aromatic or heteroaromatic side chain of an amino acid;
R₄ and R₆ are independently H or an amino acid side chain;
AA_{SC} is an amino acid side chain;
q is 1, 2, 3 or 4;
   each m is independently an integer 0, 1, 2, or 3, and
Y is

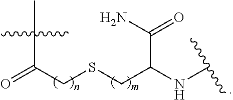

AA_{SC} can be conjugated to a linker.

### Linker

The cCPP of the disclosure can be conjugated to a linker. The linker can link an AC to the cCPP. The linker can be attached to the side chain of an amino acid of the cCPP, and the AC can be attached at a suitable position on linker.

The linker can be any appropriate moiety which can conjugate a cCPP to one or more additional moieties, e.g., an exocyclic peptide (EP) and/or an AC. Prior to conjugation to the cCPP and one or more additional moieties, the linker has two or more functional groups, each of which are independently capable of forming a covalent bond to the cCPP and one or more additional moieties. The linker can be covalently bound to the 5′ end of the AC or the 3′ end of the AC. The linker can be covalently bound to the 5′ end of the AC. The linker can be covalently bound to the 3′ end of the AC. The linker can be any appropriate moiety which conjugates a cCPP described herein to an AC.

The linker can comprise hydrocarbon linker.

The linker can comprise a cleavage site. The cleavage site can be a disulfide, or caspase-cleavage site (e.g, Val-Cit-PABC).

The linker can comprise: (i) one or more D or L amino acids, each of which is optionally substituted; (ii) optionally substituted alkylene; (iii) optionally substituted alkenylene; (iv) optionally substituted alkynylene; (v) optionally substituted carbocyclyl; (vi) optionally substituted heterocyclyl; (vii) one or more —(R¹-J-R²)z″— subunits, wherein each of R¹ and R², at each instance, are independently selected from alkylene, alkenylene, alkynylene, carbocyclyl, and heterocyclyl, each J is independently C, NR³, —NR³C(O)—, S, and O, wherein R³ is independently selected from H, alkyl, alkenyl, alkynyl, carbocyclyl, and heterocyclyl, each of which is optionally substituted, and z″ is an integer from 1 to 50; (viii) —(R¹-J)z″- or -(J-R¹)z″-, wherein each of R¹, at each instance, is independently alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ is an integer from 1 to 50; or (ix) the linker can comprise one or more of (i) through (x).

The linker can comprise one or more D or L amino acids and/or —(R¹-J-R²)z″-, wherein each of R¹ and R², at each instance, are independently alkylene, each J is independently C, NR³, —NR³C(O)—, S, and O, wherein R⁴ is independently selected from H and alkyl, and z″ is an integer from 1 to 50; or combinations thereof.

The linker can comprise a —(OCH₂CH₂)_{z′}— (e.g., as a spacer), wherein z′ is an integer from 1 to 23, e.g., 2, 3, 4, 5, 6, 7, 8, 9, 10, 11, 12, 13, 14, 15, 16, 17, 18, 19, 20, 21, 22, or 23. “—(OCH₂CH₂) z′ can also be referred to as polyethylene glycol (PEG).

The linker can comprise one or more amino acids. The linker can comprise a peptide. The linker can comprise a —(OCH₂CH₂)_{z′}—, wherein z′ is an integer from 1 to 23, and a peptide. The peptide can comprise from 2 to 10 amino acids. The linker can further comprise a functional group (FG) capable of reacting through click chemistry. FG can be an azide or alkyne, and a triazole is formed when the AC is conjugated to the linker.

The linker can comprise (i) a R alanine residue and lysine residue; (ii) -(J-R¹)z″; or (iii) a combination thereof. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O.

The linker can comprise (i) residues of β-alanine, glycine, lysine, 4-aminobutyric acid, 5-aminopentanoic acid, 6-aminohexanoic acid or combinations thereof; and (ii) —(R¹-J)z″- or -(J-R¹)z″. Each R¹ can independently be alkylene, alkenylene, alkynylene, carbocyclyl, or heterocyclyl, each J is independently C, NR³, —NR³C(O)—, S, or O, wherein R³ is H, alkyl, alkenyl, alkynyl, carbocyclyl, or heterocyclyl, each of which is optionally substituted, and z″ can be an integer from 1 to 50. Each R¹ can be alkylene and each J can be O. The linker can comprise glycine, beta-alanine, 4-aminobutyric acid, 5-aminopentanoic acid, 6-aminohexanoic acid, or a combination thereof.

The linker can be a trivalent linker. The linker can have the structure:

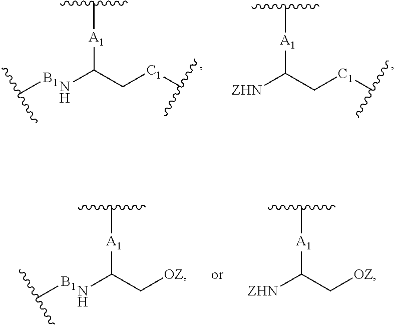

wherein A₁, B₁, and C₁, can independently be a hydrocarbon linker (e.g., NRH—(CH₂)ₙ—COOH), a PEG linker (e.g., NRH—(CH₂O)ₙ—COOH, wherein R is H, methyl or ethyl) or one or more amino acid residue, and Z is independently a protecting group. The linker can also incorporate a cleavage site, including a disulfide [NH₂—(CH₂O)ₙ—S—S—(CH₂O)ₙ—COOH], or caspase-cleavage site (Val-Cit-PABC).

The hydrocarbon can be a residue of glycine or beta-alanine.

The linker can be bivalent and link the cCPP to an AC. The linker can be bivalent and link the cCPP to an exocyclic peptide (EP).

The linker can be trivalent and link the cCPP to an AC and to an EP.

The linker can be a bivalent or trivalent C₁-C₅₀ alkylene, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heterocyclyl, heteroaryl, cycloalkyl, or cycloalkenyl. The linker can be a bivalent or trivalent C₁-C₅₀ alkylene, wherein 1-25 methylene groups are optionally and independently replaced by —N(H)—, —O—, —C(O)N(H)—, or a combination thereof.

The AC can be coupled to the glutamic acid of the cyclic peptide, which converts the glutamic acid to glutamine. The linker (L) can couple the AC to the glutamine/glutamic acid of the cyclic peptide. In embodiments, a linker (L) is covalently bound to the backbone of the AC.

The linker can have the structure:

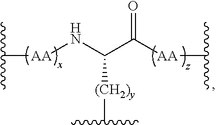

wherein: each AA is independently an amino acid residue; * is the point of attachment to the AA_{SC}, and AA_{SC} is side chain of an amino acid residue of the cCPP; x is an integer from 1-10; y is an integer from 1-5; and z is an integer from 1-10. x can be an integer from 1-5. x can be an integer from 1-3. x can be 1. y can be an integer from 2-4. y can be 4. z can be an integer from 1-5. z can be an integer from 1-3. z can be 1. Each AA can independently be selected from glycine, β-alanine, 4-aminobutyric acid, 5-aminopentanoic acid, and 6-aminohexanoic acid.

The cCPP can be attached to the AC through a linker (“L”). The linker can be conjugated to the AC through a bonding group (“M”).

The linker can have the structure:

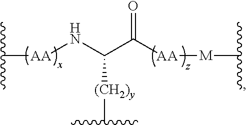

wherein: x is an integer from 1-10; y is an integer from 1-5; z is an integer from 1-10; each AA is independently an amino acid residue; * is the point of attachment to the AA_{SC}, and AA_{SC} is side chain of an amino acid residue of the cCPP; and M is a bonding group defined herein.

The linker can have the structure:

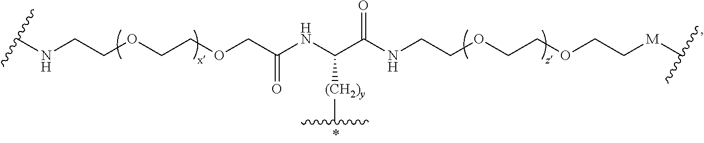

wherein: x′ is an integer from 1-23; y is an integer from 1-5; z′ is an integer from 1-23; * is the point of attachment to the AA_{SC}, and AA_{SC} is a side chain of an amino acid residue of the cCPP; and M is a bonding group defined herein.

The linker can have the structure:

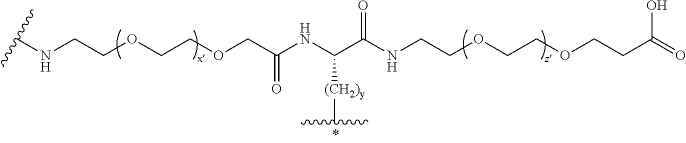

wherein: x′ is an integer from 1-23; y is an integer from 1-5; and z′ is an integer from 1-23; * is the point of attachment to the AA_{SC}, and AA_{SC} is a side chain of an amino acid residue of the cCPP.

x can be an integer from 1-10, e.g., 1, 2, 3, 4, 5, 6, 7, 8, 9, or 10, inclusive of all ranges and subranges therebetween.

x′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. x′ can be an integer from 5-15. x′ can be an integer from 9-13. x′ can be an integer from 1-5. x′ can be 1.

y can be an integer from 1-5, e.g., 1, 2, 3, 4, or 5, inclusive of all ranges and subranges therebetween. y can be an integer from 2-5. y can be an integer from 3-5. y can be 3 or 4. y can be 4 or 5. y can be 3. y can be 4. y can be 5.

z can be an integer from 1-10, e.g., 1, 2, 3, 4, 5, 6, 7, 8, 9, or 10, inclusive of all ranges and subranges therebetween.

z′ can be an integer from 1-23, e.g., 1, 2, 3, 4, 5, 6, 7, 8, 9, 10, 11, 12, 13, 14, 15, 16, 17, 18, 19, 20, 21, 22, or 23, inclusive of all ranges and subranges therebetween. z′ can be an integer from 5-15. z′ can be an integer from 9-13. z′ can be 11.

As discussed above, the linker or M (wherein M is part of the linker) can be covalently bound to AC at any suitable location on the AC. The linker or M (wherein M is part of the linker) can be covalently bound to the 3′ end of the AC or the 5′ end of the AC. The linker or M (wherein M is part of the linker) can be covalently bound to the backbone of an AC.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

The linker can have a structure:

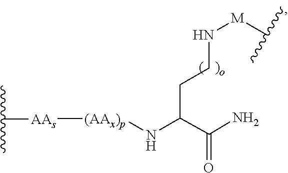

wherein
M is a group that conjugates L to an AC;
AAₛ is a side chain or terminus of an amino acid on the cCPP;
each AAₓ is independently an amino acid residue;
o is an integer from 0 to 10; and
p is an integer from 0 to 5.

The linker can have a structure:

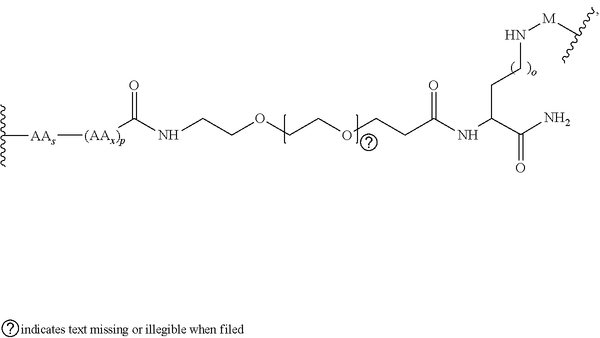

wherein
M is a group that conjugates L to an AC;
AAₛ is a side chain or terminus of an amino acid on the cCPP;
each AAₓ is independently an amino acid residue;
o is an integer from 0 to 10; and
p is an integer from 0 to 5.

M can comprise an alkylene, alkenylene, alkynylene, carbocyclyl, or heterocyclyl, each of which is optionally substituted. M can be selected from:

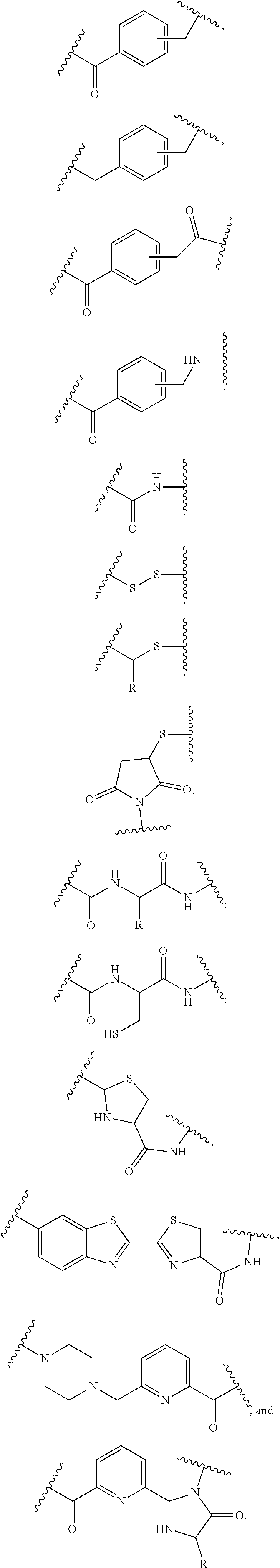

wherein R is alkyl, alkenyl, alkynyl, carbocyclyl, or heterocyclyl.

M can be selected from:

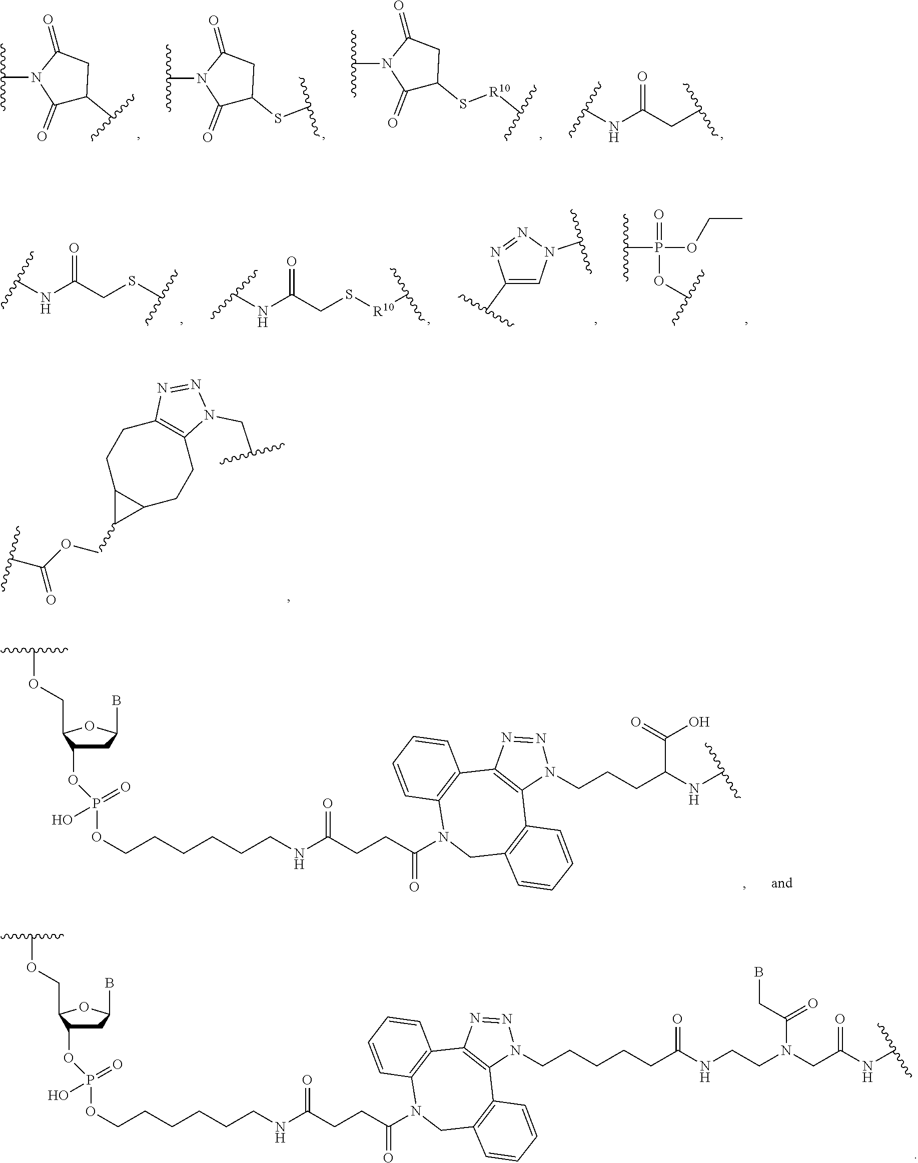

wherein: R¹⁰ is alkylene, cycloalkyl, or

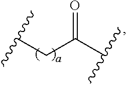

wherein a is 0 to 10.

M can be

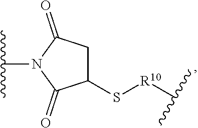

### R¹⁰ can be

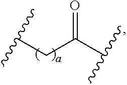

### and a is 0 to 10. M can be

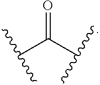 [SMILES: *C(*)=O]

M can be a heterobifunctional crosslinker, e.g.,

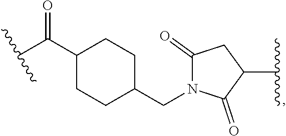 [SMILES: *C(=O)C1CCC(CN2C(=O)CC(*)C2=O)CC1]

1 which is disclosed in Williams et al. Curr. Protoc Nucleic Acid Chem. 2010, 42, 4.41.1-4.41.20, incorporated herein by reference its entirety.

M can be —C(O)—.

AAₛ can be a side chain or terminus of an amino acid on the cCPP. Non-limiting examples of AAₛ include aspartic acid, glutamic acid, glutamine, asparagine, or lysine, or a modified side chain of glutamine or asparagine (e.g., a reduced side chain having an amino group). AAₛ can be an AA_{SC} as defined herein.

Each AAₓ is independently a natural or non-natural amino acid. One or more AAₓ can be a natural amino acid. One or more AAₓ can be a non-natural amino acid. One or more AAₓ can be a β-amino acid. The β-amino acid can be β-alanine.

o can be an integer from 0 to 10, e.g., 0, 1, 2, 3, 4, 5, 6, 7, 8, 9, and 10. o can be 0, 1, 2, or 3. o can be 0. o can be 1. o can be 2. o can be 3.

p can be 0 to 5, e.g., 0, 1, 2, 3, 4, or 5. p can be 0. p can be 1. p can be 2. p can be 3. p can be 4. p can be 5.

The linker can have the structure:

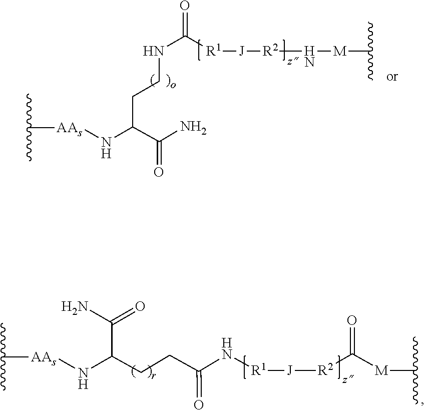

wherein M, AAₛ, each —(R¹-J-R²)z″-, o and z″ are defined herein; r can be 0 or 1.

r can be 0. r can be 1.

The linker can have the structure:

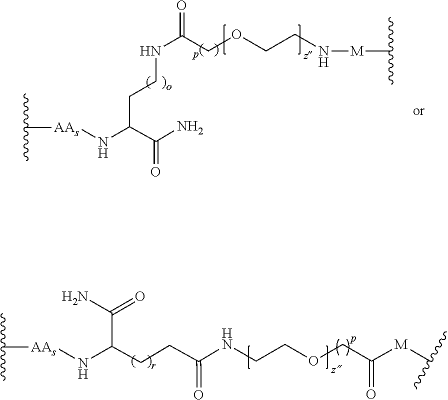

wherein each of M, AAₛ, o, p, q, r and z″ can be as defined herein.

z″ can be an integer from 1 to 50, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50, inclusive of all ranges and values therebetween. z″ can be an integer from 5-20. z″ can be an integer from 10-15.

The linker can have the structure:

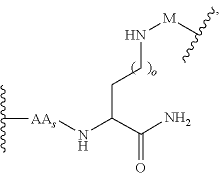

wherein:
M, AAₛ and o are as defined herein.

Other non-limiting examples of suitable linkers include:

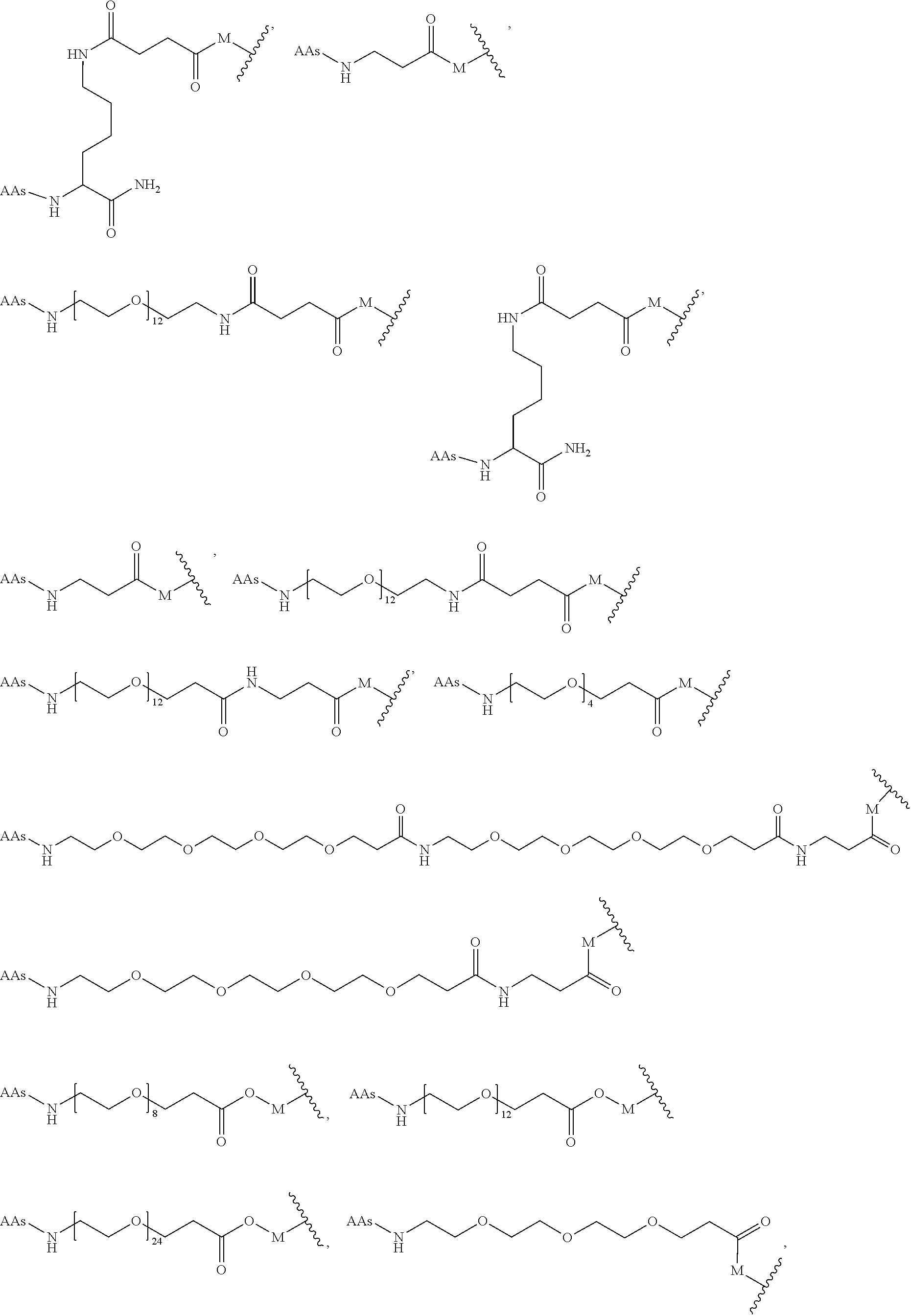

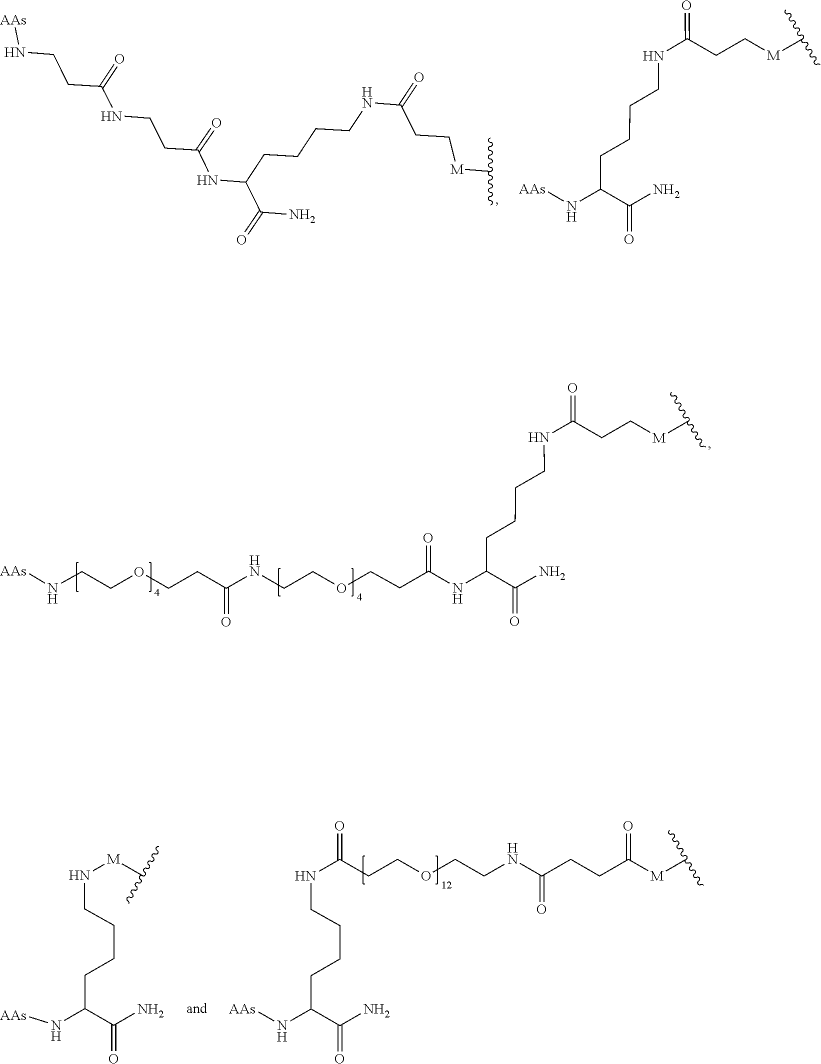

wherein M and AAₛ are as defined herein.

Provided herein is a compound comprising a cCPP and an AC that is complementary to a target in a pre-mRNA sequence further comprising L, wherein the linker is conjugated to the AC through a bonding group (M), wherein M is

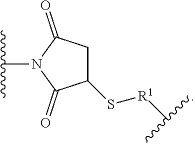

Provided herein is a compound comprising a cCPP and an antisense compound (AC), for example, an antisense oligonucleotide, that is complementary to a target in a pre-mRNA sequence, wherein the compound further comprises L, wherein the linker is conjugated to the AC through a bonding group (M), wherein M is selected from:

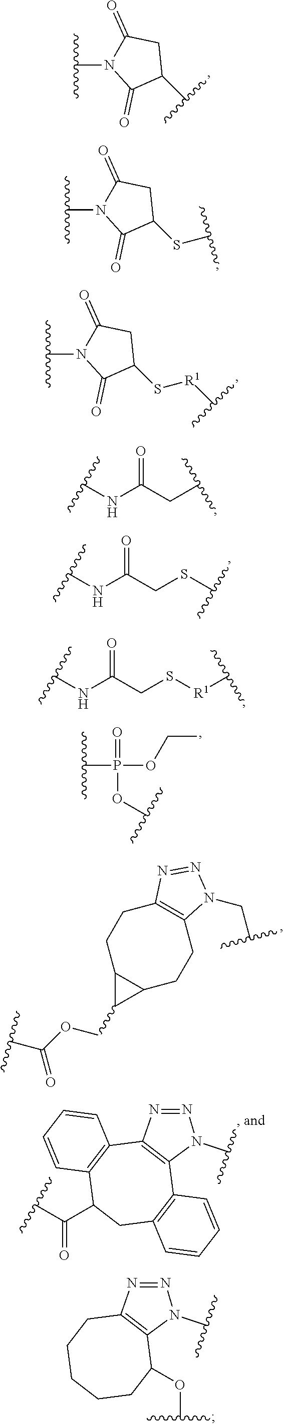

wherein: R¹ is alkylene, cycloalkyl, or

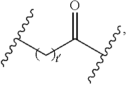

wherein t′ is 0 to 10 wherein each R is independently an alkyl, alkenyl, alkynyl, carbocyclyl, or heterocyclyl, wherein R¹ is

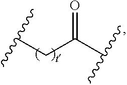

and t′ is 2.

The linker can have the structure:

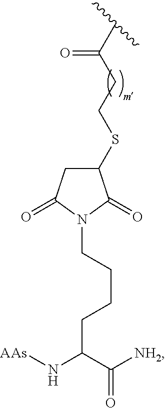

wherein AAₛ is as defined herein, and m′ is 0-10.

The linker can be of the formula:

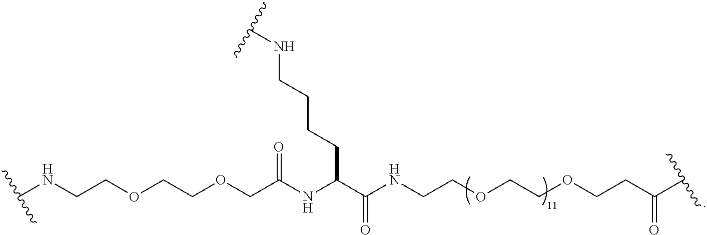

The linker can be of the formula:

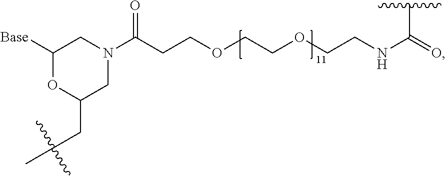

wherein “base” corresponds to a nucleobase at the 3′ end of a phosphorodiamidate morpholino oligomer.

The linker can be of the formula:

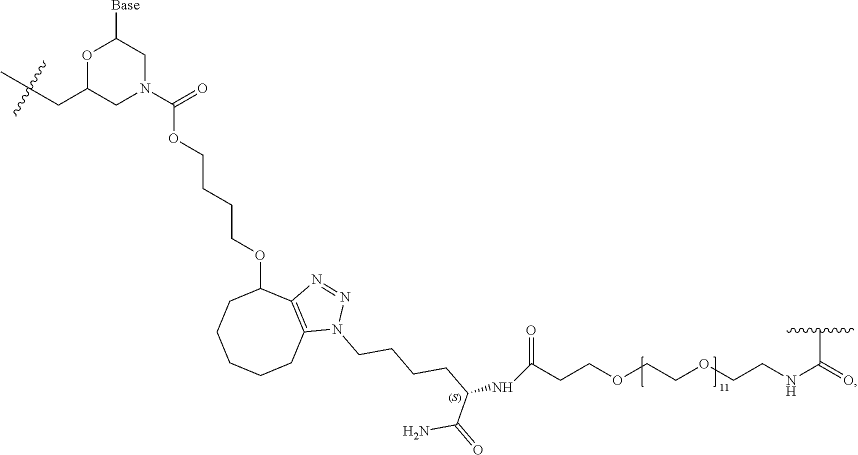

wherein “base” corresponds to a nucleobase at the 3′ end of a phosphorodiamidate morpholino oligomer.

The linker can be of the formula:

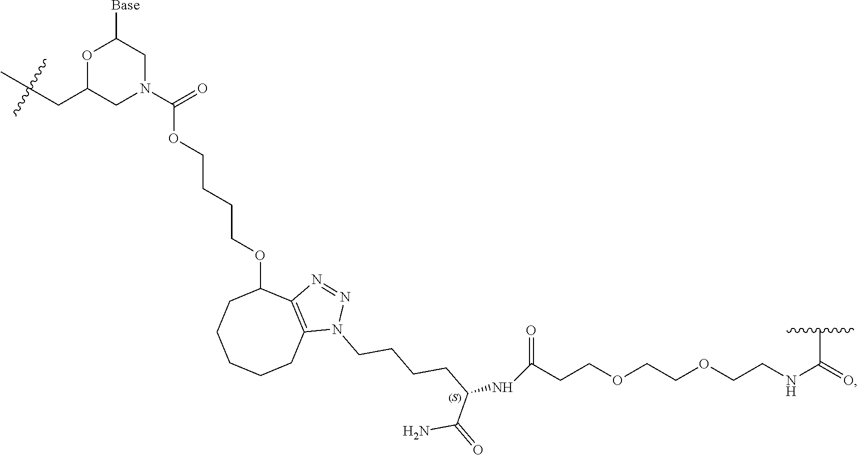

wherein “base” corresponds to a nucleobase at the 3′ end of a cargo phosphorodiamidate morpholino oligomer.

The linker can be of the formula:

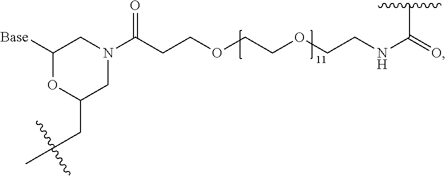

wherein “base” corresponds to a nucleobase at the 3′ end of a cargo phosphorodiamidate morpholino oligomer.

The linker can be of the formula:

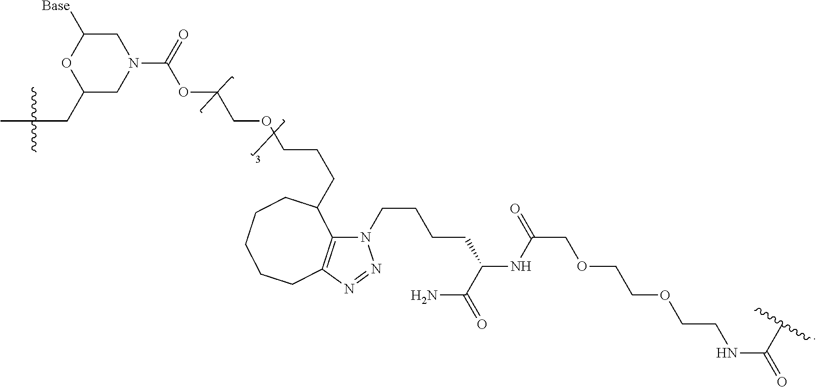

The linker can be covalently bound to a cargo at any suitable location on the AC. The linker is covalently bound to the 3′ end of cargo or the 5′ end of an AC. The linker can be covalently bound to the backbone of an AC.

The linker can be bound to the side chain of aspartic acid, glutamic acid, glutamine, asparagine, or lysine, or a modified side chain of glutamine or asparagine (e.g., a reduced side chain having an amino group), on the cCPP. The linker can be bound to the side chain of lysine on the cCPP.

cCPP-Linker Conjugates

The cCPP can be conjugated to a linker defined herein. The linker can be conjugated to an AA_{SC} of the cCPP as defined herein.

The linker can comprise a —(OCH₂CH₂)_{z′}— subunit (e.g., as a spacer), wherein z′ is an integer from 1 to 23, e.g., 1, 2, 3, 4, 5, 6, 7, 8, 9, 10, 11, 12, 13, 14, 15, 16, 17, 18, 19, 20, 21, 22 or 23. “—(OCH₂CH₂)_{z′} is also referred to as PEG. The cCPP-linker conjugate can have a structure selected from Table 4:

TABLE 4
cCPP-linker conjugates

cyclo(FfΦ-4gp-r-4gp-rQ)-PEG₄-K—NH₂
cyclo(FfΦ-Cit-r-Cit-rQ)-PEG₄-K—NH₂
cyclo(FfΦ-Pia-r-Pia-rQ)-PEG₄-K—NH₂
cyclo(FfΦ-Dml-r-Dml -rQ)-PEG₄-K—NH₂
cyclo(FfΦ-Cit-r-Cit-rQ)-PEG₁₂-OH
cyclo(fΦR-Cit-R-Cit-Q)-PEG₁₂-OH

The linker can comprise a —(OCH₂CH₂)_{z′}— subunit, wherein z′ is an integer from 1 to 23, and a peptide subunit. The peptide subunit can comprise from 2 to 10 amino acids. The cCPP-linker conjugate can have a structure selected from Table 5:

TABLE 5
Endosomal Escape Vehicle (cCPP-linker conjugate)

Ac-PKKKRKV-Lys(cyclo[FfΦ-R-r-Cit-rQ])-PEG₁₂-K(N₃)-NH₂
Ac-PKKKRKV-Lys(cyclo[FfΦ-Cit-r-R-rQ])-PEG₁₂-K(N₃)-NH₂
Ac-PKKKRKV-K(cyclo(FfΦR-cit-R-cit-Q))-PEG₁₂-K(N₃)-NH₂
Ac-PKKKRKV-PEG2-Lys(cyclo[FfΦ-Cit-r-Cit-rQ])-B-k(N₃)-NH₂
Ac-PKKKRKV-PEG2-Lys(cyclo[FfΦ-Cit-r-Cit-rQ])-PEG2-k(N₃)-NH₂
Ac-PKKKRKV-PEG2-Lys(cyclo[FfΦ-Cit-r-Cit-rQ])-PEG4-k(N₃)-NH₂
Ac-PKKKRKV-Lys(cyclo[FfΦ-Cit-r-Cit-rQ])-PEG12-k(N₃)-NH₂
Ac-pkkkrkv-PEG2-Lys(cyclo[FfΦ-Cit-r-Cit-rQ])-PEG12-k(N₃)-NH₂
Ac-rrv-PEG2-Lys(cyclo[FfΦ-Cit-r-Cit-rQ])-PEG12-OH
Ac-PKKKRKV-PEG2-Lys(cyclo[FfΦ-Cit-r-Cit-r-Q])-PEG12-k(N₃)-NH₂
Ac-PKKK-Cit-KV-PEG2-Lys(cyclo[FfΦ-Cit-r-Cit-r-Q])-PEG12-k(N₃)-NH₂
Ac-PKKKRKV-PEG2-Lys(cyclo[FfΦ-Cit-r-Cit-r-Q]-PEG12-K(N₃)-NH₂

The cCPP-linker conjugate can be Ac-PKKKRKV-K(cyclo[FfφDGrGrQ])-PEG12-K(N₃)—NH₂.

EEVs comprising a cyclic cell penetrating peptide (cCPP), linker and exocyclic peptide (EP) are provided. An EEV can comprise the structure of Formula (B):

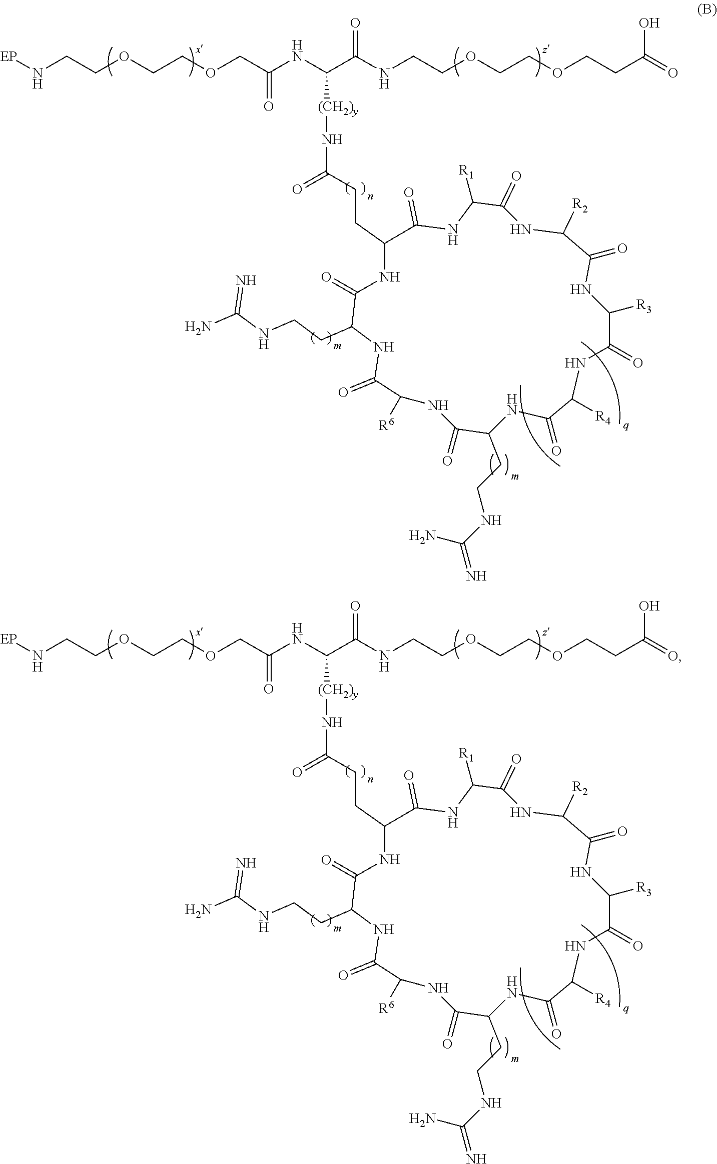

or a protonated form thereof,
wherein:
R₁, R₂, and R₃ are each independently H or an aromatic or heteroaromatic side chain of an amino acid;
R₄ and R₆ are independently H or an amino acid side chain;
EP is an exocyclic peptide as defined herein;
each m is independently an integer from 0-3;
n is an integer from 0-2;
x′ is an integer from 1-20;
y is an integer from 1-5;
q is 1-4; and
z′ is an integer from 1-23.

R₁, R₂, R₃, R₄, R₆, EP, m, q, y, x′, z′ are as described herein.

n can be 0. n can be 1. n can be 2.

The EEV can comprise the structure of Formula (B-a) or (B-b):

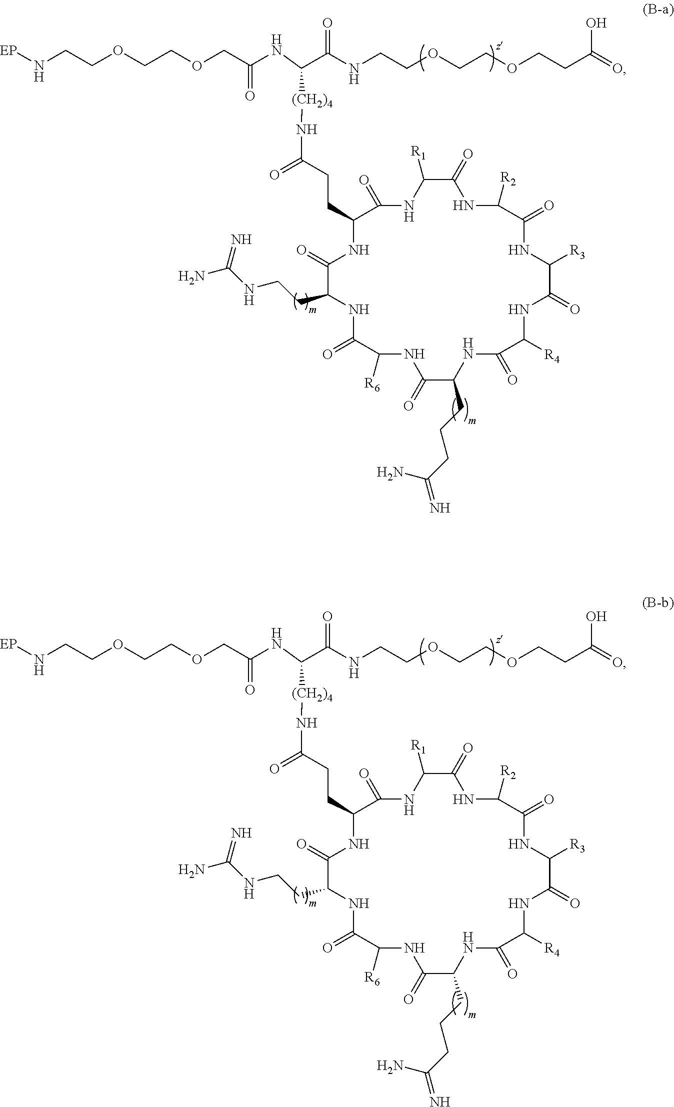

) or a protonated form thereof, wherein EP, R¹, R², R³, R⁴, m and z′ are as defined above in Formula (B).

The EEV can comprises the structure of Formula (B-c):

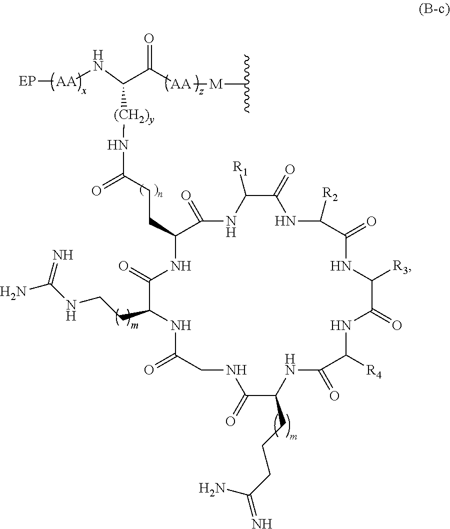

or a protonated form thereof, wherein EP, R¹, R², R³, R⁴, and m are as defined above in Formula (B); AA is an amino acid as defined herein; M is as defined herein; n is an integer from 0-2; x is an integer from 1-10; y is an integer from 1-5; and z is an integer from 1-10.

The EEV can have the structure of Formula (B-1), (B-2), (B-3), or (B-4):

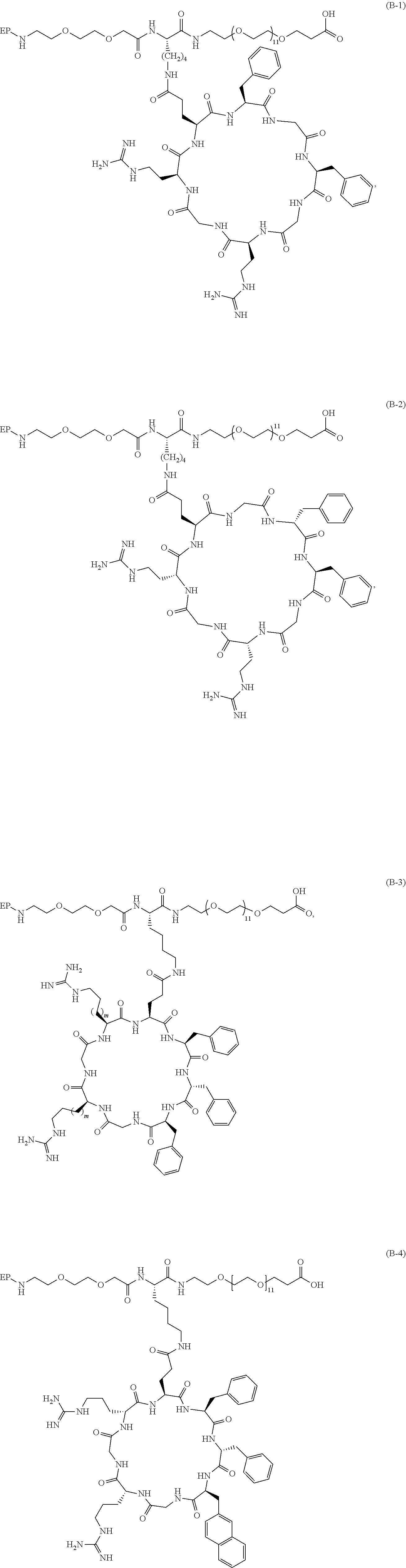

or a protonated form thereof, wherein EP is as defined above in Formula (B).

The EEV can comprise Formula (B) and can have the structure: Ac-PKKKRKV-AEEA-K(cyclo[FGFGRGRQ])-PEG₁₂-OH or Ac-PKKKRKV-AEEA-K(cyclo[GfFGrGrQ])-PEG₁₂-OH.

The EEV can comprise a cCPP of formula:

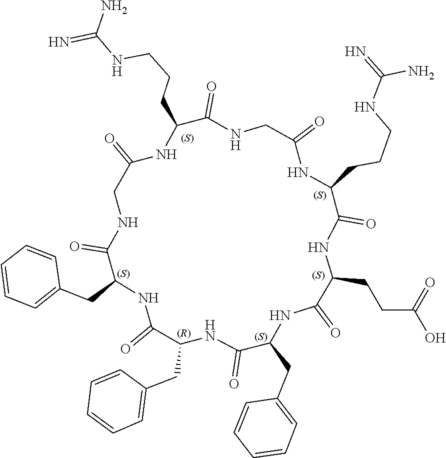

The EEV can comprise formula: Ac-PKKKRKV-miniPEG2-Lys(cyclo(FfFGRGRQ)-miniPEG2-K(N3).

The EEV can be:

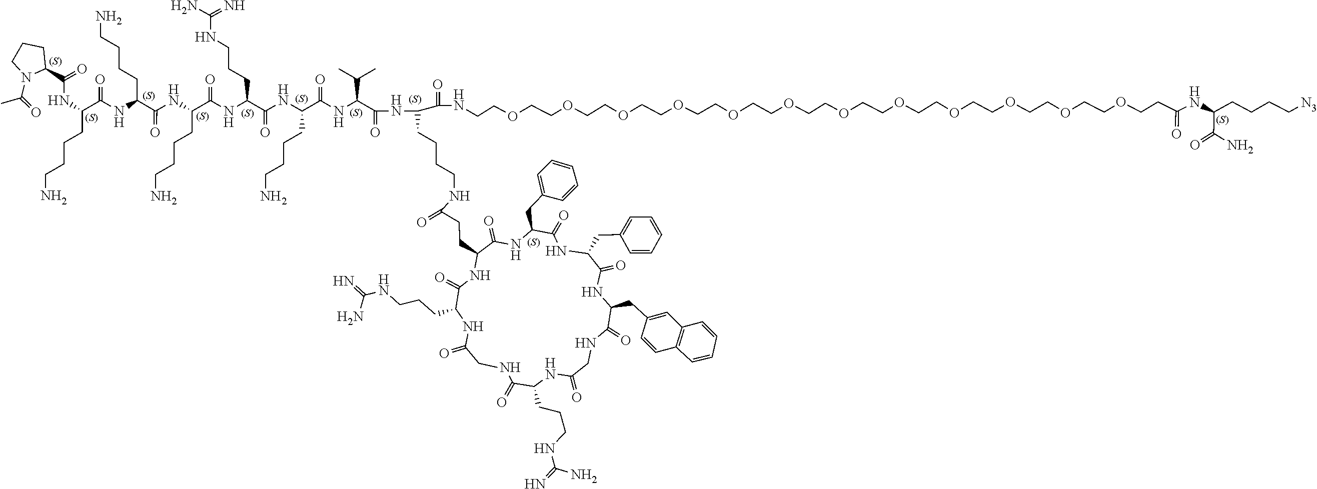

The EEV can be: Ac-PKKKRKV-K(cyclo(Ff-Nal-GrGrQ)-PEG₁₂-K(N₃)—NH₂.

The EEV can be

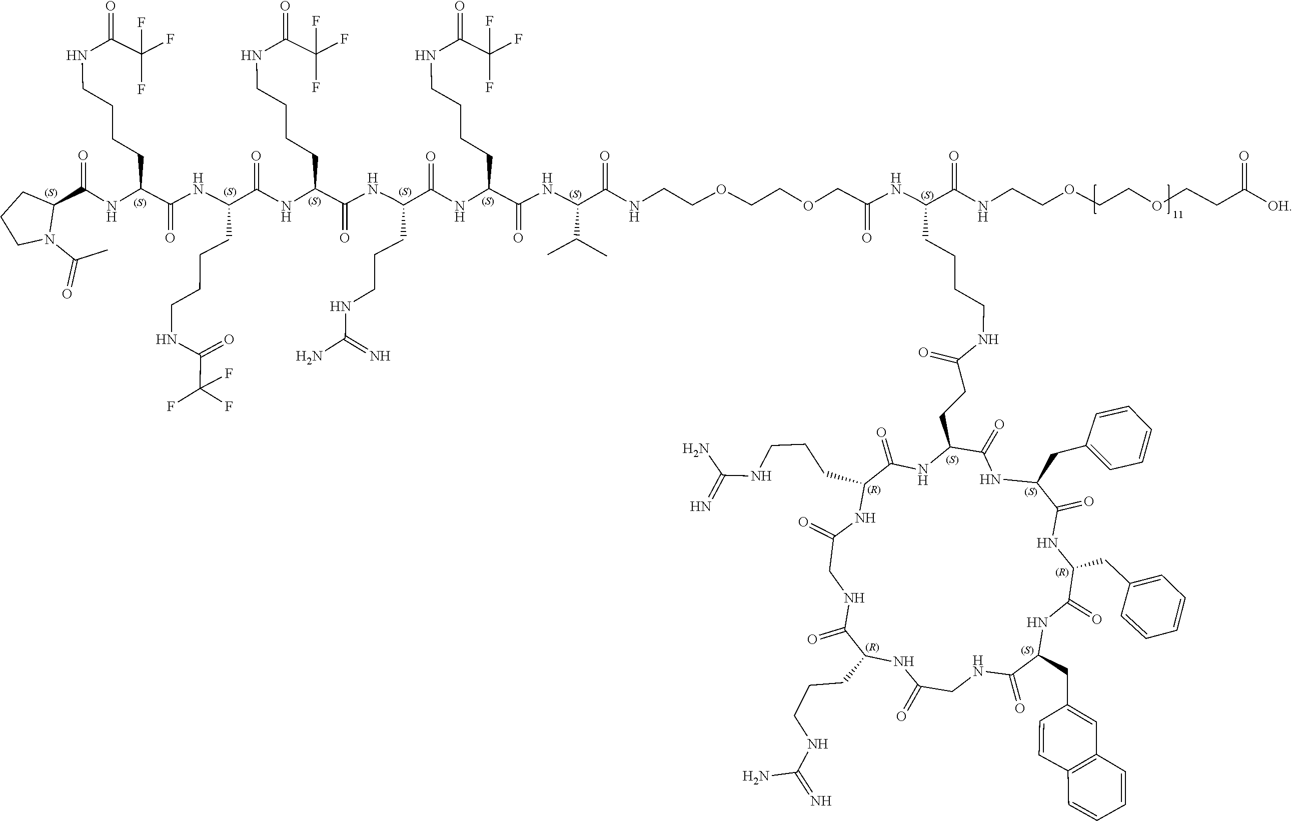

The EEV can be Ac-P-K(Tfa)-K(Tfa)-K(Tfa)-R-K(Tfa)-V-AEEA-K(cyclo(Ff-Nal-GrGrQ)-PEG₁₂-OH or Ac-P-K(Tfa)-K(Tfa)-K(Tfa)-R-K(Tfa)-V-AEEA-K(cyclo(FGFGRGRQ)-PEG₁₂-OH.

The EEV can be

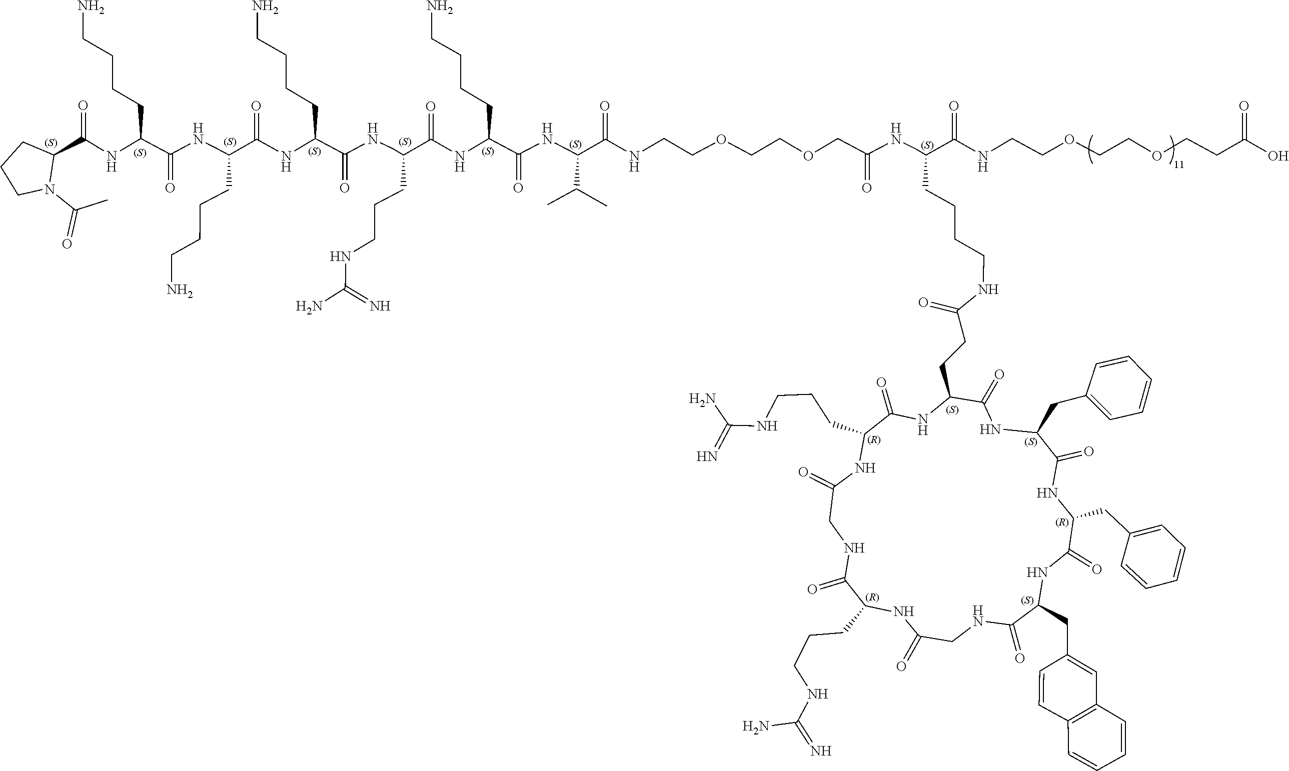

The EEV can be Ac-PKKKRKV-miniPEG-K(cyclo(Ff-Nal-GrGrQ)-PEG12-OH.

The EEV can be

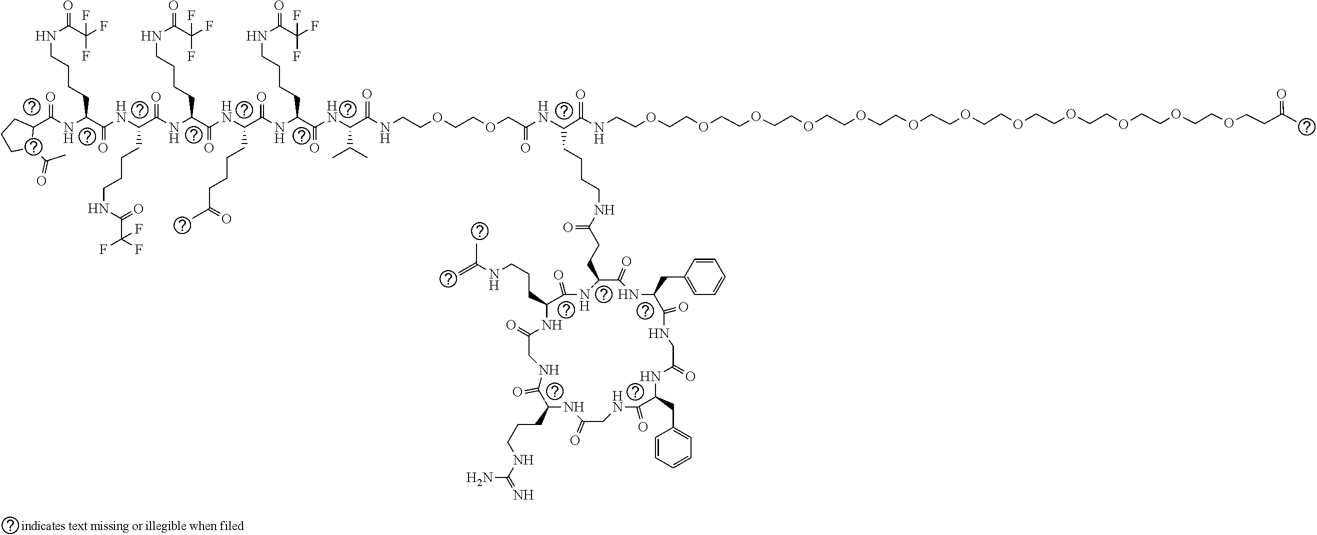

The EEV can be

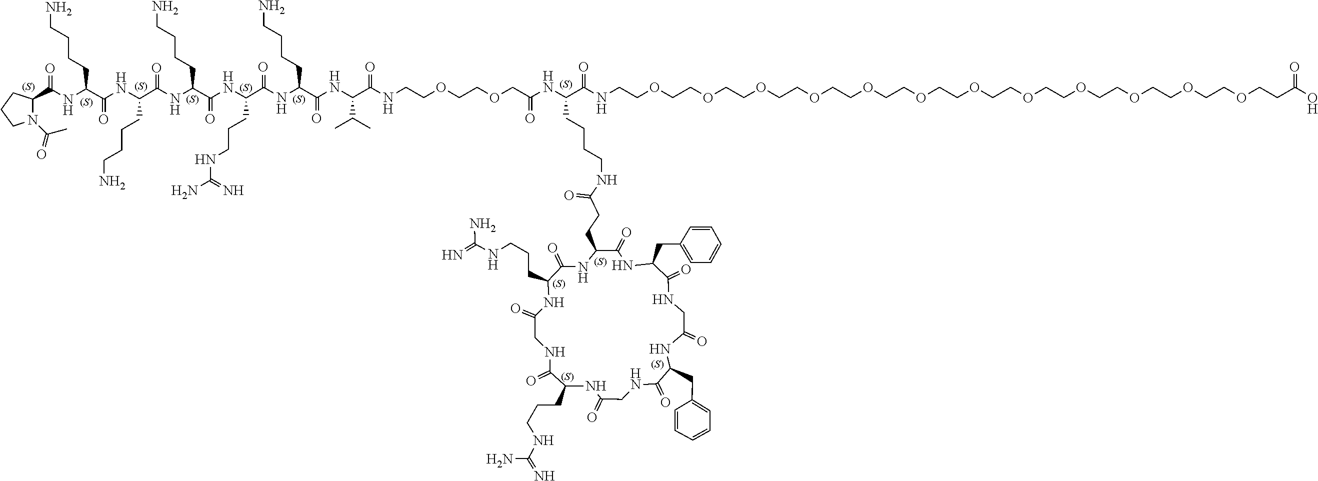

The EEV can be

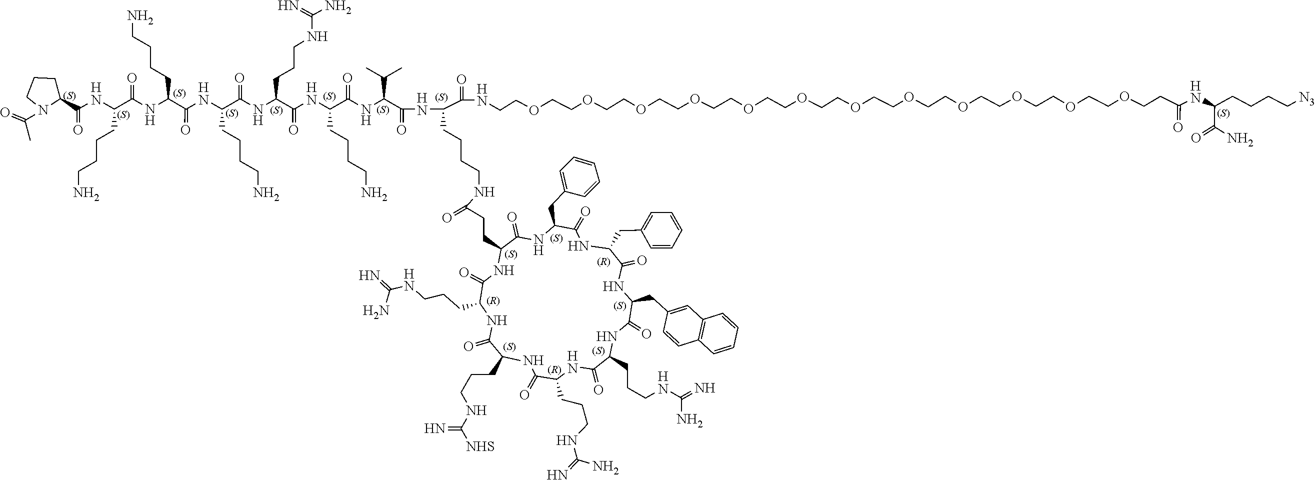

The EEV can be

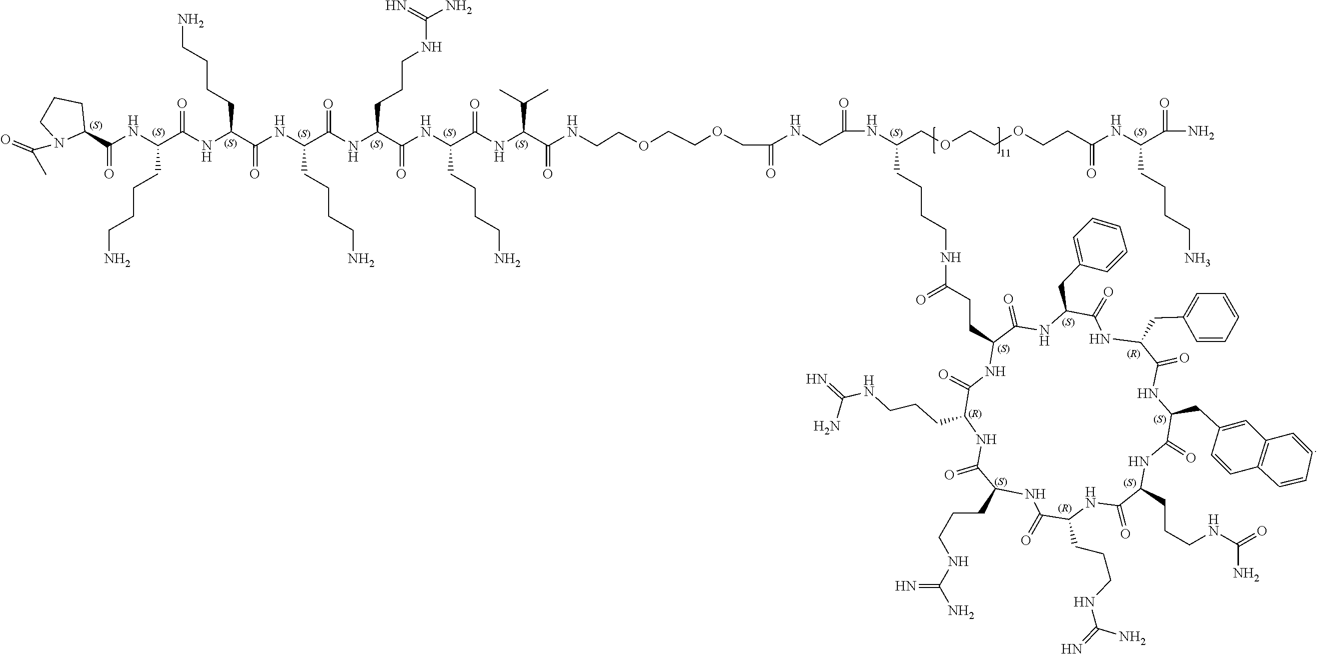

The EEV can be

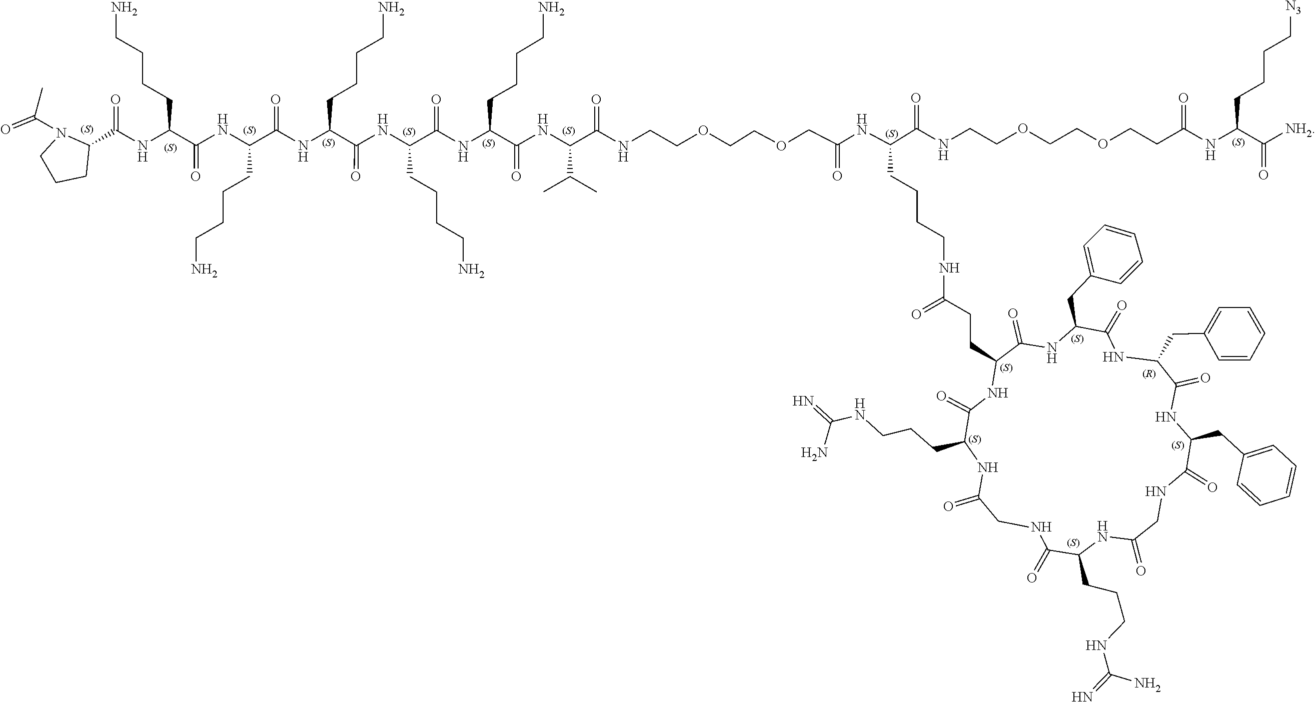

The EEV can be

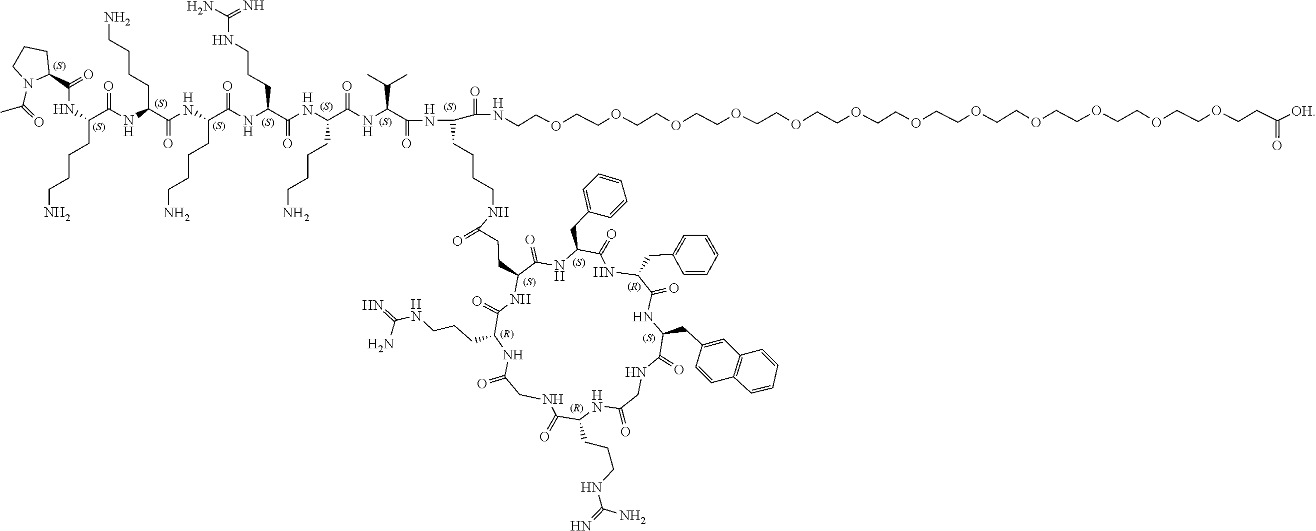

The EEV can be

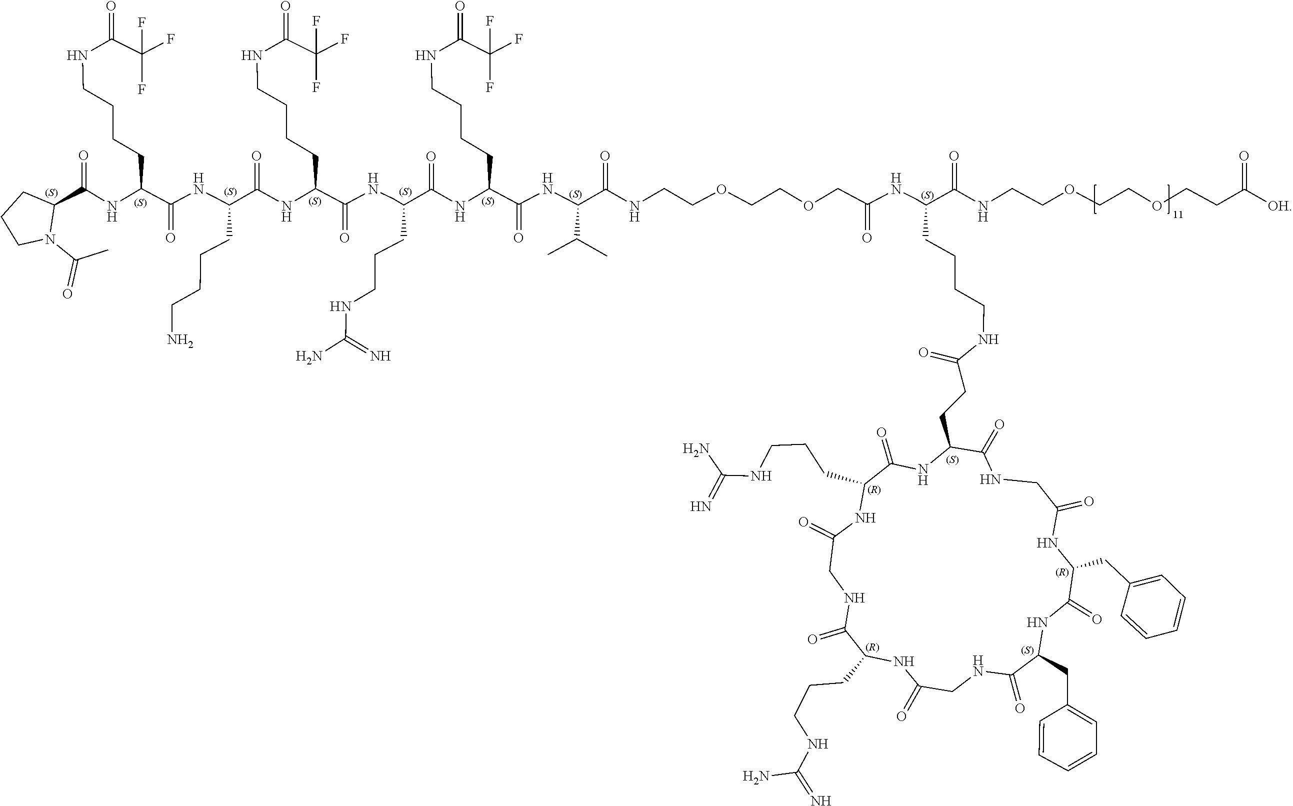

The EEV can be

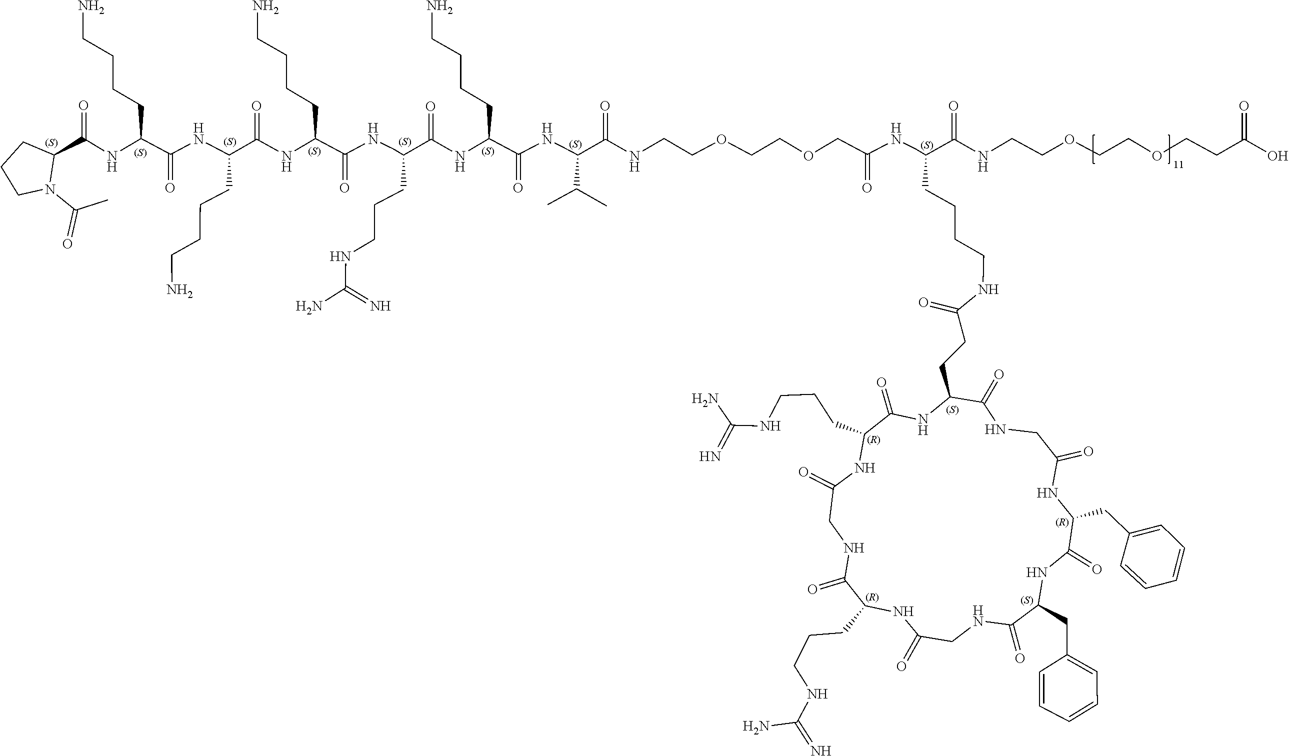

The EEV can be

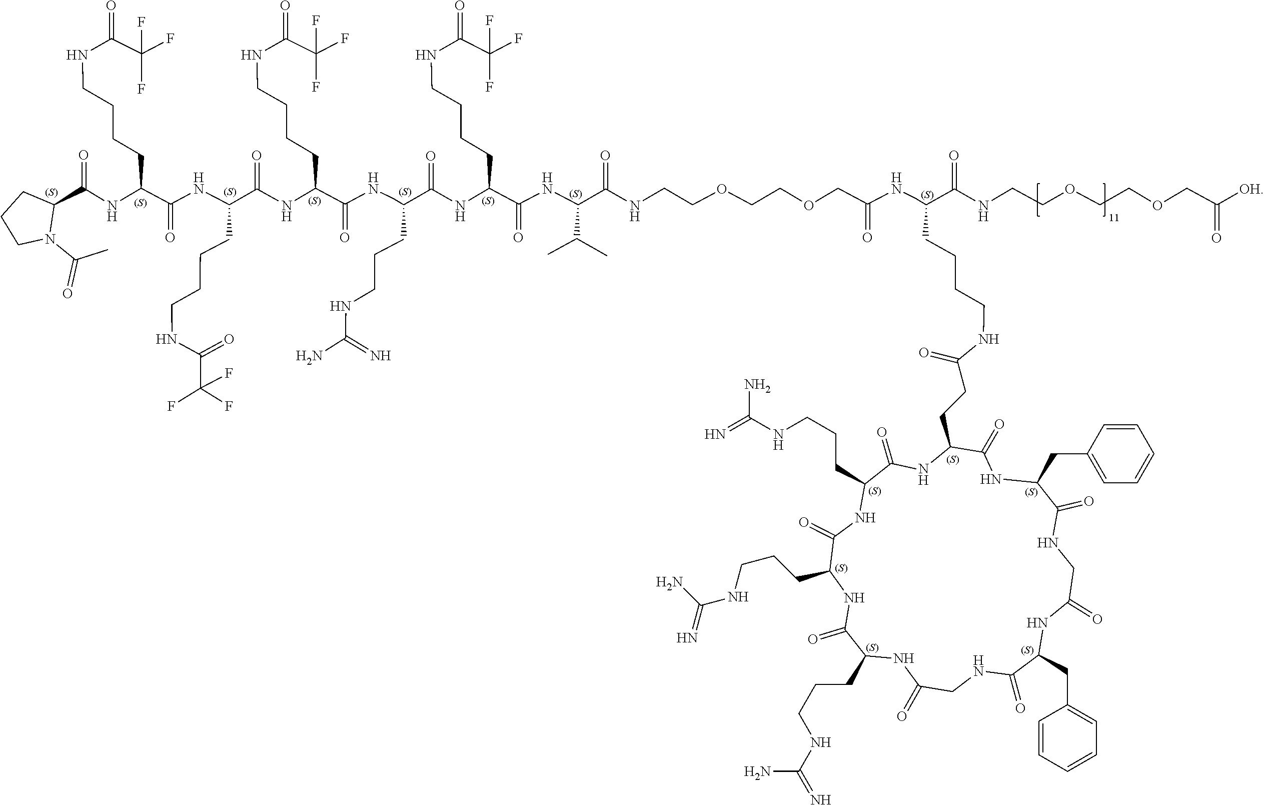

The EEV can be

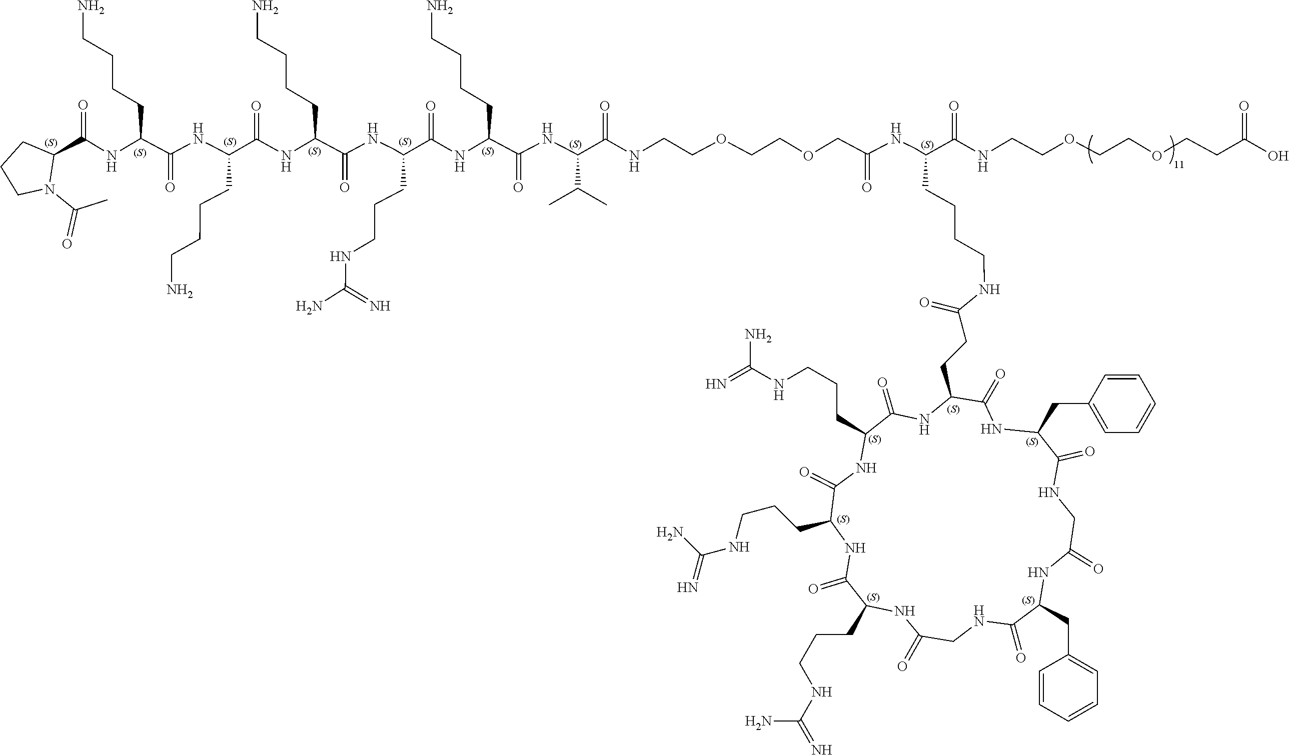

The EEV can be

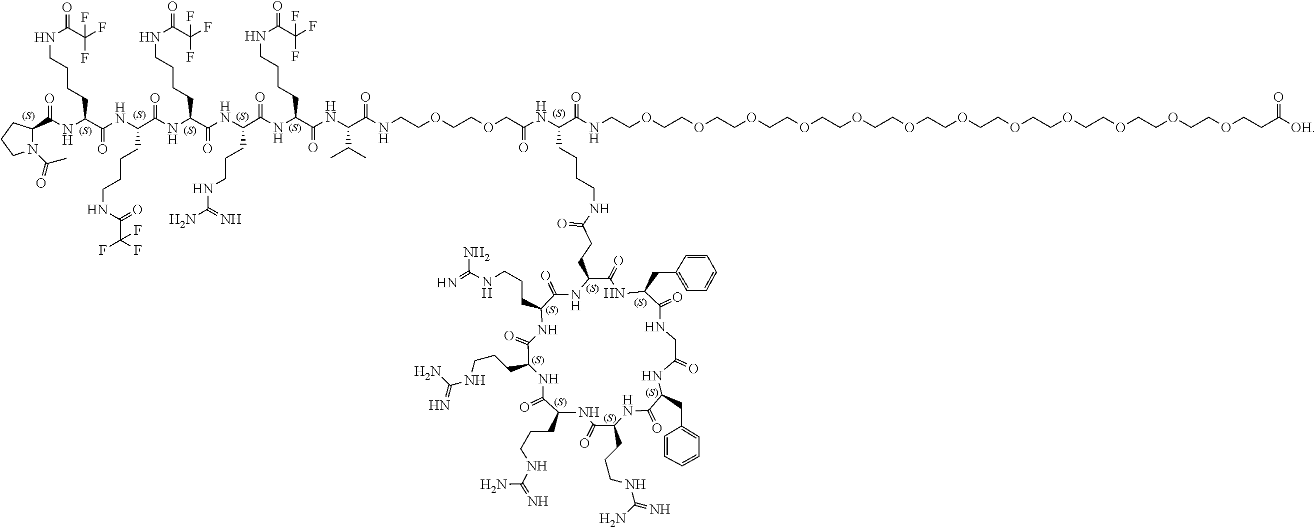

The EEV can be selected from
Ac-rr-miniPEG2-Dap[cyclo(FfΦ-Cit-r-Cit-rQ)]-PEG12-OH
Ac-frr-PEG2-Dap(cyclo(FfΦ-Cit-r-Cit-rQ))-PEG12-OH
Ac-rfr-PEG2-Dap(cyclo(FfΦ-Cit-r-Cit-rQ))-PEG12-OH
Ac-rbfbr-PEG2-Dap(cyclo(FfΦ-Cit-r-Cit-rQ))-PEG12-OH
Ac-rrr-PEG2-Dap(cyclo(FfΦ-Cit-r-Cit-rQ))-PEG12-OH
Ac-rbr-PEG2-Dap(cyclo(FfΦ-Cit-r-Cit-rQ))-PEG12-OH
Ac-rbrbr-PEG2-Dap(cyclo(FfΦ-Cit-r-Cit-rQ))-PEG12-OH
Ac-hh-PEG2-Dap(cyclo(FfΦ-Cit-r-Cit-rQ))-PEG12-OH
Ac-hbh-PEG2-Dap(cyclo(FfΦ-Cit-r-Cit-rQ))-PEG12-OH
Ac-hbhbh-PEG2-Dap(cyclo(FfΦ-Cit-r-Cit-rQ))-PEG12-OH
Ac-rbhbh-PEG2-Dap(cyclo(FfΦ-Cit-r-Cit-rQ))-PEG12-OH
Ac-hbrbh-PEG2-Dap(cyclo(FfΦ-Cit-r-Cit-rQ))-PEG12-OH
Ac-rr-Dap(cyclo(FfΦ-Cit-r-Cit-rQ))-b-OH
Ac-frr-Dap(cyclo(FfΦ-Cit-r-Cit-rQ))-b-OH
Ac-rfr-Dap(cyclo(FfΦ-Cit-r-Cit-rQ))-b-OH
Ac-rbfbr-Dap(cyclo(FfΦ-Cit-r-Cit-rQ))-b-OH
Ac-rrr-Dap(cyclo(FfΦ-Cit-r-Cit-rQ))-b-OH
Ac-rbr-Dap(cyclo(FfΦ-Cit-r-Cit-rQ))-b-OH
Ac-rbrbr-Dap(cyclo(FfΦ-Cit-r-Cit-rQ))-b-OH
Ac-hh-Dap(cyclo(FfΦ-Cit-r-Cit-rQ))-b-OH
Ac-hbh-Dap(cyclo(FfΦ-Cit-r-Cit-rQ))-b-OH
Ac-hbhbh-Dap(cyclo(FfΦ-Cit-r-Cit-rQ))-b-OH
Ac-rbhbh-Dap(cyclo(FfΦ-Cit-r-Cit-rQ))-b-OH
Ac-hbrbh-Dap(cyclo(FfΦ-Cit-r-Cit-rQ))-b-OH
Ac-KKKK-miniPEG2-Lys(cyclo(Ff-Nal-GrGrQ))-miniPEG2-K(N3)-NH2
Ac-KGKK-miniPEG2-Lys(cyclo(Ff-Nal-GrGrQ))-miniPEG2-K(N3)-NH2
Ac-KKGK-miniPEG2-Lys(cyclo(Ff-Nal-GrGrQ))-miniPEG2-K(N3)-NH2
Ac-KKK-miniPEG2-Lys(cyclo(Ff-Nal-GrGrQ))-miniPEG2-K(N3)-NH2
Ac-KK-miniPEG2-Lys(cyclo(Ff-Nal-GrGrQ))-miniPEG2-K(N3)-NH2
Ac-KGK-miniPEG2-Lys(cyclo(Ff-Nal-GrGrQ))-miniPEG2-K(N3)-NH2
Ac-KBK-miniPEG2-Lys(cyclo(Ff-Nal-GrGrQ))-miniPEG2-K(N3)-NH2
Ac-KBKBK-miniPEG2-Lys(cyclo(Ff-Nal-GrGrQ))-miniPEG2-K(N3)-NH2
Ac-KR-miniPEG2-Lys(cyclo(Ff-Nal-GrGrQ))-miniPEG2-K(N3)-NH2
Ac-KBR-miniPEG2-Lys(cyclo(Ff-Nal-GrGrQ))-miniPEG2-K(N3)-NH2
Ac-PKKKRKV-miniPEG2-Lys(cyclo(Ff-Nal-GrGrQ))-miniPEG2-K(N3)-NH2
Ac-PKKKRKV-miniPEG2-Lys(cyclo(Ff-Nal-GrGrQ))-miniPEG2-K(N3)-NH2
Ac-PGKKRKV-miniPEG2-Lys(cyclo(Ff-Nal-GrGrQ))-miniPEG2-K(N3)-NH2
Ac-PKGKRKV-miniPEG2-Lys(cyclo(Ff-Nal-GrGrQ))-miniPEG2-K(N3)-NH2
Ac-PKKGRKV-miniPEG2-Lys(cyclo(Ff-Nal-GrGrQ))-miniPEG2-K(N3)-NH2
Ac-PKKKGKV-miniPEG2-Lys(cyclo(Ff-Nal-GrGrQ))-miniPEG2-K(N3)-NH2
Ac-PKKKRGV-miniPEG2-Lys(cyclo(Ff-Nal-GrGrQ))-miniPEG2-K(N3)-NH2
Ac-PKKKRKG-miniPEG2-Lys(cyclo(Ff-Nal-GrGrQ))-miniPEG2-K(N3)-NH2
Ac-KKKRK-miniPEG2-Lys(cyclo(Ff-Nal-GrGrQ))-miniPEG2-K(N3)-NH2
Ac-KKRK-miniPEG2-Lys(cyclo(Ff-Nal-GrGrQ))-miniPEG2-K(N3)-NH2 and
Ac-KRK-miniPEG2-Lys(cyclo(Ff-Nal-GrGrQ))-miniPEG2-K(N3)-NH2.

The EEV can be selected from:
Ac-PKKKRKV-Lys(cyclo[FfΦGrGrQ])-PEG₁₂-K(N₃)—NH₂
Ac-PKKKRKV-miniPEG2-Lys(cyclo[FfΦGrGrQ])-miniPEG₂-K(N₃)—NH₂
Ac-PKKKRKV-miniPEG₂-Lys(cyclo[FGFGRGRQ])-miniPEG₂-K(N₃)—NH₂
Ac-KR-PEG₂-K(cyclo[FGFGRGRQ])-PEG₂-K(N₃)—NH₂
Ac-PKKKGKV-PEG₂-K(cyclo[FGFGRGRQ])-PEG₂-K(N₃)—NH₂
Ac-PKKKRKG-PEG₂-K(cyclo[FGFGRGRQ])-PEG₂-K(N₃)—NH₂
Ac-KKKRK-PEG₂-K(cyclo[FGFGRGRQ])-PEG₂-K(N₃)—NH₂
Ac-PKKKRKV-miniPEG₂-Lys(cyclo[FFΦGRGRQ])-miniPEG₂-K(N₃)—NH₂
Ac-PKKKRKV-miniPEG₂-Lys(cyclo[βhFfΦGrGrQ])-miniPEG₂-K(N₃)—NH₂ and
Ac-PKKKRKV-miniPEG₂-Lys(cyclo[FfΦSrSrQ])-miniPEG₂-K(N₃)—NH₂.

The EEV can be selected from:
Ac-PKKKRKV-miniPEG₂-Lys(cyclo(GfFGrGrQ])-PEG₁₂-OH
Ac-PKKKRKV-miniPEG₂-Lys(cyclo[FGFKRKRQ])-PEG₁₂-OH
Ac-PKKKRKV-miniPEG₂-Lys(cyclo[FGFRGRGQ])-PEG₁₂-OH
Ac-PKKKRKV-miniPEG₂-Lys(cyclo[FGFGRGRGRQ])-PEG₁₂-OH
Ac-PKKKRKV-miniPEG₂-Lys(cyclo[FGFGRrRQ])-PEG₁₂-OH
Ac-PKKKRKV-miniPEG₂-Lys(cyclo[FGFGRRRQ])-PEG₁₂-OH and
Ac-PKKKRKV-miniPEG₂-Lys(cyclo[FGFRRRRQ])-PEG₁₂-OH.

The EEV can be selected from:
Ac-K-K-K-R-K-G-miniPEG₂-K(cyclo[FGFGRGRQ])-PEG₁₂-OH
Ac-K-K-K-R-K-miniPEG₂-K(cyclo[FGFGRGRQ])-PEG₁₂-OH
Ac-K-K-R-K-K-PEG₄-K(cyclo[FGFGRGRQ])-PEG₁₂-OH
Ac-K-R-K-K-K-PEG₄-K(cyclo[FGFGRGRQ])-PEG₁₂-OH
Ac-K-K-K-K-R-PEG₄-K(cyclo[FGFGRGRQ])-PEG₁₂-OH
Ac-R-K-K-K-K-PEG₄-K(cyclo[FGFGRGRQ])-PEG₁₂-OH and
Ac-K-K-K-R-K-PEG₄-K(cyclo[FGFGRGRQ])-PEG₁₂-OH.

The EEV can be selected from:
Ac-PKKKRKV-PEG₂-K(cyclo[FGFGRGRQ])-PEG₂-K(N₃)—NH₂
Ac-PKKKRKV-PEG₂-K(cyclo[FGFGRGRQ])-PEG₁₂-OH
Ac-PKKKRKV-PEG₂-K(cyclo[GfFGrGrQ])-PEG₂-K(N₃)—NH₂ and
Ac-PKKKRKV-PEG₂-K(cyclo[GfFGrGrQ])-PEG₁₂-OH.

The cargo can be an AC and the EEV can be selected from:
Ac-PKKKRKV-PEG₂-K(cyclo[FfΦGrGrQ])-PEG₁₂-OH
Ac-PKKKRKV-PEG₂-K(cyclo[FfΦCit-r-Cit-rQ])-PEG₁₂-OH
Ac-PKKKRKV-PEG₂-K(cyclo[FfFGRGRQ])-PEG₁₂-OH
Ac-PKKKRKV-PEG₂-K(cyclo[FGFGRGRQ])-PEG₁₂-OH
Ac-PKKKRKV-PEG₂-K(cyclo[GfFGrGrQ])-PEG₁₂-OH
Ac-PKKKRKV-PEG₂-K(cyclo[FGFGRRRQ])-PEG₁₂-OH
Ac-PKKKRKV-PEG₂-K(cyclo[FGFRRRRQ])-PEG₁₂-OH
Ac-rr-PEG₂-K(cyclo[FfΦGrGrQ])-PEG₁₂-OH
Ac-rr-PEG₂-K(cyclo[FfΦCit-r-Cit-rQ])-PEG₁₂-OH
Ac-rr-PEG₂-K(cyclo[FfF-GRGRQ])-PEG₁₂-OH
Ac-rr-PEG₂-K(cyclo[FGFGRGRQ])-PEG₁₂-OH
Ac-rr-PEG₂-K(cyclo[GfFGrGrQ])-PEG₁₂-OH
Ac-rr-PEG₂-K(cyclo[FGFGRRRQ])-PEG₁₂-OH
Ac-rr-PEG₂-K(cyclo[FGFRRRRQ])-PEG₁₂-OH
Ac-rrr-PEG₂-K(cyclo[FfΦGrGrQ])-PEG₁₂-OH
Ac-rrr-PEG₂-K(cyclo[FfΦCit-r-Cit-rQ])-PEG₁₂-OH
Ac-rrr-PEG₂-K(cyclo[FfFGRGRQ])-PEG₁₂-OH
Ac-rrr-PEG₂-K(cyclo[FGFGRGRQ])-PEG₁₂-OH
Ac-rrr-PEG₂-K(cyclo[GfFGrGrQ])-PEG₁₂-OH
Ac-rrr-PEG₂-K(cyclo[FGFGRRRQ])-PEG₁₂-OH
Ac-rrr-PEG₂-K(cyclo[FGFRRRRQ])-PEG₁₂-OH
Ac-rhr-PEG₂-K(cyclo[FfΦGrGrQ])-PEG₁₂-OH
Ac-rhr-PEG₂-K(cyclo[FfΦCit-r-Cit-rQ])-PEG₁₂-OH
Ac-rhr-PEG₂-K(cyclo[FfFGRGRQ])-PEG₁₂-OH
Ac-rhr-PEG₂-K(cyclo[FGFGRGRQ])-PEG₁₂-OH
Ac-rhr-PEG₂-K(cyclo[GfFGrGrQ])-PEG₁₂-OH
Ac-rhr-PEG₂-K(cyclo[FGFGRRRQ])-PEG₁₂-OH
Ac-rhr-PEG₂-K(cyclo[FGFRRRRQ])-PEG₁₂-OH
Ac-rbr-PEG₂-K(cyclo[FfΦGrGrQ])-PEG₁₂-OH
Ac-rbr-PEG₂-K(cyclo[FfΦCit-r-Cit-rQ])-PEG₁₂-OH
Ac-rbr-PEG₂-K(cyclo[FfFGRGRQ])-PEG₁₂-OH
Ac-rbr-PEG₂-K(cyclo[FGFGRGRQ])-PEG₁₂-OH
Ac-rbr-PEG₂-K(cyclo[GfFGrGrQ])-PEG₁₂-OH
Ac-rbr-PEG₂-K(cyclo[FGFGRRRQ])-PEG₁₂-OH
Ac-rbr-PEG₂-K(cyclo[FGFRRRRQ])-PEG₁₂-OH
Ac-rbrbr-PEG₂-K(cyclo[FfΦGrGrQ])-PEG₁₂-OH
Ac-rbrbr-PEG₂-K(cyclo[FfΦCit-r-Cit-rQ])-PEG₁₂-OH
Ac-rbrbr-PEG₂-K(cyclo[FfFGRGRQ])-PEG₁₂-OH
Ac-rbrbr-PEG₂-K(cyclo[FGFGRGRQ])-PEG₁₂-OH
Ac-rbrbr-PEG₂-K(cyclo[GfFGrGrQ])-PEG₁₂-OH
Ac-rbrbr-PEG₂-K(cyclo[FGFGRRRQ])-PEG₁₂-OH
Ac-rbrbr-PEG₂-K(cyclo[FGFRRRRQ])-PEG₁₂-OH
Ac-rbhbr-PEG₂-K(cyclo[FfΦGrGrQ])-PEG₁₂-OH
Ac-rbhbr-PEG₂-K(cyclo[FfΦCit-r-Cit-rQ])-PEG₁₂-OH
Ac-rbhbr-PEG₂-K(cyclo[FfFGRGRQ])-PEG₁₂-OH
Ac-rbhbr-PEG₂-K(cyclo[FGFGRGRQ])-PEG₁₂-OH
Ac-rbhbr-PEG₂-K(cyclo[GfFGrGrQ])-PEG₁₂-OH
Ac-rbhbr-PEG₂-K(cyclo[FGFGRRRQ])-PEG₁₂-OH
Ac-rbhbr-PEG₂-K(cyclo[FGFRRRRQ])-PEG₁₂-OH
Ac-hbrbh-PEG₂-K(cyclo[FfΦGrGrQ])-PEG₁₂-OH
Ac-hbrbh-PEG₂-K(cyclo[FfΦCit-r-Cit-rQ])-PEG₁₂-OH
Ac-hbrbh-PEG₂-K(cyclo[FfFGRGRQ])-PEG₁₂-OH
Ac-hbrbh-PEG₂-K(cyclo[FGFGRGRQ])-PEG₁₂-OH
Ac-hbrbh-PEG₂-K(cyclo[GfFGrGrQ])-PEG₁₂-OH
Ac-hbrbh-PEG₂-K(cyclo[FGFGRRRQ])-PEG₁₂-OH and
Ac-hbrbh-PEG₂-K(cyclo[FGFRRRRQ])-PEG₁₂-OH,
wherein b is beta-alanine, and the exocyclic sequence can be D or L stereochemistry.

In embodiments, compounds comprising a cyclic peptide and an AC have improved cytosolic uptake efficiency compared to compounds comprising an AC alone. Cytosolic uptake efficiency can be measured by comparing the cytosolic delivery efficiency of the compound comprising the cyclic peptide and the AC to the cytosolic delivery efficiency of an AC alone.

### Antisense Compound

In various embodiments, the compounds disclosed herein comprise a CPP (e.g., cyclic peptide) conjugated to an antisense compound (AC). In embodiments, the AC comprises an antisense oligonucleotide directed to a target polynucleotide. The term “antisense oligonucleotide” or simply “antisense” is meant to include oligonucleotides that are complementary to a targeted polynucleotide sequence. Antisense oligonucleotides are single strands of DNA or RNA that are complementary to a chosen sequence, e.g., a target gene mRNA.

The antisense oligonucleotides may modulate one or more aspects of protein transcription, translation, and expression. In embodiments, the antisense oligonucleotide is directed to a target sequence within a target pre-mRNA modulates one or more aspects of pre-mRNA splicing. As used herein, modulation of splicing refers to altering the processing of a pre-mRNA transcript such that the spliced mRNA molecule contains either a different combination of exons as a result of exon skipping or exon inclusion, a deletion in one or more exons, or the deletion or addition of a sequence not normally found in the spliced mRNA (e.g., an intron sequence). In embodiments, antisense oligonucleotides hybridization to a target sequence in a pre-mRNA molecule restores native splicing to a mutated pre-mRNA sequence. In embodiments, antisense oligonucleotides hybridization results in alternative splicing of the target pre-mRNA. In embodiments, antisense oligonucleotides hybridization results in exon inclusion or exon skipping of one or more exons. In embodiments, the skipped exon sequence comprises a frameshift mutation, a nonsense mutation, or a missense mutation. In embodiments, the skipped exon sequence comprises a nucleic acid deletion, substitution, or insertion. In embodiments, the skipped exon itself does not comprise a sequence mutation, but a neighboring exon comprises a mutation leading to a frameshift mutation or a nonsense mutation. In embodiments, antisense oligonucleotides hybridization to a target sequence within a target pre-mRNA prevents inclusion of an exon sequence in the mature mRNA molecule. In embodiments, antisense oligonucleotides hybridization to a target sequence within a target pre-mRNA results in preferential expression of a wild type target protein isomer. In embodiments, antisense oligonucleotides hybridization to a target sequence within a target pre-mRNA results in expression of a re-spliced target protein comprising an active fragment of a wild type target protein.

The antisense mechanism functions via hybridization of an antisense oligonucleotide compound with a target nucleic acid. In embodiments, the antisense oligonucleotide hybridizing to its target sequence suppresses expression of the target protein. In embodiments, hybridization of the antisense oligonucleotide to its target sequence suppresses expression of one or more wild type target protein isomers. In embodiments, hybridization of the antisense oligonucleotide to its target sequence upregulates expression of the target protein. In embodiments, hybridization of the antisense oligonucleotide to its target sequence increases expression of one or more wild type target protein isomers.

In embodiments, the antisense compound can inhibit gene expression by binding to a complementary mRNA. Binding to the target mRNA can lead to inhibition of gene expression either by preventing translation of complementary mRNA strands by sterically blocking RNA binding proteins involved in translation or by leading to degradation of the target mRNA. Antisense DNA can be used to target a specific, complementary (coding or non-coding) RNA. If binding takes place, the DNA/RNA hybrid can be degraded by the enzyme RNase H. In embodiments, antisense oligonucleotides contain from about 10 to about 50 nucleotides, or about to about 30 nucleotides. In embodiments, antisense oligonucleotides may not be fully complementary to the target nucleotide sequence.

Antisense oligonucleotides have been demonstrated to be effective and targeted inhibitors of protein synthesis, and, consequently, can be used to specifically inhibit protein synthesis by a targeted gene. The efficacy of antisense oligonucleotides for inhibiting protein synthesis is well established. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829). Further, examples of antisense inhibition have been demonstrated with the nuclear protein cyclin, the multiple drug resistance gene (MDG1), ICAM-1, E-selectin, STK-1, striatal GABAA receptor and human EGF (Jaskulski et ai, Science. 1988 Jun. 10; 240(4858): 1544-6; Vasanthakumar and Ahmed, Cancer Commun. 1989; 1(4):225-32; Peris et ai, Brain Res Mol Brain Res. 1998 Jun. 15; 57(2):310-20; U.S. Pat. Nos. 5,801,154; 5,789,573; 5,718,709 and 5,610,288). Furthermore, antisense constructs have also been described that inhibit and can be used to treat a variety of abnormal cellular proliferations, e.g., cancer (U.S. Pat. Nos. 5,747,470; 5,591,317 and 5,783,683).

Methods of producing antisense oligonucleotides are known in the art and can be readily adapted to produce an antisense oligonucleotide that targets any polynucleotide sequence. Selection of antisense oligonucleotide sequences specific for a given target sequence is based upon analysis of the chosen target sequence and determination of secondary structure, Tm, binding energy, and relative stability. Antisense oligonucleotides may be selected based upon their relative inability to form dimers, hairpins, or other secondary structures that would reduce or prohibit specific binding to the target mRNA in a host cell. Target regions of the mRNA can include those regions at or near the AUG translation initiation codon and those sequences that are substantially complementary to 5′ regions of the mRNA. These secondary structure analyses and target site selection considerations can be performed, for example, using v.4 of the OLIGO primer analysis software (Molecular Biology Insights) and/or the BLASTN 2.0.5 algorithm software (Altschul et ai, Nucleic Acids Res. 1997, 25(17):3389-402).

According to the present disclosure, an antisense compound (AC) alters one or more aspects of the splicing, translation, or expression of a target gene, e.g., by altering the splicing of a eukaryotic target pre-mRNA. The AC according to the disclosure comprises a nucleic acid sequence that is complementary to a sequence found within a target pre-mRNA sequence, for example, at sequence that includes at least a portion of an exon, at least a portion of an intron, or both. The use of these ACs provides a direct genetic approach that has the ability to modulate splicing of specific disease-causing genes. The principle behind antisense technology is that an antisense compound, which hybridizes to a target nucleic acid, modulates gene expression activities such as splicing or translation through one of a number of antisense mechanisms. The sequence-specificity of the AC makes this technique extremely attractive as a therapeutic to selectively modulate the splicing of pre-mRNA involved in the pathogenesis of any one of a variety of diseases. Antisense technology is an effective means for changing the expression of one or more specific gene products and can therefore prove to be useful in a number of therapeutic, diagnostic, and research applications.

The compounds described herein may contain one or more asymmetric centers and thus give rise to enantiomers, diastereomers, and other stereoisomeric configurations that may be defined, in terms of absolute stereochemistry, as (R) or (S), α or β, or as (D) or (L). Included in the antisense compounds provided herein are all such possible isomers, as well as their racemic and optically pure forms.

### Antisense Compound Hybridization Site

Antisense mechanisms rely on hybridization of the antisense compound to the target nucleic acid. In embodiments, the present disclosure provides antisense compounds that are complementary to a target nucleic acid. In embodiments, the target nucleic acid sequence is present in a pre-mRNA molecule. In embodiments, the target nucleic acid sequence is present in an exon of a pre-mRNA molecule. In embodiments, the target nucleic acid sequence is present in an intron of a pre-mRNA molecule.

Pre-mRNA molecules are made in the nucleus and are processed before or during transport to the cytoplasm for translation. Processing of the pre-mRNAs includes addition of a 5′ methylated cap and an approximately 200-250 base poly(A) tail to the 3′ end of the transcript. The next step in mRNA processing is splicing of the pre-mRNA, which occurs in the maturation of 90-95% of mammalian mRNAs. Introns (or intervening sequences) are regions of a primary transcript (or the DNA encoding it) that are not included in the coding sequence of the mature mRNA. Exons are regions of a primary transcript that remain in the mature mRNA when it reaches the cytoplasm. The exons are spliced together to form the mature mRNA sequence. Splice junctions are also referred to as splice sites with the 5′ side of the junction often called the “5′ splice site,” or “splice donor site” and the 3′ side called the “3′ splice site” or “splice acceptor site.” In splicing, the 3′ end of an upstream exon is joined to the 5′ end of the downstream exon. Thus, the unspliced RNA (or pre-mRNA) has an exon/intron junction at the 5′ end of an intron and an intron/exon junction at the 3′ end of an intron. After the intron is removed, the exons are contiguous at what is sometimes referred to as the exon/exon junction or boundary in the mature mRNA. Cryptic splice sites are those which are less often used but may be used when the usual splice site is blocked or unavailable. Alternative splicing, defined as the splicing together of different combinations of exons, often results in multiple mRNA transcripts from a single gene.

In embodiments, the AC hybridizes with a sequence in a splice site. In embodiments, the AC hybridizes with a sequence comprising part of a splice site. In embodiments, the AC hybridizes with a sequence comprising part or all of a splice site. In embodiments, the AC hybridizes with a sequence comprising part or all of a splice donor site. In embodiments, the AC hybridizes with a sequence comprising part or all of a splice acceptor site. In embodiments, the AC hybridizes with a sequence comprising part or all of a cryptic splice site. In embodiments, the AC hybridizes with a sequence comprising an exon/intron junction.

Pre-mRNA splicing involves two sequential biochemical reactions. Both reactions involve the spliceosomal transesterification between RNA nucleotides. In a first reaction, the 2′-OH of a specific branch-point nucleotide within an intron, which is defined during spliceosome assembly, performs a nucleophilic attack on the first nucleotide of the intron at the 5′ splice site forming a lariat intermediate. In a second reaction, the 3′-OH of the released 5′ exon performs a nucleophilic attack at the last nucleotide of the intron at the 3′ splice site thus joining the exons and releasing the intron lariat. Pre-mRNA splicing is regulated by intronic silencer sequence (ISS) and terminal stem loop (TSL) sequences. As used herein, the terms “intronic silencer sequences (ISS)” and “terminal stem loop (TSL)” refer to sequence elements within introns and exons, respectively, that control alternative splicing by the binding of trans-acting protein factors within a pre-mRNA thereby resulting in differential use of splice sites. Typically, intronic silencer sequences are between 8 and 16 nucleotides and are less conserved than the splice sites at exon-intron junctions. Terminal stem loop sequences are typically between 12 and 24 nucleotides and form a secondary loop structure due to the complementarity, and hence binding, within the 12-24 nucleotide sequence.

In embodiments, the AC hybridizes with a sequence comprising part or all of an intronic silencer sequence. In embodiments, the AC hybridizes with a sequence comprising part or all of a terminal stem loop.

Up to 50% of human genetic diseases resulting from a point mutation are caused by aberrant splicing. Such point mutations can either disrupt a current splice site or create a new splice site, resulting in mRNA transcripts comprised of a different combination of exons or with deletions in exons. Point mutations also can result in activation of a cryptic splice site or disrupt regulatory cis elements (i.e., splicing enhancers or silencers).

In embodiments, the AC hybridizes with a sequence comprising part or all of an aberrant splice site resulting from a mutation in the target gene. In embodiments, the AC hybridizes with a sequence comprising part or all of a regulatory element. Also provided are antisense compounds targeted to cis regulatory elements. In embodiments, the regulatory element is in an exon. In embodiments, the regulatory element is in an intron.

In embodiments, the AC may be specifically hybridizable with a translation initiation codon region, a 5′ cap region, an intron/exon junction, a coding sequence, a translation termination codon region or sequences in the 5′- or 3′-untranslated region. In embodiments, the AC may hybridize with part or all of a pre-mRNA splice site, an exon-exon junction, or an intron-exon junction. In embodiments, the AC may hybridize with an aberrant fusion junction due to a rearrangement or a deletion. In embodiments, the AC may hybridize with particular exons in alternatively spliced mRNAs.

In embodiments, the AC hybridizes with a sequence between 5 and 50 nucleotides in length, which can also be referred to as the length of the AC. In embodiments, the AC is between and 50 nucloetides in length, for example, between 5 and 10, 10 and 15, 15 and 20, 20 and 25, and 30, 30 and 35, 35 and 40, 40 and 45, or 45 and 50 nucleotides in length. In embodiments, the AC is about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In embodiments, the AC is at least about 5, about 6, about 7, about 8, about 9, about 10, about 11, about 12, about 13, about 14, about 15, about 16, about 17, about 18, about 19, or about and up to about 21, about 22, about 23, about 24, or about 25, and up to about 26, about 27, about 28, about 29, about 30, about 31, about 32, about 33, about 34, about 35, about 36, about 37, about 38, about 39, or about 40, and up to about 41, about 42, about 43, about 44, about 45, about 46, about 47, about 48, about 49, or about 50 nucleotides in length. In embodiments, the AC is about 10 nucleotides in length. In embodiments, the AC is about 15 nucleotides in length. In embodiments, the AC is about 16 nucleotides in length. In embodiments, the AC is about 17 nucleotides in length. In embodiments, the AC is about 18 nucleotides in length. In embodiments, the AC is about 19 nucleotides in length. In embodiments, the AC is about 20 nucleotides in length. In embodiments, the AC is about 21 nucleotides in length. In embodiments, the AC is about 22 nucleotides in length. In embodiments, the AC is about 23 nucleotides in length. In embodiments, the AC is about 24 nucleotides in length. In embodiments, the AC is about 25 nucleotides in length. In embodiments, the AC is about 26 nucleotides in length. In embodiments, the AC is about 27 nucleotides in length. In embodiments, the AC is about 28 nucleotides in length. In embodiments, the AC is about 29 nucleotides in length. In embodiments, the AC is about 30 nucleotides in length.

In embodiments, the AC may be less than 100 percent complementary to a target nucleic acid sequence. As used herein, the term “percent complementary” refers to the number of nucleobases of an AC that have nucleobase complementarity with a corresponding nucleobase of an oligomeric compound or nucleic acid divided by the total length (number of nucleobases) of the AC. One skilled in the art recognizes that the inclusion of mismatches is possible without eliminating the activity of the antisense compound. In embodiments, an AC may contain up to about 20% nucleotides that disrupt base pairing of the AC to the target nucleic acid. In embodiments, the ACs contain no more than about 15%, no more than about 10%, no more than 5%, or no mismatches. In embodiments, the ACs contain no more than 1, 2, 3, 4 or 5 mismatches. In embodiments, the ACs are at least 80%, at least 85%, at least 90%, at least 95%, at least 96%, at least 97%, at least 98%, at least 99% or 100% complementary to a target nucleic acid. Percent complementarity of an oligonucleotide is calculated by dividing the number of complementary nucleobases by the total number of nucleobases of the oligonucleotide. Percent complementarity of a region of an oligonucleotide is calculated by dividing the number of complementary nucleobases in the region by the total number of nucleobases region.

In embodiments, incorporation of nucleotide affinity modifications allows for a greater number of mismatches compared to an unmodified compound. Similarly, certain oligonucleotide sequences may be more tolerant to mismatches than other oligonucleotide sequences. One of ordinary skill in the art is capable of determining an appropriate number of mismatches between oligonucleotides, or between an oligonucleotide and a target nucleic acid, such as by determining melting temperature (Tm). Tm or ΔTm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

### Antisense Mechanisms

The ACs according to the present disclosure may modulate one or more aspects of protein transcription, translation, and expression. In embodiments, the AC hybridizing to a target sequence within a target pre-mRNA modulates one or more aspects of pre-mRNA splicing. As used herein, modulation of splicing refers to altering the processing of a pre-mRNA transcript such that the spliced mRNA molecule contains either a different combination of exons as a result of exon skipping or exon inclusion, a deletion in one or more exons, or the deletion or addition of a sequence not normally found in the spliced mRNA (e.g., an intron sequence). In embodiments, AC hybridization to a target sequence within a pre-mRNA molecule restores native splicing to a mutated pre-mRNA sequence. In embodiments, AC hybridization results in alternative splicing of the target pre-mRNA. In embodiments, AC hybridization results in exon inclusion or exon skipping of one or more exons. In embodiments, the skipped exon sequence comprises a frameshift mutation, a nonsense mutation, or a missense mutation. In embodiments, the skipped exon sequence comprises a nucleic acid deletion, substitution, or insertion. In embodiments, the skipped exon itself does not comprise a sequence mutation, but a neighboring exon comprises a mutation leading to a frameshift mutation or a nonsense mutation. In embodiments, deletion of an exon that does not comprise a sequence mutation restores the reading frame of the mature mRNA. In embodiments, AC hybridization to a target sequence within a target pre-mRNA results in preferential expression of a wild type target protein isomer. In embodiments, AC hybridization to a target sequence within a target pre-mRNA results in expression of a re-spliced target protein comprising an active fragment of a wild type target protein.

The antisense mechanism functions via hybridization of an antisense compound with a target nucleic acid. In embodiments, the AC hybridizing to its target sequence suppresses expression of the target protein. In embodiments, the AC hybridizing to its target sequence suppresses expression of one or more wild type target protein isomers. In embodiments, the AC hybridizing to its target sequence upregulates expression of the target protein. In embodiments, the AC hybridizing to its target sequence increases expression of one or more wild type target protein isomers.

The efficacy of the ACs of the present disclosure may be assessed by evaluating the antisense activity effected by their administration. As used herein, the term “antisense activity” refers to any detectable and/or measurable activity attributable to the hybridization of an antisense compound to its target nucleic acid. Such detection and or measuring may be direct or indirect. In embodiments, antisense activity is assessed by detecting and or measuring the amount of target protein. In embodiments, antisense activity is assessed by detecting and or measuring the amount of re-spliced target protein. In embodiments, antisense activity is assessed by detecting and/or measuring the amount of target nucleic acids and/or cleaved target nucleic acids and/or alternatively spliced target nucleic acids

### Antisense Compound Design

Design of ACs according to the present disclosure will depend upon the sequence being targeted. Targeting an AC to a particular target nucleic acid molecule can be a multistep process. The process usually begins with the identification of a target nucleic acid whose expression is to be modulated. As used herein, the terms “target nucleic acid” and “nucleic acid encoding a target gene” encompass DNA encoding a selected target gene, RNA (including pre-mRNA and mRNA) transcribed from such DNA, and also cDNA derived from such RNA. For example, the target nucleic acid can be a cellular gene (or mRNA transcribed from the gene) whose expression is associated with a particular disorder or disease state, or a nucleic acid molecule from an infectious agent.

One of skill in the art will be able to design, synthesize, and screen antisense compounds of different nucleobase sequences to identify a sequence that results in antisense activity. For example, one may design an antisense compound that alters splicing of a target pre-mRNA or inhibits expression of a target protein. Methods for designing, synthesizing and screening antisense compounds for antisense activity against a preselected target nucleic acid can be found, for example in “Antisense Drug Technology, Principles, Strategies, and Applications” Edited by Stanley T. Crooke, CRC Press, Boca Raton, Florida, which is incorporated by reference in its entirety for any purpose.

In embodiments, the antisense compounds comprise modified nucleosides, modified internucleoside linkages and/or conjugate groups.

In embodiments, the antisense compound is a “tricyclo-DNA (tc-DNA)”, which refers to a class of constrained DNA analogs in which each nucleotide is modified by the introduction of a cyclopropane ring to restrict conformational flexibility of the backbone and to optimize the backbone geometry of the torsion angle γ. Homobasic adenine- and thymine-containing tc-DNAs form extraordinarily stable A-T base pairs with complementary RNAs.

### Nucleosides

In embodiments, antisense compounds are provided comprising linked nucleosides. In embodiments, some or all of the nucleosides are modified nucleosides. In embodiments, one or more nucleosides comprise a modified nucleobase. In embodiments, one or more nucleosides comprises a modified sugar. Chemically modified nucleosides are routinely used for incorporation into antisense compounds to enhance one or more properties, such as nuclease resistance, pharmacokinetics or affinity for a target RNA.

In general, a nucleobase is any group that contains one or more atom or groups of atoms capable of hydrogen bonding to a base of another nucleic acid. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The terms modified nucleobase and nucleobase mimetic can overlap but generally a modified nucleobase refers to a nucleobase that is similar in structure to the parent nucleobase, such as for example a 7-deaza purine, a 5-methyl cytosine, or a G-clamp, whereas a nucleobase mimetic would include more complicated structures, such as for example a tricyclic phenoxazine nucleobase mimetic. Methods for preparation of the above noted modified nucleobases are well known to those skilled in the art.

In embodiments, ACs provided herein comprise one or more nucleosides having a modified sugar moiety. In embodiments, the furanosyl sugar ring of a natural nucleoside can be modified in a number of ways including, but not limited to, addition of a substituent group, bridging of two non-geminal ring atoms to form a bicyclic nucleic acid (BNA) and substitution of an atom or group such as —S—, —N(R)— or —C(R₁)(R₂) for the ring oxygen at the 4′-position. Modified sugar moieties are well known and can be used to alter, typically increase, the affinity of the antisense compound for its target and/or increase nuclease resistance. A representative list of modified sugars includes but is not limited to non-bicyclic substituted sugars, especially non-bicyclic 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others, for example, the furanose ring can be replaced with a morpholine ring. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and 6,600,032; and WO 2005/121371.

In embodiments, nucleosides comprise bicyclic modified sugars (BNA's), including LNA (4′-(CH₂)—O-2′ bridge), 2′-thio-LNA (4′-(CH₂)—S-2′ bridge), 2′-amino-LNA (4′-(CH₂)—NR-2′ bridge), ENA (4′-(CH₂)2-O-2′ bridge), 4′-(CH₂)₃-2′ bridged BNA, 4′-(CH₂CH(CH₃))-2′ bridged BNA″ cEt (4′-(CH(CH₃)—O-2′ bridge), and cMOE BNAs (4′-(CH(CH₂OCH₃)—O-2′ bridge). Certain such BNA's have been prepared and disclosed in the patent literature as well as in scientific literature (See, e.g., Srivastava, et al. J. Am. Chem. Soc. 2007, ACS Advanced online publication, 10.1021/ja071106y, Albaek et al. J. Org. Chem., 2006, 71, 7731-7740, Fluiter, et al. Chembiochem 2005, 6, 1104-1109, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039, WO 2007/090071; Examples of issued US patents and published applications that disclose BNAs include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

An isomer of LNA that has also been studied is alpha-L-LNA which has been shown to have improved stability against a 3′-exonuclease. The alpha-L-LNA's were incorporated into antisense gapmers and chimeras that showed potent antisense activity (Frieden et al., Nucleic Acids Research, 2003, 21, 6365-6372).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., WO 99/14226). Synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

### Internucleoside Linkages

Described herein are internucleoside linking groups that link the nucleosides or otherwise modified monomer units together thereby forming an antisense compound. The two main classes of internucleoside linking groups are defined by the presence or absence of a phosphorus atom. Representative phosphorus containing internucleoside linkages include, but are not limited to, phosphodiesters, phosphotriesters, methylphosphonates, phosphoramidate (including phosphorodiamidate), and phosphorothioates. Representative non-phosphorus containing internucleoside linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Antisense compounds having non-phosphorus internucleoside linking groups are referred to as oligonucleosides. Modified internucleoside linkages, compared to natural phosphodiester linkages, can be used to alter, typically increase, nuclease resistance of the antisense compound. Internucleoside linkages having a chiral atom can be prepared racemic, chiral, or as a mixture. Representative chiral internucleoside linkages include, but are not limited to, alkylphosphonates and phosphorothioates. Methods of preparation of phosphorous-containing and non-phosphorous-containing linkages are well known to those skilled in the art.

In embodiments, a phosphate group can be linked to the 2′, 3′ or 5′ hydroxyl moiety of the sugar. In forming oligonucleotides, the phosphate groups covalently link adjacent nucleosides to one another to form a linear polymeric compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Conjugate Groups

In embodiments, ACs are modified by covalent attachment of one or more conjugate groups. In general, conjugate groups modify one or more properties of the attached AC including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an AC. Conjugate groups include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. In embodiments, the conjugate group is a polyethylene glycol (PEG), and the PEG is conjugated to either the AC or the cyclic peptide.

Conjugate groups include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996,277,923).

Linking groups or bifunctional linking moieties such as those known in the art can be included with the compounds provided herein. Linking groups are useful for attachment of chemical functional groups, conjugate groups, reporter groups and other groups to selective sites in a parent compound such as for example an AC. In embodiments, a bifunctional linking moiety comprises a hydrocarbyl moiety having two functional groups. In embodiments, one of the functional groups is selected to bind to a parent molecule or compound of interest and the other is selected to bind essentially any selected group such as chemical functional group or a conjugate group. Any of the linkers described here may be used. In embodiments, the linker comprises a chain structure or an oligomer of repeating units such as ethylene glycol or amino acid units. Examples of functional groups that are used in a bifunctional linking moiety include, but are not limited to, electrophiles for reacting with nucleophilic groups and nucleophiles for reacting with electrophilic groups. In embodiments, bifunctional linking moieties include amino, hydroxyl, carboxylic acid, thiol, unsaturations (e.g., double or triple bonds), and the like. Some nonlimiting examples of bifunctional linking moieties include 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linking groups include, but are not limited to, substituted C₁-C₁₀ alkyl, substituted or unsubstituted C₂-C₁₀ alkenyl or substituted or unsubstituted C₂-C₁₀ alkynyl, wherein a nonlimiting list of substituent groups includes hydroxyl, amino, alkoxy, carboxy, benzyl, phenyl, nitro, thiol, thioalkoxy, halogen, alkyl, aryl, alkenyl and alkynyl.

In embodiments, the AC may be linked to a 10 arginine-serine dipeptide repeat. ACs linked to 10 arginine-serine dipeptide repeats for the artificial recruitment of splicing enhancer factors have been applied in vitro to induce inclusion of mutated BRCA1 and SMN2 exons that otherwise would be skipped. See Cartegni and Krainer 2003, incorporated by reference herein.

In embodiments, the AC may be from 5 to 50 nucleotides in length (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, inclusive of all values and ranges therein). In embodiments, the AC may be 5-10 nucleotides in length. In embodiments, the AC may be 10-nucleotides in length. In embodiments, the AC may be 15-20 nucleotides in length. In embodiments, the AC may be 20-25 nucleotides in length. In embodiments, the AC may be 25-30 nucleotides in length. In embodiments, the AC may be 30-35 nucleotides in length. In embodiments, the AC may be 35-40 nucleotides in length. In embodiments, the AC may be 40-45 nucleotides in length. In embodiments, the AC may be 45-50 nucleotides in length.

In embodiments, the AC binds to the human DMD gene, which encodes for dystrophin. In embodiments, the AC binds to at least a portion of exon 44 of DMD. In embodiments, the AC binds to at least a portion of a 3′ flanking of exon 44 of DMD. In embodiments, the AC binds to at least a portion of a 5′ flanking intron of exon 44 of DMD. In embodiments, the AC that binds to exon 44 of DMD is from about 18 to about 30 nucleic acids in length, for example, about 18, about 19, about 20, about 21, about 22, about 23, about 24, about 25, about 26, about 27, about 28, about 29, or about 30 nucleic acids in length.

In embodiments, the nucleic acid sequence of exon 44 of DMD from 5′ to 3′ is:

(SEQ ID NO: 1)
GCGATTTGACAGATCTGTTGAGAAATGGCGGCGTTTTCATTATGATATA
AAGATATTTAATCAGTGGCTAACAGAAGCTGAACAGTTTCTCAGAAAGA
CACAAATTCCTGAGAATTGGGAACATGCTAAATACAAATGGTATCTTAA
G.

In embodiments, the nucleic acid sequence of exon 44 of DMD from 5′ to 3′ is: GGCGATTTGACAGATCTGTTGAGAAATGGCGGCGTTTTCATTATGATATAAAGATAT TTAATCAGTGGCTAACAGAAGCTGAACAGTTTCTCAGAAAGACACAAATTCCTGAG AATTGGGAACATGCTAAATACAAATGGTATCTTAAG (SEQ ID NO: 2). In embodiments, the sequence of exon 44 comprises 1, 2, 3, 4, or 5 nucleotides, or more, at the 5′ end of SEQ ID NO: 1. In embodiments, the sequence of exon 44 comprises 1, 2, 3, 4, or 5 nucleotides, or more, at the 5′ end of SEQ ID NO: 2. In embodiments, the sequence of exon 44 comprises 1, 2, 3, 4, or nucleotides, or more, at the 3′ end of SEQ ID NO: 1. In embodiments, the sequence of exon 44 comprises 1, 2, 3, 4, or 5 nucleotides, or more, at the 3′ end of SEQ ID NO: 2.

In embodiments, the AC comprises 18 consecutive nucleotides (e.g., the AC is an 18-mer) of SEQ ID NO: 1, wherein the first nucleotide of the 18-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, or 131 of SEQ ID NO: 1. In embodiments, the AC comprises 18 consecutive nucleotides (e.g., the AC is an 18-mer) of SEQ ID NO: 2, wherein the first nucleotide of the 18-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 of SEQ ID NO: 2.

In embodiments, the AC comprises 19 consecutive nucleotides (e.g., the AC is an 19-mer) of SEQ ID NO: 1, wherein the first nucleotide of the 19-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of SEQ ID NO: 1. In embodiments, the AC comprises 19 consecutive nucleotides (e.g., the AC is an 19-mer) of SEQ ID NO: 2, wherein the first nucleotide of the 19-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, or 131 of SEQ ID NO: 2.

In embodiments, the AC comprises 20 consecutive nucleotides (e.g., the AC is an 20-mer) of SEQ ID NO: 1, wherein the first nucleotide of the 20-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 of SEQ ID NO: 1. In embodiments, the AC comprises 20 consecutive nucleotides (e.g., the AC is an 20-mer) of SEQ ID NO: 2, wherein the first nucleotide of the 20-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, or 130 of SEQ ID NO: 2.

In embodiments, the AC comprises 21 consecutive nucleotides (e.g., the AC is an 21-mer) of SEQ ID NO: 1, wherein the first nucleotide of the 21-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128 of SEQ ID NO: 1. In embodiments, the AC comprises 21 consecutive nucleotides (e.g., the AC is an 21-mer) of SEQ ID NO: 2, wherein the first nucleotide of the 21-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, or 129 of SEQ ID NO: 2.

In embodiments, the AC comprises 22 consecutive nucleotides (e.g., the AC is an 22-mer) of SEQ ID NO: 1, wherein the first nucleotide of the 22-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or 127 of SEQ ID NO: 1. In embodiments, the AC comprises 22 consecutive nucleotides (e.g., the AC is an 22-mer) of SEQ ID NO: 2, wherein the first nucleotide of the 22-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or 128 of SEQ ID NO: 2.

In embodiments, the AC comprises 23 consecutive nucleotides (e.g., the AC is an 23-mer) of SEQ ID NO: 1, wherein the first nucleotide of the 23-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126 of SEQ ID NO: 1. In embodiments, the AC comprises 23 consecutive nucleotides (e.g., the AC is an 23-mer) of SEQ ID NO: 2, wherein the first nucleotide of the 23-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, or 127 of SEQ ID NO: 2.

In embodiments, the AC comprises 24 consecutive nucleotides (e.g., the AC is an 24-mer) of SEQ ID NO: 1, wherein the first nucleotide of the 24-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 of SEQ ID NO: 1. In embodiments, the AC comprises 24 consecutive nucleotides (e.g., the AC is an 24-mer) of SEQ ID NO: 2, wherein the first nucleotide of the 24-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, or 126 of SEQ ID NO: 2.

In embodiments, the AC comprises 25 consecutive nucleotides (e.g., the AC is an 25-mer) of SEQ ID NO: 1, wherein the first nucleotide of the 25-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 of SEQ ID NO: 1. In embodiments, the AC comprises 25 consecutive nucleotides (e.g., the AC is an 25-mer) of SEQ ID NO: 2, wherein the first nucleotide of the 25-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 of SEQ ID NO: 2.

In embodiments, the AC comprises 26 consecutive nucleotides (e.g., the AC is an 26-mer) of SEQ ID NO: 1, wherein the first nucleotide of the 26-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, or 123 of SEQ ID NO: 1. In embodiments, the AC comprises 26 consecutive nucleotides (e.g., the AC is an 26-mer) of SEQ ID NO: 2, wherein the first nucleotide of the 26-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, or 124 of SEQ ID NO: 2.

In embodiments, the AC comprises 27 consecutive nucleotides (e.g., the AC is an 27-mer) of SEQ ID NO: 1, wherein the first nucleotide of the 27-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, or 122 of SEQ ID NO: 1. In embodiments, the AC comprises 27 consecutive nucleotides (e.g., the AC is an 27-mer) of SEQ ID NO: 2, wherein the first nucleotide of the 27-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, or 123 of SEQ ID NO: 2.

In embodiments, the AC comprises 28 consecutive nucleotides (e.g., the AC is an 28-mer) of SEQ ID NO: 1, wherein the first nucleotide of the 28-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or 121 of SEQ ID NO: 1. In embodiments, the AC comprises 28 consecutive nucleotides (e.g., the AC is an 28-mer) of SEQ ID NO: 2, wherein the first nucleotide of the 28-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, or 122 of SEQ ID NO: 2.

In embodiments, the AC comprises 29 consecutive nucleotides (e.g., the AC is an 29-mer) of SEQ ID NO: 1, wherein the first nucleotide of the 29-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 of SEQ ID NO: 1. In embodiments, the AC comprises 29 consecutive nucleotides (e.g., the AC is an 29-mer) of SEQ ID NO: 2, wherein the first nucleotide of the 29-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or 121 of SEQ ID NO: 2.

In embodiments, the AC comprises 30 consecutive nucleotides (e.g., the AC is an 30-mer) of SEQ ID NO: 1, wherein the first nucleotide of the 30-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, or 119 of SEQ ID NO: 1. In embodiments, the AC comprises 30 consecutive nucleotides (e.g., the AC is an 30-mer) of SEQ ID NO: 2, wherein the first nucleotide of the 30-mer starts at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, or 120 of SEQ ID NO: 2.

In embodiments, the AC that binds to exon 44 of DMD is selected from any one of the nucleic acid sequences shown in Tables 6A-6M, the reverse complement thereof, or a sequence with at least 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity thereto.

In embodiments, the AC binds to a sequence of exon 44 of DMD selected from any one of the nucleic acid sequences shown in Tables 6A-6M. In embodiments, the AC that binds to exon 44 of DMD is selected from any one of the nucleic acid sequences within Tables 6A-6M, the reverse complement thereof, or a sequence with at least 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity thereto. In embodiments, the AC that binds to exon 44 of DMD comprises one or more modified nucleic acids, one or more modified internucleotide linkages, or a combination thereof. In embodiments, the AC that binds to exon 44 comprises one or more morpholine rings, one or more phosphorodiamidate linkages, or a combination thereof. In embodiments, the AC that binds to exon 44 of DMD is an antisense phosphorodiamidate morpholino oligomer (PMO) with a sequence selected from any one of the nucleic acid sequences within Tables 6A-6M, the reverse complement thereof, or a sequence with at least 80%, 81%, 82%, 83%, 84%, 85%, 86%, 87%, 88%, 89%, 90%, 91%, 92%, 93%>94%>95%>96%, 97%, 98% N or 99% nucleic acid sequence identity thereto.

In embodiments, the AC that binds to exon 44 of DMD is 5′-TGAAAACGCCGCCATTTCTCAACAG-3′. In embodiments, the AC that binds to exon 44 of DMD is 5′-ACTGTTCAGCTTCTGTTAGCCACTG-3′. In embodiments, the AC that binds to exon 44 of DMD comprises one or more modified nucleic acids, one or more modified internucleotide linkages, or a combination thereof. In embodiments, the AC that binds to exon 44 comprises one or more morpholine rings, one or more phosphorodiamidate linkages, or combination thereof. In embodiments, the AC that binds to exon 44 of DMD is an antisense phosphorodiamidate morpholino oligomer (PMO) with a sequence that is 5′-TGAAAACGCCGCCATTTCTCAACAG-3′. In embodiments, the AC that binds to exon 44 of DMD is an antisense phosphorodiamidate morpholino oligomer (PMO) with a sequence that is 5′-ACTGTTCAGCTTCTGTTAGCCACTG-3′.

TABLE 6A
18-merACs that bind to exon 44 of DMD
Nucleic Acid Sequence (5′-3′)

GGCGATTTGACAGATCTG
GCGATTTGACAGATCTGT
CGATTTGACAGATCTGTT
GATTTGACAGATCTGTTG
ATTTGACAGATCTGTTGA
TTTGACAGATCTGTTGAG
TTGACAGATCTGTTGAGA
TGACAGATCTGTTGAGAA
GACAGATCTGTTGAGAAA
ACAGATCTGTTGAGAAAT
CAGATCTGTTGAGAAATG
AGATCTGTTGAGAAATGG
GATCTGTTGAGAAATGGC
ATCTGTTGAGAAATGGCG
TCTGTTGAGAAATGGCGG
CTGTTGAGAAATGGCGGC
TGTTGAGAAATGGCGGCG
GTTGAGAAATGGCGGCGT
TTGAGAAATGGCGGCGTT
TGAGAAATGGCGGCGTTT
GAGAAATGGCGGCGTTTT
AGAAATGGCGGCGTTTTC
GAAATGGCGGCGTTTTCA
AAATGGCGGCGTTTTCAT
AATGGCGGCGTTTTCATT
ATGGCGGCGTTTTCATTA
TGGCGGCGTTTTCATTAT
GGCGGCGTTTTCATTATG
GCGGCGTTTTCATTATGA
CGGCGTTTTCATTATGAT
GGCGTTTTCATTATGATA
GCGTTTTCATTATGATAT
CGTTTTCATTATGATATA
GTTTTCATTATGATATAA
TTTTCATTATGATATAAA
TTTCATTATGATATAAAG
TTCATTATGATATAAAGA
TCATTATGATATAAAGAT
CATTATGATATAAAGATA
ATTATGATATAAAGATAT
TTATGATATAAAGATATT
TATGATATAAAGATATTT
ATGATATAAAGATATTTA
TGATATAAAGATATTTAA
GATATAAAGATATTTAAT
ATATAAAGATATTTAATC
TATAAAGATATTTAATCA
ATAAAGATATTTAATCAG
TAAAGATATTTAATCAGT
AAAGATATTTAATCAGTG
AAGATATTTAATCAGTGG
AGATATTTAATCAGTGGC
GATATTTAATCAGTGGCT
ATATTTAATCAGTGGCTA
TATTTAATCAGTGGCTAA
ATTTAATCAGTGGCTAAC
TTTAATCAGTGGCTAACA
TTAATCAGTGGCTAACAG
TAATCAGTGGCTAACAGA
AATCAGTGGCTAACAGAA
ATCAGTGGCTAACAGAAG
TCAGTGGCTAACAGAAGC
CAGTGGCTAACAGAAGCT
AGTGGCTAACAGAAGCTG
GTGGCTAACAGAAGCTGA
TGGCTAACAGAAGCTGAA
GGCTAACAGAAGCTGAAC
GCTAACAGAAGCTGAACA
CTAACAGAAGCTGAACAG
TAACAGAAGCTGAACAGT
AACAGAAGCTGAACAGTT
ACAGAAGCTGAACAGTTT
CAGAAGCTGAACAGTTTC
AGAAGCTGAACAGTTTCT
GAAGCTGAACAGTTTCTC
AAGCTGAACAGTTTCTCA
AGCTGAACAGTTTCTCAG
GCTGAACAGTTTCTCAGA
CTGAACAGTTTCTCAGAA
TGAACAGTTTCTCAGAAA
GAACAGTTTCTCAGAAAG
AACAGTTTCTCAGAAAGA
ACAGTTTCTCAGAAAGAC
CAGTTTCTCAGAAAGACA
AGTTTCTCAGAAAGACAC
GTTTCTCAGAAAGACACA
TTTCTCAGAAAGACACAA
TTCTCAGAAAGACACAAA
TCTCAGAAAGACACAAAT
CTCAGAAAGACACAAATT
TCAGAAAGACACAAATTC
CAGAAAGACACAAATTCC
AGAAAGACACAAATTCCT
GAAAGACACAAATTCCTG
AAAGACACAAATTCCTGA
AAGACACAAATTCCTGAG
AGACACAAATTCCTGAGA
GACACAAATTCCTGAGAA
ACACAAATTCCTGAGAAT
CACAAATTCCTGAGAATT
ACAAATTCCTGAGAATTG
CAAATTCCTGAGAATTGG
AAATTCCTGAGAATTGGG
AATTCCTGAGAATTGGGA
ATTCCTGAGAATTGGGAA
TTCCTGAGAATTGGGAAC
TCCTGAGAATTGGGAACA
CCTGAGAATTGGGAACAT
CTGAGAATTGGGAACATG
TGAGAATTGGGAACATGC
GAGAATTGGGAACATGCT
AGAATTGGGAACATGCTA
GAATTGGGAACATGCTAA
AATTGGGAACATGCTAAA
ATTGGGAACATGCTAAAT
TTGGGAACATGCTAAATA
TGGGAACATGCTAAATAC
GGGAACATGCTAAATACA
GGAACATGCTAAATACAA
GAACATGCTAAATACAAA
AACATGCTAAATACAAAT
ACATGCTAAATACAAATG
CATGCTAAATACAAATGG
ATGCTAAATACAAATGGT
TGCTAAATACAAATGGTA
GCTAAATACAAATGGTAT
CTAAATACAAATGGTATC
TAAATACAAATGGTATCT
AAATACAAATGGTATCTT
AATACAAATGGTATCTTA
ATACAAATGGTATCTTAA
TACAAATGGTATCTTAAG

TABLE 6B
19-mer ACs that bind to exon 44 of DMD
Nucleic Acid Sequence (5′-3′)

GGCGATTTGACAGATCTGT
GCGATTTGACAGATCTGTT
CGATTTGACAGATCTGTTG
GATTTGACAGATCTGTTGA
ATTTGACAGATCTGTTGAG
TTTGACAGATCTGTTGAGA
TTGACAGATCTGTTGAGAA
TGACAGATCTGTTGAGAAA
GACAGATCTGTTGAGAAAT
ACAGATCTGTTGAGAAATG
CAGATCTGTTGAGAAATGG
AGATCTGTTGAGAAATGGC
GATCTGTTGAGAAATGGCG
ATCTGTTGAGAAATGGCGG
TCTGTTGAGAAATGGCGGC
CTGTTGAGAAATGGCGGCG
TGTTGAGAAATGGCGGCGT
GTTGAGAAATGGCGGCGTT
TTGAGAAATGGCGGCGTTT
TGAGAAATGGCGGCGTTTT
GAGAAATGGCGGCGTTTTC
AGAAATGGCGGCGTTTTCA
GAAATGGCGGCGTTTTCAT
AAATGGCGGCGTTTTCATT
AATGGCGGCGTTTTCATTA
ATGGCGGCGTTTTCATTAT
TGGCGGCGTTTTCATTATG
GGCGGCGTTTTCATTATGA
GCGGCGTTTTCATTATGAT
CGGCGTTTTCATTATGATA
GGCGTTTTCATTATGATAT
GCGTTTTCATTATGATATA
CGTTTTCATTATGATATAA
GTTTTCATTATGATATAAA
TTTTCATTATGATATAAAG
TTTCATTATGATATAAAGA
TTCATTATGATATAAAGAT
TCATTATGATATAAAGATA
CATTATGATATAAAGATAT
ATTATGATATAAAGATATT
TTATGATATAAAGATATTT
TATGATATAAAGATATTTA
ATGATATAAAGATATTTAA
TGATATAAAGATATTTAAT
GATATAAAGATATTTAATC
ATATAAAGATATTTAATCA
TATAAAGATATTTAATCAG
ATAAAGATATTTAATCAGT
TAAAGATATTTAATCAGTG
AAAGATATTTAATCAGTGG
AAGATATTTAATCAGTGGC
AGATATTTAATCAGTGGCT
GATATTTAATCAGTGGCTA
ATATTTAATCAGTGGCTAA
TATTTAATCAGTGGCTAAC
ATTTAATCAGTGGCTAACA
TTTAATCAGTGGCTAACAG
TTAATCAGTGGCTAACAGA
TAATCAGTGGCTAACAGAA
AATCAGTGGCTAACAGAAG
ATCAGTGGCTAACAGAAGC
TCAGTGGCTAACAGAAGCT
CAGTGGCTAACAGAAGCTG
AGTGGCTAACAGAAGCTGA
GTGGCTAACAGAAGCTGAA
TGGCTAACAGAAGCTGAAC
GGCTAACAGAAGCTGAACA
GCTAACAGAAGCTGAACAG
CTAACAGAAGCTGAACAGT
TAACAGAAGCTGAACAGTT
AACAGAAGCTGAACAGTTT
ACAGAAGCTGAACAGTTTC
CAGAAGCTGAACAGTTTCT
AGAAGCTGAACAGTTTCTC
GAAGCTGAACAGTTTCTCA
AAGCTGAACAGTTTCTCAG
AGCTGAACAGTTTCTCAGA
GCTGAACAGTTTCTCAGAA
CTGAACAGTTTCTCAGAAA
TGAACAGTTTCTCAGAAAG
GAACAGTTTCTCAGAAAGA
AACAGTTTCTCAGAAAGAC
ACAGTTTCTCAGAAAGACA
CAGTTTCTCAGAAAGACAC
AGTTTCTCAGAAAGACACA
GTTTCTCAGAAAGACACAA
TTTCTCAGAAAGACACAAA
TTCTCAGAAAGACACAAAT
TCTCAGAAAGACACAAATT
CTCAGAAAGACACAAATTC
TCAGAAAGACACAAATTCC
CAGAAAGACACAAATTCCT
AGAAAGACACAAATTCCTG
GAAAGACACAAATTCCTGA
AAAGACACAAATTCCTGAG
AAGACACAAATTCCTGAGA
AGACACAAATTCCTGAGAA
GACACAAATTCCTGAGAAT
ACACAAATTCCTGAGAATT
CACAAATTCCTGAGAATTG
ACAAATTCCTGAGAATTGG
CAAATTCCTGAGAATTGGG
AAATTCCTGAGAATTGGGA
AATTCCTGAGAATTGGGAA
ATTCCTGAGAATTGGGAAC
TTCCTGAGAATTGGGAACA
TCCTGAGAATTGGGAACAT
CCTGAGAATTGGGAACATG
CTGAGAATTGGGAACATGC
TGAGAATTGGGAACATGCT
GAGAATTGGGAACATGCTA
AGAATTGGGAACATGCTAA
GAATTGGGAACATGCTAAA
AATTGGGAACATGCTAAAT
ATTGGGAACATGCTAAATA
TTGGGAACATGCTAAATAC
TGGGAACATGCTAAATACA
GGGAACATGCTAAATACAA
GGAACATGCTAAATACAAA
GAACATGCTAAATACAAAT
AACATGCTAAATACAAATG
ACATGCTAAATACAAATGG
CATGCTAAATACAAATGGT
ATGCTAAATACAAATGGTA
TGCTAAATACAAATGGTAT
GCTAAATACAAATGGTATC
CTAAATACAAATGGTATCT
TAAATACAAATGGTATCTT
AAATACAAATGGTATCTTA
AATACAAATGGTATCTTAA
ATACAAATGGTATCTTAAG

TABLE 6C
20-mer ACs that bind to exon 44 of DMD
Nucleic Acid Sequence (5′-3′)

GGCGATTTGACAGATCTGTT
GCGATTTGACAGATCTGTTG
CGATTTGACAGATCTGTTGA
GATTTGACAGATCTGTTGAG
ATTTGACAGATCTGTTGAGA
TTTGACAGATCTGTTGAGAA
TTGACAGATCTGTTGAGAAA
TGACAGATCTGTTGAGAAAT
GACAGATCTGTTGAGAAATG
ACAGATCTGTTGAGAAATGG
CAGATCTGTTGAGAAATGGC
AGATCTGTTGAGAAATGGCG
GATCTGTTGAGAAATGGCGG
ATCTGTTGAGAAATGGCGGC
TCTGTTGAGAAATGGCGGCG
CTGTTGAGAAATGGCGGCGT
TGTTGAGAAATGGCGGCGTT
GTTGAGAAATGGCGGCGTTT
TTGAGAAATGGCGGCGTTTT
TGAGAAATGGCGGCGTTTTC
GAGAAATGGCGGCGTTTTCA
AGAAATGGCGGCGTTTTCAT
GAAATGGCGGCGTTTTCATT
AAATGGCGGCGTTTTCATTA
AATGGCGGCGTTTTCATTAT
ATGGCGGCGTTTTCATTATG
TGGCGGCGTTTTCATTATGA
GGCGGCGTTTTCATTATGAT
GCGGCGTTTTCATTATGATA
CGGCGTTTTCATTATGATAT
GGCGTTTTCATTATGATATA
GCGTTTTCATTATGATATAA
CGTTTTCATTATGATATAAA
GTTTTCATTATGATATAAAG
TTTTCATTATGATATAAAGA
TTTCATTATGATATAAAGAT
TTCATTATGATATAAAGATA
TCATTATGATATAAAGATAT
CATTATGATATAAAGATATT
ATTATGATATAAAGATATTT
TTATGATATAAAGATATTTA
TATGATATAAAGATATTTAA
ATGATATAAAGATATTTAAT
TGATATAAAGATATTTAATC
GATATAAAGATATTTAATCA
ATATAAAGATATTTAATCAG
TATAAAGATATTTAATCAGT
ATAAAGATATTTAATCAGTG
TAAAGATATTTAATCAGTGG
AAAGATATTTAATCAGTGGC
AAGATATTTAATCAGTGGCT
AGATATTTAATCAGTGGCTA
GATATTTAATCAGTGGCTAA
ATATTTAATCAGTGGCTAAC
TATTTAATCAGTGGCTAACA
ATTTAATCAGTGGCTAACAG
TTTAATCAGTGGCTAACAGA
TTAATCAGTGGCTAACAGAA
TAATCAGTGGCTAACAGAAG
AATCAGTGGCTAACAGAAGC
ATCAGTGGCTAACAGAAGCT
TCAGTGGCTAACAGAAGCTG
CAGTGGCTAACAGAAGCTGA
AGTGGCTAACAGAAGCTGAA
GTGGCTAACAGAAGCTGAAC
TGGCTAACAGAAGCTGAACA
GGCTAACAGAAGCTGAACAG
GCTAACAGAAGCTGAACAGT
CTAACAGAAGCTGAACAGTT
TAACAGAAGCTGAACAGTTT
AACAGAAGCTGAACAGTTTC
ACAGAAGCTGAACAGTTTCT
CAGAAGCTGAACAGTTTCTC
AGAAGCTGAACAGTTTCTCA
GAAGCTGAACAGTTTCTCAG
AAGCTGAACAGTTTCTCAGA
AGCTGAACAGTTTCTCAGAA
GCTGAACAGTTTCTCAGAAA
CTGAACAGTTTCTCAGAAAG
TGAACAGTTTCTCAGAAAGA
GAACAGTTTCTCAGAAAGAC
AACAGTTTCTCAGAAAGACA
ACAGTTTCTCAGAAAGACAC
CAGTTTCTCAGAAAGACACA
AGTTTCTCAGAAAGACACAA
GTTTCTCAGAAAGACACAAA
TTTCTCAGAAAGACACAAAT
TTCTCAGAAAGACACAAATT
TCTCAGAAAGACACAAATTC
CTCAGAAAGACACAAATTCC
TCAGAAAGACACAAATTCCT
CAGAAAGACACAAATTCCTG
AGAAAGACACAAATTCCTGA
GAAAGACACAAATTCCTGAG
AAAGACACAAATTCCTGAGA
AAGACACAAATTCCTGAGAA
AGACACAAATTCCTGAGAAT
GACACAAATTCCTGAGAATT
ACACAAATTCCTGAGAATTG
CACAAATTCCTGAGAATTGG
ACAAATTCCTGAGAATTGGG
CAAATTCCTGAGAATTGGGA
AAATTCCTGAGAATTGGGAA
AATTCCTGAGAATTGGGAAC
ATTCCTGAGAATTGGGAACA
TTCCTGAGAATTGGGAACAT
TCCTGAGAATTGGGAACATG
CCTGAGAATTGGGAACATGC
CTGAGAATTGGGAACATGCT
TGAGAATTGGGAACATGCTA
GAGAATTGGGAACATGCTAA
AGAATTGGGAACATGCTAAA
GAATTGGGAACATGCTAAAT
AATTGGGAACATGCTAAATA
ATTGGGAACATGCTAAATAC
TTGGGAACATGCTAAATACA
TGGGAACATGCTAAATACAA
GGGAACATGCTAAATACAAA
GGAACATGCTAAATACAAAT
GAACATGCTAAATACAAATG
AACATGCTAAATACAAATGG
ACATGCTAAATACAAATGGT
CATGCTAAATACAAATGGTA
ATGCTAAATACAAATGGTAT
TGCTAAATACAAATGGTATC
GCTAAATACAAATGGTATCT
CTAAATACAAATGGTATCTT
TAAATACAAATGGTATCTTA
AAATACAAATGGTATCTTAA
AATACAAATGGTATCTTAAG

TABLE 6D
21-mer ACs that bind to exon 44 of DMD
Nucleic Acid Sequence (5′-3′)

GGCGATTTGACAGATCTGTTG
GCGATTTGACAGATCTGTTGA
CGATTTGACAGATCTGTTGAG
GATTTGACAGATCTGTTGAGA
ATTTGACAGATCTGTTGAGAA
TTTGACAGATCTGTTGAGAAA
TTGACAGATCTGTTGAGAAAT
TGACAGATCTGTTGAGAAATG
GACAGATCTGTTGAGAAATGG
ACAGATCTGTTGAGAAATGGC
CAGATCTGTTGAGAAATGGCG
AGATCTGTTGAGAAATGGCGG
GATCTGTTGAGAAATGGCGGC
ATCTGTTGAGAAATGGCGGCG
TCTGTTGAGAAATGGCGGCGT
CTGTTGAGAAATGGCGGCGTT
TGTTGAGAAATGGCGGCGTTT
GTTGAGAAATGGCGGCGTTTT
TTGAGAAATGGCGGCGTTTTC
TGAGAAATGGCGGCGTTTTCA
GAGAAATGGCGGCGTTTTCAT
AGAAATGGCGGCGTTTTCATT
GAAATGGCGGCGTTTTCATTA
AAATGGCGGCGTTTTCATTAT
AATGGCGGCGTTTTCATTATG
ATGGCGGCGTTTTCATTATGA
TGGCGGCGTTTTCATTATGAT
GGCGGCGTTTTCATTATGATA
GCGGCGTTTTCATTATGATAT
CGGCGTTTTCATTATGATATA
GGCGTTTTCATTATGATATAA
GCGTTTTCATTATGATATAAA
CGTTTTCATTATGATATAAAG
GTTTTCATTATGATATAAAGA
TTTTCATTATGATATAAAGAT
TTTCATTATGATATAAAGATA
TTCATTATGATATAAAGATAT
TCATTATGATATAAAGATATT
CATTATGATATAAAGATATTT
ATTATGATATAAAGATATTTA
TTATGATATAAAGATATTTAA
TATGATATAAAGATATTTAAT
ATGATATAAAGATATTTAATC
TGATATAAAGATATTTAATCA
GATATAAAGATATTTAATCAG
ATATAAAGATATTTAATCAGT
TATAAAGATATTTAATCAGTG
ATAAAGATATTTAATCAGTGG
TAAAGATATTTAATCAGTGGC
AAAGATATTTAATCAGTGGCT
AAGATATTTAATCAGTGGCTA
AGATATTTAATCAGTGGCTAA
GATATTTAATCAGTGGCTAAC
ATATTTAATCAGTGGCTAACA
TATTTAATCAGTGGCTAACAG
ATTTAATCAGTGGCTAACAGA
TTTAATCAGTGGCTAACAGAA
TTAATCAGTGGCTAACAGAAG
TAATCAGTGGCTAACAGAAGC
AATCAGTGGCTAACAGAAGCT
ATCAGTGGCTAACAGAAGCTG
TCAGTGGCTAACAGAAGCTGA
CAGTGGCTAACAGAAGCTGAA
AGTGGCTAACAGAAGCTGAAC
GTGGCTAACAGAAGCTGAACA
TGGCTAACAGAAGCTGAACAG
GGCTAACAGAAGCTGAACAGT
GCTAACAGAAGCTGAACAGTT
CTAACAGAAGCTGAACAGTTT
TAACAGAAGCTGAACAGTTTC
AACAGAAGCTGAACAGTTTCT
ACAGAAGCTGAACAGTTTCTC
CAGAAGCTGAACAGTTTCTCA
AGAAGCTGAACAGTTTCTCAG
GAAGCTGAACAGTTTCTCAGA
AAGCTGAACAGTTTCTCAGAA
AGCTGAACAGTTTCTCAGAAA
GCTGAACAGTTTCTCAGAAAG
CTGAACAGTTTCTCAGAAAGA
TGAACAGTTTCTCAGAAAGAC
GAACAGTTTCTCAGAAAGACA
AACAGTTTCTCAGAAAGACAC
ACAGTTTCTCAGAAAGACACA
CAGTTTCTCAGAAAGACACAA
AGTTTCTCAGAAAGACACAAA
GTTTCTCAGAAAGACACAAAT
TTTCTCAGAAAGACACAAATT
TTCTCAGAAAGACACAAATTC
TCTCAGAAAGACACAAATTCC
CTCAGAAAGACACAAATTCCT
TCAGAAAGACACAAATTCCTG
CAGAAAGACACAAATTCCTGA
AGAAAGACACAAATTCCTGAG
GAAAGACACAAATTCCTGAGA
AAAGACACAAATTCCTGAGAA
AAGACACAAATTCCTGAGAAT
AGACACAAATTCCTGAGAATT
GACACAAATTCCTGAGAATTG
ACACAAATTCCTGAGAATTGG
CACAAATTCCTGAGAATTGGG
ACAAATTCCTGAGAATTGGGA
CAAATTCCTGAGAATTGGGAA
AAATTCCTGAGAATTGGGAAC
AATTCCTGAGAATTGGGAACA
ATTCCTGAGAATTGGGAACAT
TTCCTGAGAATTGGGAACATG
TCCTGAGAATTGGGAACATGC
CCTGAGAATTGGGAACATGCT
CTGAGAATTGGGAACATGCTA
TGAGAATTGGGAACATGCTAA
GAGAATTGGGAACATGCTAAA
AGAATTGGGAACATGCTAAAT
GAATTGGGAACATGCTAAATA
AATTGGGAACATGCTAAATAC
ATTGGGAACATGCTAAATACA
TTGGGAACATGCTAAATACAA
TGGGAACATGCTAAATACAAA
GGGAACATGCTAAATACAAAT
GGAACATGCTAAATACAAATG
GAACATGCTAAATACAAATGG
AACATGCTAAATACAAATGGT
ACATGCTAAATACAAATGGTA
CATGCTAAATACAAATGGTAT
ATGCTAAATACAAATGGTATC
TGCTAAATACAAATGGTATCT
GCTAAATACAAATGGTATCTT
CTAAATACAAATGGTATCTTA
TAAATACAAATGGTATCTTAA
AAATACAAATGGTATCTTAAG

TABLE 6E
22-mer ACs that bind to exon 44 of DMD
Nucleic Acid Sequence (5′-3′)

GGCGATTTGACAGATCTGTTGA
GCGATTTGACAGATCTGTTGAG
CGATTTGACAGATCTGTTGAGA
GATTTGACAGATCTGTTGAGAA
ATTTGACAGATCTGTTGAGAAA
TTTGACAGATCTGTTGAGAAAT
TTGACAGATCTGTTGAGAAATG
TGACAGATCTGTTGAGAAATGG
GACAGATCTGTTGAGAAATGGC
ACAGATCTGTTGAGAAATGGCG
CAGATCTGTTGAGAAATGGCGG
AGATCTGTTGAGAAATGGCGGC
GATCTGTTGAGAAATGGCGGCG
ATCTGTTGAGAAATGGCGGCGT
TCTGTTGAGAAATGGCGGCGTT
CTGTTGAGAAATGGCGGCGTTT
TGTTGAGAAATGGCGGCGTTTT
GTTGAGAAATGGCGGCGTTTTC
TTGAGAAATGGCGGCGTTTTCA
TGAGAAATGGCGGCGTTTTCAT
GAGAAATGGCGGCGTTTTCATT
AGAAATGGCGGCGTTTTCATTA
GAAATGGCGGCGTTTTCATTAT
AAATGGCGGCGTTTTCATTATG
AATGGCGGCGTTTTCATTATGA
ATGGCGGCGTTTTCATTATGAT
TGGCGGCGTTTTCATTATGATA
GGCGGCGTTTTCATTATGATAT
GCGGCGTTTTCATTATGATATA
CGGCGTTTTCATTATGATATAA
GGCGTTTTCATTATGATATAAA
GCGTTTTCATTATGATATAAAG
CGTTTTCATTATGATATAAAGA
GTTTTCATTATGATATAAAGAT
TTTTCATTATGATATAAAGATA
TTTCATTATGATATAAAGATAT
TTCATTATGATATAAAGATATT
TCATTATGATATAAAGATATTT
CATTATGATATAAAGATATTTA
ATTATGATATAAAGATATTTAA
TTATGATATAAAGATATTTAAT
TATGATATAAAGATATTTAATC
ATGATATAAAGATATTTAATCA
TGATATAAAGATATTTAATCAG
GATATAAAGATATTTAATCAGT
ATATAAAGATATTTAATCAGTG
TATAAAGATATTTAATCAGTGG
ATAAAGATATTTAATCAGTGGC
TAAAGATATTTAATCAGTGGCT
AAAGATATTTAATCAGTGGCTA
AAGATATTTAATCAGTGGCTAA
AGATATTTAATCAGTGGCTAAC
GATATTTAATCAGTGGCTAACA
ATATTTAATCAGTGGCTAACAG
TATTTAATCAGTGGCTAACAGA
ATTTAATCAGTGGCTAACAGAA
TTTAATCAGTGGCTAACAGAAG
TTAATCAGTGGCTAACAGAAGC
TAATCAGTGGCTAACAGAAGCT
AATCAGTGGCTAACAGAAGCTG
ATCAGTGGCTAACAGAAGCTGA
TCAGTGGCTAACAGAAGCTGAA
CAGTGGCTAACAGAAGCTGAAC
AGTGGCTAACAGAAGCTGAACA
GTGGCTAACAGAAGCTGAACAG
TGGCTAACAGAAGCTGAACAGT
GGCTAACAGAAGCTGAACAGTT
GCTAACAGAAGCTGAACAGTTT
CTAACAGAAGCTGAACAGTTTC
TAACAGAAGCTGAACAGTTTCT
AACAGAAGCTGAACAGTTTCTC
ACAGAAGCTGAACAGTTTCTCA
CAGAAGCTGAACAGTTTCTCAG
AGAAGCTGAACAGTTTCTCAGA
GAAGCTGAACAGTTTCTCAGAA
AAGCTGAACAGTTTCTCAGAAA
AGCTGAACAGTTTCTCAGAAAG
GCTGAACAGTTTCTCAGAAAGA
CTGAACAGTTTCTCAGAAAGAC
TGAACAGTTTCTCAGAAAGACA
GAACAGTTTCTCAGAAAGACAC
AACAGTTTCTCAGAAAGACACA
ACAGTTTCTCAGAAAGACACAA
CAGTTTCTCAGAAAGACACAAA
AGTTTCTCAGAAAGACACAAAT
GTTTCTCAGAAAGACACAAATT
TTTCTCAGAAAGACACAAATTC
TTCTCAGAAAGACACAAATTCC
TCTCAGAAAGACACAAATTCCT
CTCAGAAAGACACAAATTCCTG
TCAGAAAGACACAAATTCCTGA
CAGAAAGACACAAATTCCTGAG
AGAAAGACACAAATTCCTGAGA
GAAAGACACAAATTCCTGAGAA
AAAGACACAAATTCCTGAGAAT
AAGACACAAATTCCTGAGAATT
AGACACAAATTCCTGAGAATTG
GACACAAATTCCTGAGAATTGG
ACACAAATTCCTGAGAATTGGG
CACAAATTCCTGAGAATTGGGA
ACAAATTCCTGAGAATTGGGAA
CAAATTCCTGAGAATTGGGAAC
AAATTCCTGAGAATTGGGAACA
AATTCCTGAGAATTGGGAACAT
ATTCCTGAGAATTGGGAACATG
TTCCTGAGAATTGGGAACATGC
TCCTGAGAATTGGGAACATGCT
CCTGAGAATTGGGAACATGCTA
CTGAGAATTGGGAACATGCTAA
TGAGAATTGGGAACATGCTAAA
GAGAATTGGGAACATGCTAAAT
AGAATTGGGAACATGCTAAATA
GAATTGGGAACATGCTAAATAC
AATTGGGAACATGCTAAATACA
ATTGGGAACATGCTAAATACAA
TTGGGAACATGCTAAATACAAA
TGGGAACATGCTAAATACAAAT
GGGAACATGCTAAATACAAATG
GGAACATGCTAAATACAAATGG
GAACATGCTAAATACAAATGGT
AACATGCTAAATACAAATGGTA
ACATGCTAAATACAAATGGTAT
CATGCTAAATACAAATGGTATC
ATGCTAAATACAAATGGTATCT
TGCTAAATACAAATGGTATCTT
GCTAAATACAAATGGTATCTTA
CTAAATACAAATGGTATCTTAA
TAAATACAAATGGTATCTTAAG

TABLE 6F
23-mer ACs that bind to exon 44 of DMD
Nucleic Acid Sequence (5′-3′)

GGCGATTTGACAGATCTGTTGAG
GCGATTTGACAGATCTGTTGAGA
CGATTTGACAGATCTGTTGAGAA
GATTTGACAGATCTGTTGAGAAA
ATTTGACAGATCTGTTGAGAAAT
TTTGACAGATCTGTTGAGAAATG
TTGACAGATCTGTTGAGAAATGG
TGACAGATCTGTTGAGAAATGGC
GACAGATCTGTTGAGAAATGGCG
ACAGATCTGTTGAGAAATGGCGG
CAGATCTGTTGAGAAATGGCGGC
AGATCTGTTGAGAAATGGCGGCG
GATCTGTTGAGAAATGGCGGCGT
ATCTGTTGAGAAATGGCGGCGTT
TCTGTTGAGAAATGGCGGCGTTT
CTGTTGAGAAATGGCGGCGTTTT
TGTTGAGAAATGGCGGCGTTTTC
GTTGAGAAATGGCGGCGTTTTCA
TTGAGAAATGGCGGCGTTTTCAT
TGAGAAATGGCGGCGTTTTCATT
GAGAAATGGCGGCGTTTTCATTA
AGAAATGGCGGCGTTTTCATTAT
GAAATGGCGGCGTTTTCATTATG
AAATGGCGGCGTTTTCATTATGA
AATGGCGGCGTTTTCATTATGAT
ATGGCGGCGTTTTCATTATGATA
TGGCGGCGTTTTCATTATGATAT
GGCGGCGTTTTCATTATGATATA
GCGGCGTTTTCATTATGATATAA
CGGCGTTTTCATTATGATATAAA
GGCGTTTTCATTATGATATAAAG
GCGTTTTCATTATGATATAAAGA
CGTTTTCATTATGATATAAAGAT
GTTTTCATTATGATATAAAGATA
TTTTCATTATGATATAAAGATAT
TTTCATTATGATATAAAGATATT
TTCATTATGATATAAAGATATTT
TCATTATGATATAAAGATATTTA
CATTATGATATAAAGATATTTAA
ATTATGATATAAAGATATTTAAT
TTATGATATAAAGATATTTAATC
TATGATATAAAGATATTTAATCA
ATGATATAAAGATATTTAATCAG
TGATATAAAGATATTTAATCAGT
GATATAAAGATATTTAATCAGTG
ATATAAAGATATTTAATCAGTGG
TATAAAGATATTTAATCAGTGGC
ATAAAGATATTTAATCAGTGGCT
TAAAGATATTTAATCAGTGGCTA
AAAGATATTTAATCAGTGGCTAA
AAGATATTTAATCAGTGGCTAAC
AGATATTTAATCAGTGGCTAACA
GATATTTAATCAGTGGCTAACAG
ATATTTAATCAGTGGCTAACAGA
TATTTAATCAGTGGCTAACAGAA
ATTTAATCAGTGGCTAACAGAAG
TTTAATCAGTGGCTAACAGAAGC
TTAATCAGTGGCTAACAGAAGCT
TAATCAGTGGCTAACAGAAGCTG
AATCAGTGGCTAACAGAAGCTGA
ATCAGTGGCTAACAGAAGCTGAA
TCAGTGGCTAACAGAAGCTGAAC
CAGTGGCTAACAGAAGCTGAACA
AGTGGCTAACAGAAGCTGAACAG
GTGGCTAACAGAAGCTGAACAGT
TGGCTAACAGAAGCTGAACAGTT
GGCTAACAGAAGCTGAACAGTTT
GCTAACAGAAGCTGAACAGTTTC
CTAACAGAAGCTGAACAGTTTCT
TAACAGAAGCTGAACAGTTTCTC
AACAGAAGCTGAACAGTTTCTCA
ACAGAAGCTGAACAGTTTCTCAG
CAGAAGCTGAACAGTTTCTCAGA
AGAAGCTGAACAGTTTCTCAGAA
GAAGCTGAACAGTTTCTCAGAAA
AAGCTGAACAGTTTCTCAGAAAG
AGCTGAACAGTTTCTCAGAAAGA
GCTGAACAGTTTCTCAGAAAGAC
CTGAACAGTTTCTCAGAAAGACA
TGAACAGTTTCTCAGAAAGACAC
GAACAGTTTCTCAGAAAGACACA
AACAGTTTCTCAGAAAGACACAA
ACAGTTTCTCAGAAAGACACAAA
CAGTTTCTCAGAAAGACACAAAT
AGTTTCTCAGAAAGACACAAATT
GTTTCTCAGAAAGACACAAATTC
TTTCTCAGAAAGACACAAATTCC
TTCTCAGAAAGACACAAATTCCT
TCTCAGAAAGACACAAATTCCTG
CTCAGAAAGACACAAATTCCTGA
TCAGAAAGACACAAATTCCTGAG
CAGAAAGACACAAATTCCTGAGA
AGAAAGACACAAATTCCTGAGAA
GAAAGACACAAATTCCTGAGAAT
AAAGACACAAATTCCTGAGAATT
AAGACACAAATTCCTGAGAATTG
AGACACAAATTCCTGAGAATTGG
GACACAAATTCCTGAGAATTGGG
ACACAAATTCCTGAGAATTGGGA
CACAAATTCCTGAGAATTGGGAA
ACAAATTCCTGAGAATTGGGAAC
CAAATTCCTGAGAATTGGGAACA
AAATTCCTGAGAATTGGGAACAT
AATTCCTGAGAATTGGGAACATG
ATTCCTGAGAATTGGGAACATGC
TTCCTGAGAATTGGGAACATGCT
TCCTGAGAATTGGGAACATGCTA
CCTGAGAATTGGGAACATGCTAA
CTGAGAATTGGGAACATGCTAAA
TGAGAATTGGGAACATGCTAAAT
GAGAATTGGGAACATGCTAAATA
AGAATTGGGAACATGCTAAATAC
GAATTGGGAACATGCTAAATACA
AATTGGGAACATGCTAAATACAA
ATTGGGAACATGCTAAATACAAA
TTGGGAACATGCTAAATACAAAT
TGGGAACATGCTAAATACAAATG
GGGAACATGCTAAATACAAATGG
GGAACATGCTAAATACAAATGGT
GAACATGCTAAATACAAATGGTA
AACATGCTAAATACAAATGGTAT
ACATGCTAAATACAAATGGTATC
CATGCTAAATACAAATGGTATCT
ATGCTAAATACAAATGGTATCTT
TGCTAAATACAAATGGTATCTTA
GCTAAATACAAATGGTATCTTAA
CTAAATACAAATGGTATCTTAAG
GCGATTTGACAGATCTGTTGAGAA

TABLE 6G
24-mer ACs that bind to exon 44 of DMD
Nucleic Acid Sequence (5′-3′)

GCGATTTGACAGATCTGTTGAGAA
CGATTTGACAGATCTGTTGAGAAA
GATTTGACAGATCTGTTGAGAAAT
ATTTGACAGATCTGTTGAGAAATG
TTTGACAGATCTGTTGAGAAATGG
TTGACAGATCTGTTGAGAAATGGC
TGACAGATCTGTTGAGAAATGGCG
GACAGATCTGTTGAGAAATGGCGG
ACAGATCTGTTGAGAAATGGCGGC
CAGATCTGTTGAGAAATGGCGGCG
AGATCTGTTGAGAAATGGCGGCGT
GATCTGTTGAGAAATGGCGGCGTT
ATCTGTTGAGAAATGGCGGCGTTT
TCTGTTGAGAAATGGCGGCGTTTT
CTGTTGAGAAATGGCGGCGTTTTC
TGTTGAGAAATGGCGGCGTTTTCA
GTTGAGAAATGGCGGCGTTTTCAT
TTGAGAAATGGCGGCGTTTTCATT
TGAGAAATGGCGGCGTTTTCATTA
GAGAAATGGCGGCGTTTTCATTAT
AGAAATGGCGGCGTTTTCATTATG
GAAATGGCGGCGTTTTCATTATGA
AAATGGCGGCGTTTTCATTATGAT
AATGGCGGCGTTTTCATTATGATA
ATGGCGGCGTTTTCATTATGATAT
TGGCGGCGTTTTCATTATGATATA
GGCGGCGTTTTCATTATGATATAA
GCGGCGTTTTCATTATGATATAAA
CGGCGTTTTCATTATGATATAAAG
GGCGTTTTCATTATGATATAAAGA
GCGTTTTCATTATGATATAAAGAT
CGTTTTCATTATGATATAAAGATA
GTTTTCATTATGATATAAAGATAT
TTTTCATTATGATATAAAGATATT
TTTCATTATGATATAAAGATATTT
TTCATTATGATATAAAGATATTTA
TCATTATGATATAAAGATATTTAA
CATTATGATATAAAGATATTTAAT
ATTATGATATAAAGATATTTAATC
TTATGATATAAAGATATTTAATCA
TATGATATAAAGATATTTAATCAG
ATGATATAAAGATATTTAATCAGT
TGATATAAAGATATTTAATCAGTG
GATATAAAGATATTTAATCAGTGG
ATATAAAGATATTTAATCAGTGGC
TATAAAGATATTTAATCAGTGGCT
ATAAAGATATTTAATCAGTGGCTA
TAAAGATATTTAATCAGTGGCTAA
AAAGATATTTAATCAGTGGCTAAC
AAGATATTTAATCAGTGGCTAACA
AGATATTTAATCAGTGGCTAACAG
GATATTTAATCAGTGGCTAACAGA
ATATTTAATCAGTGGCTAACAGAA
TATTTAATCAGTGGCTAACAGAAG
ATTTAATCAGTGGCTAACAGAAGC
TTTAATCAGTGGCTAACAGAAGCT
TTAATCAGTGGCTAACAGAAGCTG
TAATCAGTGGCTAACAGAAGCTGA
AATCAGTGGCTAACAGAAGCTGAA
ATCAGTGGCTAACAGAAGCTGAAC
TCAGTGGCTAACAGAAGCTGAACA
CAGTGGCTAACAGAAGCTGAACAG
AGTGGCTAACAGAAGCTGAACAGT
GTGGCTAACAGAAGCTGAACAGTT
TGGCTAACAGAAGCTGAACAGTTT
GGCTAACAGAAGCTGAACAGTTTC
GCTAACAGAAGCTGAACAGTTTCT
CTAACAGAAGCTGAACAGTTTCTC
TAACAGAAGCTGAACAGTTTCTCA
AACAGAAGCTGAACAGTTTCTCAG
ACAGAAGCTGAACAGTTTCTCAGA
CAGAAGCTGAACAGTTTCTCAGAA
AGAAGCTGAACAGTTTCTCAGAAA
GAAGCTGAACAGTTTCTCAGAAAG
AAGCTGAACAGTTTCTCAGAAAGA
AGCTGAACAGTTTCTCAGAAAGAC
GCTGAACAGTTTCTCAGAAAGACA
CTGAACAGTTTCTCAGAAAGACAC
TGAACAGTTTCTCAGAAAGACACA
GAACAGTTTCTCAGAAAGACACAA
AACAGTTTCTCAGAAAGACACAAA
ACAGTTTCTCAGAAAGACACAAAT
CAGTTTCTCAGAAAGACACAAATT
AGTTTCTCAGAAAGACACAAATTC
GTTTCTCAGAAAGACACAAATTCC
TTTCTCAGAAAGACACAAATTCCT
TTCTCAGAAAGACACAAATTCCTG
TCTCAGAAAGACACAAATTCCTGA
CTCAGAAAGACACAAATTCCTGAG
TCAGAAAGACACAAATTCCTGAGA
CAGAAAGACACAAATTCCTGAGAA
AGAAAGACACAAATTCCTGAGAAT
GAAAGACACAAATTCCTGAGAATT
AAAGACACAAATTCCTGAGAATTG
AAGACACAAATTCCTGAGAATTGG
AGACACAAATTCCTGAGAATTGGG
GACACAAATTCCTGAGAATTGGGA
ACACAAATTCCTGAGAATTGGGAA
CACAAATTCCTGAGAATTGGGAAC
ACAAATTCCTGAGAATTGGGAACA
CAAATTCCTGAGAATTGGGAACAT
AAATTCCTGAGAATTGGGAACATG
AATTCCTGAGAATTGGGAACATGC
ATTCCTGAGAATTGGGAACATGCT
TTCCTGAGAATTGGGAACATGCTA
TCCTGAGAATTGGGAACATGCTAA
CCTGAGAATTGGGAACATGCTAAA
CTGAGAATTGGGAACATGCTAAAT
TGAGAATTGGGAACATGCTAAATA
GAGAATTGGGAACATGCTAAATAC
AGAATTGGGAACATGCTAAATACA
GAATTGGGAACATGCTAAATACAA
AATTGGGAACATGCTAAATACAAA
ATTGGGAACATGCTAAATACAAAT
TTGGGAACATGCTAAATACAAATG
TGGGAACATGCTAAATACAAATGG
GGGAACATGCTAAATACAAATGGT
GGAACATGCTAAATACAAATGGTA
GAACATGCTAAATACAAATGGTAT
AACATGCTAAATACAAATGGTATC
ACATGCTAAATACAAATGGTATCT
CATGCTAAATACAAATGGTATCTT
ATGCTAAATACAAATGGTATCTTA
TGCTAAATACAAATGGTATCTTAA
GCTAAATACAAATGGTATCTTAAG

TABLE 6H
25-mer ACs that bind to exon 44 of DMD
Nucleic Acid Sequence (5′-3′)

GGCGATTTGACAGATCTGTTGAGAA
GCGATTTGACAGATCTGTTGAGAAA
CGATTTGACAGATCTGTTGAGAAAT
GATTTGACAGATCTGTTGAGAAATG
ATTTGACAGATCTGTTGAGAAATGG
TTTGACAGATCTGTTGAGAAATGGC
TTGACAGATCTGTTGAGAAATGGCG
TGACAGATCTGTTGAGAAATGGCGG
GACAGATCTGTTGAGAAATGGCGGC
ACAGATCTGTTGAGAAATGGCGGCG
CAGATCTGTTGAGAAATGGCGGCGT
AGATCTGTTGAGAAATGGCGGCGTT
GATCTGTTGAGAAATGGCGGCGTTT
ATCTGTTGAGAAATGGCGGCGTTTT
TCTGTTGAGAAATGGCGGCGTTTTC
CTGTTGAGAAATGGCGGCGTTTTCA
TGTTGAGAAATGGCGGCGTTTTCAT
GTTGAGAAATGGCGGCGTTTTCATT
TTGAGAAATGGCGGCGTTTTCATTA
TGAGAAATGGCGGCGTTTTCATTAT
GAGAAATGGCGGCGTTTTCATTATG
AGAAATGGCGGCGTTTTCATTATGA
GAAATGGCGGCGTTTTCATTATGAT
AAATGGCGGCGTTTTCATTATGATA
AATGGCGGCGTTTTCATTATGATAT
ATGGCGGCGTTTTCATTATGATATA
TGGCGGCGTTTTCATTATGATATAA
GGCGGCGTTTTCATTATGATATAAA
GCGGCGTTTTCATTATGATATAAAG
CGGCGTTTTCATTATGATATAAAGA
GGCGTTTTCATTATGATATAAAGAT
GCGTTTTCATTATGATATAAAGATA
CGTTTTCATTATGATATAAAGATAT
GTTTTCATTATGATATAAAGATATT
TTTTCATTATGATATAAAGATATTT
TTTCATTATGATATAAAGATATTTA
TTCATTATGATATAAAGATATTTAA
TCATTATGATATAAAGATATTTAAT
CATTATGATATAAAGATATTTAATC
ATTATGATATAAAGATATTTAATCA
TTATGATATAAAGATATTTAATCAG
TATGATATAAAGATATTTAATCAGT
ATGATATAAAGATATTTAATCAGTG
TGATATAAAGATATTTAATCAGTGG
GATATAAAGATATTTAATCAGTGGC
ATATAAAGATATTTAATCAGTGGCT
TATAAAGATATTTAATCAGTGGCTA
ATAAAGATATTTAATCAGTGGCTAA
TAAAGATATTTAATCAGTGGCTAAC
AAAGATATTTAATCAGTGGCTAACA
AAGATATTTAATCAGTGGCTAACAG
AGATATTTAATCAGTGGCTAACAGA
GATATTTAATCAGTGGCTAACAGAA
ATATTTAATCAGTGGCTAACAGAAG
TATTTAATCAGTGGCTAACAGAAGC
ATTTAATCAGTGGCTAACAGAAGCT
TTTAATCAGTGGCTAACAGAAGCTG
TTAATCAGTGGCTAACAGAAGCTGA
TAATCAGTGGCTAACAGAAGCTGAA
AATCAGTGGCTAACAGAAGCTGAAC
ATCAGTGGCTAACAGAAGCTGAACA
TCAGTGGCTAACAGAAGCTGAACAG
CAGTGGCTAACAGAAGCTGAACAGT
AGTGGCTAACAGAAGCTGAACAGTT
GTGGCTAACAGAAGCTGAACAGTTT
TGGCTAACAGAAGCTGAACAGTTTC
GGCTAACAGAAGCTGAACAGTTTCT
GCTAACAGAAGCTGAACAGTTTCTC
CTAACAGAAGCTGAACAGTTTCTCA
TAACAGAAGCTGAACAGTTTCTCAG
AACAGAAGCTGAACAGTTTCTCAGA
ACAGAAGCTGAACAGTTTCTCAGAA
CAGAAGCTGAACAGTTTCTCAGAAA
AGAAGCTGAACAGTTTCTCAGAAAG
GAAGCTGAACAGTTTCTCAGAAAGA
AAGCTGAACAGTTTCTCAGAAAGAC
AGCTGAACAGTTTCTCAGAAAGACA
GCTGAACAGTTTCTCAGAAAGACAC
CTGAACAGTTTCTCAGAAAGACACA
TGAACAGTTTCTCAGAAAGACACAA
GAACAGTTTCTCAGAAAGACACAAA
AACAGTTTCTCAGAAAGACACAAAT
ACAGTTTCTCAGAAAGACACAAATT
CAGTTTCTCAGAAAGACACAAATTC
AGTTTCTCAGAAAGACACAAATTCC
GTTTCTCAGAAAGACACAAATTCCT
TTTCTCAGAAAGACACAAATTCCTG
TTCTCAGAAAGACACAAATTCCTGA
TCTCAGAAAGACACAAATTCCTGAG
CTCAGAAAGACACAAATTCCTGAGA
TCAGAAAGACACAAATTCCTGAGAA
CAGAAAGACACAAATTCCTGAGAAT
AGAAAGACACAAATTCCTGAGAATT
GAAAGACACAAATTCCTGAGAATTG
AAAGACACAAATTCCTGAGAATTGG
AAGACACAAATTCCTGAGAATTGGG
AGACACAAATTCCTGAGAATTGGGA
GACACAAATTCCTGAGAATTGGGAA
ACACAAATTCCTGAGAATTGGGAAC
CACAAATTCCTGAGAATTGGGAACA
ACAAATTCCTGAGAATTGGGAACAT
CAAATTCCTGAGAATTGGGAACATG
AAATTCCTGAGAATTGGGAACATGC
AATTCCTGAGAATTGGGAACATGCT
ATTCCTGAGAATTGGGAACATGCTA
TTCCTGAGAATTGGGAACATGCTAA
TCCTGAGAATTGGGAACATGCTAAA
CCTGAGAATTGGGAACATGCTAAAT
CTGAGAATTGGGAACATGCTAAATA
TGAGAATTGGGAACATGCTAAATAC
GAGAATTGGGAACATGCTAAATACA
AGAATTGGGAACATGCTAAATACAA
GAATTGGGAACATGCTAAATACAAA
AATTGGGAACATGCTAAATACAAAT
ATTGGGAACATGCTAAATACAAATG
TTGGGAACATGCTAAATACAAATGG
TGGGAACATGCTAAATACAAATGGT
GGGAACATGCTAAATACAAATGGTA
GGAACATGCTAAATACAAATGGTAT
GAACATGCTAAATACAAATGGTATC
AACATGCTAAATACAAATGGTATCT
ACATGCTAAATACAAATGGTATCTT
CATGCTAAATACAAATGGTATCTTA
ATGCTAAATACAAATGGTATCTTAA
TGCTAAATACAAATGGTATCTTAAG
TGAAAACGCCGCCATTTCTCAACAG
AAACGCCGCCATTTCTCAACAGATC
AACGCCGCCATTTCTCAACAGATCT

TABLE 6I
26-mer ACs that bind to exon 44 of DMD
Nucleic Acid Sequence (5′-3′)

GGCGATTTGACAGATCTGTTGAGAAA
GCGATTTGACAGATCTGTTGAGAAAT
CGATTTGACAGATCTGTTGAGAAATG
GATTTGACAGATCTGTTGAGAAATGG
ATTTGACAGATCTGTTGAGAAATGGC
TTTGACAGATCTGTTGAGAAATGGCG
TTGACAGATCTGTTGAGAAATGGCGG
TGACAGATCTGTTGAGAAATGGCGGC
GACAGATCTGTTGAGAAATGGCGGCG
ACAGATCTGTTGAGAAATGGCGGCGT
CAGATCTGTTGAGAAATGGCGGCGTT
AGATCTGTTGAGAAATGGCGGCGTTT
GATCTGTTGAGAAATGGCGGCGTTTT
ATCTGTTGAGAAATGGCGGCGTTTTC
TCTGTTGAGAAATGGCGGCGTTTTCA
CTGTTGAGAAATGGCGGCGTTTTCAT
TGTTGAGAAATGGCGGCGTTTTCATT
GTTGAGAAATGGCGGCGTTTTCATTA
TTGAGAAATGGCGGCGTTTTCATTAT
TGAGAAATGGCGGCGTTTTCATTATG
GAGAAATGGCGGCGTTTTCATTATGA
AGAAATGGCGGCGTTTTCATTATGAT
GAAATGGCGGCGTTTTCATTATGATA
AAATGGCGGCGTTTTCATTATGATAT
AATGGCGGCGTTTTCATTATGATATA
ATGGCGGCGTTTTCATTATGATATAA
TGGCGGCGTTTTCATTATGATATAAA
GGCGGCGTTTTCATTATGATATAAAG
GCGGCGTTTTCATTATGATATAAAGA
CGGCGTTTTCATTATGATATAAAGAT
GGCGTTTTCATTATGATATAAAGATA
GCGTTTTCATTATGATATAAAGATAT
CGTTTTCATTATGATATAAAGATATT
GTTTTCATTATGATATAAAGATATTT
TTTTCATTATGATATAAAGATATTTA
TTTCATTATGATATAAAGATATTTAA
TTCATTATGATATAAAGATATTTAAT
TCATTATGATATAAAGATATTTAATC
CATTATGATATAAAGATATTTAATCA
ATTATGATATAAAGATATTTAATCAG
TTATGATATAAAGATATTTAATCAGT
TATGATATAAAGATATTTAATCAGTG
ATGATATAAAGATATTTAATCAGTGG
TGATATAAAGATATTTAATCAGTGGC
GATATAAAGATATTTAATCAGTGGCT
ATATAAAGATATTTAATCAGTGGCTA
TATAAAGATATTTAATCAGTGGCTAA
ATAAAGATATTTAATCAGTGGCTAAC
TAAAGATATTTAATCAGTGGCTAACA
AAAGATATTTAATCAGTGGCTAACAG
AAGATATTTAATCAGTGGCTAACAGA
AGATATTTAATCAGTGGCTAACAGAA
GATATTTAATCAGTGGCTAACAGAAG
ATATTTAATCAGTGGCTAACAGAAGC
TATTTAATCAGTGGCTAACAGAAGCT
ATTTAATCAGTGGCTAACAGAAGCTG
TTTAATCAGTGGCTAACAGAAGCTGA
TTAATCAGTGGCTAACAGAAGCTGAA
TAATCAGTGGCTAACAGAAGCTGAAC
AATCAGTGGCTAACAGAAGCTGAACA
ATCAGTGGCTAACAGAAGCTGAACAG
TCAGTGGCTAACAGAAGCTGAACAGT
CAGTGGCTAACAGAAGCTGAACAGTT
AGTGGCTAACAGAAGCTGAACAGTTT
GTGGCTAACAGAAGCTGAACAGTTTC
TGGCTAACAGAAGCTGAACAGTTTCT
GGCTAACAGAAGCTGAACAGTTTCTC
GCTAACAGAAGCTGAACAGTTTCTCA
CTAACAGAAGCTGAACAGTTTCTCAG
TAACAGAAGCTGAACAGTTTCTCAGA
AACAGAAGCTGAACAGTTTCTCAGAA
ACAGAAGCTGAACAGTTTCTCAGAAA
CAGAAGCTGAACAGTTTCTCAGAAAG
AGAAGCTGAACAGTTTCTCAGAAAGA
GAAGCTGAACAGTTTCTCAGAAAGAC
AAGCTGAACAGTTTCTCAGAAAGACA
AGCTGAACAGTTTCTCAGAAAGACAC
GCTGAACAGTTTCTCAGAAAGACACA
CTGAACAGTTTCTCAGAAAGACACAA
TGAACAGTTTCTCAGAAAGACACAAA
GAACAGTTTCTCAGAAAGACACAAAT
AACAGTTTCTCAGAAAGACACAAATT
ACAGTTTCTCAGAAAGACACAAATTC
CAGTTTCTCAGAAAGACACAAATTCC
AGTTTCTCAGAAAGACACAAATTCCT
GTTTCTCAGAAAGACACAAATTCCTG
TTTCTCAGAAAGACACAAATTCCTGA
TTCTCAGAAAGACACAAATTCCTGAG
TCTCAGAAAGACACAAATTCCTGAGA
CTCAGAAAGACACAAATTCCTGAGAA
TCAGAAAGACACAAATTCCTGAGAAT
CAGAAAGACACAAATTCCTGAGAATT
AGAAAGACACAAATTCCTGAGAATTG
GAAAGACACAAATTCCTGAGAATTGG
AAAGACACAAATTCCTGAGAATTGGG
AAGACACAAATTCCTGAGAATTGGGA
AGACACAAATTCCTGAGAATTGGGAA
GACACAAATTCCTGAGAATTGGGAAC
ACACAAATTCCTGAGAATTGGGAACA
CACAAATTCCTGAGAATTGGGAACAT
ACAAATTCCTGAGAATTGGGAACATG
CAAATTCCTGAGAATTGGGAACATGC
AAATTCCTGAGAATTGGGAACATGCT
AATTCCTGAGAATTGGGAACATGCTA
ATTCCTGAGAATTGGGAACATGCTAA
TTCCTGAGAATTGGGAACATGCTAAA
TCCTGAGAATTGGGAACATGCTAAAT
CCTGAGAATTGGGAACATGCTAAATA
CTGAGAATTGGGAACATGCTAAATAC
TGAGAATTGGGAACATGCTAAATACA
GAGAATTGGGAACATGCTAAATACAA
AGAATTGGGAACATGCTAAATACAAA
GAATTGGGAACATGCTAAATACAAAT
AATTGGGAACATGCTAAATACAAATG
ATTGGGAACATGCTAAATACAAATGG
TTGGGAACATGCTAAATACAAATGGT
TGGGAACATGCTAAATACAAATGGTA
GGGAACATGCTAAATACAAATGGTAT
GGAACATGCTAAATACAAATGGTATC
GAACATGCTAAATACAAATGGTATCT
AACATGCTAAATACAAATGGTATCTT
ACATGCTAAATACAAATGGTATCTTA
CATGCTAAATACAAATGGTATCTTAA
ATGCTAAATACAAATGGTATCTTAAG

TABLE 6J
27-mer ACs that bind to exon 44 of DMD
Nucleic Acid Sequence (5′-3′)

GGCGATTTGACAGATCTGTTGAGAAAT
GCGATTTGACAGATCTGTTGAGAAATG
CGATTTGACAGATCTGTTGAGAAATGG
GATTTGACAGATCTGTTGAGAAATGGC
ATTTGACAGATCTGTTGAGAAATGGCG
TTTGACAGATCTGTTGAGAAATGGCGG
TTGACAGATCTGTTGAGAAATGGCGGC
TGACAGATCTGTTGAGAAATGGCGGCG
GACAGATCTGTTGAGAAATGGCGGCGT
ACAGATCTGTTGAGAAATGGCGGCGTT
CAGATCTGTTGAGAAATGGCGGCGTTT
AGATCTGTTGAGAAATGGCGGCGTTTT
GATCTGTTGAGAAATGGCGGCGTTTTC
ATCTGTTGAGAAATGGCGGCGTTTTCA
TCTGTTGAGAAATGGCGGCGTTTTCAT
CTGTTGAGAAATGGCGGCGTTTTCATT
TGTTGAGAAATGGCGGCGTTTTCATTA
GTTGAGAAATGGCGGCGTTTTCATTAT
TTGAGAAATGGCGGCGTTTTCATTATG
TGAGAAATGGCGGCGTTTTCATTATGA
GAGAAATGGCGGCGTTTTCATTATGAT
AGAAATGGCGGCGTTTTCATTATGATA
GAAATGGCGGCGTTTTCATTATGATAT
AAATGGCGGCGTTTTCATTATGATATA
AATGGCGGCGTTTTCATTATGATATAA
ATGGCGGCGTTTTCATTATGATATAAA
TGGCGGCGTTTTCATTATGATATAAAG
GGCGGCGTTTTCATTATGATATAAAGA
GCGGCGTTTTCATTATGATATAAAGAT
CGGCGTTTTCATTATGATATAAAGATA
GGCGTTTTCATTATGATATAAAGATAT
GCGTTTTCATTATGATATAAAGATATT
CGTTTTCATTATGATATAAAGATATTT
GTTTTCATTATGATATAAAGATATTTA
TTTTCATTATGATATAAAGATATTTAA
TTTCATTATGATATAAAGATATTTAAT
TTCATTATGATATAAAGATATTTAATC
TCATTATGATATAAAGATATTTAATCA
CATTATGATATAAAGATATTTAATCAG
ATTATGATATAAAGATATTTAATCAGT
TTATGATATAAAGATATTTAATCAGTG
TATGATATAAAGATATTTAATCAGTGG
ATGATATAAAGATATTTAATCAGTGGC
TGATATAAAGATATTTAATCAGTGGCT
GATATAAAGATATTTAATCAGTGGCTA
ATATAAAGATATTTAATCAGTGGCTAA
TATAAAGATATTTAATCAGTGGCTAAC
ATAAAGATATTTAATCAGTGGCTAACA
TAAAGATATTTAATCAGTGGCTAACAG
AAAGATATTTAATCAGTGGCTAACAGA
AAGATATTTAATCAGTGGCTAACAGAA
AGATATTTAATCAGTGGCTAACAGAAG
GATATTTAATCAGTGGCTAACAGAAGC
ATATTTAATCAGTGGCTAACAGAAGCT
TATTTAATCAGTGGCTAACAGAAGCTG
ATTTAATCAGTGGCTAACAGAAGCTGA
TTTAATCAGTGGCTAACAGAAGCTGAA
TTAATCAGTGGCTAACAGAAGCTGAAC
TAATCAGTGGCTAACAGAAGCTGAACA
AATCAGTGGCTAACAGAAGCTGAACAG
ATCAGTGGCTAACAGAAGCTGAACAGT
TCAGTGGCTAACAGAAGCTGAACAGTT
CAGTGGCTAACAGAAGCTGAACAGTTT
AGTGGCTAACAGAAGCTGAACAGTTTC
GTGGCTAACAGAAGCTGAACAGTTTCT
TGGCTAACAGAAGCTGAACAGTTTCTC
GGCTAACAGAAGCTGAACAGTTTCTCA
GCTAACAGAAGCTGAACAGTTTCTCAG
CTAACAGAAGCTGAACAGTTTCTCAGA
TAACAGAAGCTGAACAGTTTCTCAGAA
AACAGAAGCTGAACAGTTTCTCAGAAA
ACAGAAGCTGAACAGTTTCTCAGAAAG
CAGAAGCTGAACAGTTTCTCAGAAAGA
AGAAGCTGAACAGTTTCTCAGAAAGAC
GAAGCTGAACAGTTTCTCAGAAAGACA
AAGCTGAACAGTTTCTCAGAAAGACAC
AGCTGAACAGTTTCTCAGAAAGACACA
GCTGAACAGTTTCTCAGAAAGACACAA
CTGAACAGTTTCTCAGAAAGACACAAA
TGAACAGTTTCTCAGAAAGACACAAAT
GAACAGTTTCTCAGAAAGACACAAATT
AACAGTTTCTCAGAAAGACACAAATTC
ACAGTTTCTCAGAAAGACACAAATTCC
CAGTTTCTCAGAAAGACACAAATTCCT
AGTTTCTCAGAAAGACACAAATTCCTG
GTTTCTCAGAAAGACACAAATTCCTGA
TTTCTCAGAAAGACACAAATTCCTGAG
TTCTCAGAAAGACACAAATTCCTGAGA
TCTCAGAAAGACACAAATTCCTGAGAA
CTCAGAAAGACACAAATTCCTGAGAAT
TCAGAAAGACACAAATTCCTGAGAATT
CAGAAAGACACAAATTCCTGAGAATTG
AGAAAGACACAAATTCCTGAGAATTGG
GAAAGACACAAATTCCTGAGAATTGGG
AAAGACACAAATTCCTGAGAATTGGGA
AAGACACAAATTCCTGAGAATTGGGAA
AGACACAAATTCCTGAGAATTGGGAAC
GACACAAATTCCTGAGAATTGGGAACA
ACACAAATTCCTGAGAATTGGGAACAT
CACAAATTCCTGAGAATTGGGAACATG
ACAAATTCCTGAGAATTGGGAACATGC
CAAATTCCTGAGAATTGGGAACATGCT
AAATTCCTGAGAATTGGGAACATGCTA
AATTCCTGAGAATTGGGAACATGCTAA
ATTCCTGAGAATTGGGAACATGCTAAA
TTCCTGAGAATTGGGAACATGCTAAAT
TCCTGAGAATTGGGAACATGCTAAATA
CCTGAGAATTGGGAACATGCTAAATAC
CTGAGAATTGGGAACATGCTAAATACA
TGAGAATTGGGAACATGCTAAATACAA
GAGAATTGGGAACATGCTAAATACAAA
AGAATTGGGAACATGCTAAATACAAAT
GAATTGGGAACATGCTAAATACAAATG
AATTGGGAACATGCTAAATACAAATGG
ATTGGGAACATGCTAAATACAAATGGT
TTGGGAACATGCTAAATACAAATGGTA
TGGGAACATGCTAAATACAAATGGTAT
GGGAACATGCTAAATACAAATGGTATC
GGAACATGCTAAATACAAATGGTATCT
GAACATGCTAAATACAAATGGTATCTT
AACATGCTAAATACAAATGGTATCTTA
ACATGCTAAATACAAATGGTATCTTAA
CATGCTAAATACAAATGGTATCTTAAG

TABLE 6K
28-mer ACs that bind to exon 44 of DMD
Nucleic Acid Sequence (5′-3′)

GGCGATTTGACAGATCTGTTGAGAAATG
GCGATTTGACAGATCTGTTGAGAAATGG
CGATTTGACAGATCTGTTGAGAAATGGC
GATTTGACAGATCTGTTGAGAAATGGCG
ATTTGACAGATCTGTTGAGAAATGGCGG
TTTGACAGATCTGTTGAGAAATGGCGGC
TTGACAGATCTGTTGAGAAATGGCGGCG
TGACAGATCTGTTGAGAAATGGCGGCGT
GACAGATCTGTTGAGAAATGGCGGCGTT
ACAGATCTGTTGAGAAATGGCGGCGTTT
CAGATCTGTTGAGAAATGGCGGCGTTTT
AGATCTGTTGAGAAATGGCGGCGTTTTC
GATCTGTTGAGAAATGGCGGCGTTTTCA
ATCTGTTGAGAAATGGCGGCGTTTTCAT
TCTGTTGAGAAATGGCGGCGTTTTCATT
CTGTTGAGAAATGGCGGCGTTTTCATTA
TGTTGAGAAATGGCGGCGTTTTCATTAT
GTTGAGAAATGGCGGCGTTTTCATTATG
TTGAGAAATGGCGGCGTTTTCATTATGA
TGAGAAATGGCGGCGTTTTCATTATGAT
GAGAAATGGCGGCGTTTTCATTATGATA
AGAAATGGCGGCGTTTTCATTATGATAT
GAAATGGCGGCGTTTTCATTATGATATA
AAATGGCGGCGTTTTCATTATGATATAA
AATGGCGGCGTTTTCATTATGATATAAA
ATGGCGGCGTTTTCATTATGATATAAAG
TGGCGGCGTTTTCATTATGATATAAAGA
GGCGGCGTTTTCATTATGATATAAAGAT
GCGGCGTTTTCATTATGATATAAAGATA
CGGCGTTTTCATTATGATATAAAGATAT
GGCGTTTTCATTATGATATAAAGATATT
GCGTTTTCATTATGATATAAAGATATTT
CGTTTTCATTATGATATAAAGATATTTA
GTTTTCATTATGATATAAAGATATTTAA
TTTTCATTATGATATAAAGATATTTAAT
TTTCATTATGATATAAAGATATTTAATC
TTCATTATGATATAAAGATATTTAATCA
TCATTATGATATAAAGATATTTAATCAG
CATTATGATATAAAGATATTTAATCAGT
ATTATGATATAAAGATATTTAATCAGTG
TTATGATATAAAGATATTTAATCAGTGG
TATGATATAAAGATATTTAATCAGTGGC
ATGATATAAAGATATTTAATCAGTGGCT
TGATATAAAGATATTTAATCAGTGGCTA
GATATAAAGATATTTAATCAGTGGCTAA
ATATAAAGATATTTAATCAGTGGCTAAC
TATAAAGATATTTAATCAGTGGCTAACA
ATAAAGATATTTAATCAGTGGCTAACAG
TAAAGATATTTAATCAGTGGCTAACAGA
AAAGATATTTAATCAGTGGCTAACAGAA
AAGATATTTAATCAGTGGCTAACAGAAG
AGATATTTAATCAGTGGCTAACAGAAGC
GATATTTAATCAGTGGCTAACAGAAGCT
ATATTTAATCAGTGGCTAACAGAAGCTG
TATTTAATCAGTGGCTAACAGAAGCTGA
ATTTAATCAGTGGCTAACAGAAGCTGAA
TTTAATCAGTGGCTAACAGAAGCTGAAC
TTAATCAGTGGCTAACAGAAGCTGAACA
TAATCAGTGGCTAACAGAAGCTGAACAG
AATCAGTGGCTAACAGAAGCTGAACAGT
ATCAGTGGCTAACAGAAGCTGAACAGTT
TCAGTGGCTAACAGAAGCTGAACAGTTT
CAGTGGCTAACAGAAGCTGAACAGTTTC
AGTGGCTAACAGAAGCTGAACAGTTTCT
GTGGCTAACAGAAGCTGAACAGTTTCTC
TGGCTAACAGAAGCTGAACAGTTTCTCA
GGCTAACAGAAGCTGAACAGTTTCTCAG
GCTAACAGAAGCTGAACAGTTTCTCAGA
CTAACAGAAGCTGAACAGTTTCTCAGAA
TAACAGAAGCTGAACAGTTTCTCAGAAA
AACAGAAGCTGAACAGTTTCTCAGAAAG
ACAGAAGCTGAACAGTTTCTCAGAAAGA
CAGAAGCTGAACAGTTTCTCAGAAAGAC
AGAAGCTGAACAGTTTCTCAGAAAGACA
GAAGCTGAACAGTTTCTCAGAAAGACAC
AAGCTGAACAGTTTCTCAGAAAGACACA
AGCTGAACAGTTTCTCAGAAAGACACAA
GCTGAACAGTTTCTCAGAAAGACACAAA
CTGAACAGTTTCTCAGAAAGACACAAAT
TGAACAGTTTCTCAGAAAGACACAAATT
GAACAGTTTCTCAGAAAGACACAAATTC
AACAGTTTCTCAGAAAGACACAAATTCC
ACAGTTTCTCAGAAAGACACAAATTCCT
CAGTTTCTCAGAAAGACACAAATTCCTG
AGTTTCTCAGAAAGACACAAATTCCTGA
GTTTCTCAGAAAGACACAAATTCCTGAG
TTTCTCAGAAAGACACAAATTCCTGAGA
TTCTCAGAAAGACACAAATTCCTGAGAA
TCTCAGAAAGACACAAATTCCTGAGAAT
CTCAGAAAGACACAAATTCCTGAGAATT
TCAGAAAGACACAAATTCCTGAGAATTG
CAGAAAGACACAAATTCCTGAGAATTGG
AGAAAGACACAAATTCCTGAGAATTGGG
GAAAGACACAAATTCCTGAGAATTGGGA
AAAGACACAAATTCCTGAGAATTGGGAA
AAGACACAAATTCCTGAGAATTGGGAAC
AGACACAAATTCCTGAGAATTGGGAACA
GACACAAATTCCTGAGAATTGGGAACAT
ACACAAATTCCTGAGAATTGGGAACATG
CACAAATTCCTGAGAATTGGGAACATGC
ACAAATTCCTGAGAATTGGGAACATGCT
CAAATTCCTGAGAATTGGGAACATGCTA
AAATTCCTGAGAATTGGGAACATGCTAA
AATTCCTGAGAATTGGGAACATGCTAAA
ATTCCTGAGAATTGGGAACATGCTAAAT
TTCCTGAGAATTGGGAACATGCTAAATA
TCCTGAGAATTGGGAACATGCTAAATAC
CCTGAGAATTGGGAACATGCTAAATACA
CTGAGAATTGGGAACATGCTAAATACAA
TGAGAATTGGGAACATGCTAAATACAAA
GAGAATTGGGAACATGCTAAATACAAAT
AGAATTGGGAACATGCTAAATACAAATG
GAATTGGGAACATGCTAAATACAAATGG
AATTGGGAACATGCTAAATACAAATGGT
ATTGGGAACATGCTAAATACAAATGGTA
TTGGGAACATGCTAAATACAAATGGTAT
TGGGAACATGCTAAATACAAATGGTATC
GGGAACATGCTAAATACAAATGGTATCT
GGAACATGCTAAATACAAATGGTATCTT
GAACATGCTAAATACAAATGGTATCTTA
AACATGCTAAATACAAATGGTATCTTAA
ACATGCTAAATACAAATGGTATCTTAAG

TABLE 6L
29-mer d ACs that bind to exon 44 of DMD
Nucleic Acid Sequence (5′-3′)

GGCGATTTGACAGATCTGTTGAGAAATGG
GCGATTTGACAGATCTGTTGAGAAATGGC
CGATTTGACAGATCTGTTGAGAAATGGCG
GATTTGACAGATCTGTTGAGAAATGGCGG
ATTTGACAGATCTGTTGAGAAATGGCGGC
TTTGACAGATCTGTTGAGAAATGGCGGCG
TTGACAGATCTGTTGAGAAATGGCGGCGT
TGACAGATCTGTTGAGAAATGGCGGCGTT
GACAGATCTGTTGAGAAATGGCGGCGTTT
ACAGATCTGTTGAGAAATGGCGGCGTTTT
CAGATCTGTTGAGAAATGGCGGCGTTTTC
AGATCTGTTGAGAAATGGCGGCGTTTTCA
GATCTGTTGAGAAATGGCGGCGTTTTCAT
ATCTGTTGAGAAATGGCGGCGTTTTCATT
TCTGTTGAGAAATGGCGGCGTTTTCATTA
CTGTTGAGAAATGGCGGCGTTTTCATTAT
TGTTGAGAAATGGCGGCGTTTTCATTATG
GTTGAGAAATGGCGGCGTTTTCATTATGA
TTGAGAAATGGCGGCGTTTTCATTATGAT
TGAGAAATGGCGGCGTTTTCATTATGATA
GAGAAATGGCGGCGTTTTCATTATGATAT
AGAAATGGCGGCGTTTTCATTATGATATA
GAAATGGCGGCGTTTTCATTATGATATAA
AAATGGCGGCGTTTTCATTATGATATAAA
AATGGCGGCGTTTTCATTATGATATAAAG
ATGGCGGCGTTTTCATTATGATATAAAGA
TGGCGGCGTTTTCATTATGATATAAAGAT
GGCGGCGTTTTCATTATGATATAAAGATA
GCGGCGTTTTCATTATGATATAAAGATAT
CGGCGTTTTCATTATGATATAAAGATATT
GGCGTTTTCATTATGATATAAAGATATTT
GCGTTTTCATTATGATATAAAGATATTTA
CGTTTTCATTATGATATAAAGATATTTAA
GTTTTCATTATGATATAAAGATATTTAAT
TTTTCATTATGATATAAAGATATTTAATC
TTTCATTATGATATAAAGATATTTAATCA
TTCATTATGATATAAAGATATTTAATCAG
TCATTATGATATAAAGATATTTAATCAGT
CATTATGATATAAAGATATTTAATCAGTG
ATTATGATATAAAGATATTTAATCAGTGG
TTATGATATAAAGATATTTAATCAGTGGC
TATGATATAAAGATATTTAATCAGTGGCT
ATGATATAAAGATATTTAATCAGTGGCTA
TGATATAAAGATATTTAATCAGTGGCTAA
GATATAAAGATATTTAATCAGTGGCTAAC
ATATAAAGATATTTAATCAGTGGCTAACA
TATAAAGATATTTAATCAGTGGCTAACAG
ATAAAGATATTTAATCAGTGGCTAACAGA
TAAAGATATTTAATCAGTGGCTAACAGAA
AAAGATATTTAATCAGTGGCTAACAGAAG
AAGATATTTAATCAGTGGCTAACAGAAGC
AGATATTTAATCAGTGGCTAACAGAAGCT
GATATTTAATCAGTGGCTAACAGAAGCTG
ATATTTAATCAGTGGCTAACAGAAGCTGA
TATTTAATCAGTGGCTAACAGAAGCTGAA
ATTTAATCAGTGGCTAACAGAAGCTGAAC
TTTAATCAGTGGCTAACAGAAGCTGAACA
TTAATCAGTGGCTAACAGAAGCTGAACAG
TAATCAGTGGCTAACAGAAGCTGAACAGT
AATCAGTGGCTAACAGAAGCTGAACAGTT
ATCAGTGGCTAACAGAAGCTGAACAGTTT
TCAGTGGCTAACAGAAGCTGAACAGTTTC
CAGTGGCTAACAGAAGCTGAACAGTTTCT
AGTGGCTAACAGAAGCTGAACAGTTTCTC
GTGGCTAACAGAAGCTGAACAGTTTCTCA
TGGCTAACAGAAGCTGAACAGTTTCTCAG
GGCTAACAGAAGCTGAACAGTTTCTCAGA
GCTAACAGAAGCTGAACAGTTTCTCAGAA
CTAACAGAAGCTGAACAGTTTCTCAGAAA
TAACAGAAGCTGAACAGTTTCTCAGAAAG
AACAGAAGCTGAACAGTTTCTCAGAAAGA
ACAGAAGCTGAACAGTTTCTCAGAAAGAC
CAGAAGCTGAACAGTTTCTCAGAAAGACA
AGAAGCTGAACAGTTTCTCAGAAAGACAC
GAAGCTGAACAGTTTCTCAGAAAGACACA
AAGCTGAACAGTTTCTCAGAAAGACACAA
AGCTGAACAGTTTCTCAGAAAGACACAAA
GCTGAACAGTTTCTCAGAAAGACACAAAT
CTGAACAGTTTCTCAGAAAGACACAAATT
TGAACAGTTTCTCAGAAAGACACAAATTC
GAACAGTTTCTCAGAAAGACACAAATTCC
AACAGTTTCTCAGAAAGACACAAATTCCT
ACAGTTTCTCAGAAAGACACAAATTCCTG
CAGTTTCTCAGAAAGACACAAATTCCTGA
AGTTTCTCAGAAAGACACAAATTCCTGAG
GTTTCTCAGAAAGACACAAATTCCTGAGA
TTTCTCAGAAAGACACAAATTCCTGAGAA
TTCTCAGAAAGACACAAATTCCTGAGAAT
TCTCAGAAAGACACAAATTCCTGAGAATT
CTCAGAAAGACACAAATTCCTGAGAATTG
TCAGAAAGACACAAATTCCTGAGAATTGG
CAGAAAGACACAAATTCCTGAGAATTGGG
AGAAAGACACAAATTCCTGAGAATTGGGA
GAAAGACACAAATTCCTGAGAATTGGGAA
AAAGACACAAATTCCTGAGAATTGGGAAC
AAGACACAAATTCCTGAGAATTGGGAACA
AGACACAAATTCCTGAGAATTGGGAACAT
GACACAAATTCCTGAGAATTGGGAACATG
ACACAAATTCCTGAGAATTGGGAACATGC
CACAAATTCCTGAGAATTGGGAACATGCT
ACAAATTCCTGAGAATTGGGAACATGCTA
CAAATTCCTGAGAATTGGGAACATGCTAA
AAATTCCTGAGAATTGGGAACATGCTAAA
AATTCCTGAGAATTGGGAACATGCTAAAT
ATTCCTGAGAATTGGGAACATGCTAAATA
TTCCTGAGAATTGGGAACATGCTAAATAC
TCCTGAGAATTGGGAACATGCTAAATACA
CCTGAGAATTGGGAACATGCTAAATACAA
CTGAGAATTGGGAACATGCTAAATACAAA
TGAGAATTGGGAACATGCTAAATACAAAT
GAGAATTGGGAACATGCTAAATACAAATG
AGAATTGGGAACATGCTAAATACAAATGG
GAATTGGGAACATGCTAAATACAAATGGT
AATTGGGAACATGCTAAATACAAATGGTA
ATTGGGAACATGCTAAATACAAATGGTAT
TTGGGAACATGCTAAATACAAATGGTATC
TGGGAACATGCTAAATACAAATGGTATCT
GGGAACATGCTAAATACAAATGGTATCTT
GGAACATGCTAAATACAAATGGTATCTTA
GAACATGCTAAATACAAATGGTATCTTAA
AACATGCTAAATACAAATGGTATCTTAAG

TABLE 6M
30-mer ACs that bind to exon 44 of DMD
Nucleic Acid Sequence (5′-3′)

GGCGATTTGACAGATCTGTTGAGAAATGGC
GCGATTTGACAGATCTGTTGAGAAATGGCG
CGATTTGACAGATCTGTTGAGAAATGGCGG
GATTTGACAGATCTGTTGAGAAATGGCGGC
ATTTGACAGATCTGTTGAGAAATGGCGGCG
TTTGACAGATCTGTTGAGAAATGGCGGCGT
TTGACAGATCTGTTGAGAAATGGCGGCGTT
TGACAGATCTGTTGAGAAATGGCGGCGTTT
GACAGATCTGTTGAGAAATGGCGGCGTTTT
ACAGATCTGTTGAGAAATGGCGGCGTTTTC
CAGATCTGTTGAGAAATGGCGGCGTTTTCA
AGATCTGTTGAGAAATGGCGGCGTTTTCAT
GATCTGTTGAGAAATGGCGGCGTTTTCATT
ATCTGTTGAGAAATGGCGGCGTTTTCATTA
TCTGTTGAGAAATGGCGGCGTTTTCATTAT
CTGTTGAGAAATGGCGGCGTTTTCATTATG
TGTTGAGAAATGGCGGCGTTTTCATTATGA
GTTGAGAAATGGCGGCGTTTTCATTATGAT
TTGAGAAATGGCGGCGTTTTCATTATGATA
TGAGAAATGGCGGCGTTTTCATTATGATAT
GAGAAATGGCGGCGTTTTCATTATGATATA
AGAAATGGCGGCGTTTTCATTATGATATAA
GAAATGGCGGCGTTTTCATTATGATATAAA
AAATGGCGGCGTTTTCATTATGATATAAAG
AATGGCGGCGTTTTCATTATGATATAAAGA
ATGGCGGCGTTTTCATTATGATATAAAGAT
TGGCGGCGTTTTCATTATGATATAAAGATA
GGCGGCGTTTTCATTATGATATAAAGATAT
GCGGCGTTTTCATTATGATATAAAGATATT
CGGCGTTTTCATTATGATATAAAGATATTT
GGCGTTTTCATTATGATATAAAGATATTTA
GCGTTTTCATTATGATATAAAGATATTTAA
CGTTTTCATTATGATATAAAGATATTTAAT
GTTTTCATTATGATATAAAGATATTTAATC
TTTTCATTATGATATAAAGATATTTAATCA
TTTCATTATGATATAAAGATATTTAATCAG
TTCATTATGATATAAAGATATTTAATCAGT
TCATTATGATATAAAGATATTTAATCAGTG
CATTATGATATAAAGATATTTAATCAGTGG
ATTATGATATAAAGATATTTAATCAGTGGC
TTATGATATAAAGATATTTAATCAGTGGCT
TATGATATAAAGATATTTAATCAGTGGCTA
ATGATATAAAGATATTTAATCAGTGGCTAA
TGATATAAAGATATTTAATCAGTGGCTAAC
GATATAAAGATATTTAATCAGTGGCTAACA
ATATAAAGATATTTAATCAGTGGCTAACAG
TATAAAGATATTTAATCAGTGGCTAACAGA
ATAAAGATATTTAATCAGTGGCTAACAGAA
TAAAGATATTTAATCAGTGGCTAACAGAAG
AAAGATATTTAATCAGTGGCTAACAGAAGC
AAGATATTTAATCAGTGGCTAACAGAAGCT
AGATATTTAATCAGTGGCTAACAGAAGCTG
GATATTTAATCAGTGGCTAACAGAAGCTGA
ATATTTAATCAGTGGCTAACAGAAGCTGAA
TATTTAATCAGTGGCTAACAGAAGCTGAAC
ATTTAATCAGTGGCTAACAGAAGCTGAACA
TTTAATCAGTGGCTAACAGAAGCTGAACAG
TTAATCAGTGGCTAACAGAAGCTGAACAGT
TAATCAGTGGCTAACAGAAGCTGAACAGTT
AATCAGTGGCTAACAGAAGCTGAACAGTTT
ATCAGTGGCTAACAGAAGCTGAACAGTTTC
TCAGTGGCTAACAGAAGCTGAACAGTTTCT
CAGTGGCTAACAGAAGCTGAACAGTTTCTC
AGTGGCTAACAGAAGCTGAACAGTTTCTCA
GTGGCTAACAGAAGCTGAACAGTTTCTCAG
TGGCTAACAGAAGCTGAACAGTTTCTCAGA
GGCTAACAGAAGCTGAACAGTTTCTCAGAA
GCTAACAGAAGCTGAACAGTTTCTCAGAAA
CTAACAGAAGCTGAACAGTTTCTCAGAAAG
TAACAGAAGCTGAACAGTTTCTCAGAAAGA
AACAGAAGCTGAACAGTTTCTCAGAAAGAC
ACAGAAGCTGAACAGTTTCTCAGAAAGACA
CAGAAGCTGAACAGTTTCTCAGAAAGACAC
AGAAGCTGAACAGTTTCTCAGAAAGACACA
GAAGCTGAACAGTTTCTCAGAAAGACACAA
AAGCTGAACAGTTTCTCAGAAAGACACAAA
AGCTGAACAGTTTCTCAGAAAGACACAAAT
GCTGAACAGTTTCTCAGAAAGACACAAATT
CTGAACAGTTTCTCAGAAAGACACAAATTC
TGAACAGTTTCTCAGAAAGACACAAATTCC
GAACAGTTTCTCAGAAAGACACAAATTCCT
AACAGTTTCTCAGAAAGACACAAATTCCTG
ACAGTTTCTCAGAAAGACACAAATTCCTGA
CAGTTTCTCAGAAAGACACAAATTCCTGAG
AGTTTCTCAGAAAGACACAAATTCCTGAGA
GTTTCTCAGAAAGACACAAATTCCTGAGAA
TTTCTCAGAAAGACACAAATTCCTGAGAAT
TTCTCAGAAAGACACAAATTCCTGAGAATT
TCTCAGAAAGACACAAATTCCTGAGAATTG
CTCAGAAAGACACAAATTCCTGAGAATTGG
TCAGAAAGACACAAATTCCTGAGAATTGGG
CAGAAAGACACAAATTCCTGAGAATTGGGA
AGAAAGACACAAATTCCTGAGAATTGGGAA
GAAAGACACAAATTCCTGAGAATTGGGAAC
AAAGACACAAATTCCTGAGAATTGGGAACA
AAGACACAAATTCCTGAGAATTGGGAACAT
AGACACAAATTCCTGAGAATTGGGAACATG
GACACAAATTCCTGAGAATTGGGAACATGC
ACACAAATTCCTGAGAATTGGGAACATGCT
CACAAATTCCTGAGAATTGGGAACATGCTA
ACAAATTCCTGAGAATTGGGAACATGCTAA
CAAATTCCTGAGAATTGGGAACATGCTAAA
AAATTCCTGAGAATTGGGAACATGCTAAAT
AATTCCTGAGAATTGGGAACATGCTAAATA
ATTCCTGAGAATTGGGAACATGCTAAATAC
TTCCTGAGAATTGGGAACATGCTAAATACA
TCCTGAGAATTGGGAACATGCTAAATACAA
CCTGAGAATTGGGAACATGCTAAATACAAA
CTGAGAATTGGGAACATGCTAAATACAAAT
TGAGAATTGGGAACATGCTAAATACAAATG
GAGAATTGGGAACATGCTAAATACAAATGG
AGAATTGGGAACATGCTAAATACAAATGGT
GAATTGGGAACATGCTAAATACAAATGGTA
AATTGGGAACATGCTAAATACAAATGGTAT
ATTGGGAACATGCTAAATACAAATGGTATC
TTGGGAACATGCTAAATACAAATGGTATCT
TGGGAACATGCTAAATACAAATGGTATCTT
GGGAACATGCTAAATACAAATGGTATCTTA
GGAACATGCTAAATACAAATGGTATCTTAA
GAACATGCTAAATACAAATGGTATCTTAAG
XGAAAACGCCGCCAXXXCXCAACAGAXCXG,
wherein X = U or T
TGAAAACGCCGCCATTTCTCAACAGATCTG

In embodiments, any AC described herein, including the AC in Tables 6A-6M, the reverse complement thereof, or a sequence with at least 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity thereto. comprise at least one modified nucleotide or nucleic acid selected from a phosphorothioate (PS) nucleotide, a phosphorodiamidate morpholino (PMO) nucleotide, a locked nucleic acid (LNA), a peptide nucleic acid (PNA), a nucleotide comprising a 2′-O-methyl (2′-OMe) modified backbone, a 2′O-methoxy-ethyl (2′-MOE) nucleotide, a 2′,4′ constrained ethyl (cEt) nucleotide, and a 2′-deoxy-2′-fluoro-beta-D-arabinonucleic acid (2′F-ANA). In embodiments, hybridization of the AC with the target sequence promotes or induces splicing of exon 44. In embodiments, AC comprises at least one phosphorodiamidate morpholino (PMO) nucleotide. In embodiments, 1, 2, 3, 4, 5, 6, 7, 8, 9, 10, 11, 12, 13, 1, 4 15, 16, 17, 18, 19, 20, 21, 22, 23, 24, 25, 26, 2, 7 28, 29, 30 or more of the nucleoties are modified. In embodiments, each nucleotide in the AC is a phosphorodiamidate morpholino (PMO) nucleotide.

In embodiments, the compound has the following the structure:

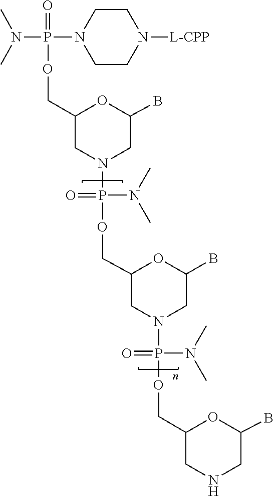

wherein:
CPP is a cyclic peptide described herein (also referred to as a cell penetrating peptide);
L is a linker;
B is each independently a nucleobase that is complementary to a base in the target sequence; and
n is an integer from 1 to 50. In embodiments, the sum of B and n correspond to a sequence shown in Tables 6A-6M, the reverse complement thereof, or a sequence with at least 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity thereto.
   Cyclic Cell Penetrating Peptides (cCPPs) Conjugated to an AC

The cyclic cell penetrating peptide (cCPP) can be conjugated to an AC.

The AC can be conjugated to cCPP through a linker. The cargo moiety can be conjugated to the linker at the terminal carbonyl group to provide the following structure:

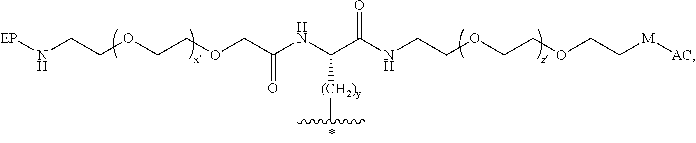

wherein:
EP is an exocyclic peptide and M, AA_{SC}, AC, x′, y, and z′ are as defined above, * is the point of attachment to the AA_{SC}. x′ can be 1. y can be 4. z′ can be 11. —(OCH₂CH₂)_{x′}— and/or —(OCH₂CH₂)_{z′}— can be independently replaced with one or more amino acids, including, for example, glycine, beta-alanine, 4-aminobutyric acid, 5-aminopentanoic acid, 6-aminohexanoic acid, or combinations thereof.

An endosomal escape vehicle (EEV) can comprise a cyclic cell penetrating peptide (cCPP), an exocyclic peptide (EP) and linker, and can be conjugated to an AC to form an EEV-conjugate comprising the structure of Formula (C):

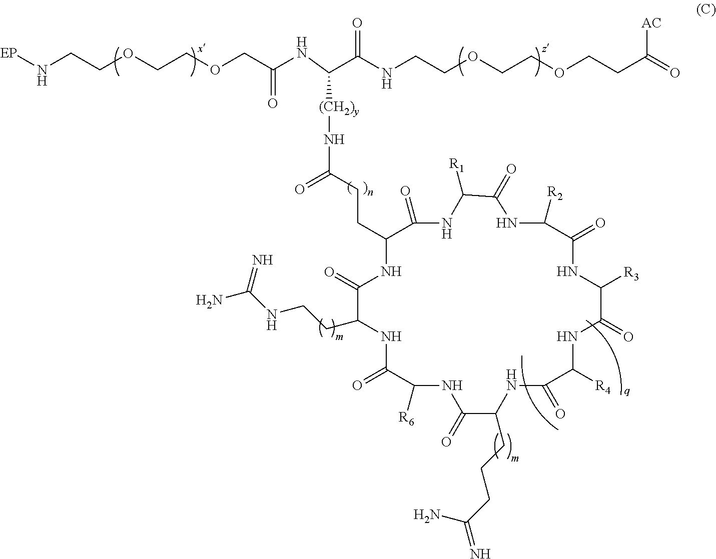

or a protonated form thereof,
   wherein.
R₁, R₂, and R₃ can each independently be H or an amino acid residue having a side chain comprising an aromatic group;
R₄ and R₆ are independently H or an amino acid side chain;
EP is an exocyclic peptide as defined herein;
AC is as defined herein;
each m is independently an integer from 0-3;
n is an integer from 0-2;
x′ is an integer from 2-20;
y is an integer from 1-5;
q is an integer from 1-4; and
z′ is an integer from 2-20.

R₁, R₂, R₃, R₄, EP, AC, m, n, x′, y, q, and z′ are as defined herein.

The EEV can be conjugated to an AC and the EEV-conjugate can comprise the structure of Formula (C-a) or (C-b):

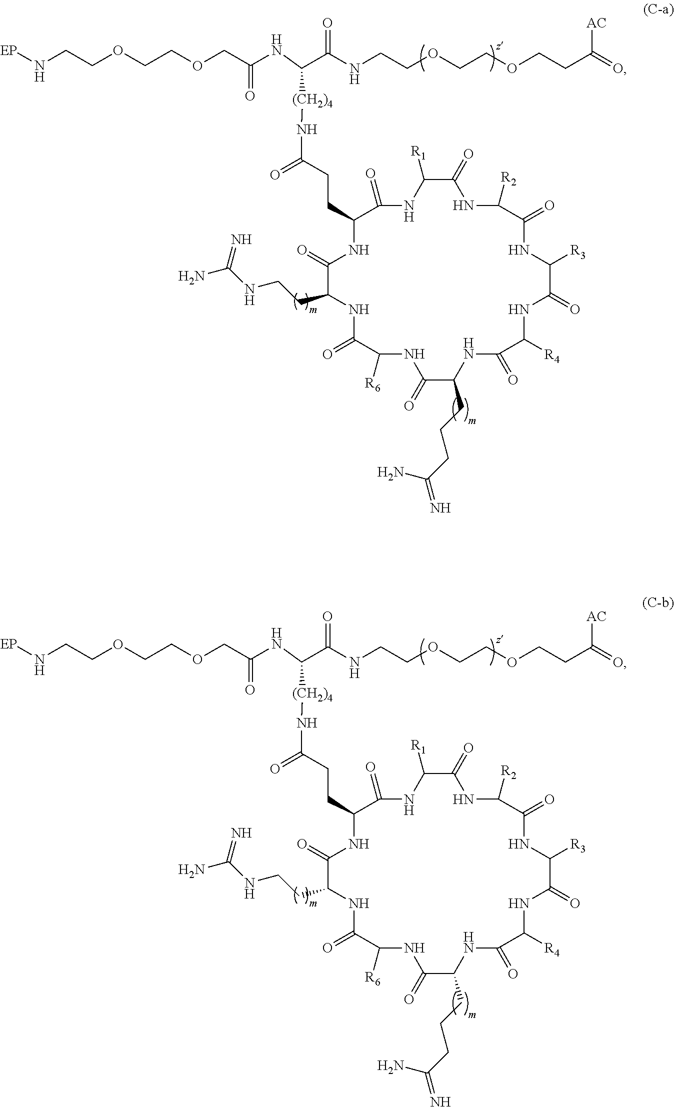

or a protonated form thereof, wherein EP, m and z are as defined above in Formula (C).

The EEV can be conjugated to an AC and the EEV-conjugate can comprise the structure of Formula (C-c):

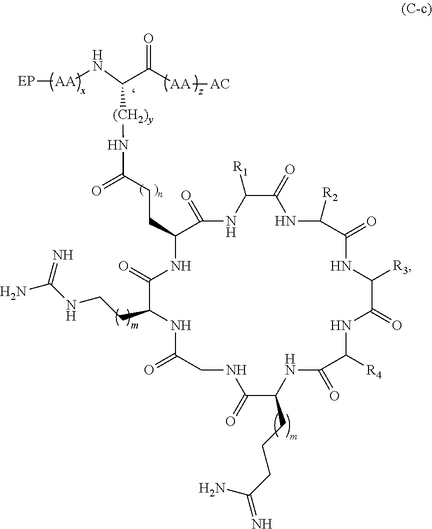

or a protonated form thereof, wherein EP, R¹, R², R³, R⁴, and m are as defined above in Formula (III); AA can be an amino acid as defined herein; n can be an integer from 0-2; x can be an integer from 1-10; y can be an integer from 1-5; and z can be an integer from 1-10.

The EEV can be conjugated to an oligonucleotide AC and the EEV-oligonucleotide conjugate can comprises a structure of Formula (C-1), (C-2), (C-3), or (C-4):

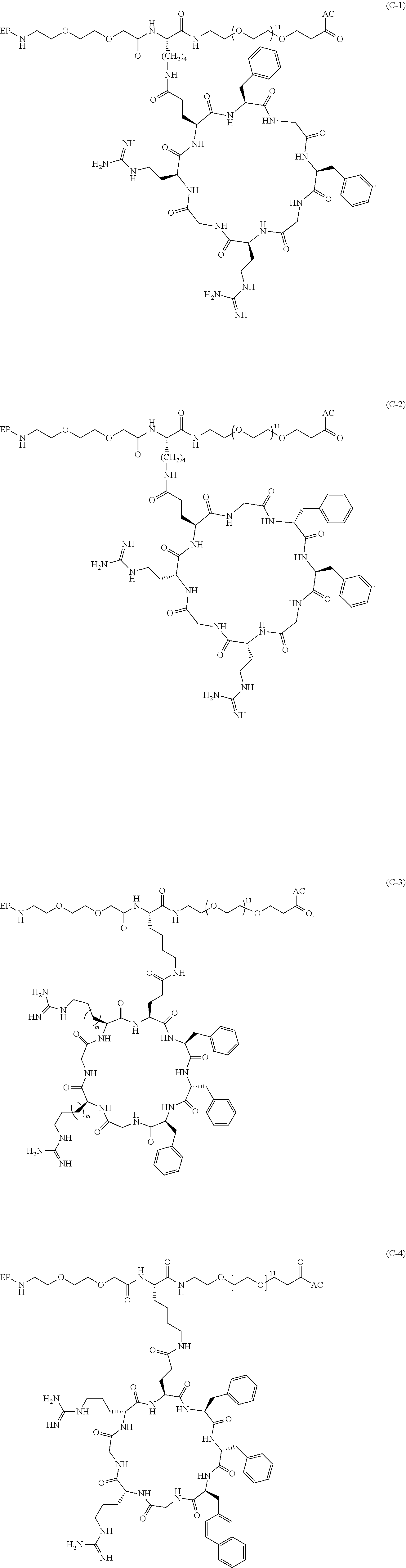

In the formulae above, EP is an exocylic peptide and the AC can have a sequence of 15-30 nucleotides that is a complementary to a target sequence comprising at least a portion of exon 44 of DMD gene in a pre-mRNA sequence. In embodiments, the AC can be selected from an oligonucleotide shown Tables 6A-6M, the reverse complement thereof, or a sequence with at least 80%, 81%, 82%, 83%, 84%, 85%, 86%, 87%, 88%, 89%, 90%, 91%, 92%, 93%, 94%, 95%, 96%, 97%, 98%, or 99% nucleic acid sequence identity thereto.

In embodiments, the compounds described herein form a multimer. In embodiments, multimerization occurs via non-covalent interactions, for example, through hydrophobic interactions, ionic interactions, hydrogen bonding, or dipole-dipole interactions. In embodiments, the compounds form a dimer, trimer, tetramer, pentamer, hexamer, heptamer, octamer, or nonamer. In embodiments, the compounds comprise 1, 2, 3, 4, 5, 6, 7, 8, 9, or 10 cyclic peptides. In embodiments, the compounds comprise 1, 2, 3, 4, 5, 6, 7, 8, 9, or 10 ACs. In embodiments, the compounds comprise 1, 2, 3, 4, 5, 6, 7, 8, 9, or 10 EPs. In embodiments, the compounds comprise from 1 to 10 cyclic peptides and from 1 to 10 ACs. In In embodiments, the compounds comprise from 1 to 10 cyclic peptides, from 1 to 10 ACs, or from 1 to 10 EPs.

In embodiments, the compounds of the disclosure comprise any one of the following structures. The compounds below are illustrative only and any one of the cyclic peptides, linkers, and AC in any one of the structures below may be replaced with any one of the cyclic peptides, linkers, or ACs described herein.

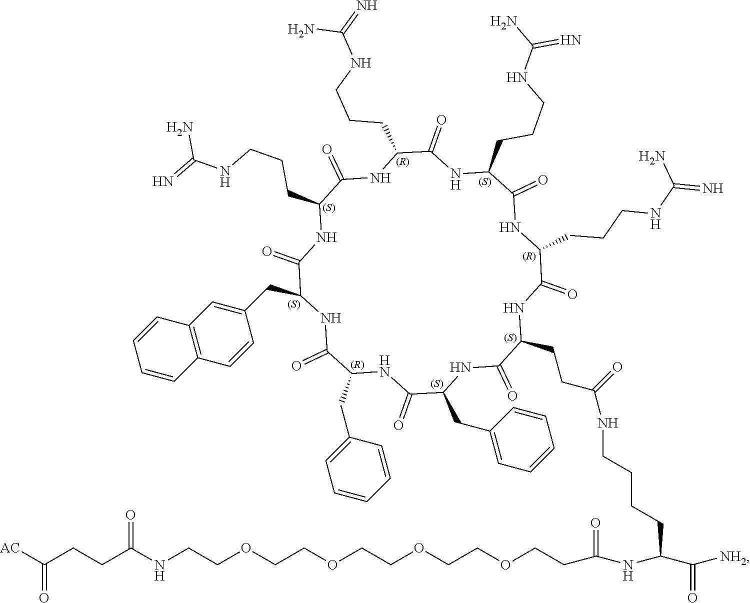

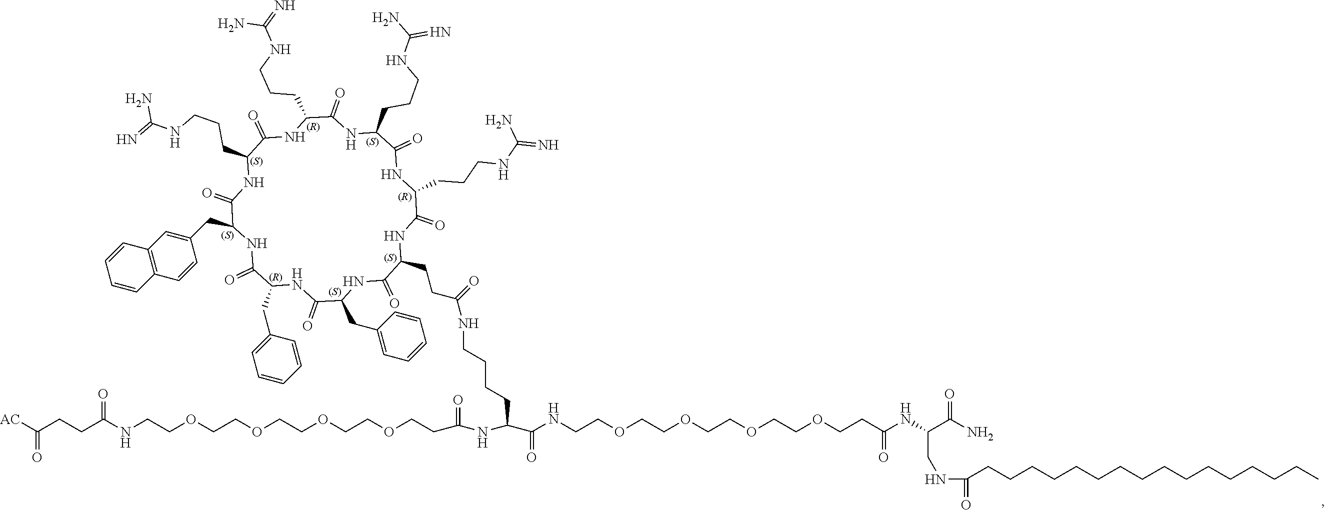

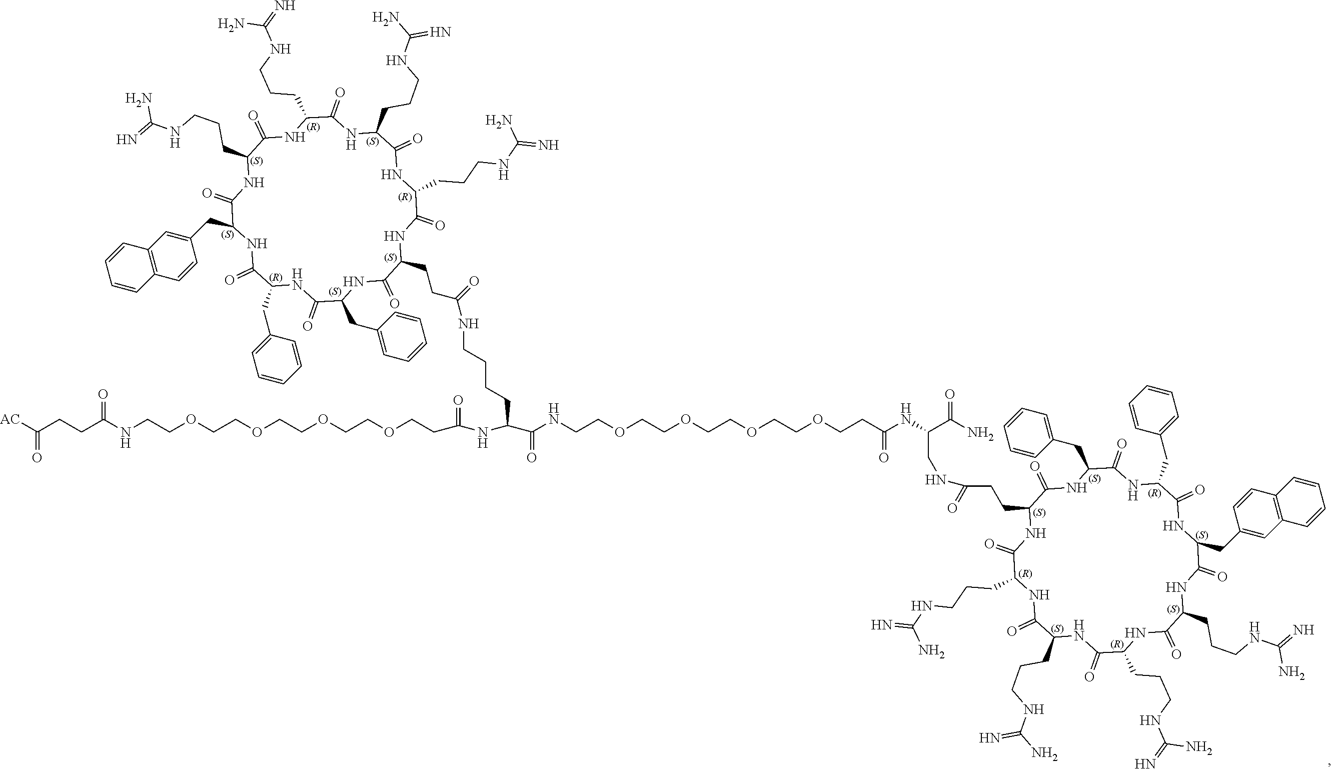

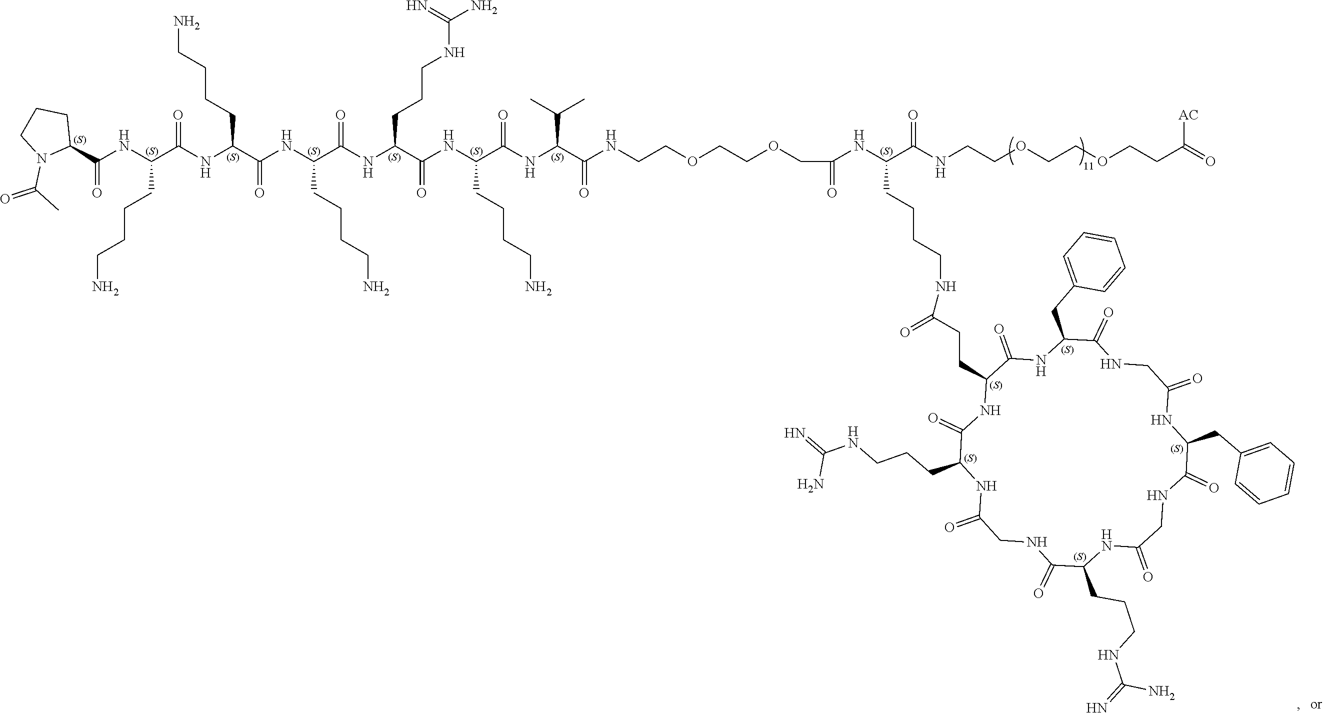

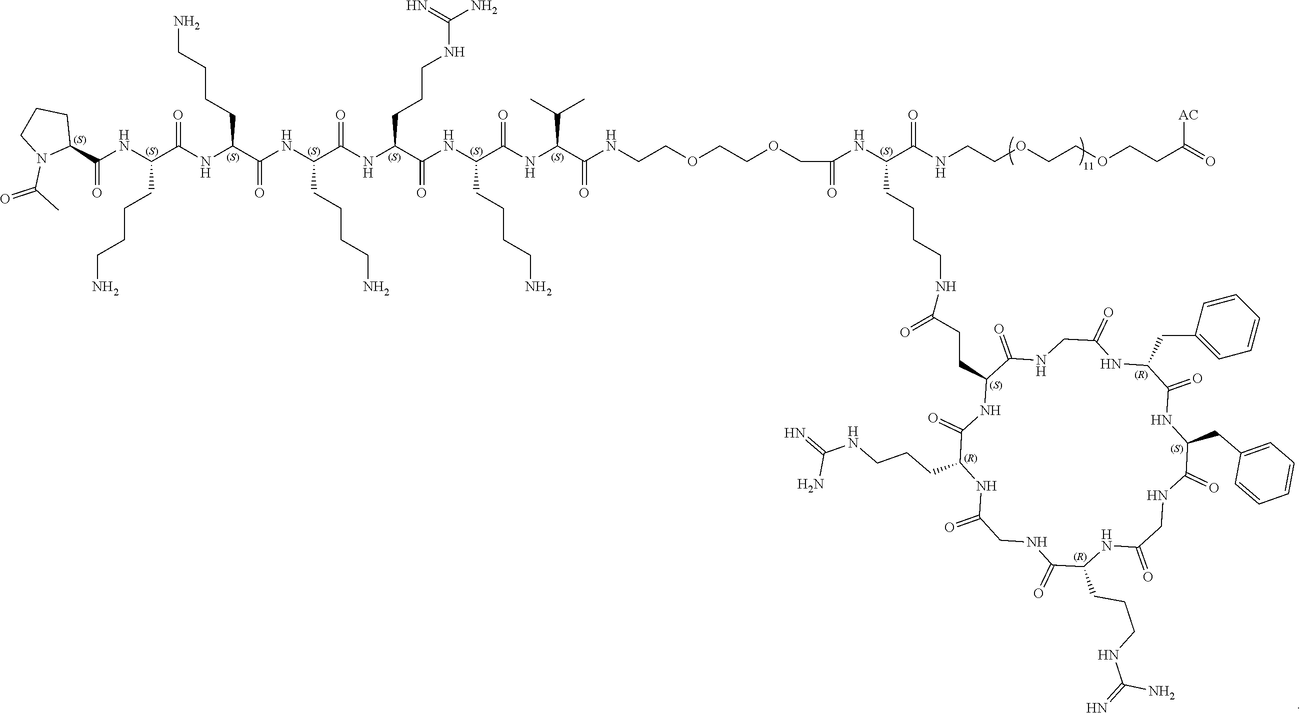

### Cytosolic Delivery Efficiency

Modifications to a cyclic cell penetrating peptide (cCPP) may improve cytosolic delivery efficiency. Improved cytosolic uptake efficiency can be measured by comparing the cytosolic delivery efficiency of a cCPP having a modified sequence to a control sequence. The control sequence does not include a particular replacement amino acid residue in the modified sequence (including, but not limited to arginine, phenylalanine, and/or glycine), but is otherwise identical.

In embodiments, compounds comprising a cyclic peptide and an AC have improved cytosolic uptake efficiency compared to compounds comprising an AC alone. Cytosolic uptake efficiency can be measured by comparing the cytosolic delivery efficiency of the compound comprising the cyclic peptide and the AC to the cytosolic delivery efficiency of an AC alone.

As used herein cytosolic delivery efficiency refers to the ability of a cCPP to traverse a cell membrane and enter the cytosol of a cell. Cytosolic delivery efficiency of the cCPP is not necessarily dependent on a receptor or a cell type. Cytosolic delivery efficiency can refer to absolute cytosolic delivery efficiency or relative cytosolic delivery efficiency.

Absolute cytosolic delivery efficiency is the ratio of cytosolic concentration of a cCPP (or a cCPP-AC conjugate) over the concentration of the cCPP (or the cCPP-AC conjugate) in the growth medium. Relative cytosolic delivery efficiency refers to the concentration of a cCPP in the cytosol compared to the concentration of a control cCPP in the cytosol. Quantification can be achieved by fluorescently labeling the cCPP (e.g., with a FITC dye) and measuring the fluorescence intensity using techniques well-known in the art.

Relative cytosolic delivery efficiency is determined by comparing (i) the amount of a cCPP of the invention internalized by a cell type (e.g., HeLa cells) to (ii) the amount of a control cCPP internalized by the same cell type. To measure relative cytosolic delivery efficiency, the cell type may be incubated in the presence of a cCPP for a specified period of time (e.g., 30 minutes, 1 hour, 2 hours, etc.) after which the amount of the cCPP internalized by the cell is quantified using methods known in the art, e.g., fluorescence microscopy. Separately, the same concentration of the control cCPP is incubated in the presence of the cell type over the same period of time, and the amount of the control cCPP internalized by the cell is quantified.

Relative cytosolic delivery efficiency can be determined by measuring the IC₅₀ of a cCPP having a modified sequence for an intracellular target and comparing the IC₅₀ of the cCPP having the modified sequence to a control sequence (as described herein).

The relative cytosolic delivery efficiency of the cCPPs can be in the range of from about 50% to about 450% compared to cyclo(FfΦRrRrQ), e.g., about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 310%, about 320%, about 330%, about 340%, about 350%, about 360%, about 370%, about 380%, about 390%, about 400%, about 410%, about 420%, about 430%, about 440%, about 450%, about 460%, about 470%, about 480%, about 490%, about 500%, about 510%, about 520%, about 530%, about 540%, about 550%, about 560%, about 570%, about 580%, or about 590%, inclusive of all values and subranges therebetween. The relative cytosolic delivery efficiency of the cCPPs can be improved by greater than about 600% compared to a cyclic peptide comprising cyclo(FfΦRrRrQ).

The absolute cytosolic delivery efficacy of from about 40% to about 100%, e.g., about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, inclusive of all values and subranges therebetween.

The cCPPs of the present disclosure can improve the cytosolic delivery efficiency by about 1.1 fold to about 30 fold, compared to an otherwise identical sequence, e.g., about 1.2, about 1.3, about 1.4, about 1.5, about 1.6, about 1.7, about 1.8, about 1.9, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 10, about 10.5, about 11.0, about 11.5, about 12.0, about 12.5, about 13.0, about 13.5, about 14.0, about 14.5, about 15.0, about 15.5, about 16.0, about 16.5, about 17.0, about 17.5, about 18.0, about 18.5, about 19.0, about 19.5, about 20, about 20.5, about 21.0, about 21.5, about 22.0, about 22.5, about 23.0, about 23.5, about 24.0, about 24.5, about 25.0, about 25.5, about 26.0, about 26.5, about 27.0, about 27.5, about 28.0, about 28.5, about 29.0, or about 29.5 fold, inclusive of all values and subranges therebetween.

### Re-Spliced Target Proteins

The “target protein” is the amino acid sequence resulting from transcription and translation of the target gene. The “re-spliced target protein” as used herein refers to the protein encoded as a result of binding of the AC to the target pre-mRNA transcribed from the target gene. The “wild type target protein” refers to a naturally occurring, correctly translated protein isomer resulting from proper splicing of the target pre-mRNA encoded by a wild-type target gene. The present compounds and methods may result in a re-spliced target protein containing one or more amino acid substitutions, deletions, and/or insertions as compared to a wild-type target protein. In embodiments, the re-spliced target protein retains some wild-type target protein activity. In embodiments, the re-spliced target protein produced by administration of the present compounds is homologous to a wild-type target protein. In embodiments, the re-spliced target protein has an amino acid sequence that is at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% and up to 100% identical to a wild type target protein. In embodiments, the re-spliced target protein is substantially identical to a wild-type target protein. In embodiments, the amino acid sequence of the re-spliced target protein is at least 50% identical to the amino acid sequence of a wild-type target protein. In embodiments, the amino acid sequence of the re-spliced target protein is at least 75% identical to the amino acid sequence of a wild-type target protein. In embodiments, the amino acid sequence of the re-spliced target protein is at least 90% identical to the amino acid sequence of a wild-type target protein. In embodiments, the re-spliced target protein is a shortened version of a wild-type target protein.

In embodiments, the re-spliced target protein can rescue one or more phenotypes or symptoms of a disease associated with the transcription and translation of the target gene. In embodiments, the re-spliced target protein can rescue one or more phenotypes or symptoms of a disease associated with the expression of the target protein. In embodiments, the re-spliced target protein is an active fragment of a wild-type target protein. In embodiments, the re-spliced target protein functions in a substantially similar manner to the wild-type target protein. In embodiments, the re-spliced target protein allows the cell to function substantially similar to a similar cell which expresses a wild-type target protein. In embodiments, the re-spliced target protein does not cure the disease associated with the target gene or with the target protein but ameliorates one or more symptoms of the disease. In embodiments, the re-spliced target protein results in an improvement of target protein function of at least about 1%, about 2%, about 3%, about 4%, about 5%, about 6%, about 7%, about 8%, about 9%, about 10%, about 15%, about 205, about 25%, about 30%, about 35%, about 40%, about 45%, about 50%, about 55%, about 60%, about 65%, about 70%, about 75%, about 80%, about 85%, about 90%, or about 95%, and up to about 100%.

In embodiments, the re-spliced target protein may have an amino acid sequence that is reduced from the size of a wild type target protein by about 1 or more amino acids, e.g., from about 5, about 10, about 15, about 20, about 25, about 30, about 35, about 40, about 45, about 50, about 55, about 60, about 65, about 70, about 75, about 80, about 90, about 95, about 100, about 105, about 110, about 115, about 120, about 125, about 130, about 135, about 140, about 145, about 150, about 155, about 160, about 165, about 170, about 175, or about 180 or more amino acids.

In embodiments, the re-spliced target protein may have one or more properties that are improved relative to the target protein. In embodiments, the re-spliced target protein may have one or more properties that are improved relative to a wild-type target protein. In embodiments, the enzymatic activity or stability may be enhanced by promoting different splicing of the target pre-mRNA. In embodiments, the re-spliced target protein may have a sequence identical or substantially similar to a wild-type target protein isomer having improved properties compared to another wild-type target protein isomer.

In embodiments, one or more properties of the target protein are either not present (eliminated) or are reduced in the re-spliced target protein. In embodiments, one or more properties of the wild-type target protein are either not present (eliminated) or are reduced in the re-spliced target protein. Non-limiting examples of properties that may be reduced or eliminated include immunogenic, angiogenic, thrombogenic, aggregation, and ligand-binding activity.

In embodiments, the re-spliced target protein contains one or more amino acid substitutions compared to a wild-type target protein. In embodiments, the substitutions may be conservative substitutions or non-conservative substitutions. Examples of conservative amino acid substitutions include substitution of one amino acid for another amino acid within one from one of the following groups: basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). In embodiments, structurally similar amino acids are substituted to reverse the charge of a residue (e.g., glutamine for glutamic acid or vice-versa, aspartic acid for asparagine or vice-versa). In embodiments, tyrosine is substituted for phenylalanine or vice-versa. Other non-limiting examples of amino acid substitutions are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

In embodiments, the re-spliced target protein may comprise a substitution, deletion, and/or insertion at one or more (e.g., several) positions compared to a wild-type target protein. In embodiments, the number of amino acid substitutions, deletions and/or insertions in the re-spliced target protein amino acid sequence is not more than 200, not more than 150, not more than 100, not more than 50, not more than 40, not more than 30, not more than 20, or not more than 10, e.g., 1, 2, 3, 4, 5, 6, 7, 8, 9, or 10.

### Methods of Treatment

In embodiments, an AC of the disclosure is administered to a patient diagnosed with Duchenne muscular dystrophy (DMD) at a dose from about 0.1 mg/kg to about 1000 mg/kg, for example, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 61 mg/kg, about 62 mg/kg, about 63 mg/kg, about 64 mg/kg, about 65 mg/kg, about 66 mg/kg, about 67 mg/kg, about 68 mg/kg, about 69 mg/kg, about 70 mg/kg, about 71 mg/kg, about 72 mg/kg, about 73 mg/kg, about 74 mg/kg, about 75 mg/kg, about 76 mg/kg, about 77 mg/kg, about 78 mg/kg, about 79 mg/kg, about 80 mg/kg, about 81 mg/kg, about 82 mg/kg, about 83 mg/kg, about 84 mg/kg, about 85 mg/kg, about 86 mg/kg, about 87 mg/kg, about 88 mg/kg, about 89 mg/kg, about 90 mg/kg, about 91 mg/kg, about 92 mg/kg, about 93 mg/kg, about 94 mg/kg, about 95 mg/kg, about 96 mg/kg, about 97 mg/kg, about 98 mg/kg, about 99 mg/kg, about 100 mg/kg, about 110 mg/kg, about 120 mg/kg, about 130 mg/kg, about 140 mg/kg, about 150 mg/kg, about 160 mg/kg, about 170 mg/kg, about 180 mg/kg, about 190 mg/kg, about 200 mg/kg, about 210 mg/kg, about 220 mg/kg, about 230 mg/kg, about 240 mg/kg, about 250 mg/kg, about 260 mg/kg, about 270 mg/kg, about 280 mg/kg, about 290 mg/kg, about 300 mg/kg, about 310 mg/kg, about 320 mg/kg, about 330 mg/kg, about 340 mg/kg, about 350 mg/kg, about 360 mg/kg, about 370 mg/kg, about 380 mg/kg, about 390 mg/kg, about 400 mg/kg, about 410 mg/kg, about 420 mg/kg, about 430 mg/kg, about 440 mg/kg, about 450 mg/kg, about 460 mg/kg, about 470 mg/kg, about 480 mg/kg, about 490 mg/kg, about 500 mg/kg, about 510 mg/kg, about 520 mg/kg, about 530 mg/kg, about 540 mg/kg, about 550 mg/kg, about 560 mg/kg, about 570 mg/kg, about 580 mg/kg, about 590 mg/kg, about 600 mg/kg, about 610 mg/kg, about 620 mg/kg, about 630 mg/kg, about 640 mg/kg, about 650 mg/kg, about 660 mg/kg, about 670 mg/kg, about 680 mg/kg, about 690 mg/kg, about 700 mg/kg, about 710 mg/kg, about 720 mg/kg, about 730 mg/kg, about 740 mg/kg, about 750 mg/kg, about 760 mg/kg, about 770 mg/kg, about 780 mg/kg, about 790 mg/kg, about 800 mg/kg, about 810 mg/kg, about 820 mg/kg, about 830 mg/kg, about 840 mg/kg, about 850 mg/kg, about 860 mg/kg, about 870 mg/kg, about 880 mg/kg, about 890 mg/kg, about 900 mg/kg, about 910 mg/kg, about 920 mg/kg, about 930 mg/kg, about 940 mg/kg, about 950 mg/kg, about 960 mg/kg, about 970 mg/kg, about 980 mg/kg, about 990 mg/kg, or about 1000 mg/kg, including all values and ranges therein and in between.

The present disclosure provides a method of treating Duchenne Muscular Dystrophy (DMD) in a subject in need thereof, comprising administering a compound disclosed herein. In embodiments, the target gene is DMD. In embodiments, the target sequence includes at least a portion of Exon 44 of DMD, at least a portion of a 3′ intron flanking Exon 44 of DMD, at least a portion of a 5′ intron flanking Exon 44 of DMD, or a combination thereof.

In various embodiments, treatment refers to partial or complete alleviation, amelioration, relief, inhibition, delaying onset, reducing severity and/or incidence of one or more symptoms in a subject.

In embodiments, a method is provided for altering the expression of a target gene in a subject in need thereof, comprising administering a compound disclosed herein. In embodiments, the treatment results in the lowered expression of a target protein. In embodiments, the treatment results in the expression of a re-spliced target protein. In embodiments, the treatment results in the preferential expression of a wild-type target protein isomer.

In embodiments, treatment according to the present disclosure results in decreased expression of a target protein in a subject by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the target protein in the subject before the treatment or of one or more control individuals with similar disease without treatment. In embodiments, treatment according to the present disclosure results in increased expression of a re-spliced target protein in a subject by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the target protein in the subject before the treatment or of one or more control individuals with similar disease without treatment. In embodiments, treatment according to the present disclosure results in increased or decreased expression of a wild type target protein isomer in a subject by more than about 5%, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as compared to the average level of the target protein in the subject before the treatment or of one or more control individuals with similar disease without treatment

The terms, “improve,” “increase,” “reduce,” “decrease,” and the like, as used herein, indicate values that are relative to a control. In embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same disease, who is about the same age and/or gender as the individual being treated (to ensure that the stages of the disease in the treated individual and the control individual(s) are comparable).

The individual (also referred to as “patient” or “subject”) being treated is an individual (fetus, infant, child, adolescent, or adult human) having a disease or having the potential to develop a disease. The individual may have a disease mediated by aberrant gene expression or aberrant gene splicing. In various embodiments, the individual having the disease may have wild type target protein expression or activity levels that are from about 1% to 99% of normal protein expression or activity levels in an individual not afflicted with the disease. In embodiments, the range includes, but is not limited to, about 80-99%, about 65-80%, about 50-65%, about 30-50%, about 25-30%, about 20-25%, about 15-20%, about 10-15%, about 5-10%, or about 1-5% of normal thymidine phosphorylase expression or activity levels. In embodiments, the individual may have target protein expression or activity levels that are from about 1% to about 500% higher than normal wild type target protein expression or activity levels. In embodiments, the range includes, but is not limited to, about 1-10%, about 10-50%, about 50-100%, about 100-200%, about 200-300%, about 300-400%, about 400-500%, or about 500-1000% higher target protein expression or activity level.

In embodiments, the individual is an individual who has been recently diagnosed with the disease. Typically, early treatment (treatment commencing as soon as possible after diagnosis) is important to minimize the effects of the disease and to maximize the benefits of treatment.

In embodiments, the efficacy of the compounds and ACs of the disclosure on DMD is evaluated in an animal model of DMD. Animal models are valuable resources for studying the pathogenesis of disease and provide a means to test dystrophin-related activity. In embodiments, the mdx mouse and the golden retriever muscular dystrophy (GRMD) dog, both of which are dystrophin negative (see, e.g., Collins & Morgan, Int J Exp Pathol 84: 165-172, 2003), are utilized to evaluate the compounds of this disclosure. In embodiments, the C₅₇BL/10ScSn-Dmdmdx/J (Bl10/mdx) or the D2.B10-Dmdmdx/J (D2/mdx) mouse model is utilized to evaluate the compounds of this disclosure. In embodiments, a transgenic mouse harboring the human DMD gene and lacking the mouse Dmd gene (hDMD/Dmd-null mouse) is used to evaluate the compounds of this disclosure. This mouse can be generated by cross-breeding male hDMD mice (available from Jackson Laboratory, Bar Harbor, ME) with female DMD-null mice. Each of the following references describe these models and are incorporated by reference in their entirety herein: J Neuromuscul Dis. 2018; 5(4): 407-417.; Proc Natl Acad Sci USA. 1984; 81(4):1189-92.; Am J Pathol. 2010; 176(5):2414-24.; J Clin Invest. 2009; 119(12):3703-12; International Publication No. WO2019014772. These and other animal models can be used to measure the functional activity of various dystrophin proteins.

In embodiments, an in vitro model is used to evaluate the efficacy of the compositions of the disclosure. In embodiments, the in vitro model is an immortalized muscle cell model. This model is described in the following articles which is incorporated by reference in its entirety herein: Nguyen et al. J Pers Med. 2017 December; 7(4):13.

### Methods of Making

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one skilled in the art.

Variations on the compounds described herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, WI), Acros Organics (Morris Plains, NJ), Fisher Scientific (Pittsburgh, PA), Sigma (St. Louis, MO), Pfizer (New York, NY), GlaxoSmithKline (Raleigh, NC), Merck (Whitehouse Station, NJ), Johnson & Johnson (New Brunswick, NJ), Aventis (Bridgewater, NJ), AstraZeneca (Wilmington, DE), Novartis (Basel, Switzerland), Wyeth (Madison, NJ), Bristol-Myers-Squibb (New York, NY), Roche (Basel, Switzerland), Lilly (Indianapolis, IN), Abbott (Abbott Park, IL), Schering Plough (Kenilworth, NJ), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

The disclosed compounds can be prepared by solid phase peptide synthesis wherein the amino acid α-N-terminal is protected by an acid or base protecting group. Such protecting groups should have the properties of being stable to the conditions of peptide linkage formation while being readily removable without destruction of the growing peptide chain or racemization of any of the chiral centers contained therein. Suitable protecting groups are 9-fluorenylmethyloxycarbonyl (Fmoc), t-butyloxycarbonyl (Boc), benzyloxycarbonyl (Cbz), biphenylisopropyloxycarbonyl, t-amyloxycarbonyl, isobornyloxycarbonyl, α,α-dimethyl-3,5-dimethoxybenzyloxycarbonyl, o-nitrophenylsulfenyl, 2-cyano-t-butyloxycarbonyl, and the like. The 9-fluorenylmethyloxycarbonyl (Fmoc) protecting group is particularly preferred for the synthesis of the disclosed compounds. Other preferred side chain protecting groups are, for side chain amino groups like lysine and arginine, 2,2,5,7,8-pentamethylchroman-6-sulfonyl (pmc), nitro, p-toluenesulfonyl, 4-methoxybenzene-sulfonyl, Cbz, Boc, and adamantyloxycarbonyl; for tyrosine, benzyl, o-bromobenzyloxy-carbonyl, 2,6-dichlorobenzyl, isopropyl, t-butyl (t-Bu), cyclohexyl, cyclopentyl and acetyl (Ac); for serine, t-butyl, benzyl and tetrahydropyranyl; for histidine, trityl, benzyl, Cbz, p-toluenesulfonyl and 2,4-dinitrophenyl; for tryptophan, formyl; for asparticacid and glutamic acid, benzyl and t-butyl and for cysteine, triphenylmethyl (trityl). In the solid phase peptide synthesis method, the α-C-terminal amino acid is attached to a suitable solid support or resin. Suitable solid supports useful for the above synthesis are those materials which are inert to the reagents and reaction conditions of the stepwise condensation-deprotection reactions, as well as being insoluble in the media used. Solid supports for synthesis of α-C-terminal carboxy peptides is 4-hydroxymethylphenoxymethyl-copoly(styrene-1% divinylbenzene) or 4-(2′,4′-dimethoxyphenyl-Fmoc-aminomethyl)phenoxyacetamidoethyl resin available from Applied Biosystems (Foster City, Calif). The α-C-terminal amino acid is coupled to the resin by means of N,N′-dicyclohexylcarbodiimide (DCC), N,N′-diisopropylcarbodiimide (DIC) or O-benzotriazol-1-yl-N,N,N′,N′-tetramethyluroniumhexafluorophosphate (HBTU), with or without 4-dimethylaminopyridine (DMAP), 1-hydroxybenzotriazole (HOBT), benzotriazol-1-yloxy-tris(dimethylamino)phosphoniumhexafluorophosphate (BOP) or bis(2-oxo-3-oxazolidinyl)phosphine chloride (BOPCl), mediated coupling for from about 1 to about 24 hours at a temperature of between 10° C. and 50° C. in a solvent such as dichloromethane or DMF. When the solid support is 4-(2′,4′-dimethoxyphenyl-Fmoc-aminomethyl)phenoxy-acetamidoethyl resin, the Fmoc group is cleaved with a secondary amine, preferably piperidine, prior to coupling with the α-C-terminal amino acid as described above. One method for coupling to the deprotected 4 (2′,4′-dimethoxyphenyl-Fmoc-aminomethyl)phenoxy-acetamidoethyl resin is O-benzotriazol-1-yl-N,N,N′,N′-tetramethyluroniumhexafluorophosphate (HBTU, 1 equiv.) and 1-hydroxybenzotriazole (HOBT, 1 equiv.) in DMF. The coupling of successive protected amino acids can be carried out in an automatic polypeptide synthesizer. In one example, the α-N-terminal in the amino acids of the growing peptide chain are protected with Fmoc. The removal of the Fmoc protecting group from the α-N-terminal side of the growing peptide is accomplished by treatment with a secondary amine, preferably piperidine. Each protected amino acid is then introduced in about 3-fold molar excess, and the coupling is preferably carried out in DMF. The coupling agent can be O-benzotriazol-1-yl-N,N,N′,N′-tetramethyluroniumhexafluorophosphate (HBTU, 1 equiv.) and 1-hydroxybenzotriazole (HOBT, 1 equiv.). At the end of the solid phase synthesis, the polypeptide is removed from the resin and deprotected, either in successively or in a single operation. Removal of the polypeptide and deprotection can be accomplished in a single operation by treating the resin-bound polypeptide with a cleavage reagent comprising thioanisole, water, ethanedithiol and trifluoroacetic acid. In cases wherein the α-C-terminal of the polypeptide is an alkylamide, the resin is cleaved by aminolysis with an alkylamine. Alternatively, the peptide can be removed by transesterification, e.g., with methanol, followed by aminolysis or by direct transamidation. The protected peptide can be purified at this point or taken to the next step directly. The removal of the side chain protecting groups can be accomplished using the cleavage cocktail described above. The fully deprotected peptide can be purified by a sequence of chromatographic steps employing any or all of the following types: ion exchange on a weakly basic resin (acetate form); hydrophobic adsorption chromatography on underivatized polystyrene-divinylbenzene (for example, Amberlite XAD); silica gel adsorption chromatography; ion exchange chromatography on carboxymethylcellulose; partition chromatography, e.g., on Sephadex G-25, LH-20 or countercurrent distribution; high performance liquid chromatography (HPLC), especially reverse-phase HPLC on octyl- or octadecylsilyl-silica bonded phase column packing.

The above polymers, such as PEG groups, can be attached to the AC under any suitable conditions used to react a protein with an activated polymer molecule. Any means known in the art can be used, including via acylation, reductive alkylation, Michael addition, thiol alkylation or other chemoselective conjugation/ligation methods through a reactive group on the PEG moiety (e.g., an aldehyde, amino, ester, thiol, α-haloacetyl, maleimido or hydrazino group) to a reactive group on the AC (e.g., an aldehyde, amino, ester, thiol, α-haloacetyl, maleimido or hydrazino group). Activating groups which can be used to link the water soluble polymer to one or more proteins include without limitation sulfone, maleimide, sulfhydryl, thiol, triflate, tresylate, azidirine, oxirane, 5-pyridyl, and alpha-halogenated acyl group (e.g., α-iodo acetic acid, α-bromoacetic acid, α-chloroacetic acid). If attached to the AC by reductive alkylation, the polymer selected should have a single reactive aldehyde so that the degree of polymerization is controlled. See, for example, Kinstler et al., Adv. Drug. Delivery Rev. 54: 477-485 (2002); Roberts et al., Adv. Drug Delivery Rev. 54: 459-476 (2002); and Zalipsky et al., Adv. Drug Delivery Rev. 16: 157-182 (1995).

In order to direct covalently link the AC to the CPP, appropriate amino acid residues of the CPP may be reacted with an organic derivatizing agent that is capable of reacting with a selected side chain or the N- or C-termini of an amino acids. Reactive groups on the peptide or conjugate moiety include, e.g., an aldehyde, amino, ester, thiol, α-haloacetyl, maleimido or hydrazino group. Derivatizing agents include, for example, maleimidobenzoyl sulfosuccinimide ester (conjugation through cysteine residues), N-hydroxysuccinimide (through lysine residues), glutaraldehyde, succinic anhydride or other agents known in the art.

Methods of synthesizing oligomeric antisense compounds are known in the art. The present disclosure is not limited by the method of synthesizing the AC. In embodiments, provided herein are compounds having reactive phosphorus groups useful for forming internucleoside linkages including for example phosphodiester and phosphorothioate internucleoside linkages. Methods of preparation and/or purification of precursors or antisense compounds are not a limitation of the compositions or methods provided herein. Methods for synthesis and purification of DNA, RNA, and the antisense compounds are well known to those skilled in the art.

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Antisense compounds provided herein can be conveniently and routinely made through the well-known technique of solid phase synthesis. Equipment for such synthesis is sold by several vendors including, for example, Applied Biosystems (Foster City, CA). Any other means for such synthesis known in the art may additionally or alternatively be employed. It is well known to use similar techniques to prepare oligonucleotides such as the phosphorothioates and alkylated derivatives. The invention is not limited by the method of antisense compound synthesis.

Methods of oligonucleotide purification and analysis are known to those skilled in the art. Analysis methods include capillary electrophoresis (CE) and electrospray-mass spectroscopy. Such synthesis and analysis methods can be performed in multi-well plates. The method of the invention is not limited by the method of oligomer purification.

### Methods of Administration

In vivo application of the disclosed compounds, and compositions containing them, can be accomplished by any suitable method and technique presently or prospectively known to those skilled in the art. For example, the disclosed compounds can be formulated in a physiologically- or pharmaceutically-acceptable form and administered by any suitable route known in the art including, for example, oral and parenteral routes of administration. As used herein, the term parenteral includes subcutaneous, intradermal, intravenous, intramuscular, intraperitoneal, intrasternal, and intrathecal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 100% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

### Certain Definitions

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “cyclic cell penetrating peptide” or “cCPP” refers to a peptide that facilitates the delivery of an antisense.

The terms “miniPEG”, “PEG₂” and “AEEA” are used interchangeably herein to refer to 2-[2-[2-aminoethoxy]ethoxy]acetic acid.

As used herein, the term “endosomal escape vehicle” (EEV) refers to a cCPP that is conjugated by a chemical linkage (i.e., a covalent bond or non-covalent interaction) to a linker and/or an exocyclic peptide (EP). The EEV can be an EEV of Formula (B).

As used herein, the term “EEV-conjugate” refers to an endosomal escape vehicle defined herein conjugated by a chemical linkage (i.e., a covalent bond or non-covalent interaction) to an AC. The AC can be delivered into a cell by the EEV. The EEV-conjugate can be an EEV-conjugate of Formula (C).

As used herein, the term “exocyclic peptide” (EP) and “modulatory peptide” (MP) may be used interchangeably to refer to two or more amino acid residues linked by a peptide bond that can be conjugated to a cyclic cell penetrating peptide (cCPP) disclosed herein. The EP, when conjugated to a cyclic peptide disclosed herein, may alter the tissue distribution and/or retention of the compound. Typically, the EP comprises at least one positively charged amino acid residue, e.g., at least one lysine residue and/or at least one arginine residue. Non-limiting examples of EP are described herein. The EP can be a peptide that has been identified in the art as a “nuclear localization sequence” (NLS). Non-limiting examples of nuclear localization sequences include the nuclear localization sequence of the SV40 virus large T-antigen, the minimal functional unit of which is the seven amino acid sequence PKKKRKV, the nucleoplasmin bipartite NLS with the sequence NLSKRPAAIKKAGQAKKKK, the c-myc nuclear localization sequence having the amino acid sequence PAAKRVKLD or RQRRNELKRSF, the sequence RMRKFKNKGKDTAELRRRRVEVSVELRKAKKDEQILKRRNV of the IBB domain from importin-alpha, the sequences VSRKRPRP and PPKKARED of the myoma T protein, the sequence PQPKKKPL of human p53, the sequence SALIKKKKKMAP of mouse c-abl IV, the sequences DRLRR and PKQKKRK of the influenza virus NS1, the sequence RKLKKKIKKL of the Hepatitis virus delta antigen and the sequence REKKKFLKRR of the mouse Mxl protein, the sequence KRKGDEVDGVDEVAKKKSKK of the human poly(ADP-ribose) polymerase and the sequence RKCLQAGMNLEARKTKK of the steroid hormone receptors (human) glucocorticoid. International Publication No. 2001/038547 describes additional examples of NLSs and is incorporated by reference herein in its entirety.

As used herein, “linker” or “L” refers to a moiety that covalently bonds one or more moieties (e.g., an exocyclic peptide (EP) and an AC to the cyclic cell penetrating peptide (cCPP). The linker can comprise a natural or non-natural amino acid or polypeptide. The linker can be a synthetic compound containing two or more appropriate functional groups suitable to bind the cCPP to an AC, to thereby form the compounds disclosed herein. The linker can comprise a polyethylene glycol (PEG) moiety. The linker can comprise one or more amino acids. The cCPP may be covalently bound to an AC via a linker.

As used herein, the term “oligonucleotide” refers to an oligomeric compound comprising a plurality of linked nucleotides or nucleosides. One or more nucleotides of an oligonucleotide can be modified. An oligonucleotide can comprise ribonucleic acid (RNA) or deoxyribonucleic acid (DNA). Oligonucleotides can be composed of natural and/or modified nucleobases, sugars and covalent internucleoside linkages, and can further include non-nucleic acid conjugates.

The terms “peptide,” “protein,” and “polypeptide” are used interchangeably to refer to a natural or synthetic molecule comprising two or more amino acids linked by the carboxyl group of one amino acid to the alpha amino group of another. Two or more amino acid residues can be linked by the carboxyl group of one amino acid to the alpha amino group. Two or more amino acids of the polypeptide can be joined by a peptide bond. The polypeptide can include a peptide backbone modification in which two or more amino acids are covalently attached by a bond other than a peptide bond. The polypeptide can include one or more non-natural amino acids, amino acid analogs, or other synthetic molecules that are capable of integrating into a polypeptide. The term polypeptide includes naturally occurring and artificially occurring amino acids. The term polypeptide includes peptides, for example, that include from about 2 to about 100 amino acid residues as well as proteins, that include more than about 100 amino acid residues, or more than about 1000 amino acid residues, including, but not limited to therapeutic proteins such as antibodies, enzymes, receptors, soluble proteins and the like.

The term “therapeutic polypeptide” refers to a polypeptide that has therapeutic, prophylactic or other biological activity. The therapeutic polypeptide can be produced in any suitable manner. For example, the therapeutic polypeptide may isolated or purified from a naturally occurring environment, may be chemically synthesized, may be recombinantly produced, or a combination thereof.

The term “small molecule” refers to an organic compound with pharmacological activity and a molecular weight of less than about 2000 Daltons, or less than about 1000 Daltons, or less than about 500 Daltons. Small molecule therapeutics are typically manufactured by chemical synthesis.

As used herein, the term “contiguous” refers to two amino acids, which are connected by a covalent bond. For example, in the context of a representative cyclic cell penetrating peptide (cCPP) such as

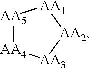

AA₁/AA₂, AA₂/AA₃, AA₃/AA₄, and AA₅/AA₁ exemplify pairs of contiguous amino acids.

A residue of a chemical species, as used herein, refers to a derivative of the chemical species that is present in a particular product. To form the product, at least one atom of the species is replaced by a bond to another moiety, such that the product contains a derivative, or residue, of the chemical species. For example, the cyclic cell penetrating peptides (cCPP) described herein have amino acids (e.g., arginine) incorporated therein through formation of one or more peptide bonds. The amino acids incorporated into the cCPP may be referred to residues, or simply as an amino acid. Thus, arginine or an arginine residue refers to

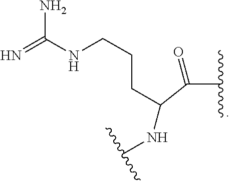 [SMILES: *NC(CCCNC(=N)N)C(*)=O]

The term “protonated form thereof” refers to a protonated form of an amino acid. For example, the guanidine group on the side chain of arginine may be protonated to form a guanidinium group. The structure of a protonated form of arginine is

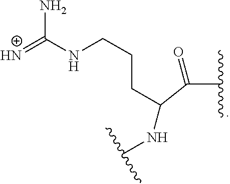 [SMILES: *NC(CCCNC(N)=[NH2+])C(*)=O]

As used herein, the term “chirality” refers to a molecule that has more than one stereoisomer that differs in the three-dimensional spatial arrangement of atoms, in which one stereoisomer is a non-superimposable mirror image of the other. Amino acids, except for glycine, have a chiral carbon atom adjacent to the carboxyl group. The term “enantiomer” refers to stereoisomers that are chiral. The chiral molecule can be an amino acid residue having a “D” and “L” enantiomer. Molecules without a chiral center, such as glycine, can be referred to as “achiral.”

As used herein, the term “hydrophobic” refers to a moiety that is not soluble in water or has minimal solubility in water. Generally, neutral moieties and/or non-polar moieties, or moieties that are predominately neutral and/or non-polar are hydrophobic. Hydrophobicity can be measured by one of the methods disclosed herein below.

As used herein “aromatic” refers to an unsaturated cyclic molecule having 4n+2 π electrons, wherein n is any integer. The term “non-aromatic” refers to any unsaturated cyclic molecule which does not fall within the definition of aromatic.

“Alkyl”, “alkyl chain” or “alkyl group” refer to a fully saturated, straight or branched hydrocarbon chain radical having from one to forty carbon atoms, and which is attached to the rest of the molecule by a single bond. Alkyls comprising any number of carbon atoms from 1 to 40 are included. An alkyl comprising up to 40 carbon atoms is a C₁-C₄₀ alkyl, an alkyl comprising up to carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene”, “alkylene chain” or “alkylene group” refers to a fully saturated, straight or branched divalent hydrocarbon chain radical, having from one to forty carbon atoms. Non-limiting examples of C₂-C₄₀ alkylene include ethylene, propylene, n-butylene, ethenylene, propenylene, n-butenylene, propynylene, n-butynylene, and the like. Unless stated otherwise specifically in the specification, an alkylene chain can be optionally substituted.

“Alkenyl”, “alkenyl chain” or “alkenyl group” refers to a straight or branched hydrocarbon chain radical having from two to forty carbon atoms and having one or more carbon-carbon double bonds. Each alkenyl group is attached to the rest of the molecule by a single bond. Alkenyl groups comprising any number of carbon atoms from 2 to 40 are included. An alkenyl group comprising up to 40 carbon atoms is a C₂-C₄₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene”, “alkenylene chain” or “alkenylene group” refers to a straight or branched divalent hydrocarbon chain radical, having from two to forty carbon atoms, and having one or more carbon-carbon double bonds. Non-limiting examples of C₂-C₄₀ alkenylene include ethene, propene, butene, and the like. Unless stated otherwise specifically in the specification, an alkenylene chain can be optionally.

“Alkoxy” or “alkoxy group” refers to the group —OR, where R is alkyl, alkenyl, alkynyl, cycloalkyl, or heterocyclyl as defined herein. Unless stated otherwise specifically in the specification, an alkoxy group can be optionally substituted.

“Acyl” or “acyl group” refers to groups —C(O)R, where R is hydrogen, alkyl, alkenyl, alkynyl, carbocyclyl, or heterocyclyl, as defined herein. Unless stated otherwise specifically in the specification, acyl can be optionally substituted.

“Alkylcarbamoyl” or “alkylcarbamoyl group” refers to the group —O—C(O)—NRₐR_{b}, where Rₐ and R_{b} are the same or different and are independently an alkyl, alkenyl, alkynyl, aryl, heteroaryl, as defined herein, or RₐR_{b} can be taken together to form a cycloalkyl group or heterocyclyl group, as defined herein. Unless stated otherwise specifically in the specification, an alkylcarbamoyl group can be optionally substituted.

“Alkylcarboxamidyl” or “alkylcarboxamidyl group” refers to the group —C(O)—NRₐR_{b}, where Rₐ and R_{b} are the same or different and are independently an alkyl, alkenyl, alkynyl, aryl, heteroaryl, cycloalkyl, cycloalkenyl, cycloalkynyl, or heterocyclyl group, as defined herein, or RₐR_{b} can be taken together to form a cycloalkyl group, as defined herein. Unless stated otherwise specifically in the specification, an alkylcarboxamidyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

The term “substituted” used herein means any of the above groups (i.e., alkyl, alkenyl, alkynyl, cycloalkyl, cycloalkenyl, cycloalkynyl, heterocyclyl, aryl, heteroaryl, alkoxy, aryloxy, acyl, alkylcarbamoyl, alkylcarboxamidyl, alkoxycarbonyl, alkylthio, or arylthio) wherein at least one atom is replaced by a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more atoms are replaced by an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. “Substituted” can also mean an amino acid in which one or more atoms on the side chain are replaced by alkyl, alkenyl, alkynyl, acyl, alkylcarboxamidyl, alkoxycarbonyl, carbocyclyl, heterocyclyl, aryl, or heteroaryl. In addition, each of the foregoing substituents can also be optionally substituted with one or more of the above substituents.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

As used herein, the term “sequence identity” refers to the percentage of amino acids between two polypeptide sequences that are the same and in the same relative position. As such one polypeptide sequence has a certain percentage of sequence identity compared to another polypeptide sequence. For sequence comparison, typically one sequence acts as a reference sequence, to which test sequences are compared. Those of ordinary skill in the art will appreciate that two sequences are generally considered to be “substantially identical” if they contain identical residues in corresponding positions. In embodiments, the sequence identity between two amino acid sequences may be determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), in the version that exists as of the date of filing. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows: (Identical Residues×100)/(Length of Alignment-Total Number of Gaps in Alignment)

In embodiments, sequence identity may be determined using the Smith-Waterman algorithm, in the version that exists as of the date of filing.

As used herein, “sequence homology” refers to the percentage of amino acids between two polypeptide sequences that are homologous and in the same relative position. As such one polypeptide sequence has a certain percentage of sequence homology compared to another polypeptide sequence. As will be appreciated by those of ordinary skill in the art, two sequences are generally considered to be “substantially homologous” if they contain homologous residues in corresponding positions. Homologous residues may be identical residues. Alternatively, homologous residues may be non-identical residues with appropriately similar structural and/or functional characteristics. For example, as is well known by those of ordinary skill in the art, certain amino acids are typically classified as “hydrophobic” or “hydrophilic” amino acids, and/or as having “polar” or “non-polar” side chains, and substitution of one amino acid for another of the same type may often be considered a “homologous” substitution.

As is well known in this art, amino acid sequences may be compared using any of a variety of algorithms, including those available in commercial computer programs such as BLASTP, gapped BLAST, and PSI-BLAST, in existence as of the date of filing. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying homologous sequences, the programs mentioned above typically provide an indication of the degree of homology.

As used herein, the terms “antisense compound” and “AC” are used interchangeably to refer to a polymeric nucleic acid structure (which can also be referred to as an oligonucleotide or polynucleotide) which is at least partially complementary to a target nucleic acid molecule to which it (the AC) hybridizes. The AC may be a short (In embodiments, less than 50 base pair) polynucleotide or polynucleotide homologue comprising a sequence complimentary to a target sequence in a target pre-mRNA strand. The AC may be formed of natural nucleic acids, synthetic nucleic acids, nucleic acid homologues, or any combination thereof. In embodiments, the AC comprises oligonucleosides. In embodiments, AC comprises antisense oligonucleotides. In embodiments, the AC comprises conjugate groups. Nonlimiting examples of ACs include, but are not limited to, primers, probes, antisense oligonucleotides, external guide sequence (EGS) oligonucleotides, alternate splicers, siRNAs, oligonucleotides, oligonucleosides, oligonucleotide analogs, oligonucleotide mimetics, and chimeric combinations of these. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound. In embodiments, an AC modulates (increases, decreases, or changes) expression of a target nucleic acid. Various modifications may be made to the polymeric nucleic acid structure, such as phosphorodiamidate morpholino (PMO). Therefore, AC as used herein encompasses any modification described herein, such as a PMO.

The terms “pre-mRNA” and “primary transcript” as used herein refer to a newly synthesized eukaryotic mRNA molecule directly after DNA transcription. A pre-mRNA must be capped with a 5′ cap, modified with a 3′ poly-A tail, and spliced to produce a mature mRNA sequence.

As used herein, the terms “targeting” or “targeted to” refer to the association of an antisense compound (AC) with a target nucleic acid molecule or a region of a target nucleic acid molecule. In embodiments, the AC is capable of hybridizing to a target nucleic acid under physiological conditions. In embodiments, the AC targets a specific portion or site within the target nucleic acid, for example, a portion of the target nucleic acid having at least one identifiable structure, function, or characteristic such as a particular exon or intron, or selected nucleobases or motifs within an exon or intron.

As used herein, the terms “target nucleic acid” and “target sequence” refer to a nucleic acid molecule having a nucleic acid sequence to which the antisense compound binds or hybridizes. Target nucleic acids include, but are not limited to, RNA (including, but not limited to pre-mRNA and mRNA or portions thereof), cDNA derived from such RNA, as well as non-translated RNA, such as miRNA. For example, In embodiments, a target nucleic acid can be a cellular gene (or mRNA transcribed from such gene) whose expression is associated with a particular disorder or disease state, or a nucleic acid molecule from an infectious agent. In embodiments, the target nucleic acid is a target RNA. In embodiments, the target nucleic acid is a target mRNA. In embodiments, the target nucleic acid is a target pre-mRNA.

As used herein, the terms “splicing” and “processing” refer to the modification of a pre-mRNA following transcription, in which introns are removed and exons are joined. Splicing occurs in a series of reactions that are catalyzed by a large RNA-protein complex composed of five small nuclear ribonucleoproteins (snRNPs) referred to as a spliceosome. Within an intron, a 3′ splice site, a 5′ splice site, and a branch site are required for splicing. The RNA components of snRNPs interact with the intron and may be involved in catalysis

As used herein, the term “exon” refers to a portion of a pre-mRNA which, after splicing, is typically included in the mature mRNA.

As used herein, the term “intron” refers to a portion of a pre-mRNA which, after splicing, is typically excluded from the mature mRNA.

As used herein, the term “flanking” refers to an intron located immediately upstream (5′) or immediately downstream (3′) of an associated exon. For example, the 5′ flanking intron of exon 44 refers to the intron that is immediately upstream of (i.e., directly coupled to the 5′ end of) exon 44. For example, the 3′ flanking intron of exon 44 refers to the intron that is immediately downstream of (i.e., directly coupled to the 5′ end of) exon 44.

The “target pre-mRNA” is the pre-mRNA comprising the target sequence to which the AC hybridizes.

The “target mRNA” is the mRNA sequence resulting from splicing of the target pre-mRNA sequence. In embodiments, the target mRNA does not encode a functional protein. In embodiments, the target mRNA retains one or more intron sequences.

As used herein, the term “gene” refers to a nucleic acid molecule having a nucleic acid sequence that encompasses a 5′ promoter region associated with the expression of the gene product, and any intron and exon regions and 3′ untranslated regions (“UTR”) associated with the expression of the gene product.

The “target gene” of the present disclosure refers to the gene that encodes the target pre-mRNA.

The “target protein” refers to the amino acid sequence encoded by the target mRNA. In embodiments, the target protein may not be a functional protein.

“Wild type target protein” refers to a native, functional protein isomer produced by a wild type, normal, or unmutated version of the target gene. The wild type target protein also refers to the protein resulting from a target pre-mRNA that has been properly spliced.

As used herein, the term “transcript” refers an RNA molecule transcribed from DNA and includes, but is not limited to mRNA, mature mRNA, pre-mRNA, and partially processed RNA.

A “re-spliced target protein”, as used herein, refers to the protein encoded by the mRNA resulting from the splicing of the target pre-mRNA to which the AC hybridizes. Re-spliced target protein may be identical to a wild type target protein, may be homologous to a wild type target protein, may be a functional variant of a wild type target protein, or may be an active fragment of a wild type target protein.

As used herein, “functional fragment” or “active fragment” refers to a portion of a eukaryotic wild type target protein that exhibits an activity, such as one or more activities of a full-length wild type target protein, or that possesses another activity. In embodiments, a re-spliced target protein that shares at least one biological activity of wild type target protein is considered to be an active fragment of the wild type target protein. Activity can be any percentage of activity (i.e., more or less) of the full-length wild type target protein, including but not limited to, about 1% of the activity, about 2%, about 3%, about 4%, about 5%, about 10%, about 20%, about 30%, about 40%, about 50%, about 60%, about 70%, about 80%, about 90%, about 95%, about 96%, about 97%, about 98%, about 99%, about 100%, about 200%, about 300%, about 400%, about 500%, or more (including all values and ranges inbetween these values) activity compared to the wild type target protein. Thus, In embodiments, the active fragment may retain at least a portion of one or more biological activities of wild type target protein. In embodiments, the active fragment may enhance one or more biological activities of wild type target protein.

As used herein, the term “nucleoside” means a glycosylamine comprising a nucleobase and a sugar. Nucleosides includes, but are not limited to, natural nucleosides, abasic nucleosides, modified nucleosides, and nucleosides having mimetic bases and/or sugar groups. A “natural nucleoside” or “unmodified nucleoside” is a nucleoside comprising a natural nucleobase and a natural sugar. Natural nucleosides include RNA and DNA nucleosides.

As used herein, the term “natural sugar” refers to a sugar of a nucleoside that is unmodified from its naturally occurring form in RNA (2′-OH) or DNA (2′-H).

As used herein, the term “nucleotide” refers to a nucleoside having a phosphate group covalently linked to the sugar. Nucleotides may be modified with any of a variety of substituents.

As used herein, the term “nucleobase” refers to the base portion of a nucleoside or nucleotide. A nucleobase may comprise any atom or group of atoms capable of hydrogen bonding to a base of another nucleic acid. A natural nucleobase is a nucleobase that is unmodified from its naturally occurring form in RNA or DNA.

As used herein, the term “heterocyclic base moiety” refers to a nucleobase comprising a heterocycle.

As used herein “oligonucleoside” refers to an oligonucleotide in which the internucleoside linkages do not contain a phosphorus atom.

As used herein, the term “oligonucleotide” refers to an oligomeric compound comprising a plurality of linked nucleotides or nucleosides. In certain embodiment, one or more nucleotides of an oligonucleotide is modified. In embodiments, an oligonucleotide comprises ribonucleic acid (RNA) or deoxyribonucleic acid (DNA). In embodiments, oligonucleotides are composed of natural and/or modified nucleobases, sugars and covalent internucleoside linkages, and may further include non-nucleic acid conjugates.

As used herein “internucleoside linkage” refers to a covalent linkage between adjacent nucleosides.

As used herein “natural internucleotide linkage” refers to a 3′ to 5′ phosphodiester linkage.

As used herein, the term “modified internucleoside linkage” refers to any linkage between nucleosides or nucleotides other than a naturally occurring internucleoside linkage.

As used herein the term “chimeric antisense compound” or “chimeric AC” refers to an antisense compound, having at least one sugar, nucleobase and/or internucleoside linkage that is differentially modified as compared to the other sugars, nucleobases and internucleoside linkages within the same oligomeric compound. The remainder of the sugars, nucleobases and internucleoside linkages can be independently modified or unmodified. In general a chimeric oligomeric compound will have modified nucleosides that can be in isolated positions or grouped together in regions that will define a particular motif. Any combination of modifications and or mimetic groups can comprise a chimeric oligomeric compound as described herein.

As used herein, the term “mixed-backbone antisense oligonucleotide” refers to an antisense oligonucleotide wherein at least one internucleoside linkage of the antisense oligonucleotide is different from at least one other internucleotide linkage of the antisense oligonucleotide.

As used herein, the term “nucleobase complementarity” refers to a nucleobase that is capable of base pairing with another nucleobase. For example, in DNA, adenine (A) is complementary to thymine (T). For example, in RNA, adenine (A) is complementary to uracil (U). In embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

As used herein, the term “non-complementary nucleobase” refers to a pair of nucleobases that do not form hydrogen bonds with one another or otherwise support hybridization.

As used herein, the term “complementary” refers to the capacity of an oligomeric compound to hybridize to another oligomeric compound or nucleic acid through nucleobase complementarity. In embodiments, an antisense compound and its target are complementary to each other when a sufficient number of corresponding positions in each molecule are occupied by nucleobases that can bond with each other to allow stable association between the antisense compound and the target. One skilled in the art recognizes that the inclusion of mismatches is possible without eliminating the ability of the oligomeric compounds to remain in association. Therefore, described herein are antisense compounds that may comprise up to about 20% nucleotides that are mismatched (i.e., are not nucleobase complementary to the corresponding nucleotides of the target). Preferably the antisense compounds contain no more than about 15%, more preferably not more than about 10%, most preferably not more than 5% or no mismatches. The remaining nucleotides are nucleobase complementary or otherwise do not disrupt hybridization (e.g., universal bases). One of ordinary skill in the art would recognize the compounds provided herein are at least 80%, at least 85%, at least 90%, at least 95%, at least 96%, at least 97%, at least 98%, at least 99% or 100% nucleobase complementary to a target nucleic acid.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymidine and uracil which pair through the formation of hydrogen bonds. The natural base guanine is nucleobase complementary to the natural bases cytosine and 5-methyl cytosine. Hybridization can occur under varying circumstances.

As used herein, the term “specifically hybridizes” refers to the ability of an oligomeric compound to hybridize to one nucleic acid site with greater affinity than it hybridizes to another nucleic acid site. In embodiments, an antisense oligonucleotide specifically hybridizes to more than one target site. In embodiments, an oligomeric compound specifically hybridizes with its target under stringent hybridization conditions.

The terms “modulate”, “modulating” and “modulation” refer to a perturbation of expression, function or activity when compared to the level of expression, function or activity prior to modulation. Modulation can include an increase (stimulation or induction) or a decrease (inhibition or reduction) in expression, function or activity. In one embodiment, modulation can include perturbation of splice site selection during pre-mRNA processing.

The terms “inhibit”, “inhibiting” or “inhibition” refer to a decrease in an activity, expression, function or other biological parameter and can include, but does not require complete ablation of the activity, expression, function or other biological parameter. Inhibition can include, for example, at least about a 10% reduction in the activity, response, condition, or disease as compared to a control. In embodiments, expression, activity or function of a gene or protein is decreased by a statistically significant amount.

As used herein, the term “expression” refers to all the functions and steps by which a gene's coded information is converted into structures present and operating in a cell. Such structures include, but are not limited to the products of transcription and translation.

As used herein, the term “2-modified” or “2′-substituted” means a sugar comprising substituent at the 2′ position other than H or OH. 2-modified monomers, include, but are not limited to, BNA's and monomers (e.g., nucleosides and nucleotides) with 2′-substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rm and Rn is, independently, H or substituted or unsubstituted C₁-C₁₀ alkyl.

As used herein, the term “MOE” refers to a 2′-O-methoxyethyl substituent.

As used herein, the term “high-affinity modified nucleotide” refers to a nucleotide having at least one modified nucleobase, internucleoside linkage or sugar moiety, such that the modification increases the affinity of an antisense compound comprising the modified nucleotide to a target nucleic acid. High-affinity modifications include, but are not limited to, BNAs, LNAs and 2′-MOE.

As used herein the term “mimetic” refers to groups that are substituted for a sugar, a nucleobase, and/or internucleoside linkage in an AC. Generally, a mimetic is used in place of the sugar or sugar-internucleoside linkage combination, and the nucleobase is maintained for hybridization to a selected target. Representative examples of a sugar mimetic include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances, a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

As used herein, the term “bicyclic nucleoside” or “BNA” refers to a nucleoside wherein the furanose portion of the nucleoside includes a bridge connecting two atoms on the furanose ring, thereby forming a bicyclic ring system. BNAs include, but are not limited to, α-L-LNA, β-D-LNA, ENA, Oxyamino BNA (2′-O—N(CH₃)—CH₂-4′) and Aminooxy BNA (2′-N(CH₃)—O—CH₂-4′).

As used herein, the term “4′ to 2′ bicyclic nucleoside” refers to a BNA wherein the bridge connecting two atoms of the furanose ring bridges the 4′ carbon atom and the 2′ carbon atom of the furanose ring, thereby forming a bicyclic ring system.

As used herein, a “locked nucleic acid” or “LNA” refers to a nucleotide modified such that the 2′-hydroxyl group of the ribosyl sugar ring is linked to the 4′ carbon atom of the sugar ring via a methylene groups, thereby forming a 2′-C,4′-C-oxymethylene linkage. LNAs include, but are not limited to, α-L-LNA, and β-D-LNA.

As used herein, the term “cap structure” or “terminal cap moiety” refers to chemical modifications, which have been incorporated at either end of an AC.

As used herein, the term “dosage unit” refers to a form in which a pharmaceutical agent is provided. In embodiments, a dosage unit is a vial comprising lyophilized antisense oligonucleotide. In embodiments, a dosage unit is a vial comprising reconstituted antisense oligonucleotide.

A number of embodiments of the invention have been described. Nevertheless, it will be understood that various modifications may be made without departing from the spirit and scope of the invention. Other embodiments are within the scope of the following claims.

All publications, patents and patent applications mentioned in the specification are indicative of the level of skill of those skilled in the art to which this invention pertains. All publications, patents and patent applications are herein incorporated by reference to the same extent as if each individual publication or patent application was specifically and individually indicated to be incorporated by reference.

### EXAMPLES

### Example 1. Conjugation of Oligonucleotide with Cell Penetrating Peptide

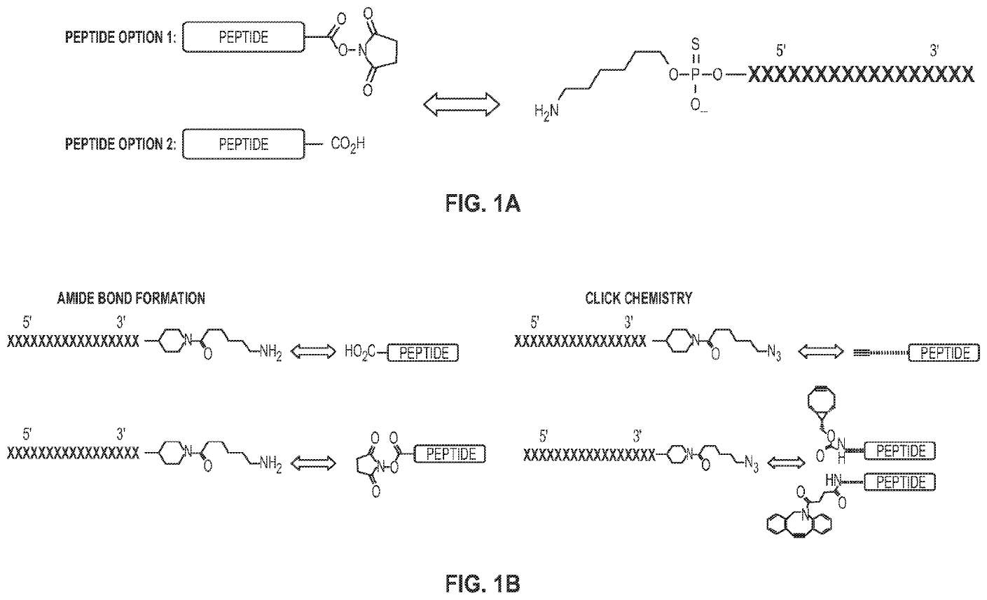

Conjugation of oligonucleotides to cell penetrating peptides. As shown in FIG. 1A, oligonucleotide with a (NH₂—(CH₂)₅—CH₂—) linker on the 5′ phosphorothioate end is conjugated to a CPP via a carboxylate or an N-hydroxysuccinimide ester (NHS ester) functional group on the peptide. As shown in FIG. 1B, oligonucleotide is conjugated to cell penetrating peptide (CPP) via either amide bond formation (left) or click chemistry. The linker/CPP is installed either on the 5′ end, or on the 3′ end of the oligonucleotide.

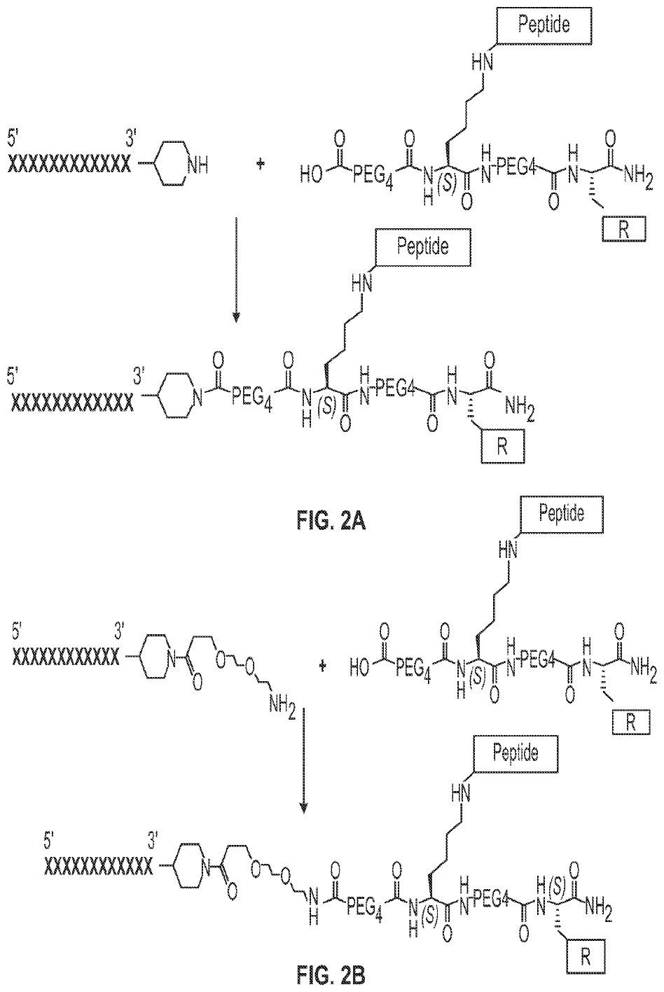

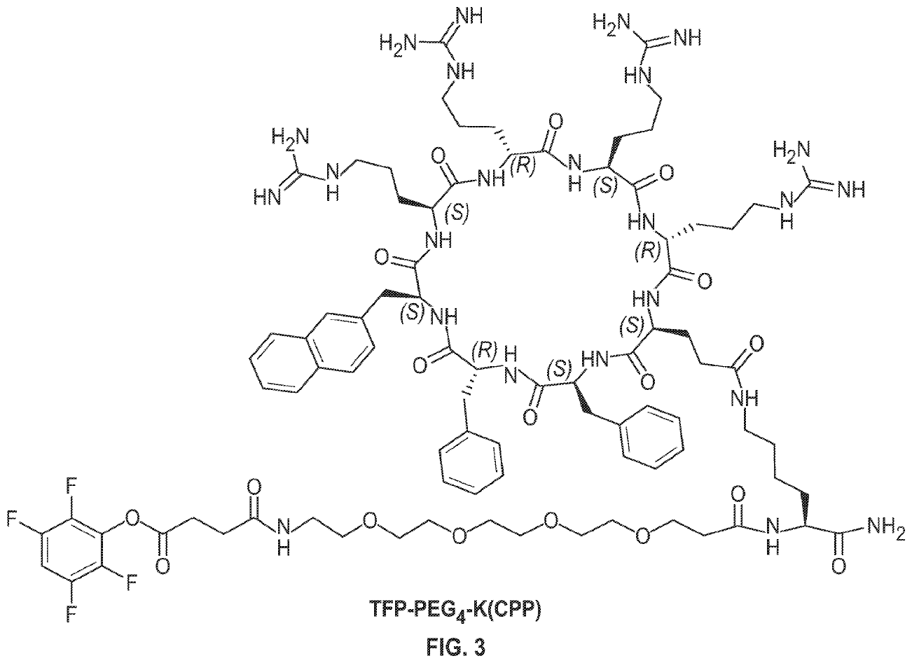

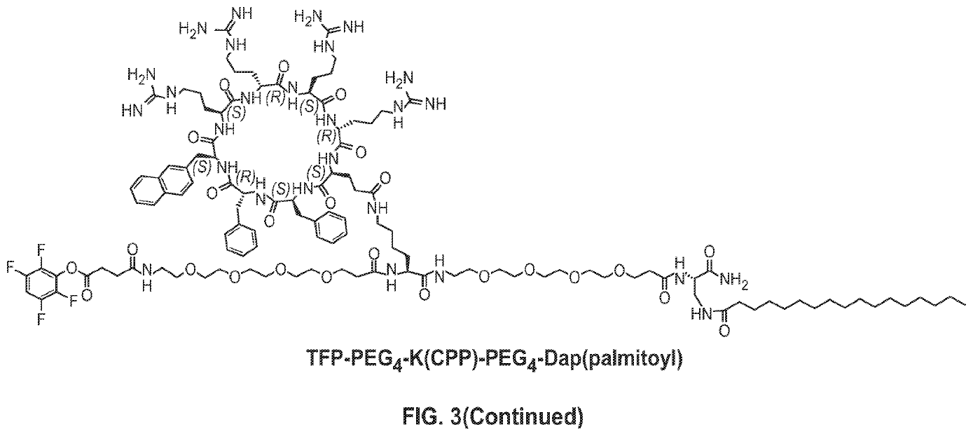

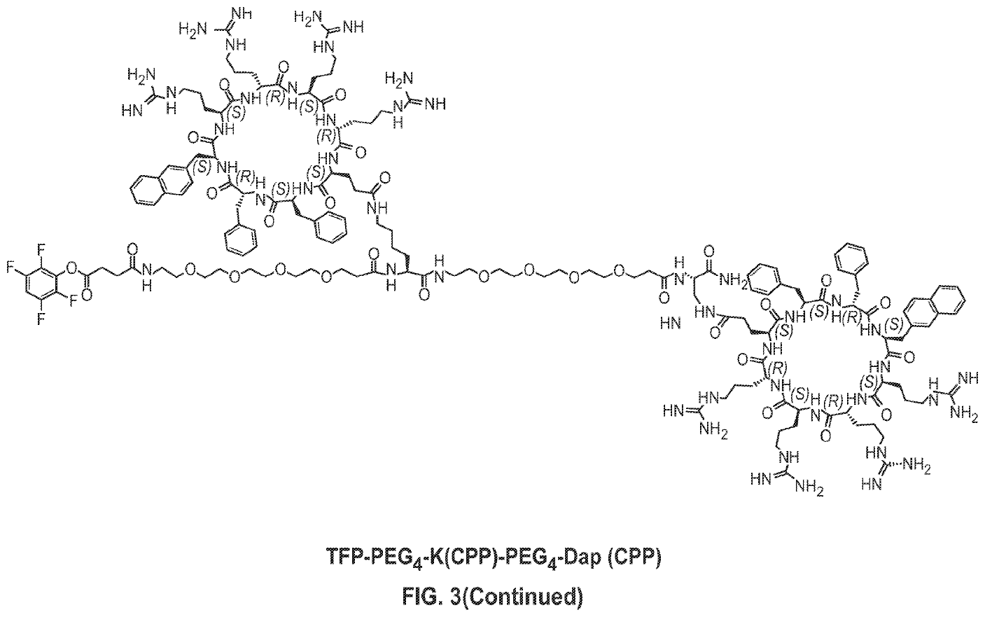

Synthesis of oligonucleotide-peptide conjugate with PEG spacer. As shown in FIGS. 2A and 2B, an oligonucleotide-peptide conjugate is synthesized without (FIG. 2A) and with (FIG. 2B) a PEG (polyethylene glycol) linker inserted between oligonucleotide moiety and peptide. “R” in the figure represents a palmitoyl group.

Synthesis of oligonucleotide-peptide conjugate for various gene targets. Exemplary antisense compounds (AC), for example, those in Tables 6A-6M, or the reverse complement thereof, that target exon 44 of DMD or AC shown in Table 7 are conjugated to a CPP or EEV disclosed herein. In embodiments, the AC is conjugated to a cyclic peptide having the amino acid sequence of FfΦRrRrQ (EEV-1). In embodiments, the AC is conjugated to a cyclic peptide having the amino acid sequence FfΦRrRrQ conjugated to a lipid group (R1) (EEV-1(R1)). In embodiments, the AC is conjugated to an Endosomal Escape Vehicle (EEV) having the sequence cyclo(FfΦRrRrQ)-PEG12-OH. In embodiments, the AC is conjugated to a cyclic peptide having the amino acid sequence FGFGRGRQ. In embodiments, the AC is conjugated to an EEV having the sequence Ac-PKKKRKV-AEEA-Lys-(cyclo[FGFGRGRQ])-PEG12-OH.

TABLE 7
AC that target exon 44 of DMD

| Oligo | |
|---|---|
| chemistry | Design |
| PMO-1 | 5′-GGCCAAACCTCGGCTTACTG AAAT-3′ |
| PMO-2 | 5′-GGCCAAACCTCGGCTTACCTGAAAT-3′ |
| PMO-3 | 5′-XGAAAACGCCGCCAXXXCXCAACAGAXCXG-3′, |
| | wherein X is U or T |
| PMO-4 | 5′-TGAAAACGCCGCCATTTCTCAACAGATCTG-3′ |
| PMO-5 | 5′-TGAAAACGCCGCCATTTCTCAACAG-3′ |
| PMO-6 | 5′-AAACGCCGCCATTTCTCAACAGATC-3′ |
| PMO-7 | 5′-AACGCCGCCATTTCTCAACAGATCT-3′ |

### Example 2. Use of Cell-Penetrating Peptides Conjugated to Oligonucleotides for Exon 23 Splicing Correction of Dystrophin in Mouse Model of DMD

Mice. This study used MDX mice (Sicinski et al. (1989) “The molecular basis of muscular dystrophy in the mdx mouse: a point mutation. Science. 244(4912):1578-80), which contain a C to T mutation resulting in a termination codon at position 2983 within exon 23 of the dystrophin muscular dystrophy gene (Dmd) on the X chromosome. Mice expressing this mutant allele produce a truncated dystrophin protein and are thus a model of Duchenne's muscular dystrophy (“DMD”).

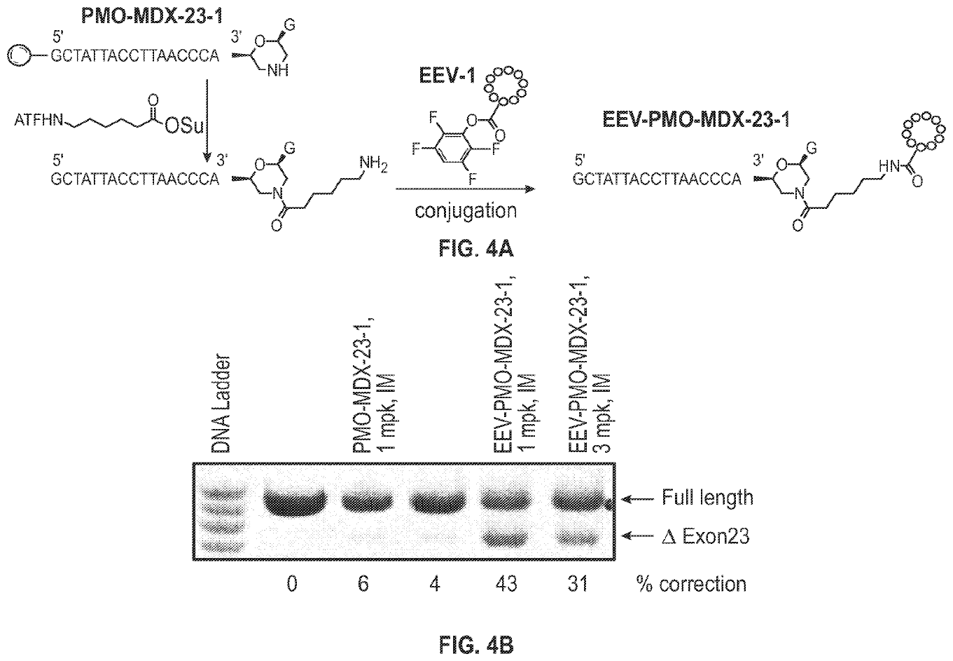

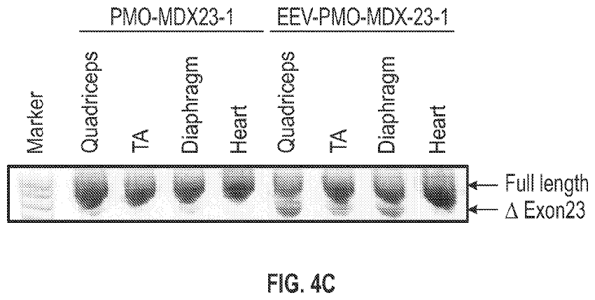

Study design. MDX mice were used to to evaluate the ability of compositions to skip exon 23 and thus treat DMD. The AC used in this study was a phosphorodiamidate morpholino oligomer (PMO) with the following sequence: 5′-GCTATTACCTTAACCCA-3′ (PMO-MDX-23), which targets exon 23. PMO-MDX-23 was conjugated to an Endosomal Escape Vehicle (EEV) with a sequence: cyclo(FfΦRrRrQ)-PEG₁₂-OH (EEV-1) to form an EEV-PMO conjugate (EEV-PMO-MDX-23). The PMO without the EEV is referred to as PMO-MDX-23. A schematic of preparation of EEV-PMO-MDX-23 is shown in FIG. 4A.

EEV-PMO-MDX-23 and PMO-MDX-23 were administered intramuscularly (IM) or intravenously (IV) to MDX mice at the following doses: 1 mpk, 3 mpk, 10 mpk, 30 mpk; (mpk: mg of compound/kg of body weight). Total RNA were extracted from tissue samples and analyzed by RT-PCR to visualize the efficiency of splicing correction.

Detection of splicing correction by RT-PCR and Western Blot. As shown in FIG. 4B, MDX mice treated with EEV-PMO-MDX-23 (1 mpk, IM or 3 mpk, IM) produced dystrophin lacking exon 23, whereas MDX mice treated with PMO-MDX-23 alone produced only dystrophin containing exon 23 (similar to untreated control).

MDX mice were administered 10 mpk PMO-MDX-23 and EEV-PMO-MDX-23 intravenously (IV). The dystrophin products in various muscle groups (e.g., the quadriceps, tibialis anterior, diaphragm, and heart) shown in FIG. 4C demonstrate that, in contrast to delivery of PMO-MDX-23, alone, delivery of EEV-PMO-MDX-23 to MDX mice resulted in corrected dystrophin splicing (e.g., dystrophin with excised exon 23) in the quadriceps, tibialis anterior, diaphragm, and heart.

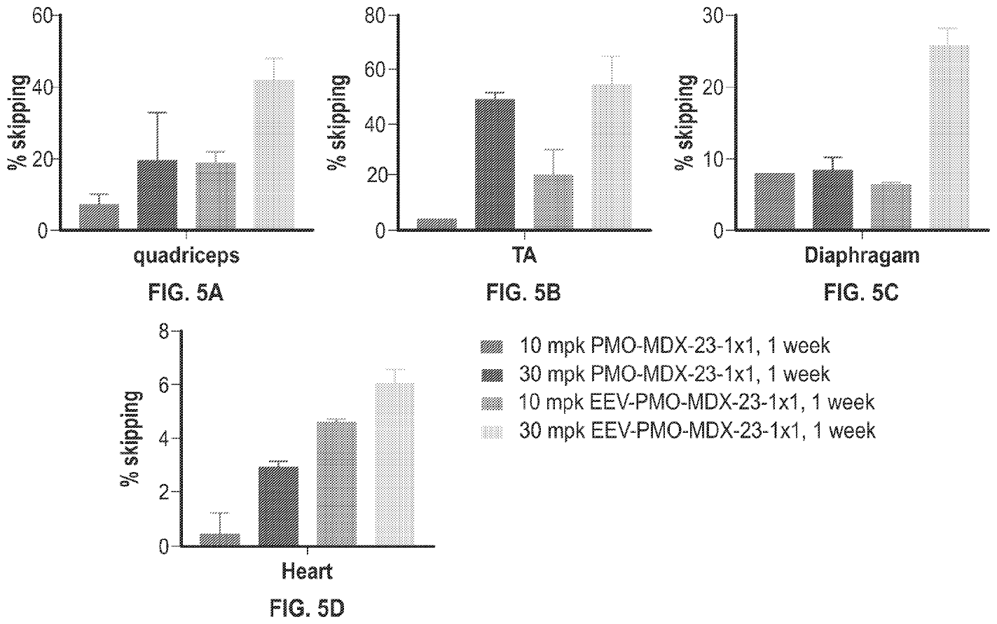

FIGS. 5A-5D show the effect of the following IV dosage regimens on exon skipping efficacy: 10 mpk or 30 mpk dosed once, 1 week. Notably, 30 mpk EEV-PMO-MDX-23 dosed once, 1 week, resulted in the highest percentage of exon skipping in all four tissues: quadriceps (FIG. 5A), tibialis anterior (FIG. 5B), diaphragm (FIG. 5C) and heart (FIG. 5D).

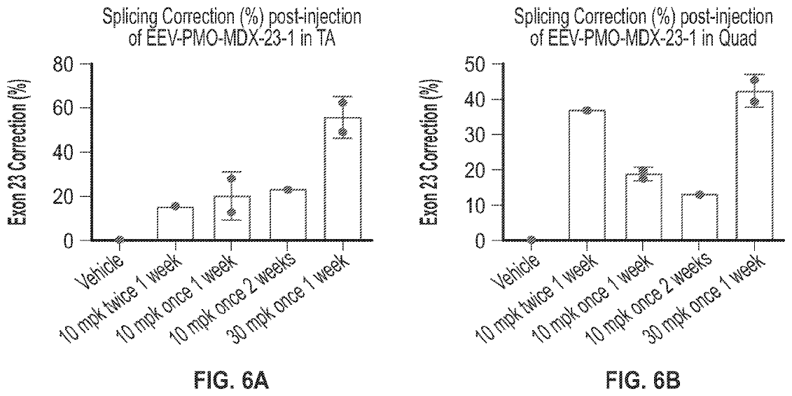

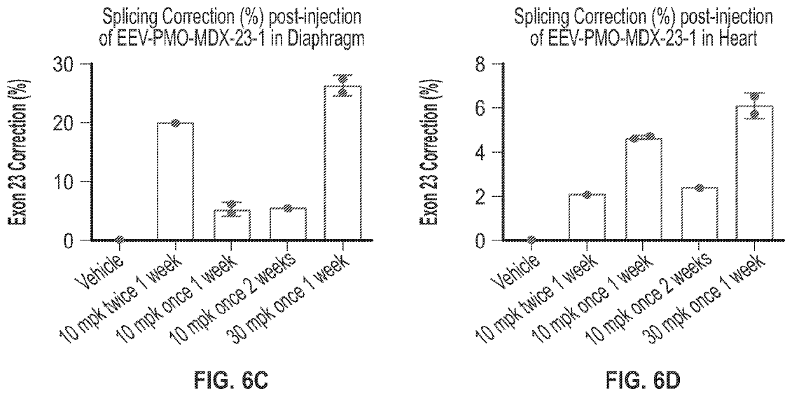

FIG. 6A-6D show the percentage of exon 23 splice corr3eection (as determined by RT-PCR) in the tibialis anterior (FIG. 6A), quadriceps (FIG. 6B), diaphragm (FIG. 6C), and heart (FIG. 6D) after dosing with EEV-PMO-MDX-23 at 10 mpk twice per week, 10 mpk once per week, 10 mpk once per two week, and 30 mpk once per week.

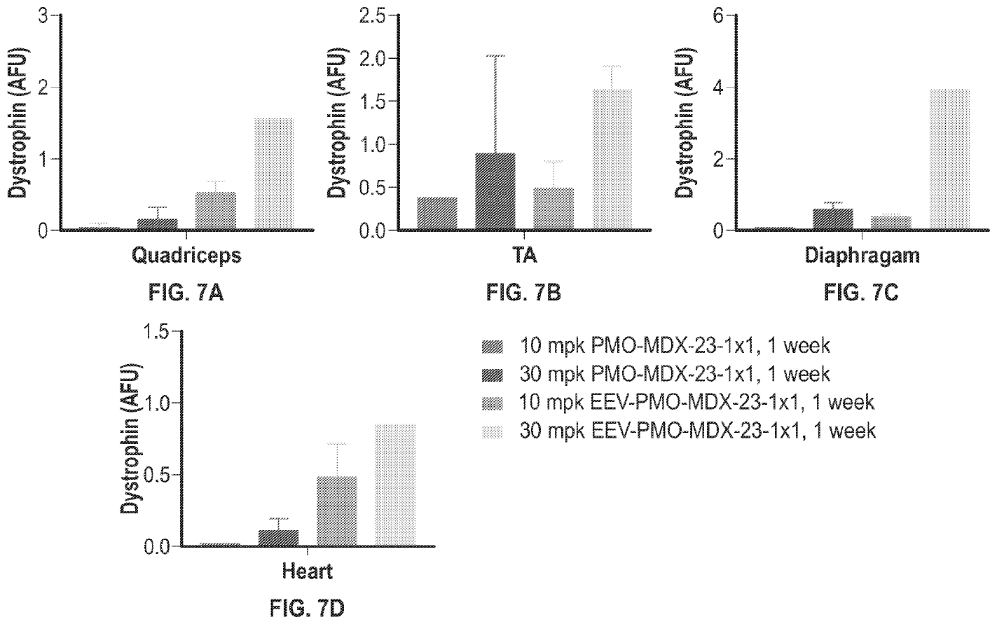

FIGS. 7A-7D show the amount of exon-23 corrected dystrophin detected by Western blot in the quadriceps (FIG. 7A), tibialis anterior (TA) (FIG. 7B), diaphragm (FIG. 7C), and heart (FIG. 7D) after delivery of PMO-MDX-23 or EEV-PMO-MDX-23.

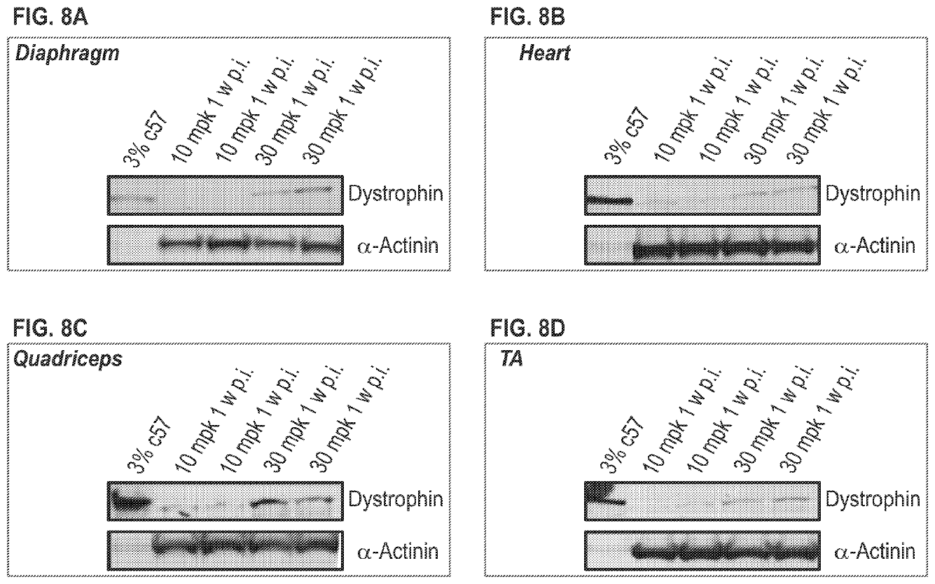

FIGS. 8A-8D show Western Blots of exon 23 corrected dystrophin and α-actinin in the diaphragm (FIG. 8A), heart (FIG. 8B), quadriceps (FIG. 8C), and tibialis anterior (FIG. 8D) after intravenous delivery of 10 mpk or 30 mpk EEV-PMO-MDX-23-1.

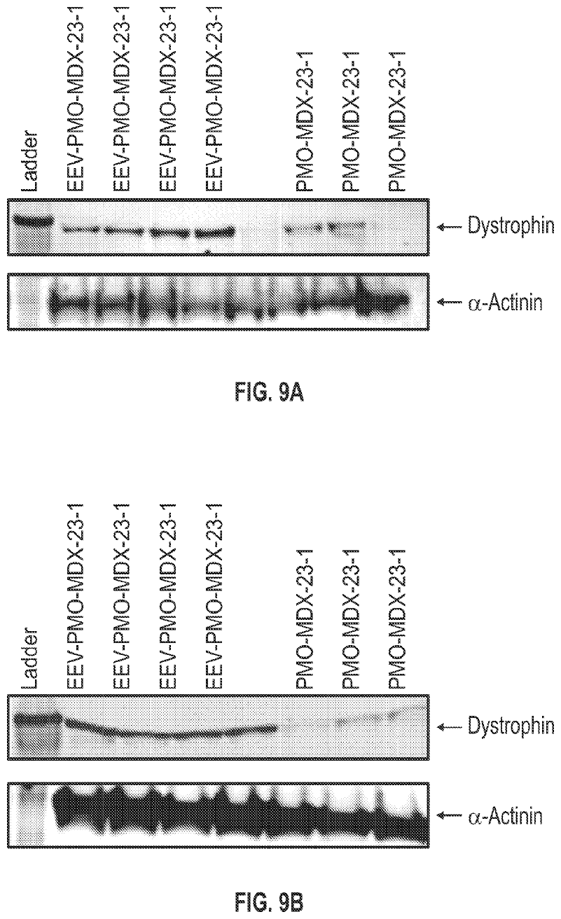

FIGS. 9A-9B show the dystrophin levels in MDX mice two weeks (FIG. 9A) and four weeks (FIG. 9B) after treatment with 30 mpk EEV-PMO-MDX-23 or 30 mpk PMO-MDX-23.

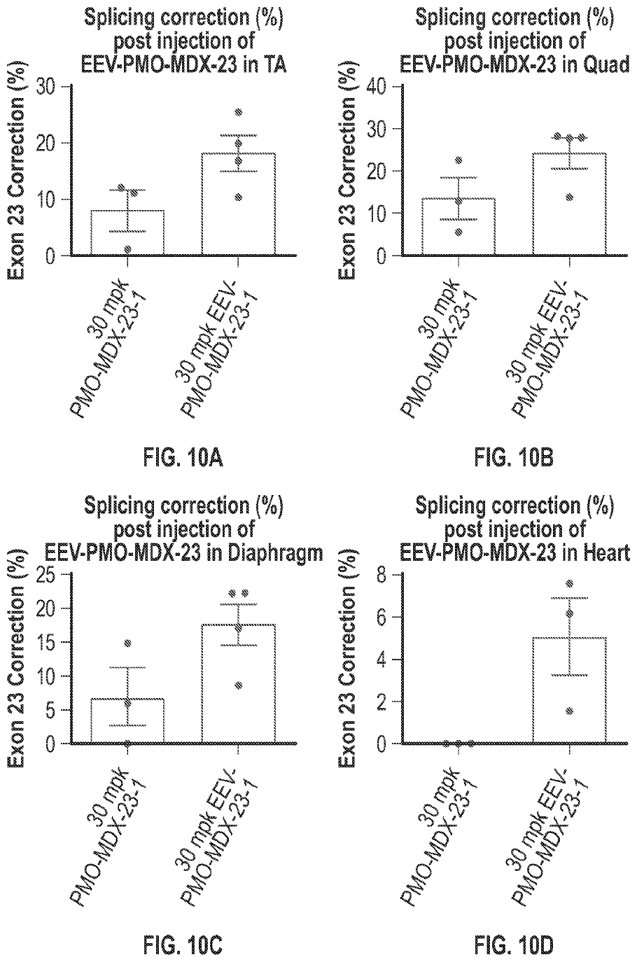

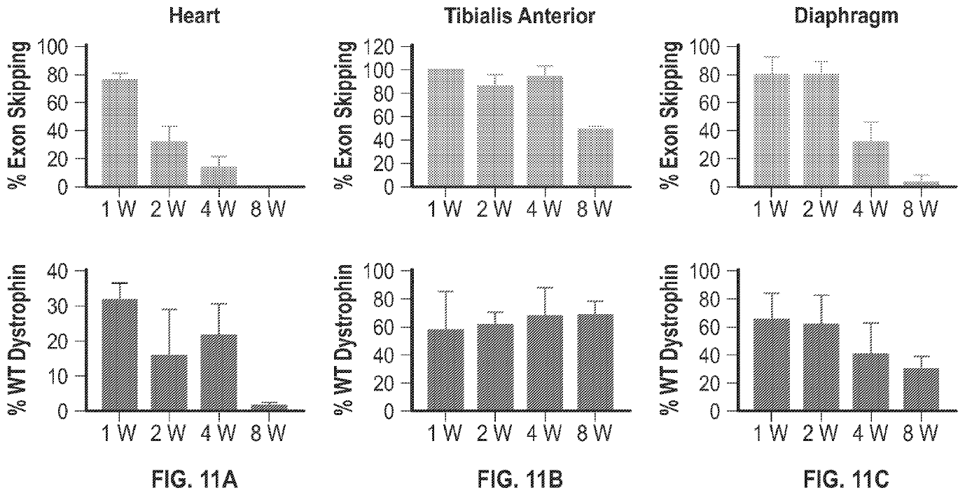

FIGS. 10A-10D show the percentage of exon 23 correction in tibialis anterior (FIG. 10A), quadriceps (FIG. 10B), diaphragm (FIG. 10C), and heart (FIG. 10D) in MDX mice that were administered either 30 mpk of PMO-MDX-23 or 30 mpk of EEV-PMO-MDX-23. Mice administered EEV-PMO-MDX-23 exhibited enhanced splicing correction, compared to mice administered PMO-MDX-23 alone.

FIGS. 11A-11C shows high and persistent levels of exon 23 skipping and dystrophin correction in heart (FIG. 11A), tibialis anterior (FIG. 11B) and diaphram (FIG. 11C) observed up to 8 weeks after a single IV dose (40 mg/kg) of EEV-PMO-MDX-23 in mdx mice.

### Example 3. Use of Cell-Penetrating Peptides Conjugated to Oligonucleotides for Exon 23 Splicing Correction of Dystrophin in the D2-Mdx Mouse Model of DMD

Mice. This study used the D2-mdx mouse model. (Fukada et al. (2010) “Genetic background affects properties of satellite cells and mdx phenotypes. Am. J. Path. 176(5):2414-24).

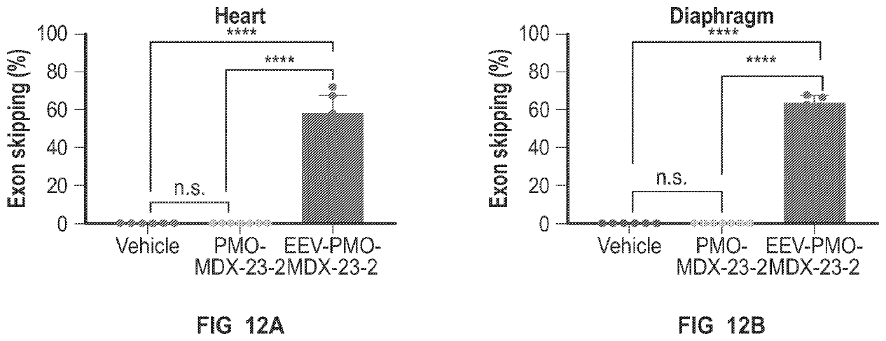

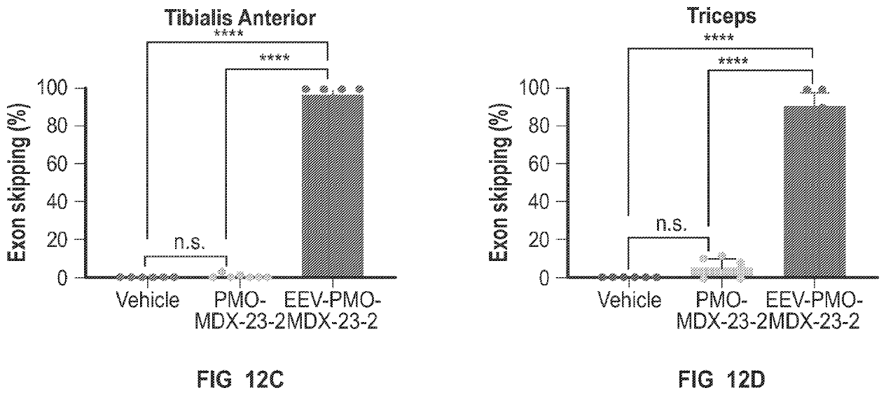

Rrobust exon 23 skipping was observed in different muscle groups (n=6) after monthly repeat 20 mg/kg doses of EEV-PMO-MDX-23-2 (EEV=Ac-PKKKRKV-miniPEG-K(cyclo(GfFGrGrQ))-PEG₁₂-OH; PMO=5′-GGCCAAACCTCGGCTTACCTGAAAT-3′) or PMO-MDX-23-2 (5′-GGCCAAACCTCGGCTTACCTGAAAT-3′). FIG. 12A-12D show the D2-mdx mice exhibited broad dystrophin expression and restoration of muscle integrity in the heart (FIG. 12A), diaphragm (FIG. 12B), tibialis anterior (FIG. 12C) and Triceps (FIG. 12D).

Sarcoglycans interact with dystrophin and other dystrophin-associated proteins to form a dystrophin-associated glycoprotein complex (DAGC). The DAGC protects the sarcolemma from contraction-induced injury. In the D2-mdx mouse model, loss of dystrophin leads to loss of alpha-sarcoglycan. D2-mdx mice were treated 4×Q4W (20 mg/kg) IV injections of PMO-MDX-23-2 or EEV-PMO-MDX-23-2. EEV-PMO-MDX-23-2 treated muscle demonstrated almost complete restoration of both dystrophin and alpha-sarcoglycan in contrast to PMO-MDX-23-2 treated mice, which showed limited restoration of dystrophin or alpha-sarcoglycan (data not shown).

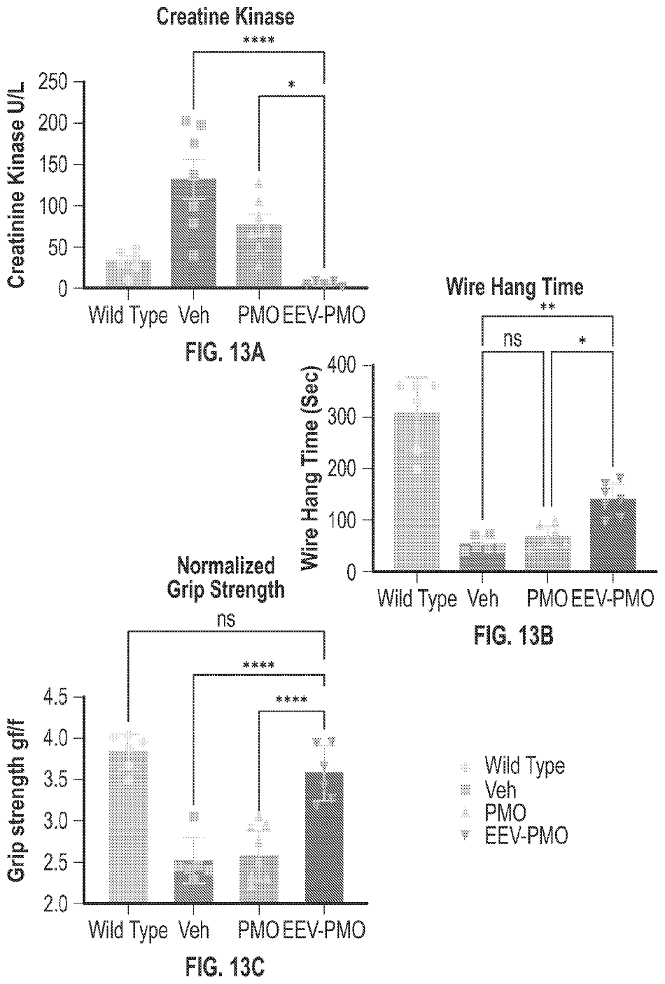

D2-mdx mice were administered 20 mg/kg of PMO-MDX-23-2 or EEV-PMO-MDX-23-2 (monthly). Creatine Kinase (CK) levels (FIG. 13A) and functional readouts (wire hang time (FIG. 13B) and normalized grip strength (FIG. 13C)) were compared to the wild-type (DBA/2J) and the vehicle (saline) treated mice. Treatment with EEV-PMO-MDX-23 normalized serum CK levels and significantly improved muscle function when compared to PMO-MDX-23-2 alone (FIGS. 13A-13C).

### Example 4: Duration and Repeated Dose Effect on D2MDX Mice after EEV-PMO-MDX-23-2 Administration

Method: D2/MDX mice were dosed with 20, 40 or 80 mpk of EEV-PMO-MDX-23-2 (EEV=Ac-PKKKRKV-miniPEG-K(cyclo(GfFGrGrQ))-PEG₁₂-OH; PMO=5′-GGCCAAACCTCGGCTTACCTGAAAT-3′) or PMO-MDX-23-2 (5′-GGCCAAACCTCGGCTTACCTGAAAT-3′) that induces Exon 23 skipping in the D2/MDX mouse model. Tissues were harvested at 1, 2, 4 and 8 weeks to test for single dose duration effects and single dose range finding. D2/MDX mice were dosed with 40 mpk weekly for 4 weeks and sacrificed 1 week after the final dose to test for the repeated dose effect.

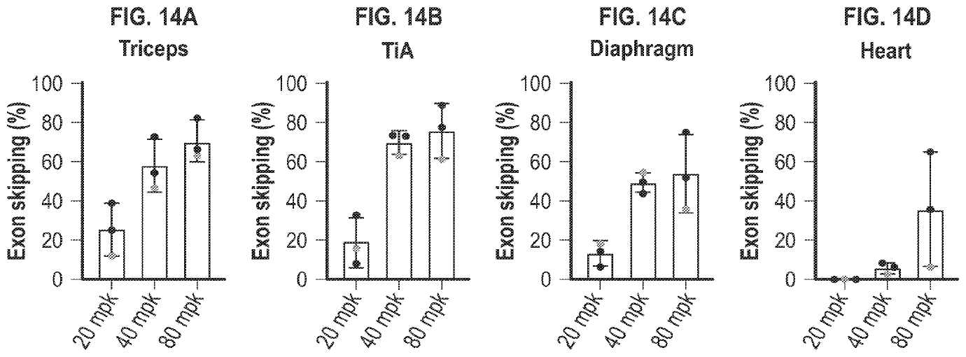

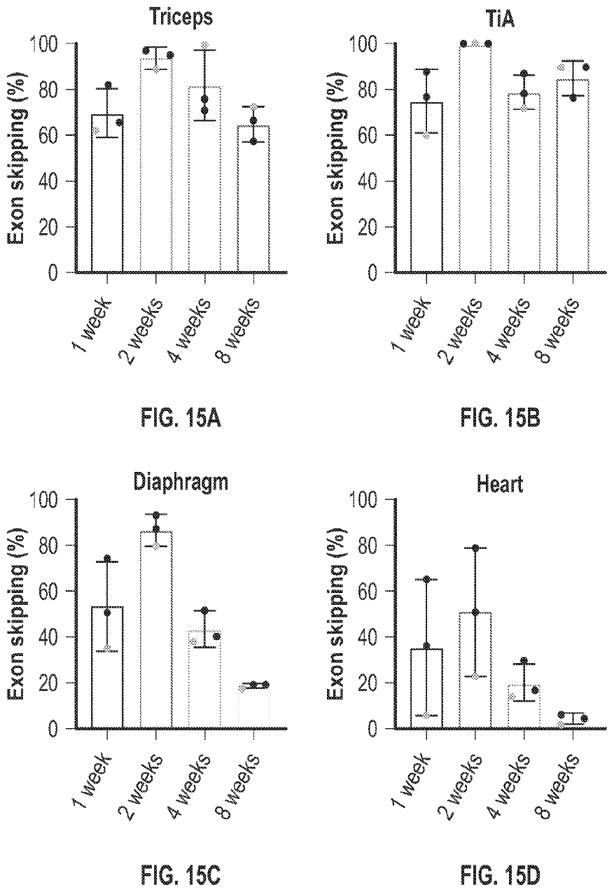

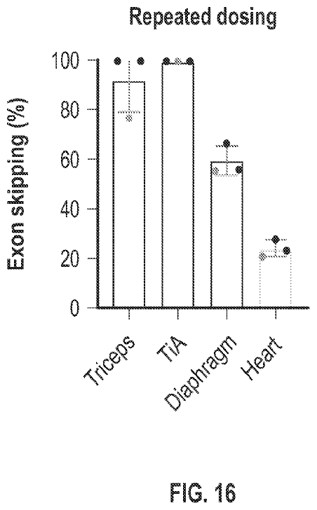

Results: Exon skipping was observed in all 4 tissues after a single dose (FIGS. 14A-14D), as well as dystrophin production. Exon skipping peaked at 2 weeks post injection and was maintained for at least 8 weeks in skeletal muscle: FIG. 15A (triceps) and FIG. 15B (tibialis anterior). A drop in exon skipping was observed after 4 and 8 weeks in diaphragm (FIG. 15C) and heart (FIG. 15D). After 4 weekly doses, a cumulative exon skipping was observed in all 4 tissues, particularly in heart muscle (FIG. 16) (tissue was collected 1 week after last dose).

### Example 5: Functional Assays in D2MDX

Method: 6 groups of male D2/MDX and DBA/2J (wild-type) mice (n=8 per group) were dosed intravenously every 2 weeks for a total of 6 doses with vehicle (saline), PMO-MDX-2 (5′-GGCCAAACCTCGGCTTACCTGAAAT-3′) or one of two EEV-PMO constructs that target Exon 23 (EEV-PMO-MDX23-2: EEV=Ac-PKKKRKV-miniPEG-K(cyclo(GfFGrGrQ))-PEG₁₂-OH; PMO=5′-GGCCAAACCTCGGCTTACCTGAAAT-3′; and EEV-PMO-MDX-23-3: EEV=Ac-PKKKRKV-Lys(FfΦGrGrQ)-PEG₁₂-K(N₃)—NH₂; PMO=5′-GGCCAAACCTCGGCTTACCTGAAAT-3′-C4COT). C4COT=cyclooct-2-yn-1-O—(CH₂)₄—O—C(O). The dosages are listed in the Figures. Creatine kinase levels, grip strength and wire-hang time were determined every 4 weeks for a total of 4 times.

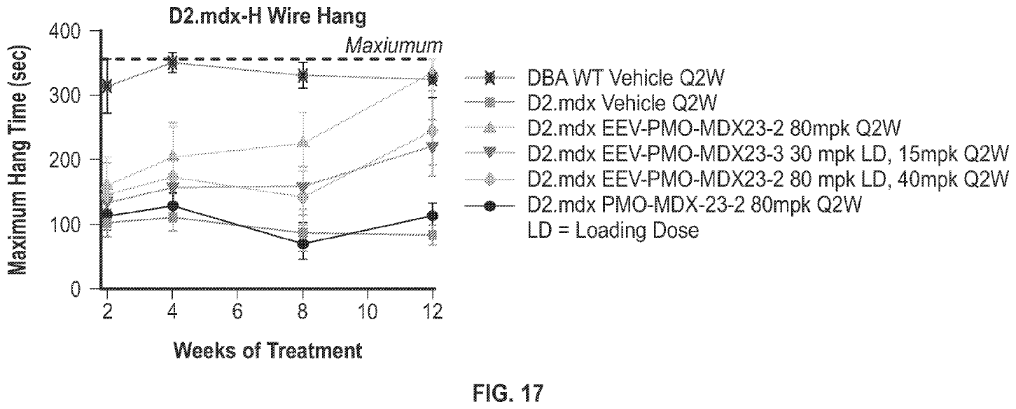

Results: Hang time for EEV-PMO-MDX-23-2 80 mpk Q2W treatment is a little higher than the rest of the groups by 2 weeks post first injection and continues to show statistically significant improvement that increases at both 4 and 8 weeks post first injection vs. the vehicle D2.mdx group (FIG. 17). After 12 weeks of treatment, EEV-PMO-MDX-23-2 80 mpk Q2W was statistically indistinguishable from the WT animals (FIG. 17). EEV-PMO-MDX-23-2 40 pmk Q2W and EEV-PMO-MDX-23-3 15 mpk Q2W treatment with a loading dose (80 mpk and 30 mpk, respectively) showed significantly higher wire hang times vs. the vehicle D2.mdx group starting at 8 weeks post first treatment and plateauing until 12 weeks of treatment where signs of phenotype improvement first become evident (FIG. 17). PMO-MDX-23-2 treatment alone appeared to follow the same trends as the vehicle D2.mdx group and the vehicle control groups.

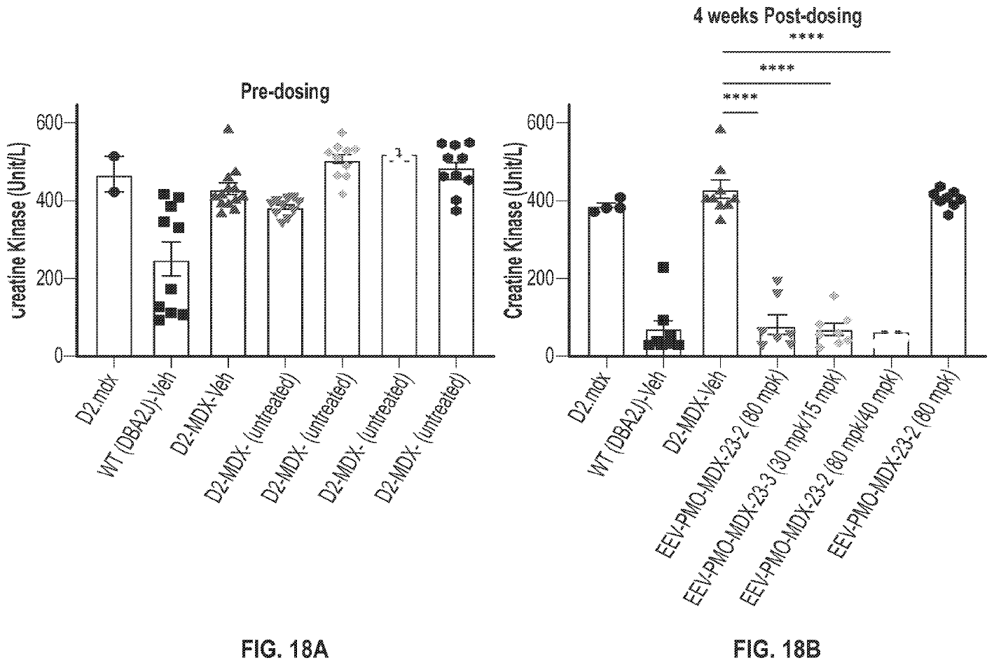

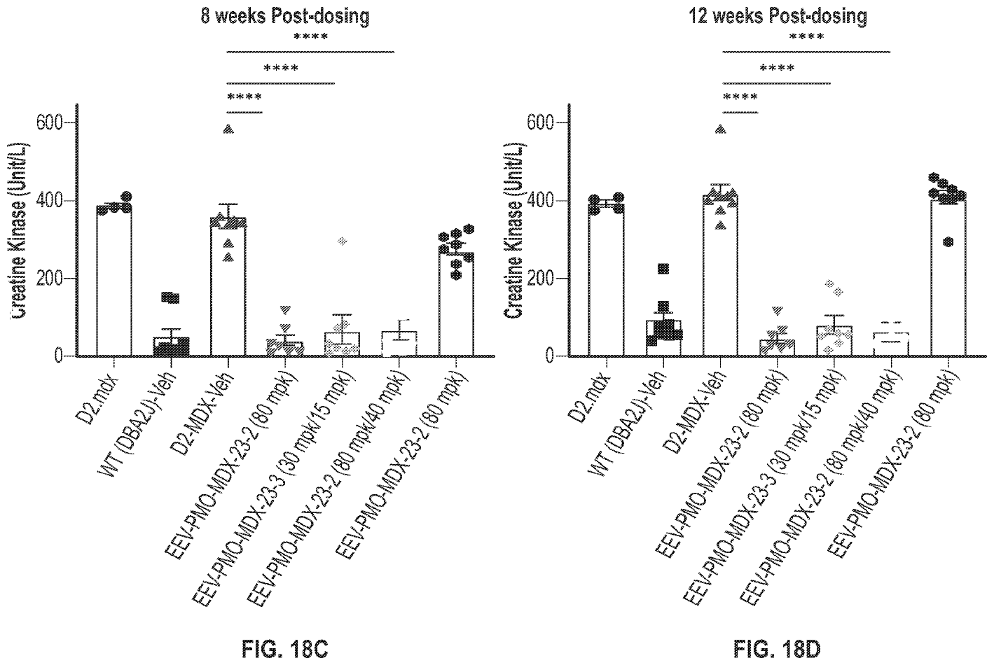

Serum creatine kinase (CK) levels were determined at 4 time points: pre-dose, and at 4, 8 and 12 weeks. Mice treated with EEV-PMO-MDX-23-2 or EEV-PMO-MDX-23-3 showed a significant reduction in serum CK, close to wild-type. Mice treated with PMO-MDX-23-2 showed no significant decrease for all time points post-treatment (FIG. 18A-18C).

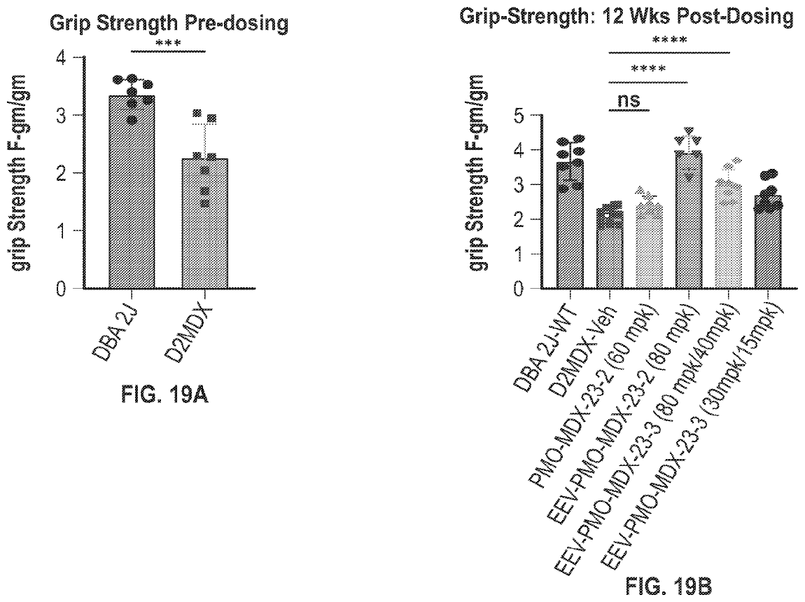

Grip strength was measured pre-dose (FIG. 19A) and at 12 weeks (FIG. 19B). A dose dependent increase in grip strength was observed for treated mice. Vehicle and PMO treated mice showed no significant improvement.

### Example 6. Use of Cell-Penetrating Peptides Conjugated to Oligonucleotides for Splicing Correction of Exon 44 of hDMD

Purpose: This study employs hDMD and CD1 mouse models and a NHP model to study the effect of compounds comprising an antisense compound and a cell penetrating peptide. Each of the compounds contained the exocyclic sequence PKKKRKV.

Compounds Evaluated: EEV-PMO-DMD44-1, EEV-PMO-DMD44-2 and EEV-PMO-DMD44-3 were evaluated in this study. Sequence information for these compounds is shown in Table 8.

TABLE 8
EEV Compounds

| Cmpd | Peptide Sequence | Nucleic Acid Sequence of AC (5′-3′) | Chem |
|---|---|---|---|
| EEV-PMO-DMD44-1 | Ac-PKKKRKV-AEEA-Lys- | 5′-AAACGCCGCCATTTCTCAACAGATC-3′ | PMO |
| | (cyclo[FGFGRGRQ])-PEG₁₂-OH | | |
| EEV-PMO-DMD44-2 | Ac-PKKKRKV-AEEA- | 5′-AAACGCCGCCATTTCTCAACAGATC-3′ | PMO |
| | Lys(cyclo[GfFGrGrQ])-PEG₁₂-OH | | |
| EEV-PMO-DMD44-3 | Ac-PKKKRKV-AEEA- | 5′-AAACGCCGCCATTTCTCAACAGATC-3′ | PMO |
| | Lys(cyclo[FfΦGrGrQ])-PEG₁₂-OH | | |
| EEV-PMO-MDX-23-3 | Ac-PKKKRKV-Lys(FfΦ-GrGrQ)- | 5′-GGCCAAACCTCGGCTTACCTGAAAT-3′- | PMO |
| | PEG12-K(N₃)-NH₂ | C4COT | |

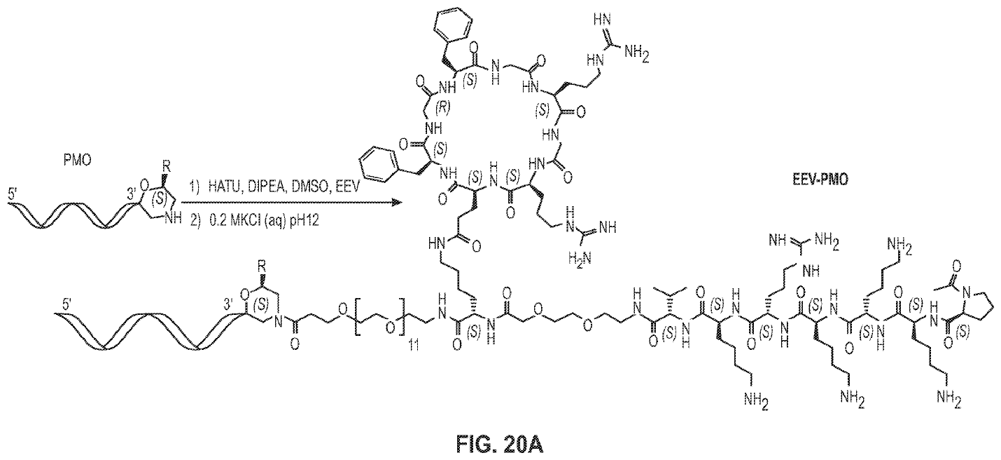

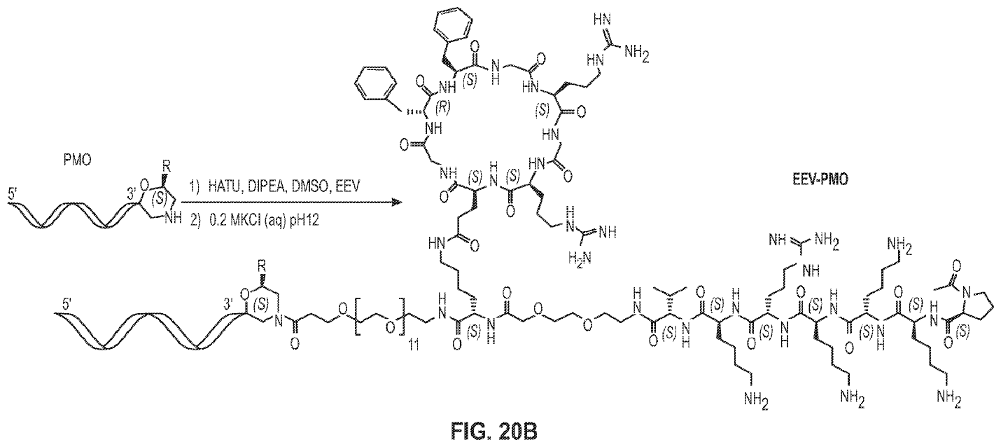

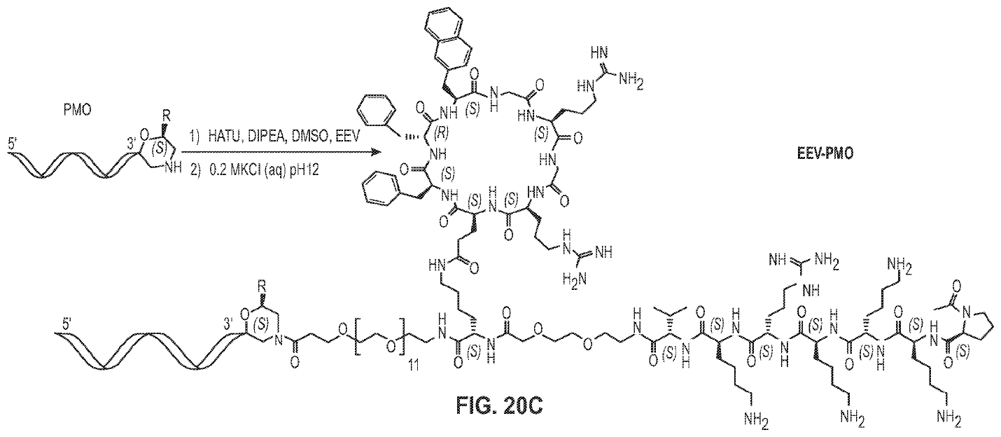

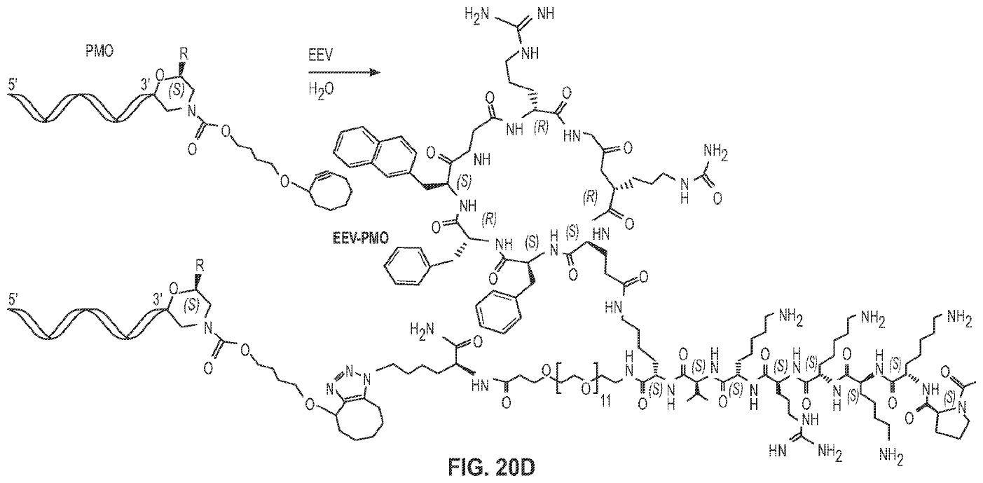

The structures of EEV-PMO-DMD44-1, EEV-PMO-DMD44-2 and EEV-PMO-DMD44-3 are provided below. FIG. 20A-20D provides synthetic schemes for EEV-PMO-DMD44-1 (FIG. 20A), EEV-PMO-DMD44-2 (FIG. 20B), EEV-PMO-DMD44-3 (FIG. 20C) and EEV-PMO-MDX-23-3 (FIG. 20D).

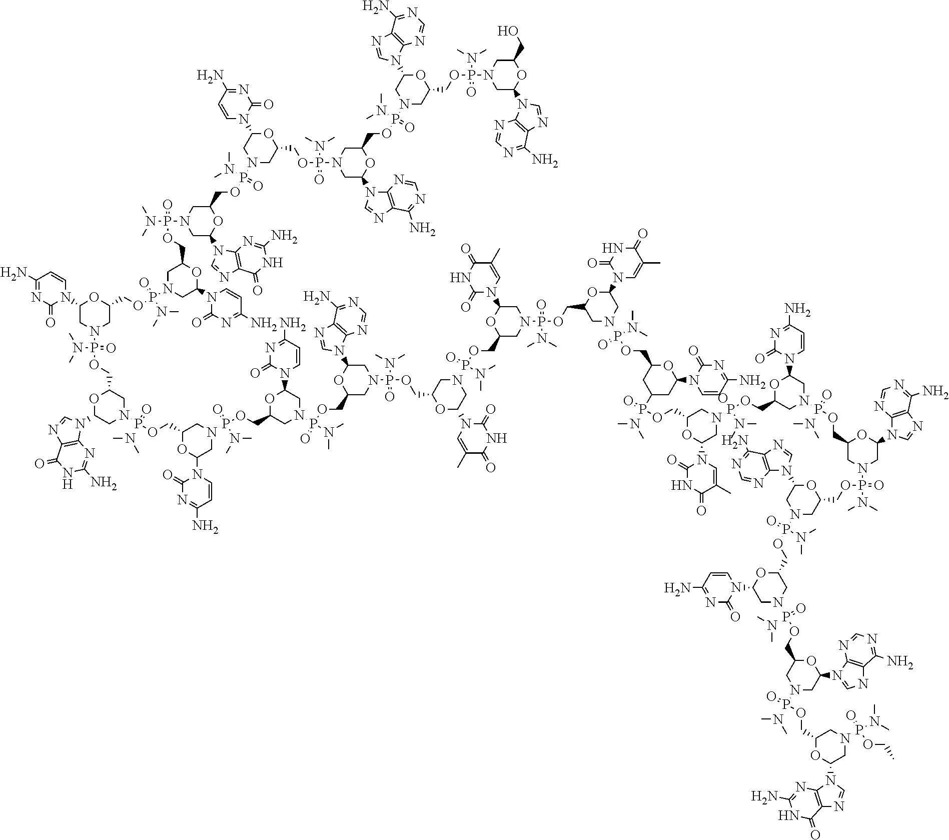

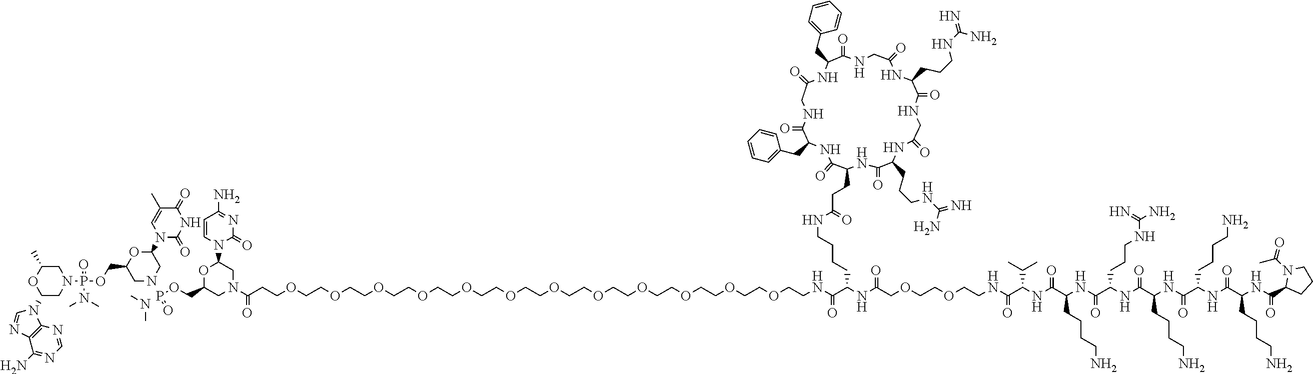 [SMILES: CC(=O)N1CCC[C@H]1C(=O)N[C@@H](CCCCN)C(=O)N[C@@H](CCCCN)C(=O)N[C@@H](CCCCN)C(=O)N[C@@H](CCCNC(=N)N)C(=O)N[C@@H](CCCCN)C(=O)N[C@H](C(=O)NCCOCCOCC(=O)N[C@@H](CCCCNC(=O)CC[C@@H]1NC(=O)[C@H](CCCNC(=N)N)NC(=O)CNC(=O)[C@H](CCCNC(=N)N)NC(=O)CNC(=O)[C@H](Cc2ccccc2)NC(=O)CNC(=O)[C@H](Cc2ccccc2)NC1=O)C(=O)NCCOCCOCCOCCOCCOCCOCCOCCOCCOCCOCCOCCOCCC(=O)N1C[C@@H](COP(=O)(N(C)C)N2C[C@@H](COP(=O)(N(C)C)N3C[C@@H](C)O[C@@H](n4cnc5c(N)ncnc54)C3)O[C@@H](n3cc(C)c(=O)[nH]c3=O)C2)O[C@@H](n2ccc(N)nc2=O)C1)C(C)C]

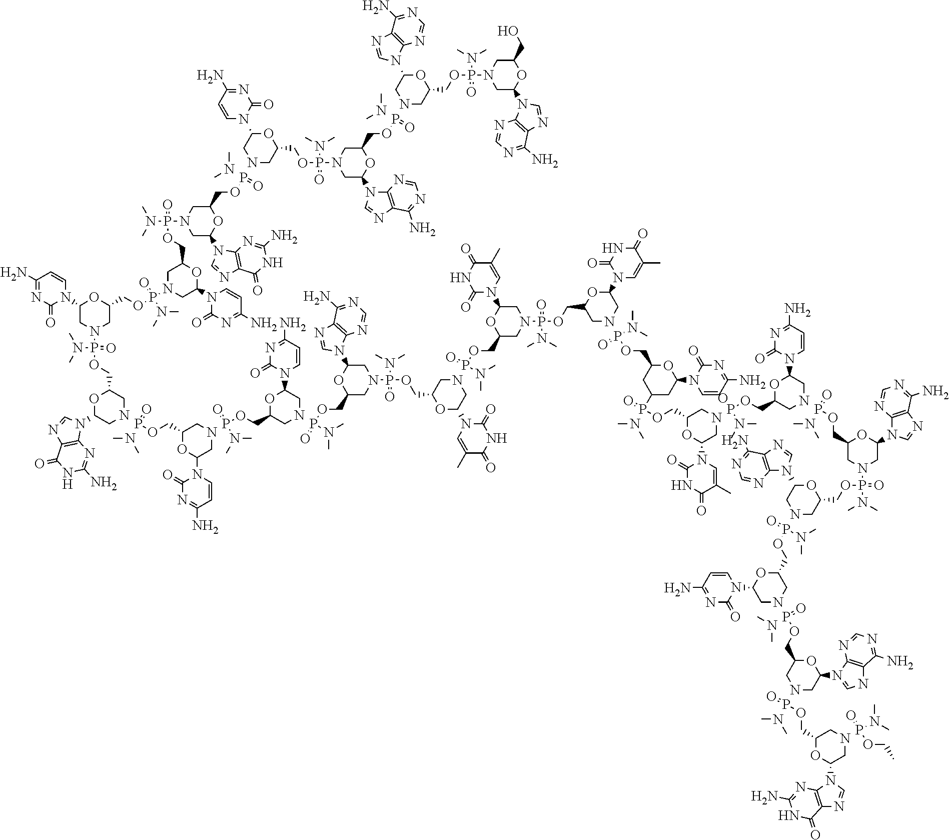

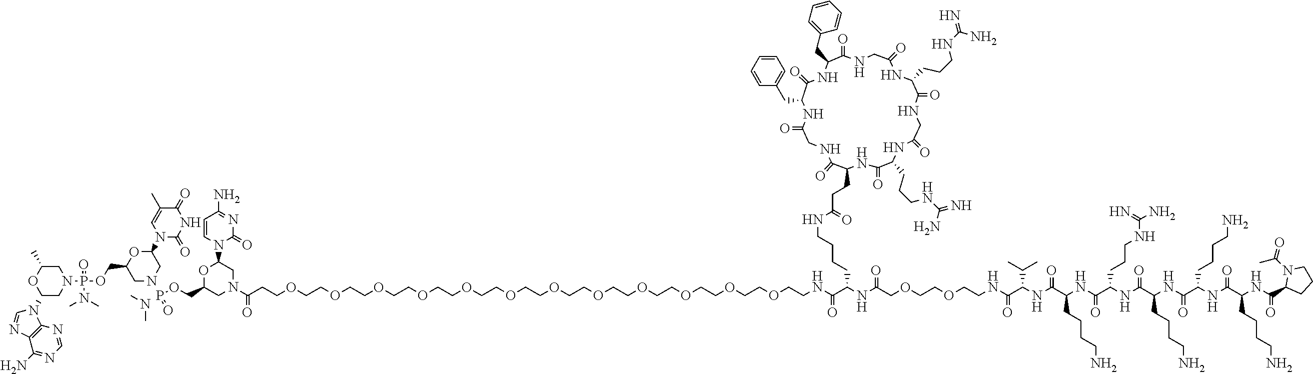 [SMILES: CC(=O)N1CCC[C@H]1C(=O)N[C@@H](CCCCN)C(=O)N[C@@H](CCCCN)C(=O)N[C@@H](CCCCN)C(=O)N[C@@H](CCCNC(=N)N)C(=O)N[C@@H](CCCCN)C(=O)N[C@H](C(=O)NCCOCCOCC(=O)N[C@@H](CCCCNC(=O)CC[C@@H]1NC(=O)[C@@H](CCCNC(=N)N)NC(=O)CNC(=O)[C@@H](CCCNC(=N)N)NC(=O)CNC(=O)[C@H](Cc2ccccc2)NC(=O)[C@@H](Cc2ccccc2)NC(=O)CNC1=O)C(=O)NCCOCCOCCOCCOCCOCCOCCOCCOCCOCCOCCOCCOCCC(=O)N1C[C@@H](COP(=O)(N(C)C)N2C[C@@H](COP(=O)(N(C)C)N3C[C@@H](C)O[C@@H](n4cnc5c(N)ncnc54)C3)O[C@@H](n3cc(C)c(=O)[nH]c3=O)C2)O[C@@H](n2ccc(N)nc2=O)C1)C(C)C]

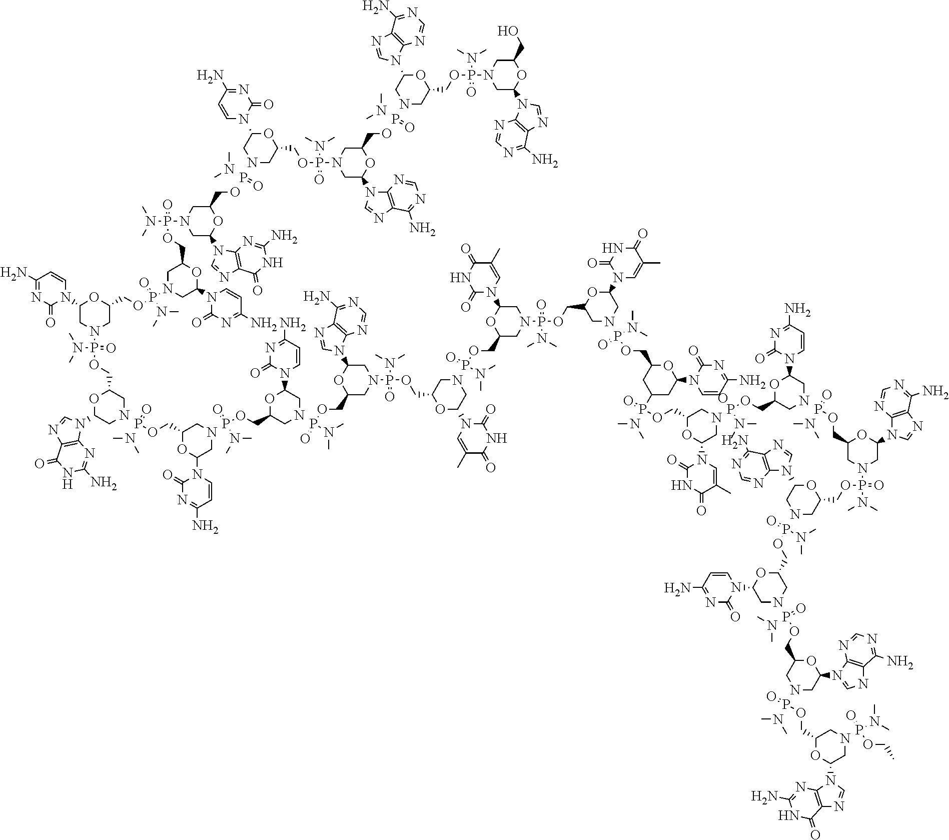

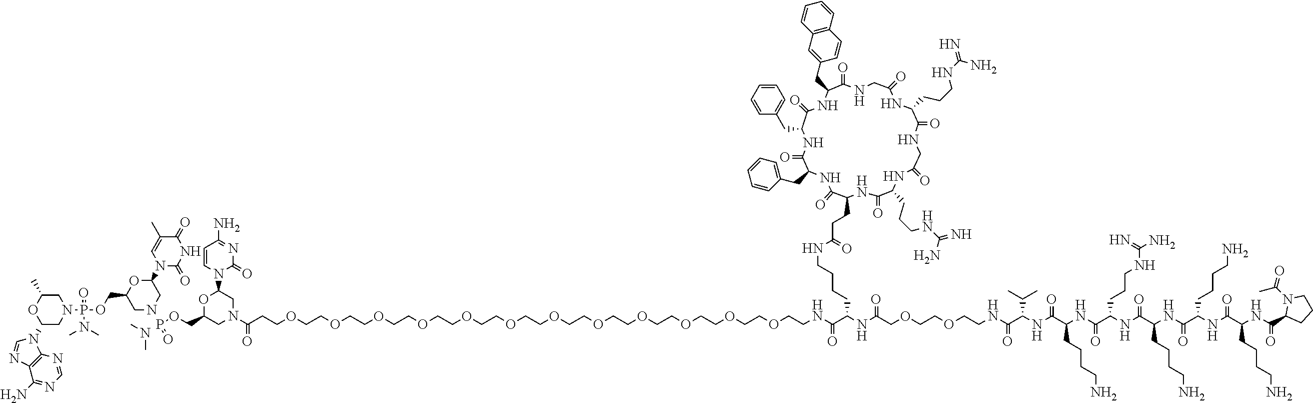

Compound Synthesis and Purification: The compounds were synthesized according to the following procedure. TFA-lysine protected cCPPs were reacted with the AC of Table 8 and subsequently deprotected to furnish a cCPP-AC conjugate. Briefly, the cCPP was pre-activated by reacting it with HATU (2.0 equiv) and DIPEA (2.0 equiv) in DMSO (10 mM, 1.8 mL). After 10 min at room temperature, the pre-activated solution was combined with a solution of AC in DMSO (10 mM, 1.8 mL) and mixed thoroughly. The reaction was incubated for 2 hours at room temperature. The reaction was monitored by LCMS (Q-TOF), using BEH C18 column (130 Å, 1.7 μm, 2.1 mm×50 mm), buffer A: water (0.1% FA), buffer B: acetonitrile (0.1% FA), flow rate: 0.4 mL/min, starting with 2% buffer B and ramping up to 98% over 3.4 min. Upon completion, in situ deprotection of TFA-protected lysines was initiated by dilution of the reaction mixture with 0.2 M KCl (aq) pH 12 (36 mL). The reaction was monitored by LCMS (Q-TOF), using the analysis method noted above. The crude mixture was loaded directly onto a C18 reverse-phase column (Oligo clarity column, 150 mm*21.2 mm). The crude product was then purified using a gradient of 5-20% over 60 min using water with 0.1% FA and acetonitrile as solvents and a flow rate of 20 mL/min. Fractions containing the desired product were pooled, and the pH of the solution was adjusted to 7 using 0.5 M NaOH. The solution was frozen and lyophilized, affording white powder. Formate salts were exchanged with chloride by reconstitution of the cCPP-AC conjugate in 1M NaCl in water and repeated washes through a 3-kD MW-cutoff amicon tube (centrifuged at 3500 rpm for 20-40 min). This process was performed three times with 1 M NaCl and three times with saline (0.9% NaCl, sterile, endotoxin-free). Conductivity of the last filtrate was assessed to confirm appropriate salt concentration. The solution was further diluted with saline to the desired formulation concentration and sterile filtered in a biosafety cabinet. The concentration of each formulation was remeasured post filtration.

EEV-PMO-DMD44-1 was obtained with 74% yield. The purity and identity of each formulation was assessed by liquid chromatography-mass spectrometry quadrupole time-of-flight mass spectrometry (QTOF-LCMS). EEV-PMO-DMD44-1 was determined to be 99% pure by RP-FA and 78% pure by CEX. The MW calcd for C₄₁₁H₆₆₁N₁₇₃O₁₃₀P₂₄, 10849.26. The MW identified by QTOF-LCMS was 10850.95. Formulations were further assayed for their endotoxin amount, residual free peptide, FA content and pH.

EEV-PMO-DMD44-2 was obtained with 70% yield. The purity and identity of each formulation was assessed by QTOF-LCMS. EEV-PMO-DMD44-2 was 99% pure by RP-FA and 78% pure by CEX. The MW calcd for C₄₁₁H₆₆₁N₁₇₃O₁₃₀P₂₄, was 10849.26. The MW identified by QTOF-LCMS was 10850.88.

EEV-PMO-DMD44-3 was obtained with 68% yield. The purity and identity of each formulation was assessed by QTOF-LCMS. EEV-PMO-DMD44-3 was 86.3% pure by RP-FA (The impurity was unreacted AC). The MW calcd for C₄₂₂H₆₆₉N₁₇₃O₁₃₀P₂₄, was 10989.45. The MW identified by QTOF-LCMS was 10990.07.

hDMD mouse model: hDMD mice were ordered from the Jackson Lab (STOCK Tg(DMD)72Thoen/J; Stock No: 018900) and bred in-house. The hemizygous mice were further genotyped at Transnetyx. All groups were dosed 5 mL/kg per animal by intravenous (iv) injection and sacrificed after 5 days post injection. All animals were euthanized by CO₂ asphyxiation followed by terminal blood collection via cardiac puncture. Maximum obtainable volume of whole blood was collected into lithium heparin tubes and processed to plasma. A portion of plasma was analyzed for clinical chemistries by the Testing Facility (IDEXX) and the rest stored frozen at nominally −70° C. Tissues (Triceps, TA, diaphragm, heart, kidney, liver, Brain) were harvested and flash frozen in liquid nitrogen and stored at −80° C. for further evaluation of exon skipping and drug concentration measurements. Animals were age matched and assigned into eight (8) treatment groups according to Table 9. Group 1-1 (3 homo hDMD mice, 6 weeks old), 1-2 (3 homo hDMD mice, 6 weeks old), 1-3 (1 male, 1 female, hemi hDMD, 11 weeks old), 1-4 (1 male, 1 female, hemi DMD, 11 weeks old) received EEV-PMO-DMD44-1 at 10, 20, 40 and 80 milligrams per kilogram of body weight (mpk), respectively. Group 2-1 (3 homo hDMD mice, 6 weeks old), 2-2 (3 homo hDMD mice, 6 weeks), 2-3 (1 male, 1 female, hemi hDMD, 11 weeks), 2-4 (1 male, 1 female, hemi DMD, 11 weeks) received EEV-PMO-DMD44-2 at 10, 20, 40 and 80 mpk, respectively. All animals survived until their scheduled euthanasia time. Tissues were collected per protocol. The amount of AC and a cCPP-AC in various tissue samples was quantified by LC-MS. Exon skipping in different tissues were analyzed by RT-PCR and the quantification of exon 44 correction.

TABLE 9
Experimental design of hDMD Experiments

| | | | Dose | | | |
|---|---|---|---|---|---|---|
| | Animal | | Level | Dose Volume | | Terminal |
| Group | No. | Test Article | mg/kg | (5 mL/kg) | Dosing Regimen | Time Point |
| 1-1 | 3 | EEV-PMO- | 10 | 5 | IV | 5 days post |
| | | DMD44-1 | | | | injection |
| 1-2 | 3 | EEV-PMO- | 20 | | | |
| | | DMD44-1 | | | | |
| 1-3 | 3 | EEV-PMO- | 40 | | | |
| | | DMD44-1 | | | | |
| 1-4 | 3 | EEV-PMO- | 80 | | | |
| | | DMD44-1 | | | | |
| 2-1 | 2 | EEV-PMO- | 10 | | | |
| | | DMD44-2 | | | | |
| 2-2 | 2 | EEV-PMO- | 20 | | | |
| | | DMD44-2 | | | | |
| 2-3 | 2 | EEV-PMO- | 40 | | | |
| | | DMD44-2 | | | | |
| 2-4 | 2 | EEV-PMO- | 80 | | | |
| | | DMD44-2 | | | | |

CD3 mouse model. Tolerability of EEV-PMO-DMD44-1 and EEV-PMO-DMD44-2 were evaluated using CD1 male-mice at 7 weeks age. They were ordered from the Charles River Lab and upon receiving, they were acclimated for 5 days prior to the injections. Animals were aged matched and assigned into nine (9) treatment groups according to Table 10. Group 1 (3 mice, saline); Group 2-1 (3 mice), 2-2 (2 mice), 2-3 (2 mice), 2-4 (2 mice), 2-5 (3 mice), 2-6 (3 mice) received EEV-PMO-DMD44-1 at 80, 100, 120, 160, 200 and 300 mpk, respectively. Group 3-1 (3 mice), 3-2 (2 mice), 3-3 (2 mice), 3-4 (2 mice), 3-5 (3 mice), 3-6 (3 mice) received EEV-PMO-DMD44-2 at 80, 100, 120, 160, 200 and 300 mpk, respectively.

TABLE 10
Experimental design of Tolerability Study in CD1 Mice

| | | | Dose | | | |
|---|---|---|---|---|---|---|
| | Animal | | Level | Dose Volume | | Terminal |
| Group | No. | Test Article | mg/kg | (5 mL/kg) | Dosing Regimen | Time Point |
| 1 | 3 | Saline | — | 5 | IV | 7 days post |
| 2-1 | 3 | EEV-PMO- | 80 | | | injection |
| | | DMD44-1 | | | | |
| 2-2 | 2 | EEV-PMO- | 100 | | | |
| | | DMD44-1 | | | | |
| 2-3 | 2 | EEV-PMO- | 120 | | | |
| | | DMD44-1 | | | | |
| 2-4 | 2 | EEV-PMO- | 160 | | | |
| | | DMD44-1 | | | | |
| 2-5 | 3 | EEV-PMO- | 200 | | | |
| | | DMD44-1 | | | | |
| 2-6 | 3 | EEV-PMO- | 300 | | | |
| | | DMD44-1 | | | | |
| 3-1 | 2 | EEV-PMO- | 80 | | | |
| | | DMD44-2 | | | | |
| 3-2 | 2 | EEV-PMO- | 100 | | | |
| | | DMD44-2 | | | | |
| 3-3 | 2 | EEV-PMO- | 120 | | | |
| | | DMD44-2 | | | | |
| 3-4 | 2 | EEV-PMO- | 160 | | | |
| | | DMD44-2 | | | | |
| 3-5 | 3 | EEV-PMO- | 200 | | | |
| | | DMD44-2 | | | | |
| 3-6 | 3 | EEV-PMO- | 300 | | | |
| | | DMD44-2 | | | | |

NHP models. One female animal per compound (EEV-PMO-DMD44-1 and EEV-PMO-DMD44-2) was administered a 60-minute IV infusion with dosing volume of 10 mL/kg according to Table 11. Each testing article was formulated in saline at 4 mg/mL. Bloods and urine were taken at times indicated in Table 12 for further PK analysis. Biopsy at 2 days post injection was performed on biceps. Animals were sacrificed at day 7 post injection and skeletal muscles (quadriceps, diaphragm, biceps, deltoid, tibialis anterior (TiA), smooth muscles (esophagus, aorta, colon) and cardiac muscles (ventricle, atrium) were taken, pulverized, and stored at −80° C. for evaluation of exon skipping and biodistribution in tissues.

TABLE 11
Experimental design of NHP Study

| | | | Dose | | | |
|---|---|---|---|---|---|---|
| | Gender | Test | Level | Dose Volume | Dosing | Terminal |
| Group | of Animal | Article | mg/kg | (5 mL/kg) | Regimen | Time Point |
| 1 | Female | EEV-PMO- | 40 | 10 | IV infusion | 7 days post |
| | | DMD44-1 | | | | injection |
| 2 | Female | EEV-PMO- | 40 | | | |
| | | DMD44-2 | | | | |

TABLE 12
NHP Study Timepoints

| | | | Blood | Sample | Sampling time points |
|---|---|---|---|---|---|
| Group | matrix | Detect factor | volume | volume | post dose |
| 1-2 | Serum | Cytokine Panel IL- | 0.5 | 2 × 100 | pre-dose (0 h), 1.5 h |
| | | 2/4/5/6/10/13/TNFα/IFN-γ | mL | μL | (30 min post full |
| | | | | | infusion), 24 h and Day |
| | | | | | 7 |
| | Blood | Hematology | 0.5 | 0.5 | pre-dose (0 h), 5 min |
| | | | mL | mL | (0.083 h) during |
| | | | | | infusion, 24 h and Day |
| | | | | | 7 |
| | Serum | Clinical Chemistry | 1 | 400 | pre-dose (0 h), 5 min |
| | | including magnesium | mL | μL | (0.083 h) during |
| | | | | | infusion, 24 h and Day |
| | | | | | 7 |
| | Blood | Coagulation | 1.8 | 1.8 | pre-dose (0 h), 5 min |
| | | | mL | mL | (0.083 h) during |
| | | | | | infusion, 24 h and Day |
| | | | | | 7 |
| | Urine | Urinalysis including | | 4 | pre-dose (0 h), and |
| | | Creatinine | | mL | 25~29 h and Day 7 |
| | Urine | Additional collection to | | 4 | pre-dose (0), 0-6 h, 6-12 h, |
| | and | sponsor | | mL | 12-24 h, 24-48 h |
| | feces* | | | | |
| | Muscle* | 2 cores per biopsy site at | | 2 × (30-50) | Day 2 |
| | | biceps brachii | | mg | |
| | Plasma* | PK analysis samples, | 0.5 | 2 × 100 | pre-dose (0 h), 30 min |
| | | sent to sponsor | mL | μL | (0.5 h) during infusion, |
| | | | | | 55 min (0.92 h) during |
| | | | | | infusion, 65 min |
| | | | | | (1.083 h), 1.5 h, 3 h, 9 h, |
| | | | | | 25 h, 39 h, 97 h, and |
| | | | | | 169 h (Day 7) |

1. Timer from the start point of the infusion. 2. Pre-dose tests of hematology, clinical
chemistry, coagulation and urinalysis are conducted one week before dosing.

Bioanalytical Sample Analysis: Tissues were thawed, weighed, and homogenized (w/v, 1/5) with RIPA buffer spiked with 1× protease inhibitor cocktail (ThermoFisher Scientific, Ref #1860932). The homogeneates centrifuged at 5000 rpm for 5 minutes at 4° C. The supernatants were precipitated with a mixture of H₂O, acetonitrile and MeOH, and centrifuged at 15000 rpm for 15 minutes at 4° C. The supernatants were transferred to an injection plate for LC-MS/MS analysis using Shimadzu UPLC integrated with Triple Quad Sciex 4500 instrument. The dynamic range of the LC-MS/MS assay was 25 to 50,000 ng/g tissue. The details of the LC-MS/MS method are outlined here and in Table 13. Briefly, the UPLC was operated using Waters Acquity UPLC BEH C4, 300 A, 1.7 um, 2.1×150 mm, buffer A: H₂O, 0.2% FA, buffer B: 95% acetonitrile in H₂O, 0.2% FA, flow rate (0.3 mL/min) and column temperature at 50° C. The 10 min run started with 2% buffer B and ramping up to 35% for 3.5 min followed by 90% for 1 min, staying at 90% gradient for 2.5 m and finally running at 20 gradient for 2 min. The MRM method was established for a duration of 7.5 min with positive polarity; Turbo Spray ion source; Curtain Gas: 25; Collision Gas: 6; ion spray voltage: 5500; temperature: 500; ion source gas1: 60; ion source gas2: 60. The underlined rows of Table 13 are used for quantifications of intact and corresponding metabolite (235-PEG12).

TABLE 13
LC-MS/MS Assay

| | Q1 | Charge | Q3 | | | | | |
|---|---|---|---|---|---|---|---|---|
| | Mass | state | Mass | Time | DP | EP | CE | CXP |
| Analyte | (Da) | (m/z) | (Da) | (msec) | (volts) | (volts) | (volts) | (volts) |
| EEV-PMO- | 835.6 | 13 | 112.0 | 50.0 | 80.0 | 8.0 | 80.0 | 10.0 |
| DMD44-1/ | | | | | | | | |
| EEV-PMO- | | | | | | | | |
| DMD44-2 | | | | | | | | |
| EEV-PMO- | 776.0 | 14 | 112.0 | 300.0 | 80.0 | 8.0 | 80.0 | 10.0 |
| DMD44-1/ | | | | | | | | |
| EEV-PMO- | | | | | | | | |
| DMD44-2 | | | | | | | | |
| EEV-PMO- | 724.3 | 15 | 112.0 | 50.0 | 80.0 | 8.0 | 80.0 | 10.0 |
| DMD44-1/ | | | | | | | | |
| EEV-PMO- | | | | | | | | |
| DMD44-2 | | | | | | | | |
| M1 | 879.0 | 10 | 112.0 | 50.0 | 80.0 | 8.0 | 80.0 | 10.0 |
| M1 | 799.2 | 11 | 112.0 | 50.0 | 80.0 | 8.0 | 80.0 | 10.0 |
| M1/245- | 732.7 | 12 | 112.0 | 300.0 | 80.0 | 8.0 | 80.0 | 10.0 |
| PEG12 | | | | | | | | |
| IS | 863.8 | 12 | 112.0 | 100.0 | 80.0 | 8.0 | 80.0 | 10.0 |

Exon Skipping Analysis by RT-PCR: hDMD mice and NHP express full-length human dystrophin mRNA. The delivery of AC can alter the splicing and result in a shortened dystrophin mRNA after exon 44 skipping. The tissues were homogenized using 1 mL of RLT lysis buffer (Qiagen, Cat #79216). The detection of splicing correction process was measured by RT-PCR where extracted RNAs from tissues were first reverse-transcribed into cDNA and further analyzed by one step RT-PCR using the following primer set: forward primer 5′-GCTCAGGTCGGATTGACATT-3′ and reverse primer 5′-GGGCAACTCTTCCACCAGTA-3′. The RT-PCR readout of tissues without splicing correction resulted in a 641 bp gene fragment and a new 493 bp gene fragment that showed up after splicing correction. Quantification of the relative intensity of the bands corresponding to skipped and unskipped transcripts were performed to assess AC-induced exon-44-skipping efficacy. The degree (percentage) of splicing correction detected by RT-PCR was calculated using the following equation: % correction=(intensity of 493 bp fragment band)/(intensity of 493 bp fragment band+intensity of 641 bp fragment band).

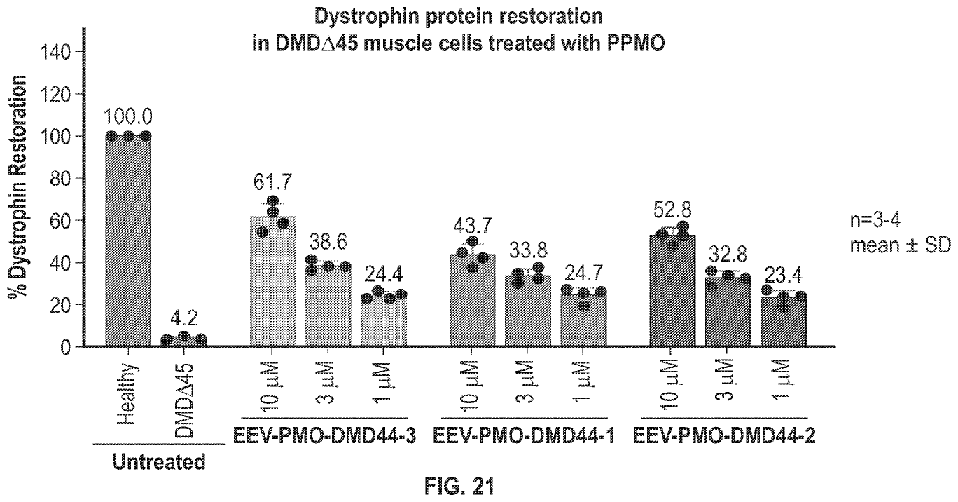

Results: Efficacy of EEV-PMO-DMD44-1, EEV-PMO-DMD44-2, and EEV-PMO-DMD44-3 in Patient Myotubes (FIG. 21): EEV-PMO-DMD44-1, EEV-PMO-DMD44-2, and EEV-PMO-DMD44-3, which each target human dystrophin (DMD) exon 44 were assessed for DMD exon 44 skipping in DMD patient derived muscle cells. Briefly, patient-derived myoblasts harboring an exon 45 deletion (DMDA45) were treated with EEV-PMO-DMD44-1, EEV-PMO-DMD44-2, and EEV-PMO-DMD44-3 at 1 μM, 3 μM, and 10 μM for 24 hours in PromoCell Skeletal Muscle Cell Growth Medium supplemented with 2% horse serum and 1% chick embryo extract. After 24 hours, the compound-containing growth medium was replaced with DMEM/2% horse serum and incubated for 5 days to promote myoblast fusion and differentiation into myotubes. Cells were washed and harvested for RNA extraction to assess exon 44 skipping, or in RIPA buffer containing protease inhibitors for protein extraction and Simple Western analysis of dystrophin protein restoration. Dystrophin levels were normalized to HSP90 and expressed relative to untreated healthy samples. Data are expressed as mean±SD, n=3-4. Untreated DMDA45 patient derived cells express ˜10% spontaneous DMD exon 44 skipping and ˜4% dystrophin protein at baseline. All three compounds resulted in robust exon skipping and dystrophin protein restoration in a dose dependent manner.

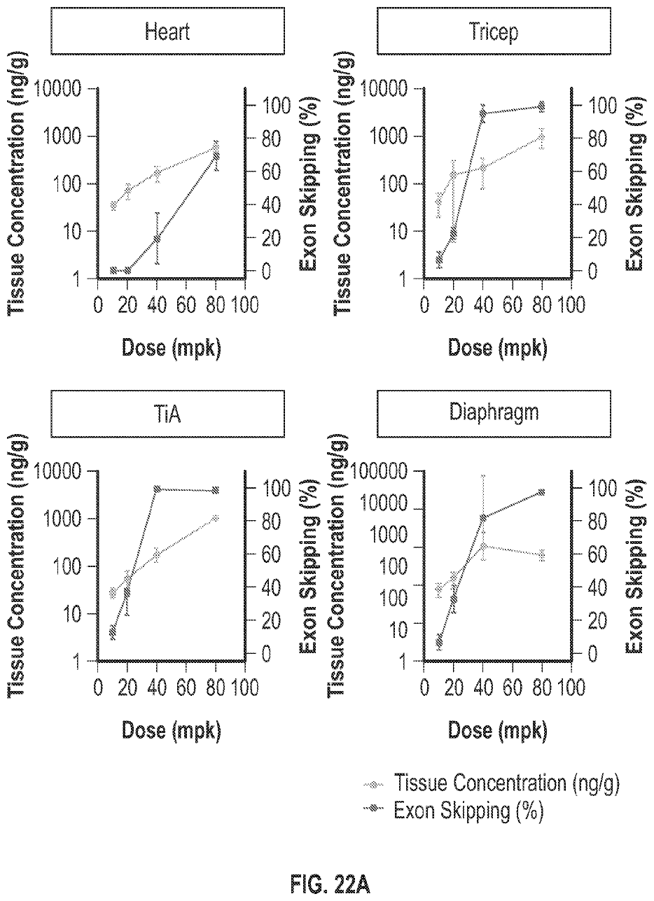

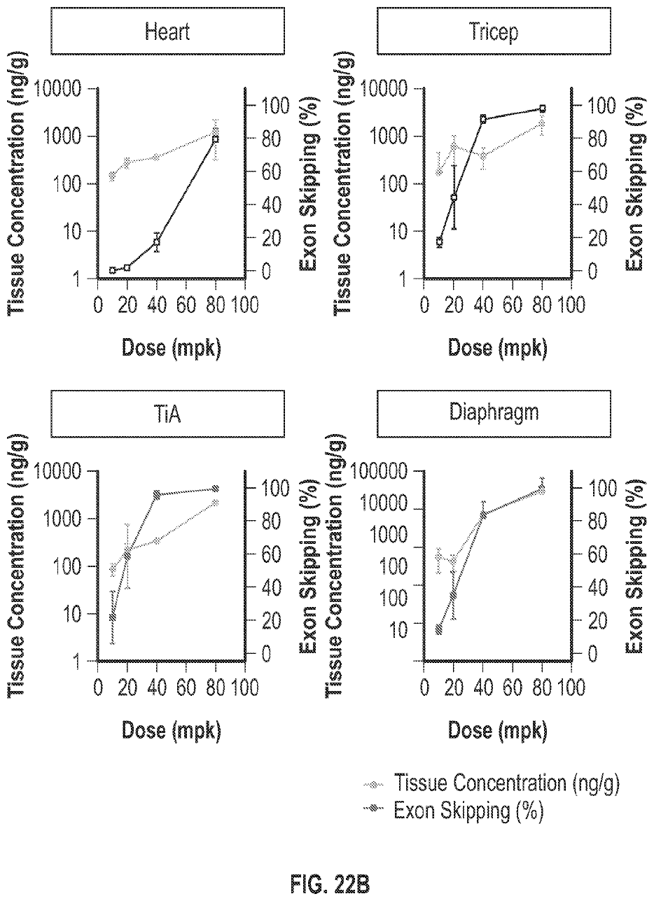

FIGS. 22A and 22B show exon skipping in hDMD mice administered EEV-PMO-DMD44-1 (FIG. 22A) and EEV-PMO-DMD44-2 (FIG. 22B) via IV injection. No severe adverse effect was observed. The mice were all normal post injection, 24 hours post injection and prior to sac date. Clinical chemistries measuring liver and kidney toxicity (Alkaline Phosphatase (ALP), Aspartate transaminase (AST), Alanine Aminotransferase (ALT), Albumin, Blood Urea nitrogen (BUN), Creatinine, Calcium, Phosphorous, Chloride, Potassium, Sodium, BUN/Creatinine, Magnesium) as well as Hemolysis and Lipemia index are evaluated 5 days post IV injection. No significant toxicity was detected by clinical chemistry evaluation in EEV-PMO-DMD44-1 and EEV-PMO-DMD44-2 treated mice. Tissue concentrations and exon skipping in various muscle groups have been assessed 5-days post 10, 20, 40, and 80 mpk IV dosage. The following exon skipping was achieved for each dose of EEV-PMO-DMD44-1 in heart/Triceps/TiA/Diaphragm tissues, respectively: 10 mpk (0%, 6%, 12%, 6%); 20 mpk (0%, 22, 36%, 33%); 40 mpk (20%, 94%, 99%, 82%); 80 mpk (79%, 97%, 99%, 98%). The following exon skipping was achieved for each dose of EEV-PMO-DMD44-2 in heart/Triceps/TiA/Diaphragm tissues, respectively: 10 mpk (0%, 17%, 22%, 14%); 20 mpk (2%, 44, 58%, 35%); 40 mpk (17%, 92%, 95%, 83%); 80 mpk (79%, 98%, 99%, 99%). Strong dose-dependent accumulation and potent exon skipping was observed for both EEV-PMO-DMD44-1 and EEV-PMO-DMD44-2 in cardiac and skeletal muscles in the transgenic murine model carrying the full-size human DMD gene. At lower doses, and 20 mpk, EEV-PMO-DMD44-2 drug exposure and efficacy were slightly higher than EEV-PMO-DMD44-1. However, this effect was started to diminish at 40 mpk dose, where both compounds resulted in same high level of exon skipping (above 80%) in all skeletal muscles. The corresponding tissue concentrations for EEV-PMO-DMD44-1 was in 100-300 ng/g tissue range while for EEV-PMO-DMD44-2 this range shifted to slightly higher number, 300-500 ng/g tissue concentration. Interestingly, the minimum efficacious dose for both EEV-PMO-DMD44-1 and EEV-PMO-DMD44-2 in the heart was achieved with 40 mpk corresponding to 170 and 350 ng per gram tissue concentration, respectively.

EEV-PMO-DMD44-1 was very well tolerated in all doses administered to CD1 mice at 80, 100, 160, 200 and 300 mpk doses. Only transient symptoms were observed which were completely resolved 1 hour post injection. No biomarker abnormalities were observed at 1- and 7-days post injection. EEV-PMO-DMD44-2 was less tolerated. At the highest dose of EEV-PMO-DMD44-2, 300 mpk, one out of three mice died within 1-3 h post injection. At lower dose of EEV-PMO-DMD44-2, 200 mpk, one out of three mice had severe symptoms (non-reactive to stimulation, ears are drawn back, slow respiration, struggles to right self). These symptoms progressively worsened, and they combined with muscle twitches. No symptoms were observed for lower doses at 160 and 80 mpk. Surprisingly, at 100 mpk, one out of three mice showed delayed symptoms 2 hours post injection; but they were completely normal 1- and 7-days post injection.

To further demonstrate the efficacy of exon skipping of cCPP-AC conjugates, NHP were utilized. Specifically, cynomolgus monkeys having intact muscle tissues were administered a 60-minute IV infusion of EEV-PMO-DMD44-1 or EEV-PMO-DMD44-2 at 40 mg/kg which were well-tolerated. More specifically, animals experienced nausea starting 45 minute of the treatment which was significantly resolved approximately 3 hours post treatment and the animal was more alert, less hunched and ate offered produce. Approximately 20 hours post dose the animal was (bright, alert, and responsive such that the animal was phenotypically “normal”) (BAR) and had zero biscuits left in the cage and was observed eating produce.

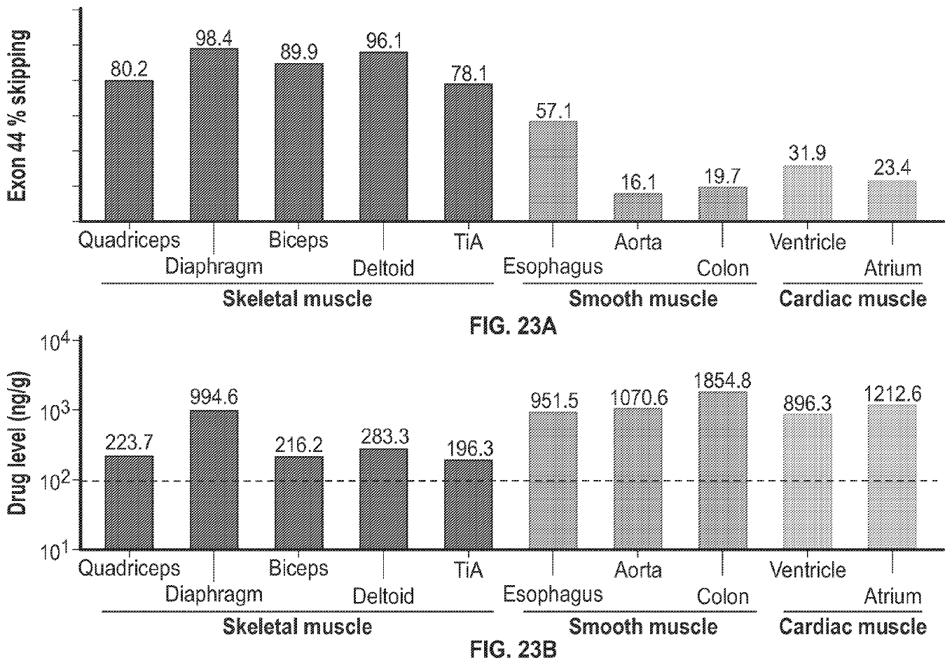

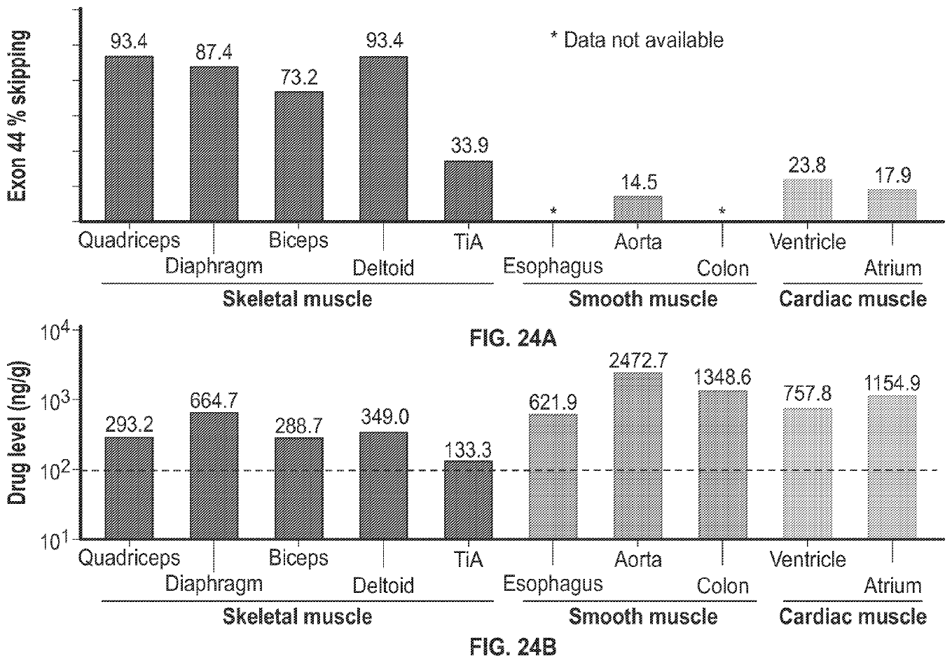

No abnormality in clinical chemistry panel at 2-d and 7-d post injection was observed. Exon 44 skipping percentage in different tissues were analyzed following by the standard protocol. FIGS. 23A-23B depict exon skipping (FIG. 23A) and drug exposure (FIG. 23B) for EEV-PMO-DMD44-1. FIGS. 24A-24B depict exon skipping (FIG. 24A) and drug exposure (FIG. 24B) for EEV-PMO-DMD44-2. Both compounds demonstrated an excellent exon skipping levels across different muscle groups with 40 mpk by IV at 7-d post injection. EEV-PMO-DMD44-1 outperformed in TiA, diaphragm, and less prominently in ventricle and atrium from efficacy standpoint. In all skeletal muscles more than 78% exon skipping achieved with maximum 98.4% in diaphragm. In cardiac tissues, EEV-PMO-DMD44-1 at 40 mpk resulted in 31.9% and 23.4% in ventricle and atrium, respectively. In smooth muscles, esophagus showed highest efficacy of 57.1%. Both EEV-PMO-DMD44-1 and EEV-PMO-DMD44-2 distributed at pharmacologically relevant concentrations to various tissues. In some cases, such as ventricle and atrium in cardiac tissues and more prominently in esophagus and colon, the same tissue concentration didn't turn into same functional delivery. This may indicate that the endosomal scape level in different tissues might be different. Nevertheless, in skeletal muscles, approximately 200 ng per gram tissue concentration correlated to a robust exon skipping of above 80%, while in cardiac tissues, 800-1000 ng per gram tissue concentration, correlated to roughly combined 50% exon skipping in atrium and ventricle. The 50% exon skipping with only single dose, 40 mpk of the cCPP-AC conjugates is very encouraging as the cardiac tissues are more challenging tissue for delivery and one that is crucial for treatment of neuromuscular disorders, such as DMD.

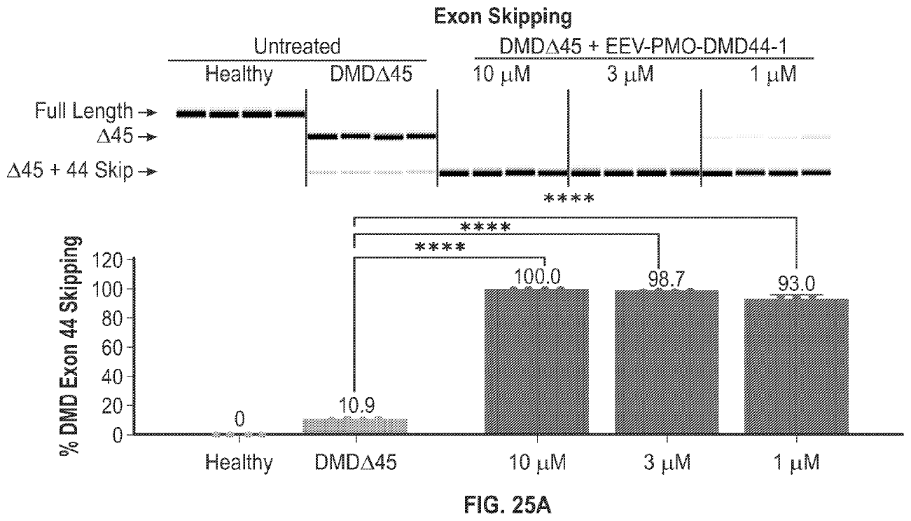

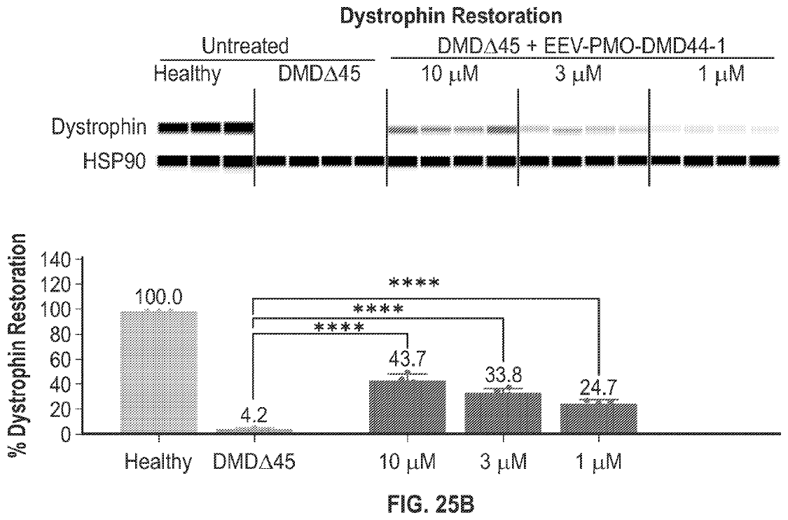

Patient-derived muscle cells: A compound for exon 44 skipping (EEV-PMO-DMD44-1) was added to DMD patient-derived muscle cells and administered to hDMD transgenic mice that express full-length human dystrophin gene to test for human sequence-specific PMO for DMD transcript correction. FIG. 25A shows robust dose-dependent exon skipping and restoration of dystyrophin in DMD patient-derived muscle cells treated with EEV-PMO-DMD44-1 Dose-dependent exon 44 skipping and dystrophin protein restoration was observed (up to 100% and 43.7% respectively) in DMD patient-derived muscle cells treated with EEV-PMO-DMD44-1 compared with both untreated patient derived cells and healthy cells. FIG. 25B shows dose-dependent tissue exposure and exon skipping in cardiac and skeletal muscles in a transgenic mouse carrying an integrated copy of the full-length human DMD gene after administering ascending IV doses of EEV-PMO-DMD44-1 at various levels ranging from 10 mg/kg to 80 mg/kg. Exon skipping and tissue exposure were each assessed five days after dosing. Dose dependent levels of tissue exposure of up to 80% and exon skipping up to 100% with translationally relevant doses were observed.

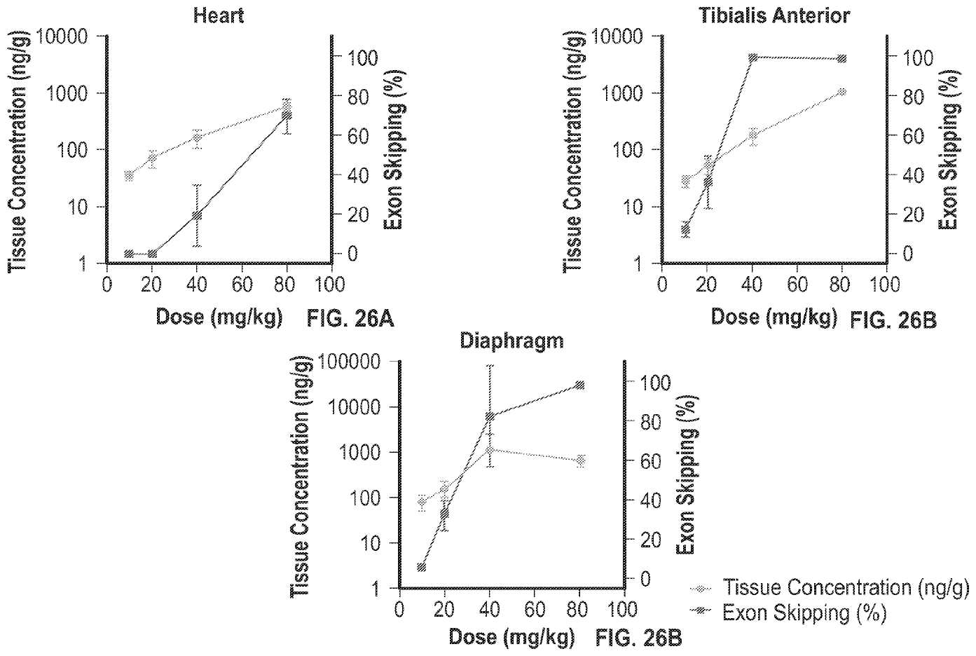

Dose-dependent tissue exposure and exon skipping was observed in the heart (FIG. 26A), tibialis anterior (FIG. 26B) and the diaphragm (FIG. 26C) of hDMD transgenic mice after intravenous (IV) administration of EEV-PMO-DMD44-1 at 10, 20, 40 and 80 mg/kg.

A single 30 mg/kg IV dose (1 hour infusion) of EEV-PMO-DMD44-1 was administered to NHP and an extended circulating half-life for EEV-PMO-DMD44-1 was observed in the plasma of the NHP for up to 50 hours (data not shown). This pharmacokinetic profile suggests an opportunity for extended tissue exposure, target engagement and pharmacodynamic effects. At 7 days post IV infusion, robust exon 44 skipping was observed across different muscle groups isolated from the EEV-PMO-DMD44-1 treated NHP (FIG. 11B). Thus, extended half-life and high levels (almost 90% in the biceps) of exon skipping in a NHP administered EEV-PMO-DMD44-1 have been observed.

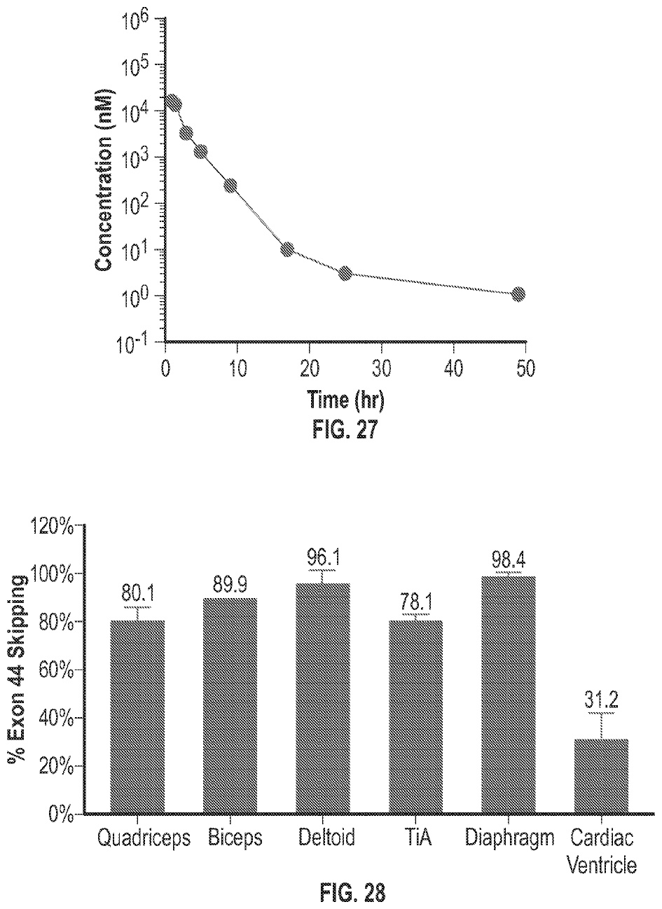

FIG. 27 shows that an extended circulating half-life for EEV-PMO-DMD44-1 was observed in the NHP.

FIG. 28 shows that a single 30 mg/kg IV dose of EEV-PMO-DMD44-1 resulted in meaningful levels of exon skipping in both skeletal muscles and the heart of the NHP which provides confidence in translational potential. At 7 days post 1 hour IV infusion at 30 mg/kg, robust exon 44 skipping observed across different muscle groups isolated from the EEV-PMO-DMD44-1 treated NHP.

These results represent a robust set of translational data. Exon skipping translates to promising dystrophin production in heart and skeletal muscles. Dystrophin production is sufficient to result in functional improvement. The dystrophin production was durable over 4+ weeks after a single injection.

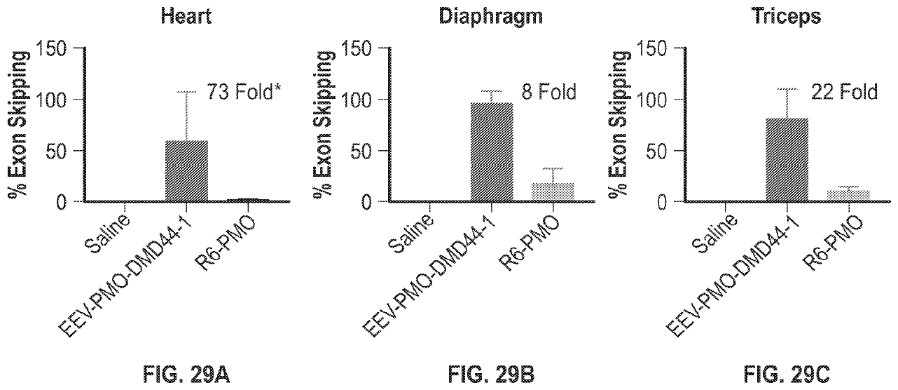

hDMD mice: Human dystrophic mice were IV dosed with 15 mg/kg of either EEV-PMO-DMD44-1 or a R6 (polyarginine) linear peptide conjugated to the same exon 44 skipping PMO. Exon skipping in the heart (FIG. 29A), diaphragm (FIG. 29B) and triceps (FIG. 29C) was between 60% to approximately 95% in mice treated with EEV-PMO-DMD-44, compared to exon skipping of less than 20% in the mice administered R6-PMO.

### Example 7. Pharmacokinetic Studies of EEV-PMO-DMD44-1 in CD1 Mice

A CD1 mouse model was used to study the plasma, kidney, and tibialis anterior drug exposure (AUC) to the EEV-PMO-DMD44-1 and PMO-DMD44-1, the major metabolite of EEV-PMO-DMD44-1.

Five- to seven-week-old mice were treated with 80 mpk of the EEV-PMO-DMD44-1 or PMO-DMD44-1 via intravenous injection. Mice were bled and/or scarified at various time points.

Table 14, Table 15, and Table 16 show the pharmacokinetic properties observed in the plasma, kidney, and tibialis anterior, respectively. For the tables: AUCₗₐₛₜ=area under the curve from zero to last quantifiable concentration; D=dose; Cₘₐₓ=maximum serum or plasma concentration; Tₘₐₓ=time to reach Cₘₐₓd; CL=total plasma, serum, or blood clearance; ti/2=elimination half-life; Vₛₛ=apparent volume of distribution at equilibrium; Qₕ=hepatic blood flow (ml/min/kg).

The AUC values for the metabolite is ˜1000-fold lower in the tibias anterior compared to the kidney. The metabolite mean residence time (MRT) values in plasma may be directly related to tissue MRT values as a result of moving from tissues to plasma before urinary excretion.

TABLE 14
Plasma pharmacokinetic properties

| | EEV-PMO-DMD44-1 | PMO-DMD44-1 |
|---|---|---|
| AUCₗₐₛₜ₍nM*hr) | 7768 | 522 |
| AUCₗₐₛₜ/D | 1053 | 71 |
| Cₘₐₓ(nM) | 9322 | 6 |
| Cₘₐₓ/D | 1264 | 0.8 |
| Tₘₐₓ(hr) | 0.1 | 48 |
| CL (mL/min/kg) | 16 | — |
| Qₕ(%) | 18 | — |
| t_{1/2}(hr) | 21 | 56 |
| MRTₗₐₛₜ(hr) | 1.0 | 60.4 |
| Vₛₛ(mL/kg) | 983 | — |

TABLE 15
Kidney pharmacokinetic properties

| | EEV-PMO-DMD44-1 | PMO-DMD44-1 |
|---|---|---|
| AUCₗₐₛₜ(pmol/g*hr) | 36171 | 8508417 |
| AUCₗₐₛₜ/D | 4905 | 1153875 |
| Cₘₐₓ(pmol/g) | 4805 | 84778 |
| Cₘₐₓ/D | 652 | 11497 |
| Tₘₐₓ(hr) | 4 | 72 |
| t_{1/2}(hr) | 6 | 102 |
| MRTₗₐₛₜ(hr) | 9 | 75 |

TABLE 16
Tibialis anterior pharmacokinetic properties

| | EEV-PMO-DMD44-1 | PMO-DMD44-1 |
|---|---|---|
| AUCₗₐₛₜ(pmol/g*hr) | — | 8126 |
| AUCₗₐₛₜ/D | — | 1102 |
| Cₘₐₓ(pmol/g) | 10 | 118 |
| Cₘₐₓ/D | 1 | 16 |
| Tₘₐₓ(hr) | 4 | 24 |
| t_{1/2}(hr) | — | 113 |
| MRTₗₐₛₜ(hr) | — | 61 |

### Example 8: Efficacy Studies on hDMD

Method: 6 groups (n=5) male hDMD mice were treated with EEV-PMO (EEV-PMO-DMD44-1) at 80 mpk. Tissues were collected at 1-, 2-, and 4-weeks post-dose. Exon skipping was determined by RT-PCR in 5 tissues: triceps, TA, diaphragm, heart and gastrocnemius.

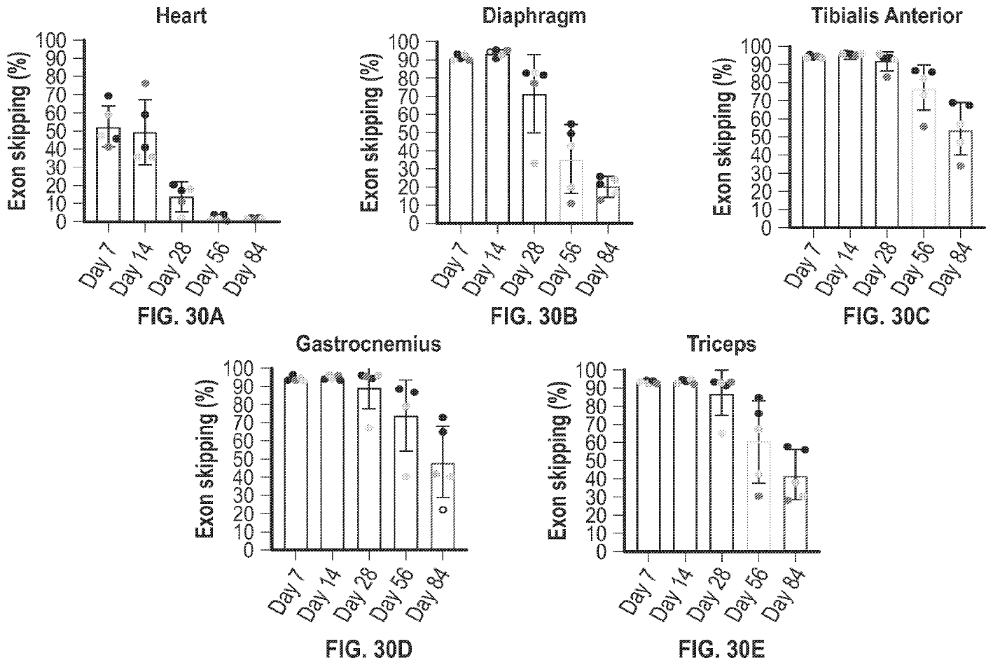

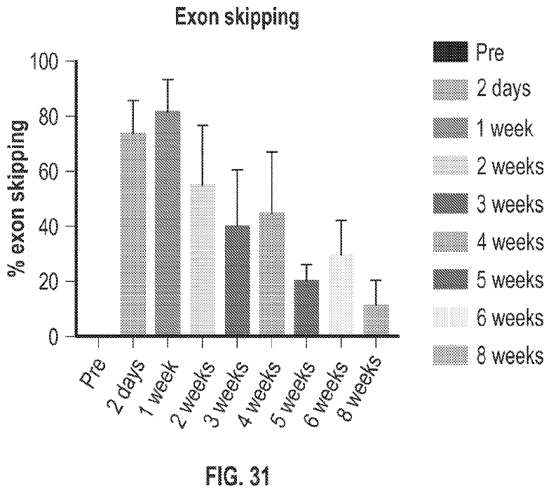

Results: In diaphragm, TA, gastroc and triceps, exon skipping was maintained for at least 12 weeks (FIG. 30B-30E). A drop in exon skipping was observed in the heart at 4 weeks (FIG. 30A).

### Example 9: Duration Effect on NHP

Method: 6 NHP were dosed with a single dose of EEV-PMO-DMD44-1 at 45 mpk via 1 hour IV infusion. Bicep biopsy was performed at the timepoints shown in FIG. 31. Exon skipping was determined by RT-PCR

Results: As shown in FIG. 31, exon skipping was observed after 2 days and peaked around 1 week. Exon skipping lasted up to 12 weeks after a single IV dose (with an apparent ti/2=14 days). Near peak efficacy was observed at 2 days post-dose.

### Example 10: Localization

Subcellular localization of three constructs a PMO alone (PMO), a PMO conjugated to an EEV (EEV-PMO) and a PMO conjugated to an EEV and a nuclear localization signal (EEV-NLS-PMO) was determined. Sequence information for these constructs is shown in Table 17.

TABLE 17
Constructs

| | |
|---|---|
| PMO: | 5′-GGCCAAACCTCGGCTTACCTGAAA T-3′ |
| EEV-PMO: | cyclo(FfΦRrRrQ)-PEG₁₂-NH-CH₂-CH₂-C(O)- |
| | ^{3′}TAAAGTCCATTCGGCTCCAAACCGG⁵ |
| EEV-NLS-PMO | Ac-PKKKRKV-Lys[Cyclo(FfΦRrRrQ)]-PEG₁₂- |
| | Lys(azide-cyclooct-2-yn-1-O-(CH₂)₄-O-C(O))- |
| | ^{3′}TAA AGT CCA TTC GGC TCC AAA CCG G⁵ |

Briefly, THP-1 monocytes were contacted with 3 μM of either the PMO, EEV-PMO or EEV-NLS-PMO and incubated for 24 hours and examined by LC-MS.

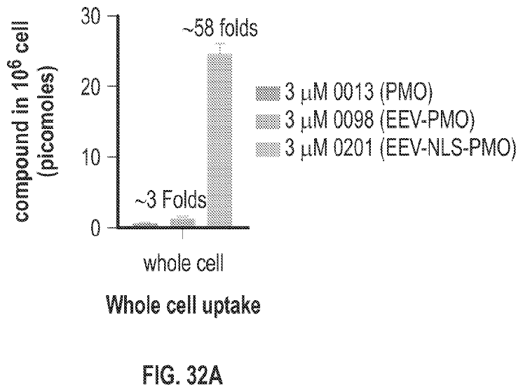

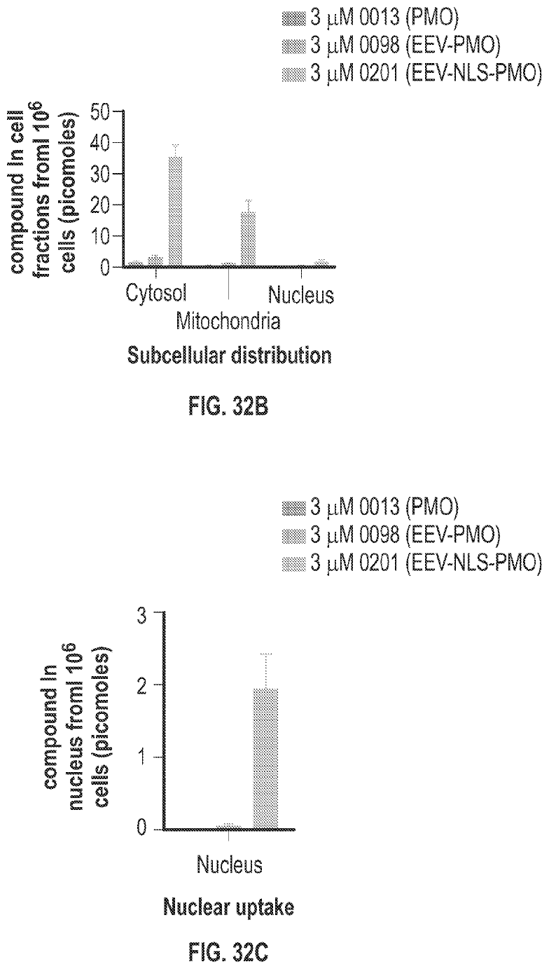

FIG. 32A shows the whole cell uptake of PMO vs EEV-PMO vs EEV-NLS-PMO. EEV-PMO and EEV-NLS-PMO both showed a significant increase in cellular update as compared to PMO alone.
EEV-PMO vs PMO: ˜3 fold
EEV-NLS-PMO vs PMO: ˜58 fold
EEV-NLS-PMO vs EEV-PMO: ˜19 fold

FIG. 32B shows the subcellular localization of PMO vs EEV-PMO vs EEV-NLS-PMO in THP cells as determined using LC-MS/MS. As shown in FIG. 32B, EEV-PMO demonstrate improved cellular permeability as compared to PMO-alone. The addition of the NLS further improved cellular permeability. FIG. 32C shows the nuclear uptake of the three constructs.

1-58. (canceled)

59. A compound comprising an Endosomal Escape Vehicle (EEV) conjugated to a therapeutic moiety comprising an antisense compound (AC) that is complementary to a target sequence comprising at least a portion of exon 44 of DMD gene in a pre-mRNA sequence; wherein the EEV comprises:
(a) a cyclic peptide of formula: 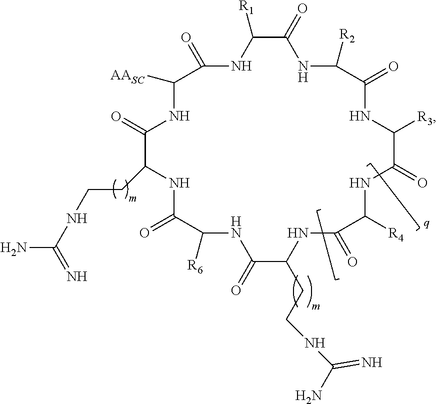
or a protonated form thereof,
wherein
R₁, R₂, and R₃ are each independently H or a side chain comprising an aryl or heteroaryl group;
at least two of R₁, R₂, and R₃ are a side chain of phenylalanine;
AA_{SC} is an amino acid side chain;
R₄ and R₇ are independently H or an amino acid side;
each m is independently an integer from 0-3; and
q is an integer from 1-4;
(b) an exocyclic peptide (EP) comprising from 2 to 10 amino acid residues, wherein 1 or 2 amino acid residues comprise a side chain of a guanidine group, or a protonated form thereof or 2, 3, or 4 lysine residues;
(c) a linker comprising:
(i) a —(OCH₂CH₂)_{z}— subunit, wherein z′ is an integer from 1 to 23;
(ii) one or more amino acid residues, such as a residue of glycine, β-alanine, 4-aminobutyric acid, 5-aminopentoic acid or 6-aminohexanoic acid, or combinations thereof; or
(iii) combinations of (i) and (ii).

60. The compound of claim 59, wherein the AC comprises a phosphorodiamidate morpholino nucleotide (PMO) comprising 15-30 nucleotides.

61. The compound of claim 59, wherein the cyclic peptide is conjugated to the 3′ end of the AC, the 5′ end of the AC, or the backbone of the AC.

62. The compound of claim 59, wherein the linker conjugates the cyclic peptide to the AC.

63. The compound of claim 59, wherein the AC comprises a sequence with at least 80%, 85%, 86%, 87%, 88%, 89%, 90%, 91%, 92%, 93%, 94%, 95%, 96%, 97%, 98%, 99% or 100% sequence identity to a sequence comprising 5′-XGAAAACGCCGCCAXXXCXCAACAGAXCXG-3′, wherein X is U or T, or a portion thereof.

64. The compound of claim 59, wherein the AC comprises a sequence with at least 80%, 85%, 86%, 87%, 88%, 89%, 90%, 91%, 92%, 93%, 94%, 95%, 96%, 97%, 98%, 99% or 100% sequence identity to a sequence comprising 5′-AAA CGC CGC CAT TTC TCA ACA GAT C-3′ or 5′-T GTT CAG CTT CTG TTA GCC ACT G AT-3′.

65. The compound of claim 64, wherein the AC comprises a sequence comprising at least 15, 16, 17, 18, 19, 20, 21, 22, 23, 24, or 25 contiguous nucleotides of a base sequence comprising: 5′-AAA CGC CGC CAT TTC TCA ACA GAT C-3′ or 5′-T GTT CAG CTT CTG TTA GCC ACT G AT-3′.

66. The compound of claim 59, wherein the AC has a sequence comprising:
a)
5′-GGCCAAACCTCGGCTTACTG AAAT-3′;
b)
5′-GGCCAAACCTCGGCTTACCTGAAAT-3′;
c)
5′-TGAAAACGCCGCCATTTCTCAACAGATCTG-3′;
d)
5′-TGAAAACGCCGCCATTTCTCAACAG-3′;
e)
5′-AAACGCCGCCATTTCTCAACAGATC-3′;
f)
5′-AACGCCGCCATTTCTCAACAGATCT-3′;
g)
5′-CCGCCATTTCTCAACAGATCTGTCA-3′;
h)
5′-TGTTCAGCTTCTGTTAGCCACTGAT-3′;
i)
5′-ACTGTTCAGCTTCTGTTAGCCACTG-3′;
or any reverse complement thereof.

67. The compound of claim 59, wherein the linker is of formula: 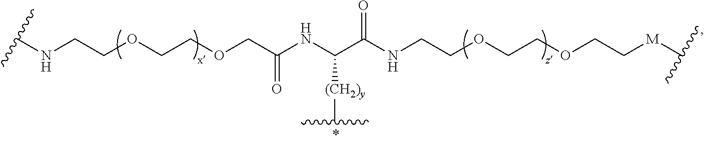
wherein
x is an integer from 1-23;
y is an integer from 1-5;
z is an integer from 1-23;
*is the point of attachment to the AA_{SC}; and
M is a bonding group.

68. The compound of claim 59, wherein M comprises 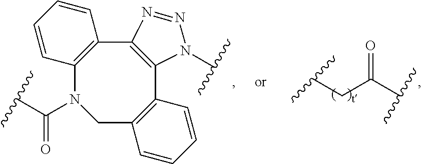
wherein t′ is an integer from 0 to 10.

69. The compound of claim 59, wherein R₄ and R₇ are independently an amino acid side chain of citrulline or serine.

70. The compound of claim 59, wherein:
(i) the exocyclic peptide comprises 1 or 2 amino acid residues comprising a side chain comprising a guanidine group, or a protonated form thereof;
(ii) the exocyclic peptide comprises 2, 3, or 4 lysine residues; and
(iii) the exocyclic peptide comprises at least 2 amino acid residues with a hydrophobic side chain, for example wherein the hydrophobic side chain is selected from valine, proline, alanine, leucine, isoleucine, and methionine.

71. The compound of claim 59, wherein the EP has the structure: Ac-PKKKRKV.

72. The compound of claim 59, wherein the cyclic peptide comprises: 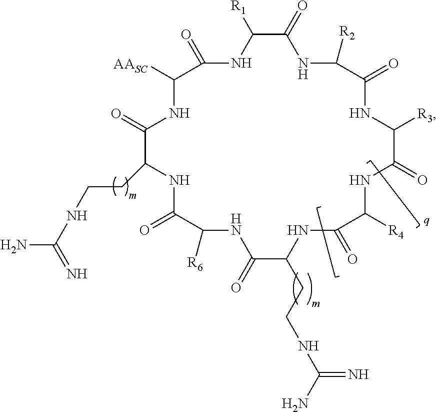
or a protonated form thereof,
wherein,
one of R₁, R₂, and R₃ is H;
two of R₁, R₂, and R₃ are CH₂Ph;
R₄ and R₇ are independently H or an amino acid side chain;
AA_{SC} is an amino acid side chain;
q is 1, 2, 3 or 4; and
each m is independently an integer 0, 1, 2, or 3.

73. The compound of claim 59, wherein the cyclic peptide comprises: 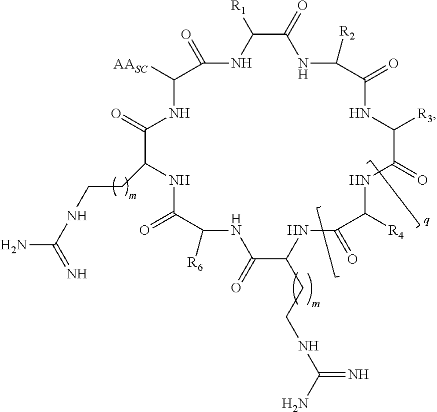
or a protonated form thereof,
wherein
R₁, R₂, and R₃ are —CH₂Ph;
R₄ and R₇ are independently H or an amino acid side chain;
AA_{sc} is an amino acid side chain;
q is 1, 2, 3 or 4; and
each m is independently an integer 0, 1, 2, or 3.

74. The compound of claim 59, wherein the cyclic peptide comprises: 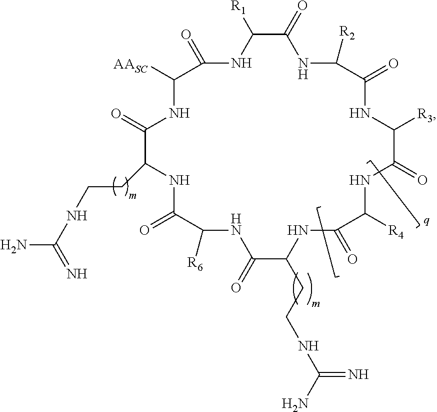
or a protonated form thereof,
wherein,
R₁, R₂, and R₃ are each independently a side chain comprising an aryl or heteroaryl group;
at least two of R₁, R₂, and R₃ are a side chain of phenylalanine;
AA_{SC} is an amino acid side chain;
R₄ and R₇ are independently H or an amino acid side chain of serine or citrulline
q is 1, 2, 3 or 4; and
each m is independently an integer 0, 1, 2, or 3.

75. The compound of claim 59, wherein the cyclic peptide is selected from Ff-Nal-GrGrQ; Ff-Nal-GRGRQ; FfFGRGRQ; FGFGRGRQ; CfFGrGrQ; FGFGRRRQ; and FGFRRRRQ.

76. The compound of claim 59, wherein the EEV has the formula:
Ac-PKKKRKV-PEGₓ-K(cyclo[FGFGRGRQ])-PEG_{y}-K(N₃)—NH₂;
Ac-PKKKRKV-PEGₓ-K(cyclo[FGFGRGRQ])-PEG_{y}-OH;
Ac-PKKKRKV-PEGₓ-K(cyclo[GfFGrGrQ])-PEG_{y}-K(N₃)—NH₂;
Ac-PKKKRKV-PEGₓ-K(cyclo[GfFGrGrQ])-PEG_{y}-OH;
Ac-PKKKRKV-PEGₓ-K(cyclo[FfFGRGRQ])-PEG_{y}-K(N₃)—NH₂;
Ac-PKKKRKV-PEGₓ-K(cyclo[FfFGRGRQ])-PEG_{y}-OH;
Ac-PKKKRKV-PEGₓ-K(cyclo[Ff-Nal-GrGrQ])-PEG_{y}-K(N₃)—NH₂; or
Ac-PKKKRKV-PEGₓ-K(cyclo[Ff-Nal-GrGrQ])-PEG_{y}-OH;
prior to conjugation to the therapeutic moiety.

77. The compound of claim 59, wherein the EEV has the formula:
Ac-PKKKRKV-PEG₂-K(cyclo[FGFGRGRQ])-PEG₂-K(N₃)—NH₂;
Ac-PKKKRKV-PEG₂-K(cyclo[FGFGRGRQ])-PEG₁₂-K(N₃)—NH₂;
Ac-PKKKRKV-PEG₂-K(cyclo[FGFGRGRQ])-PEG₂-OH; or
Ac-PKKKRKV-PEG₂-K(cyclo[FGFGRGRQ])-PEG₁₂-OH;
Ac-PKKKRKV-PEG₂-K(cyclo[GfFGrGrQ])-PEG₂-K(N₃)—NH₂;
Ac-PKKKRKV-PEG₂-K(cyclo[GfFGrGrQ])-PEG₁₂-K(N₃)—NH₂;
Ac-PKKKRKV-PEG₂-K(cyclo[GfFGrGrQ])-PEG₂-OH;
Ac-PKKKRKV-PEG₂-K(cyclo[GfFGrGrQ])-PEG₁₂-OH;
Ac-PKKKRKV-PEG₂-K(cyclo[FfFGRGRQ])-PEG₂-K(N₃)—NH₂;
Ac-PKKKRKV-PEG₂-K(cyclo[FfFGRGRQ])-PEG₁₂-K(N₃)—NH₂;
Ac-PKKKRKV-PEG₂-K(cyclo[FfFGRGRQ])-PEG₂-OH;
Ac-PKKKRKV-PEG₂-K(cyclo[FfFGRGRQ])-PEG₁₂-OH,
Ac-PKKKRKV-PEG₂-K(cyclo[Ff-Nal-GrGrQ])-PEG₂-K(N₃)—NH₂;
Ac-PKKKRKV-PEG₂-K(cyclo[Ff-Nal-GrGrQ])-PEG₁₂-K(N₃)—NH₂;
Ac-PKKKRKV-PEG₂-K(cyclo[Ff-Nal-GrGrQ])-PEG₂-OH;
Ac-PKKKRKV-PEG₂-K(cyclo[Ff-Nal-GrGrQ])-PEG₁₂-OH;
Ac-PKKKRKV-PEG₂-K(cyclo[FfΦ-Cit-r-Cit-r-Q])-PEG₁₂-K(N₃)—NH₂;
Ac-PKKKRKV-K(cyclo[FfΦ-R-r-Cit-rQ])-PEG₁₂-K(N₃)—NH₂;
Ac-PKKKRKV-K(cyclo[FfΦ-Cit-r-R-rQ])-PEG₁₂-K(N₃)—NH₂;
Ac-PKKKRKV-K(cyclo[FfΦR-cit-R-cit-Q])-PEG₁₂-K(N₃)—NH₂;
Ac-PKKKRKV-PEG₂-K(cyclo[FfΦ-Cit-r-Cit-rQ])-PEG₂-k(N₃)—NH₂;
Ac-PKKKRKV-PEG₂-K(cyclo[FfΦ-Cit-r-Cit-rQ])-PEG₄-k(N₃)—NH₂;
Ac-PKKKRKV-K(cyclo[FfΦ-Cit-r-Cit-rQ])-PEG₁₂-k(N₃)—NH₂;
Ac-PKKKRKV-PEG₂-K(cyclo[FfΦ-Cit-r-Cit-r-Q])-PEG₁₂-k(N₃)—NH₂; or
Ac-PKKKRKV-PEG₂-K(cyclo[FfΦ-Cit-r-Cit-r-Q])-PEG₁₂-K(N₃)—NH₂;
prior to conjugation to the therapeutic moiety.

78. The compound of claim 59, wherein the EEV has the formula:
Ac-PKKKRKV-PEG₂-K(cyclo[FGFGRGRQ])-PEG₁₂-OH;
Ac-PKKKRKV-PEG₂-K(cyclo[FGFGRGRQ])-PEG₂-K(N₃)—NH₂;
Ac-PKKKRKV-PEG₂-K(cyclo[GfFGrGrQ])-PEG₁₂-OH;
Ac-PKKKRKV-PEG₂-K(cyclo[GfFGrGrQ])-PEG₂-K(N₃)—NH₂;
Ac-PKKKRKV-K(cyclo[Ff-Nal-RrRrQ])-PEG₁₂-K(N₃)—NH₂;
Ac-PKKKRKV-PEG₂-K(cyclo[FfFGRGRQ])-PEG₂-K(N₃)—NH₂;
Ac-PKKKRKV-K(cyclo([Ff-Nal-GrGrQ])-PEG₁₂-K(N₃)—NH₂; or
Ac-PKKKRKV-PEG₂-K(cyclo[Ff-Nal-GrGrQ])-PEG₁₂-OH;
prior to conjugation to the therapeutic moiety.
